(19) United States
(12) Patent Application Publication
(10) Pub. No.: US 2024/0092866 A1
(43) Pub. Date: Mar. 21, 2024

(54) COMPOSITIONS AND METHODS FOR OCULAR TRANSGENE EXPRESSION

(71) Applicant: Avirmax Biopharma Inc., Hayward, CA (US)

(72) Inventors: Shengjiang LIU, Lafayette, CA (US); Haifeng CHEN, Hayward, CA (US)

(21) Appl. No.: 18/482,767

(22) Filed: Oct. 6, 2023

Related U.S. Application Data
(63) Continuation of application No. PCT/US2022/024101, filed on Apr. 8, 2022.
(60) Provisional application No. 63/173,280, filed on Apr. 9, 2021.

(51) Int. Cl. C07K 14/71; A61P 35/00; C12N 15/86

(52) U.S. Cl. CPC C07K 14/71; A61P 35/00; C12N 15/86; C07K 2319/30; C12N 2750/14143

(57) ABSTRACT

Provided herein are compositions and methods comprising non-naturally occurring nucleic acid sequences that encode biologics. Also provided are methods of utilizing the provided compositions and methods as ocular therapeutics for prevention and treatment of various diseases and conditions.

### CROSS-REFERENCE

This application is a continuation of International Application Number PCT/US2022/024101 filed Apr. 8, 2022, which claims the benefit of U.S. Provisional Application No. 63/173,280 filed on Apr. 9, 2021, the entirety of which is hereby incorporated by reference herein.

### SEQUENCE LISTING

The instant application contains a Sequence Listing which has been submitted electronically in XML format and is hereby incorporated by reference in its entirety. Said XML copy, created on Oct. 6, 2023, is named 59561-703_301_SL.xml and is 159,744 bytes in size.

### BACKGROUND

Aflibercept (VEGF-Trap) is a recombinant fusion protein that acts as a decoy receptor for vascular endothelial growth factor subtypes A (VEGF-A), VEGF-B, and placental growth factor (PIGF). By binding to these ligands, aflibercept is able to prevent these ligands from binding to vascular endothelial growth factor receptors (VEGFR), VEGFR-1 and VEGFR-2, to suppress neovascularization and decrease vascular permeability. Aflibercept consists of domain 2 of VEGFR-1 and domain 3 of VEGFR-2 fused with the Fc fragment of IgG1. Aflibercept is commercially marketed under the trade name EYLEA® (aflibercept), which is an ophthalmic intravitreal aflibercept fusion protein injection.

### SUMMARY

Current treatments employing aflibercept suffer from short durability and repeated monthly injections in order to suppress the neovascularization. Several gene therapy studies using AAV vectors carrying the coding sequence for VEGF-Trap has been investigated for long term treatment of the neovascularization. However, these studies suffer from low expression levels of the agent and therefore reduced treatment efficacy. Accordingly, it has become increasingly clear that the full potential of this technology will only be realized after modifications are made for improved delivery and expression of VEGF-Trap and other anti-angiogenic agents.

Described herein, in some aspects, is an isolated, non-naturally occurring nucleic acid comprising a sequence encoding a biologic comprising an anti-angiogenic agent, said sequence comprising a modification in a coding region of the sequence as compared to an otherwise comparable sequence lacking the modification in the coding region, said modification comprising a replacement of at least four non-AGG arginine codons with AGG. In some embodiments, the sequence that encodes the anti-angiogenic agent further comprises a second modification. In some embodiments, the second modification is in at least one codon of the coding region of the sequence, and wherein the second modification is selected from the group consisting of: (a) replacement of at least one non-CCC proline codon with CCC; (b) replacement of at least one non-TCC serine codon with TCC; (c) replacement of at least one non-CCG proline codon with CCG; and (d) any combination of (a)-(c). In some embodiments, the second modification comprises (a). In some embodiments, the at least one non-CCC proline codon is CCT. In some embodiments, the second modification comprises (b). In some embodiments, the at least one non-TCC serine codon is AGC. In some embodiments, the second modification comprises (c). In some embodiments, the at least one non-CCG proline codon is CCC. In some embodiments, the second modification comprises (d). In some embodiments, the at least one non-CCC proline codon of (a) is CCT; the at least one non-TCC serine codon of (b) is AGC; and the at least one non-CCG proline codon of (c) is CCC. In some embodiments, the anti-angiogenic agent is selected from the group consisting of: a VEGF inhibitor, a multi-tyrosine kinase inhibitor, a receptor tyrosine kinase inhibitor, an inhibitor of Akt phosphorylation, a PDGF-1 inhibitor, a PDGF-2 inhibitor, a NP-1 inhibitor, a NP-2 inhibitor, a Del 1 inhibitor, and an integrin inhibitor. In some embodiments, the anti-angiogenic agent comprises the VEGF inhibitor, and wherein the VEGF inhibitor is a non-antibody inhibitor. In some embodiments, the non-antibody inhibitor is a fusion protein that comprises human VEGF receptors 1 and 2. In some embodiments, the fusion protein comprises VEGF-Trap or a modified version thereof. In some embodiments, the isolated, non-naturally occurring nucleic acid further comprises a signal peptide. In some embodiments, the signal peptide is selected from the group consisting of: human antibody heavy chain (Vh), human antibody light chain (Vl), and VEGF-Trap. In some embodiments, the signal peptide is from the human antibody heavy chain. In some embodiments, the signal peptide is derived from VEGF-Trap. In some embodiments, the isolated, non-naturally occurring nucleic acid further comprises an intronic sequence. In some embodiments, the intronic sequence is selected from the group consisting of: hCMV intron A, adenovirus tripartite leader sequence intron, SV40 intron, hamster EF-1 alpha gene intron 1, intervening sequence intron, human growth hormone intron, and human beta globin intron. In some embodiments, the intronic sequence is the SV40 intron. In some embodiments, the isolated, non-naturally occurring nucleic acid further comprises a promoter. In some embodiments, the promoter is selected from the group consisting of: a cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, a Ca2+/calmodulin-dependent protein kinase II (CaMKIIa) promoter, a GAL1 promoter, a GAL 10 promoter, a TEF1 promoter, a glyceraldehyde 3-phosphage dehydrogenase (GDS) promoter, an ADH1 promoter, a CaMV35S promoter, a Ubi promoter, a human polymerase III RNA (H1) promoter, a U6 promoter, a polyadenylated construct thereof, and any combination thereof. In some embodiments, the promoter is the CMV promoter. In some embodiments, the sequence is modified to replace non-AGG arginine codon with AGG in at least 5, at least 6, at least 7, at least 8, at least 9, at least 10, at least 11, at least 12, at least 13, at least 14, at least 15, or at least 16 codons of the coding region of the sequence. In some embodiments, the sequence is modified to replace every non-AGG arginine codon with AGG of the coding region of the sequence. In some embodiments, the sequence is modified to replace non-AGG arginine codon with AGG in at least 5, at least 6, at least 7, at least 8, at least 9, at least 10, at least 11, at least 12, at least 13, at least 14, at least 15, or at least 16 codon positions as compared to SEQ ID NO: 70. In some embodiments, the sequence is modified to replace every non-AGG arginine codon with AGG as compared to SEQ ID NO: 70. In some embodiments, the non-AGG arginine codon comprises CGT, CGC, CGA, CGG, or AGA. In some embodiments, the non-AGG arginine codon is AGA. In some embodiments, the sequence is modified to replace AGA with AGG in at least 5, at least 6, at least 7, at least 8, at least 9, at least 10, at least 11, at least 12, at least 13, at least 14, at least 15, or at least 16 codons of the coding region of the sequence. In some embodiments, the sequence is modified to replace every AGA with AGG of the coding region of the sequence. In some embodiments, the sequence is modified to replace AGA with AGG in at least 5, at least 6, at least 7, at least 8, at least 9, at least 10, at least 11, at least 12, at least 13, at least 14, at least 15, or at least 16 codon positions as compared to SEQ ID NO: 70. In some embodiments, the sequence is modified to replace every AGA with AGG as compared to SEQ ID NO: 70. In some embodiments, the sequence is modified to replace non-CCC proline codons with CCC in at least 3, at least 6, at least 9, at least 12, at least 15, at least 18, at least 21, at least 24, at least 27, at least 29, or at least 30 codons of the coding region of the sequence. In some embodiments, the sequence is modified to replace every non-CCC proline codons with CCC of the coding region of the sequence. In some embodiments, the sequence is modified to replace non-CCC proline codon with CCC in at least 3, at least 6, at least 9, at least 12, at least 15, at least 18, at least 21, at least 24, at least 27, at least 29, or up to 30 codon positions as compared to SEQ ID NO: 70. In some embodiments, the sequence is modified to replace every non-CCC proline codon with CCC as compared to SEQ ID NO: 70. In some embodiments, the non-CCC proline codon comprises CCT or CCA. In some embodiments, the non-CCC proline codon is CCT. In some embodiments, the sequence is modified to replace CCT with CCC in at least 3, at least 6, at least 9, at least 12, at least 15, at least 18, at least 21, at least 24, at least 27, at least 29, or at least 30 codons of the coding region of the sequence. In some embodiments, the sequence is modified to replace every CCT with CCC of the coding region of the sequence. In some embodiments, the sequence is modified to replace CCT with CCC in at least 3, at least 6, at least 9, at least 12, at least 15, at least 18, at least 21, at least 24, at least 27, at least 29, or at least 30 codon positions as compared to SEQ ID NO: 70. In some embodiments, the sequence is modified to replace every CCT with CCC as compared to SEQ ID NO: 70. In some embodiments, the sequence is modified to replace non-TCC serine codon with TCC in at least 3, at least 6, at least 9, at least 12, at least 15, at least 18, at least 21, at least 24, at least 27, at least 30, at least 33, or at least 36 codons of the coding region of the sequence. In some embodiments, the sequence is modified to replace every non-TCC serine codon with TCC of the coding region of the sequence. In some embodiments, the sequence is modified to replace non-TCC serine codon with TCC in at least 3, at least 6, at least 9, at least 12, at least 15, at least 18, at least 21, at least 24, at least 27, at least 30, at least 33, or at least 36 codon positions as compared to SEQ ID NO: 70. In some embodiments, the sequence is modified to replace every non-TCC serine codon with TCC as compared to SEQ ID NO: 70. In some embodiments, the non-TCC serine codon comprises TCT, TCA, TCG, AGT, or AGC. In some embodiments, the non-TCC serine codon is AGC. In some embodiments, the sequence is modified to replace AGC with TCC in at least 3, at least 6, at least 9, at least 12, at least 15, at least 18, at least 21, at least 24, at least 27, at least 30, at least 33, or at least 36 codons of the coding region of the sequence. In some embodiments, the sequence is modified to replace every AGC with TCC of the coding region of the sequence. In some embodiments, the sequence is modified to replace AGC with TCC in at least 3, at least 6, at least 9, at least 12, at least 15, at least 18, at least 21, at least 24, at least 27, at least 30, at least 33, or at least 36 codon positions as compared to SEQ ID NO: 70. In some embodiments, the sequence is modified to replace every AGC with TCC as compared to SEQ ID NO: 70. In some embodiments, the sequence is modified to replace non-CCG proline codon with CCG in at least 3, at least 6, at least 9, at least 12, at least 15, at least 18, at least 21, at least 24, at least 27, or at least 30 codons of the coding region of the sequence. In some embodiments, the sequence is modified to replace every non-CCG proline codon with CCG of the coding region of the sequence. In some embodiments, the sequence is modified to replace non-CCG proline codon with CCG in at least 3, at least 6, at least 9, at least 12, at least 15, at least 18, at least 21, at least 24, at least 27, at least 30 codon positions as compared to SEQ ID NO: 70. In some embodiments, the sequence is modified to replace every non-CCG proline codon with CCG as compared to SEQ ID NO: 70. In some embodiments, the non-CCG proline codon comprises CCC or CCA. In some embodiments, the non-CCG proline codon is CCC. In some embodiments, the sequence is modified to replace CCC with CCG in in at least 3, at least 6, at least 9, at least 12, at least 15, at least 18, at least 21, at least 24, at least 27, or at least 30 codons of the coding region of the sequence. In some embodiments, the sequence is modified to replace every CCC with CCG of the coding region of the sequence. In some embodiments, the sequence is modified to replace CCC with CCG in at least 3, at least 6, at least 9, at least 12, at least 15, at least 18, at least 21, at least 24, at least 27, or at least 30 codon positions as compared to SEQ ID NO: 70. In some embodiments, the sequence is modified to replace every CCC with CCG as compared to SEQ ID NO: 70. In some embodiments, the nucleic acid comprises a viral vector sequence. In some embodiments, the viral vector sequence is a scAAV vector sequence. In some embodiments, the AAV vector sequence is of serotype AAV1, AAV2, AAV3, AAV4, AAV5, AAV6, AAV7, AAV8, AAV9, AAV10, AAV11, AAV12, or any combination thereof. In some embodiments, the AAV vector sequence is of the AAV2 serotype. In some embodiments, the viral vector sequence comprises sequences of at least two AAV serotypes. In some embodiments, the at least two serotypes are selected from AAV1, AAV2, AAV3, AAV4, AAV5, AAV6, AAV8, AAV9, AAV11, and AAV12. In some embodiments, the isolated, non-naturally occurring nucleic acid comprises at least about 60% sequence identity or similarity with any one of SEQ ID NOS: 13-19, 21-27, 31, 62, 64, 66, or 68. In some embodiments, the sequence identity is from about 70%, about 80%, about 85%, about 90%, about 95%, about 96%, about 97%, about 98%, about 99%, and up to about 100%. In some embodiments, the isolated, non-naturally occurring nucleic acid comprises about 70%, about 80%, about 85%, about 90%, about 95%, about 96%, about 97%, about 98%, about 99%, and up to about 100% sequence identity to SEQ ID NO: 31. In some embodiments, the isolated, non-naturally occurring nucleic acid comprises the nucleic acid sequence of SEQ ID NO: 31. In some embodiments, the isolated, non-naturally occurring nucleic acid consists of the nucleic acid sequence of SEQ ID NO: 31. In some embodiments, the isolated, non-naturally occurring nucleic acid comprises about 70%, about 80%, about 85%, about 90%, about 95%, about 96%, about 97%, about 98%, about 99%, and up to about 100% sequence identity to SEQ ID NO: 66. In some embodiments, the isolated, non-naturally occurring nucleic acid comprises the nucleic acid sequence of SEQ ID NO: 66. In some embodiments, the isolated, non-naturally occurring nucleic acid consists of the nucleic acid sequence of SEQ ID NO: 66. In some embodiments, the viral vector sequence is single-stranded. In some embodiments, the viral vector sequence is double-stranded. In some embodiments, the isolated, non-naturally occurring nucleic acid is single-stranded. In some embodiments, the isolated, non-naturally occurring nucleic acid is double-stranded. In some embodiments, the isolated, non-naturally occurring nucleic acid, upon contacting with a plurality of cells, increases expression of the biologic post transfection or post transduction in the plurality of cells as compared to an otherwise comparable isolated, non-naturally occurring nucleic acid that lacks the otherwise comparable sequence lacking the modification in a comparable plurality of cells. In some embodiments, the increased expression comprises at least a 5-fold, at least a 10-fold, at least a 20-fold, at least a 50-fold, at least a 100-fold, at least a 200-fold, or at least a 500-fold increase as determined by enzyme-linked immunoassay (ELISA) assay.

Described herein, in some aspects, is an isolated, non-naturally occurring nucleic acid that comprises at least about 60% sequence identity or similarity with any one of the nucleic acid sequences of SEQ ID NOS: 13-19, 21-27, 31, 62, 64, 66, or 68. In some embodiments, the isolated, non-naturally occurring nucleic acid comprises the nucleic acid sequence of SEQ ID NO: 31. In some embodiments, the isolated, non-naturally occurring nucleic acid comprises the nucleic acid sequence of SEQ ID NO: 66.

Described herein, in some aspects, is a biologic encoded by an isolated non-naturally occurring nucleic acid described herein. In some embodiments, the biologic comprises at least about 60% sequence identity with SEQ ID NO: 12. In some embodiments, the sequence identity is from about 70%, about 80%, about 85%, about 90%, about 95%, about 96%, about 97%, about 98%, about 99%, and up to about 100%.

Described herein, in some aspects, is an engineered cell comprising an isolated, non-naturally occurring nucleic acid described herein.

Described herein, in some aspects, is a plurality of adeno-associated viral (AAV) particles comprising an isolated, non-naturally occurring nucleic acid described herein.

Described herein, in some aspects, is a composition comprising a plurality of AAV particles described herein in unit dosage form. In some embodiments, the composition is cryopreserved.

Described herein, in some aspects, is a container comprising: an isolated, non-naturally occurring nucleic acid described herein; a biologic described herein; an engineered cell described herein, or a plurality of AAV particles described herein.

Described herein, in some aspects, is a method of modifying cells, the method comprising contacting: (a) a plurality of cells with the isolated non-naturally occurring nucleic acid of any one of claims 1-88; (b) a plurality of cells with the plurality of adeno-associated viral (AAV) particles of claim 93; or (c) both (a) and (b).

Described herein, in some aspects, is a pharmaceutical composition comprising: an isolated, non-naturally occurring nucleic acid described herein; a biologic described herein; or a plurality of AAV particles described herein. In some embodiments, the pharmaceutical composition is for treating an ocular disease or condition. In some embodiments, the ocular disease or condition is selected from the group consisting of: Achromatopsia, Age-related macular degeneration (AMD), Diabetic retinopathy (DR), Glaucoma, Bardet-Biedl Syndrome, Best Disease, Choroideremia, Leber Congenital Amaurosis, Macular degeneration, Polypoidal choroidal vasculopathy (PCV), Retinitis pigmentosa, Refsum disease, Stargardt disease, Usher syndrome, X-linked retinoschisis (XLRS), Rod-cone dystrophy, Cone-rod dystrophy, Oguchi disease, Malattia Leventinese (Familial Dominant Drusen), and Blue-cone monochromacy. In some embodiments, the ocular disease or condition is AMD.

Described herein, in some aspects, is a method of treating a disease or condition in a subject in need thereof, the method comprising administering an effective amount of a pharmaceutical composition described herein to the subject, thereby treating the disease.

Described herein, in some aspects, is a method for treating a disease or condition in a subject in need thereof, the method comprising administering a therapeutically effective amount of a pharmaceutical composition that comprises an isolated, non-naturally occurring nucleic acid that comprises a sequence that encodes a biologic that comprises an anti-angiogenic agent, wherein the sequence is modified to replace non-AGG arginine codons with AGG in at least four codons of a coding region of the sequence as compared to an otherwise comparable sequence lacking the modification in the coding region, thereby treating the disease or condition in the subject in need thereof.

Described herein, in some aspects, is a method for treating a disease or condition in a subject in need thereof, the method comprising: administering a therapeutically effective amount of a pharmaceutical composition that comprises an isolated, non-naturally occurring nucleic acid that comprises a sequence that encodes a biologic that comprises an anti-angiogenic agent, wherein the sequence is modified to replace non-AGG arginine codons with AGG in at least four codons of a coding region of the sequence as compared to an otherwise comparable sequence lacking the modification in the coding region, and wherein the modification is effective in increasing a level of the biologic in the subject in need thereof as compared to an otherwise comparable subject administered an otherwise comparable isolated, non-naturally occurring nucleic acid lacking the modification. In some embodiments, the increased level of the biologic in the subject is at least a 5-fold, at least a 10-fold, at least a 20-fold, at least a 50-fold, at least a 100-fold, at least a 200-fold, or at least a 500-fold increased, as determined by a diagnostic assay. In some embodiments, the sequence that encodes the anti-angiogenic agent further comprises a second modification. In some embodiments, the second modification is in at least one codon of the coding region of the sequence, and wherein the second modification is selected from the group consisting of: (a) replacement of at least one non-CCC proline codon with CCC; (b) replacement of at least one non-TCC serine codon with TCC; (c) replacement of at least one non-CCG proline codon with CCG; and (d) any combination of (a)-(c). In some embodiments, the second modification comprises (a). In some embodiments, the second modification comprises (b). In some embodiments, the second modification comprises (c). In some embodiments, the second modification comprises (d).

Described herein, in some aspects, is a method for treating a disease or condition in a subject in need thereof, the method comprising administering a therapeutically effective amount of a pharmaceutical composition that comprises an isolated, non-naturally occurring nucleic acid that comprises a sequence that encodes a biologic that comprises an anti-angiogenic agent, wherein the sequence is modified to replace AGA with AGG in at least four codons of a coding region of the sequence as compared to an otherwise comparable sequence lacking the modification in the coding region, thereby treating the disease or condition in the subject in need thereof.

Described herein, in some aspects, is a method for treating a disease or condition in a subject in need thereof, the method comprising: administering a therapeutically effective amount of a pharmaceutical composition that comprises an isolated, non-naturally occurring nucleic acid that comprises a sequence that encodes a biologic that comprises an anti-angiogenic agent, wherein the sequence is modified to replace AGA with AGG in at least four codons of a coding region of the sequence as compared to an otherwise comparable sequence lacking the modification in the coding region, and wherein the modification is effective in increasing a level of the biologic in the subject in need thereof as compared to an otherwise comparable subject administered an otherwise comparable isolated, non-naturally occurring nucleic acid lacking the modification. In some embodiments, the increased level of the biologic in the subject is at least a 5-fold, at least a 10-fold, at least a 20-fold, at least a 50-fold, at least a 100-fold, at least a 200-fold, or at least a 500-fold increased, as determined by a diagnostic assay. In some embodiments, the sequence that encodes the anti-angiogenic agent further comprises a second modification. In some embodiments, the second modification is in at least one codon of the coding region of the sequence, and wherein the second modification is selected from the group consisting of: (a) CCT to CCC; (b) AGC to TCC; (c) CCC to CCG; and (d) any combination of (a)-(c). In some embodiments, the second modification comprises (a). In some embodiments, the second modification comprises (b). In some embodiments, the second modification comprises (c). In some embodiments, the second modification comprises (d). In some embodiments, the anti-angiogenic agent comprises: a VEGF inhibitor, a multi-tyrosine kinase inhibitor, a receptor tyrosine kinase inhibitor, or an inhibitor of Akt phosphorylation. In some embodiments, the anti-angiogenic agent comprises the VEGF inhibitor. In some embodiments, the VEGF inhibitor is a non-antibody inhibitor. In some embodiments, the non-antibody inhibitor comprises a fusion protein that comprises human VEGF receptors 1 and 2, and wherein the fusion protein comprises VEGF-Trap or a modified version thereof. In some embodiments, the isolated, non-naturally occurring nucleic acid comprises at least about 60% sequence identity or similarity with any one SEQ ID NOS: 13-19, 21-27, 31, 62, 64, 66, or 68. In some embodiments, the sequence identity is from about 70%, about 80%, about 85%, about 90%, about 95%, about 96%, about 97%, about 98%, about 99%, and up to about 100%. In some embodiments, the isolated, non-naturally occurring nucleic acid comprises about 70%, about 80%, about 85%, about 90%, about 95%, about 96%, about 97%, about 98%, about 99%, and up to about 100% sequence identity to SEQ ID NO: 31. In some embodiments, the isolated, non-naturally occurring nucleic acid comprises the nucleic acid sequence of SEQ ID NO: 31. In some embodiments, the isolated, non-naturally occurring nucleic acid consists of the nucleic acid sequence of SEQ ID NO: 31. In some embodiments, the isolated, non-naturally occurring nucleic acid comprises about 70%, about 80%, about 85%, about 90%, about 95%, about 96%, about 97%, about 98%, about 99%, and up to about 100% sequence identity to SEQ ID NO: 66. In some embodiments, the isolated, non-naturally occurring nucleic acid comprises the nucleic acid sequence of SEQ ID NO: 66. In some embodiments, the isolated, non-naturally occurring nucleic acid consists of the nucleic acid sequence of SEQ ID NO: 66. In some embodiments, the nucleic acid comprises a viral vector sequence. In some embodiments, the viral vector sequence is a scAAV vector sequence. In some embodiments, the AAV vector sequence is of serotype AAV1, AAV2, AAV3, AAV4, AAV5, AAV6, AAV7, AAV8, AAV9, AAV10, AAV11, AAV12, or any combination thereof. In some embodiments, the AAV vector sequence is of the AAV2 serotype. In some embodiments, the administering is via intravitreal injection, subretinal injection, microinjection, or supraocular injection. In some embodiments, the administering is via intravitreal injection. In some embodiments, the ocular disease or condition is selected from the group consisting of: Achromatopsia, Age-related macular degeneration (AMD), Diabetic retinopathy (DR), Glaucoma, Bardet-Biedl Syndrome, Best Disease, Choroideremia, Leber Congenital Amaurosis, Macular degeneration, Polypoidal choroidal vasculopathy (PCV), Retinitis pigmentosa, Refsum disease, Stargardt disease, Usher syndrome, X-linked retinoschisis (XLRS), Rod-cone dystrophy, Cone-rod dystrophy, Oguchi disease, Malattia Leventinese (Familial Dominant Drusen), and Blue-cone monochromacy. In some embodiments, the ocular disease or condition is AMD. In some embodiments, the AMD is wet AMD. In some embodiments, the AMD is dry AMD. In some embodiments, the administering is sufficient to reduce at least a symptom of the disease or condition, treat the disease or condition, and/or eliminate the disease or condition. In some embodiments, the administering comprises delivering a dosage of the isolated, non-naturally occurring nucleic acid of about 1.0×10⁹ vg, about 1.0×10¹⁰, about 1.0×10¹¹ vg, about 3.0×10¹¹ vg, about 6×10¹¹ vg, about 8.0×10¹¹ vg, about 1.0×10¹² vg, about 1.0×10¹³ vg, about 1.0×10¹⁴ vg, or about 1.0×10¹⁵ vg. In some embodiments, the administering is repeated. In some embodiments, the administering is performed: twice daily, every other day, twice a week, bimonthly, trimonthly, once a month, every other month, semiannually, annually, or biannually. In some embodiments, prior to the administering, the subject undergoes genetic testing. In some embodiments, the genetic testing detects a mutation in a gene sequence as compared to an otherwise comparable wild type sequence. In some embodiments, the method further comprises administering a secondary therapy. In some embodiments, the secondary therapy comprises at least one of: photodynamic therapy (PDT), an anti-inflammatory agent, an anti-microbial agent, and laser photocoagulation therapy (LPT).

Disclosed herein are isolated non-naturally occurring nucleic acids that can comprise a sequence that encodes a biologic that can comprise an anti-angiogenic agent. In some embodiments, a sequence can be modified to replace AGA with AGG in at least one codon of a coding region of the sequence as compared to an otherwise comparable sequence lacking the modification in the coding region. In some embodiments, a sequence that encodes an anti-angiogenic agent can further comprise a second modification. In some embodiments, a second modification can be in at least one codon of the coding region of the sequence. In some embodiments, a second modification can be selected from the group comprising: a) CCT to CCC; b) AGC to TCC; c) CCC to CCG; or d) a combination of any of these. In some embodiments, a second modification can comprise CCT to CCC. In some embodiments, a second modification can comprise AGC to TCC. In some embodiments, a second modification can comprise CCC to CCG. In some embodiments, a second modification can comprise a combination of: CCT to CCC; AGC to TCC; or CCC to CCG. In some embodiments, an anti-angiogenic agent can comprise: a VEGF inhibitor, a multi-tyrosine kinase inhibitor, a receptor tyrosine kinase inhibitor, an inhibitor of Akt phosphorylation, a PDGF-1 inhibitor, a PDGF-2 inhibitor, a NP-1 inhibitor, a NP-2 inhibitor, a Del 1 inhibitor, or an integrin inhibitor. In some embodiments, an anti-angiogenic agent can comprise a VEGF inhibitor, and the VEGF inhibitor can be a non-antibody inhibitor. In some embodiments, a non-antibody inhibitor can be a fusion protein that can comprise human VEGF receptors 1 and 2. In some embodiments, a fusion protein can comprise VEGF-Trap or a modified version thereof. In some embodiments, an isolated non-naturally occurring nucleic acid can further comprise a signal peptide. In some embodiments, a signal peptide can be selected from the group consisting of: human antibody heavy chain (Vh), human antibody light chain (Vl), and VEGF-Trap. In some embodiments, a signal peptide can be from a human antibody heavy chain. In some embodiments, a signal peptide can be from an VEGF-Trap. In some embodiments, an isolated non-naturally occurring nucleic acid can further comprise an intronic sequence. In some embodiments, an intronic sequence can be selected from the group consisting of: CMV intron A, adenovirus tripartite leader sequence intron, SV40 intron, hamster EF-1 alpha gene intron 1, intervening sequence intron, human growth hormone intron, and human beta globin intron. In some embodiments, an intronic sequence can be a SV40 intron. In some embodiments, an isolated non-naturally occurring nucleic acid can further comprise a promoter. In some embodiments, a promoter can be selected from the group consisting of: a cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, a Ca2+/calmodulin-dependent protein kinase II (CaMKIIa) promoter, a GAL1 promoter, a GAL 10 promoter, a TEF1 promoter, a glyceraldehyde 3-phosphage dehydrogenase (GDS) promoter, an ADH1 promoter, a CaMV35S promoter, a Ubi promoter, a human polymerase III RNA (H1) promoter, a U6 promoter, a polyadenylated construct thereof, and any combination thereof. In some embodiments, a promoter can be a CMV promoter. In some embodiments, a sequence can be modified to replace AGA with AGG in at least 2, at least 4, at least 6, at least 8, at least 10, at least 12, at least 14, at least 16, at least 18, or up to 20 codons of the coding region of the sequence. In some embodiments, a sequence can be modified to replace AGA with AGG in 16 codons of the coding region of the sequence.

In some embodiments, a sequence can be modified to replace AGA with AGG at positions: X1-X16 as compared to SEQ ID NO: 28. In some embodiments, a sequence can be modified to replace CCT with CCC in at least 3, at least 6, at least 9, at least 12, at least 15, at least 18, at least 21, at least 24, at least 27, at least 29, or up to 30 codons of the coding region of the sequence. In some embodiments, a sequence can be modified to replace CCT with CCC in 30 codons of the coding region of the sequence. In some embodiments, a sequence can be modified to replace CCT with CCC at positions: X1-X30 as compared to SEQ ID NO: 28. In some embodiments, a sequence can be modified to replace AGC with TCC in at least 3, at least 6, at least 9, at least 12, at least 15, at least 18, at least 21, at least 24, at least 27, at least 30, at least 33, or up to 36 codons of the coding region of the sequence. In some embodiments, a sequence can be modified to replace AGC with TCC in 36 codons of the coding region of the sequence. In some embodiments, a sequence can be modified to replace AGC with TCC at positions: X1-X36 as compared to SEQ ID NO: 28. In some embodiments, a sequence can be modified to replace CCC with CCG in at least 3, at least 6, at least 9, at least 12, at least 15, at least 18, at least 21, at least 24, at least 27, at least 29, or up to 30 codons of the coding region of the sequence. In some embodiments, a sequence can be modified to replace CCC with CCG in 29 codons of the coding region of the sequence. In some embodiments, a sequence can be modified to replace CCC with CCG at positions: X1-X29 as compared to SEQ ID NO: 28. In some embodiments, a nucleic acid can comprise a viral vector sequence. In some embodiments, a viral vector sequence can be a self-complementary AAV (scAAV) vector sequence. In some embodiments, a AAV vector sequence can be of serotype AAV1, AAV2, AAV3, AAV4, AAV5, AAV6, AAV7, AAV8, AAV9, AAV10, AAV11, AAV12, or any combination thereof. In some embodiments, an AAV vector sequence can be of the AAV2 serotype. In some embodiments, a viral vector sequence can comprise sequences of at least 2 AAV serotypes. In some embodiments, at least two serotypes can be selected from AAV1, AAV2, AAV3, AAV4, AAV5, AAV6, AAV8, AAV9, AAV11, and AAV12. In some embodiments, an isolated non-naturally occurring nucleic acid can comprise a sequence having at least 60% sequence identity or similarity with any one of SEQ ID NO: 13-SEQ ID NO: 19. In some embodiments, a sequence identity can be from about 70%, about 80%, about 85%, about 90%, about 95%, about 96%, about 97%, about 98%, about 99%, and up to about 100%. In some embodiments, an isolated non-naturally occurring nucleic acid, upon contacting with a plurality of cells, can have increased expression of a biologic post transfection or post transduction in a plurality of cells as compared to an otherwise comparable isolated non-naturally occurring nucleic acid that may lack the otherwise comparable sequence lacking the modification in a comparable plurality of cells. In some embodiments, increased expression can comprise at least a 5-fold, at least a 10-fold, at least a 20-fold, at least a 50-fold, at least a 100-fold, at least a 200-fold, or at least a 500-fold increase as determined by enzyme-linked immunoassay (ELISA) assay.

Also described herein are isolated non-naturally occurring nucleic acids that can comprise at least 60% sequence identity or similarity with any one of the nucleic acid sequences of SEQ ID NO: 13-SEQ ID NO: 19 or SEQ ID NO: 21-SEQ ID NO: 27. In some embodiments, a biologic can be encoded by an isolated non-naturally occurring nucleic acid. In some embodiments, a biologic can comprise at least 60% sequence identity with SEQ ID NO: 12. In some embodiments, the sequence identity can be from about 70%, about 80%, about 85%, about 90%, about 95%, about 96%, about 97%, about 98%, about 99%, and up to about 100%.

Also described herein is an engineered cell comprising an isolated non-naturally occurring nucleic acid described herein.

Also described herein are a plurality of adeno-associated viral (AAV) particles that can be isolated from an engineered cell.

Also described herein are compositions that can comprise AAV particles in unit dosage form. In some embodiments, a composition can be cryopreserved.

Also described herein are containers that can comprise a) an isolated non-naturally occurring nucleic acid; b) a biologic; c) an engineered cell, or d) a plurality of AAV particles.

Also described herein are methods of modifying cells that can comprise contacting: a) a plurality of cells with an isolated non-naturally occurring nucleic acid; and/or b) a plurality of cells with a plurality of adeno-associated viral (AAV) particles.

Also described herein are pharmaceutical compositions that can comprise a) an isolated non-naturally occurring nucleic acid b) a biologic; c) or a plurality of AAV particles. In some embodiments, a pharmaceutical can be for treating an ocular disease or condition. In some embodiments, an ocular disease or condition can be selected from the group consisting of: Achromatopsia, Age-related macular degeneration (AMD), Diabetic retinopathy (DR), Glaucoma, Bardet-Biedl Syndrome, Best Disease, Choroideremia, Leber Congenital Amaurosis, Macular degeneration, Polypoidal choroidal vasculopathy (PCV), Retinitis pigmentosa, Refsum disease, Stargardt disease, Usher syndrome, X-linked retinoschisis (XLRS), Rod-cone dystrophy, Cone-rod dystrophy, Oguchi disease, Malattia Leventinese (Familial Dominant Drusen), and Blue-cone monochromacy. In some embodiments, an ocular disease or condition can be AMD.

Also described herein are methods of treating a disease or condition in a subject in need thereof that can comprise administering an effective amount of a pharmaceutical composition to the subject, thereby treating the disease.

Also described herein are methods for treating a disease or condition in a subject in need thereof. In some embodiments, a method can comprise administering a therapeutically effective amount of a pharmaceutical composition that can comprise an isolated non-naturally occurring nucleic acid that can comprise a sequence that encodes a biologic that can comprise an anti-angiogenic agent. In some embodiments, a sequence can be modified to replace AGA with AGG in at least one codon of a coding region of the sequence as compared to an otherwise comparable sequence lacking the modification in the coding region. In some embodiments, a method can treat a disease or condition in a subject in need thereof.

Also described herein are methods for treating a disease or condition in a subject in need thereof. In some embodiments, a method can comprise administering a therapeutically effective amount of a pharmaceutical composition that can comprise an isolated non-naturally occurring nucleic acid that can comprise a sequence that encodes a biologic that can comprise an anti-angiogenic agent. In some embodiments, a sequence can be modified to replace AGA with AGG in at least one codon of a coding region of the sequence as compared to an otherwise comparable sequence lacking the modification in the coding region. In some embodiments, a modification can be effective in increasing a level of the biologic in a subject in need thereof as compared to an otherwise comparable subject administered an otherwise comparable isolated non-naturally occurring nucleic acid lacking the modification. In some embodiments, an increased level of the biologic in the subject can be at least a 5-fold, at least a 10-fold, at least a 20-fold, at least a 50-fold, at least a 100-fold, at least a 200-fold, or at least a 500-fold increased, as determined by a diagnostic assay. In some embodiments, a sequence that can encode an antiangiogenic agent can further comprise a second modification. In some embodiments, a second modification can be in at least one codon of the coding region of the sequence. In some embodiments, a second modification can be selected from the group comprising: a) CCT to CCC; b) AGC to TCC; c) CCC to CCG; or d) a combination of any of these. In some embodiments, a second modification can comprise CCT to CCC. In some embodiments, a second modification can comprise AGC to TCC. In some embodiments, a second modification can comprise CCC to CCG. In some embodiments, a second modification can comprise any combination of: CCT to CCC, AGC to TCC, or CCC to CCG. In some embodiments, an anti-angiogenic agent can comprise: a VEGF inhibitor, a multi-tyrosine kinase inhibitor, a receptor tyrosine kinase inhibitor, or an inhibitor of Akt phosphorylation. In some embodiments, an anti-angiogenic agent can comprise a VEGF inhibitor. In some embodiments, an anti-angiogenic can comprise a VEGF inhibitor. In some embodiments, an VEGF inhibitor can be a non-antibody inhibitor. In some embodiments, a non-antibody inhibitor can comprise a fusion protein that can comprise human VEGF receptors 1 and 2. In some embodiments, a fusion protein can comprise VEGF-Trap or a modified version thereof. In some embodiments, an isolated non-naturally occurring nucleic acid can comprise at least 60% sequence identity or similarity with any one of SEQ ID NO: 13-SEQ ID NO: 19 or SEQ ID NO: 21-SEQ ID NO: 27. In some embodiments, the sequence identity can be from about 70%, about 80%, about 85%, about 90%, about 95%, about 96%, about 97%, about 98%, about 99%, and up to about 100%. In some embodiments, a nucleic acid can comprise a viral vector sequence. In some embodiments, a viral vector sequence can be a scAAV vector sequence. In some embodiments, an AAV vector sequence can be of serotype AAV1, AAV2, AAV3, AAV4, AAV5, AAV6, AAV7, AAV8, AAV9, AAV10, AAV11, AAV12, or any combination thereof. In some embodiments, an AAV vector sequence can be of the AAV2 serotype. In some embodiments, administering can be via intravitreal injection, subretinal injection, microinjection, or supraocular injection. In some embodiments, administering can be via intravitreal injection. In some embodiments, an ocular disease or condition can be selected from the group consisting of: Achromatopsia, Age-related macular degeneration (AMD), Diabetic retinopathy (DR), Glaucoma, Bardet-Biedl Syndrome, Best Disease, Choroideremia, Leber Congenital Amaurosis, Macular degeneration, Polypoidal choroidal vasculopathy (PCV), Retinitis pigmentosa, Refsum disease, Stargardt disease, Usher syndrome, X-linked retinoschisis (XLRS), Rod-cone dystrophy, Cone-rod dystrophy, Oguchi disease, Malattia Leventinese (Familial Dominant Drusen), and Blue-cone monochromacy. In some embodiments, an ocular disease or condition can be AMD.

In some embodiments, AMD can be wet AMD. In some embodiments, AMD can be dry AMD. In some embodiments, administering can be sufficient to reduce at least a symptom of a disease or condition, treat a disease or condition, and/or eliminate a disease or condition. In some embodiments, administering can comprise delivering a dosage of an isolated non-naturally occurring nucleic acid of about 1.0×10⁹ vg, about 1.0×10¹⁰, about 1.0×10¹¹ vg, about 3.0×10¹¹ vg, about 6×10¹¹ vg, about 8.0×10¹¹ vg, about 1.0×10¹² vg, about 1.0×10¹³ vg, about 1.0×10¹⁴ vg, or about 1.0×10¹⁵ vg. In some embodiments, administering can be repeated. In some embodiments, administering can be performed: twice daily, every other day, twice a week, bimonthly, trimonthly, once a month, every other month, semiannually, annually, or biannually. In some embodiments, prior to administering a subject can undergo genetic testing. In some embodiments, genetic testing can detect a mutation in a gene sequence as compared to an otherwise comparable wild type sequence. In some embodiments, a method can further comprise administering a secondary therapy. In some embodiments, a secondary therapy can comprise at least one of: photodynamic therapy (PDT), an anti-inflammatory agent, an anti-microbial agent, or laser photocoagulation therapy (LPT).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows scAAV constructs carrying the CMV enhancer/promoter, SV40 intron (INT), Kozak sequence (K), original (Af) or human IgG1 heavy chain (Vh) signal peptide (Sig), coding sequence, and synthetic polyadenylation signal (pA) flanked by a full AAV2 ITR (F-ITR) and a truncated AAV2 ITR (d-ITR). AMI059, AMI066, AMI067, AMI068, AMI119, AMI120, and AMI130 show different VEGF-Trap coding sequences with different signal peptides. AMI059 has an Af signal peptide and a low GC content coding sequence. AMI066 has a Vh signal peptide and a low GC content coding sequence. AMI067 has an Af signal peptide and a high GC content coding sequence. AMI068 has a Vh signal peptide and a high GC content coding sequence. AMI119 has an Af signal peptide and a high GC content coding sequence containing 16 Arg codon changes from AGA to AGG and 29 Pro codon changes from CCC to CCT. AMI120 has an Af signal peptide and a high GC content coding sequence containing 16 Arg codon changes from AGA to AGG and 36 Ser codon changes from AGC to TCC. AMI130 has an Af signal peptide and a high GC content coding sequence containing 16 Arg codon changes from AGA to AGG and 29 Pro codon changes from CCC to CCG.

FIG. 2 shows a Western blot analysis of VEGF-Trap expression in HEK293 cells transfected with plasmid DNA. Lanes 1-5 are shown on a non-reducing gel; lanes 6-10 are shown on a reducing gel; lanes 1 and 6 were loaded with supernatants from plasmid AMI059 transfected cells; lanes 2 and 7 were loaded with supernatants from plasmid AMI066 transfected cells; lanes 3 and 8 were loaded with supernatants from plasmid AMI067 transfected cells; lanes 4, 5, 9, and 10 were loaded with supernatants from plasmid AMI068 transfected cells.

FIGS. 3A and 3B show an SDS-PAGE and Simply Blue Staining analysis of low-GC and high-GC AAV vector production in Sf9 cells. In both figures, M indicates the lane loaded with a size marker and sizes are indicated for kilodaltons. In FIG. 3A, lane 1 shows AAV5 vector loaded as control; lanes 2, 3, 4 and 5 show AMI059, AMI066, AMI067, and AMI068, respectively. In FIG. 3B, Lane 1 shows AAV2 vector loaded as control; Lanes 2, 3, and 4 show AMI119, AMI120, and AMI130, respectively.

FIGS. 4A and 4B show a Western blot analysis of VEGF-Trap expression in HEK293 cells transduced with scAAV2 variant (AAV2.N53) vectors (AMI059, AMI066, AMI067, AMI068, AMI120, AMI119, AMI067, and AMI130). Twenty microliters of supernatants from AAV2 variant transduced HEK293 cells were loaded in each lane. In FIG. 4A, lanes 1, 2, 3 and 4 show AMI059, AMI066, AMI067, and AMI068, respectively. In FIG. 4B, lanes 1, 2, 3 and 4 show, AMI120, AMI119, AMI067, and AMI130, respectively.

FIG. 5A shows an SDS-PAGE and Simply Blue Staining analysis of VEGF-Trap purified from the supernatant of scAAV2-AMI067 transduced HEK293 cells. Commercially available aflibercept (A) was used as control. M indicates the lane loaded with a size marker and sizes are indicated for kilodaltons. 1.5 μg/lane of purified protein was loaded in the lane labeled V.

FIG. 5B and FIG. 5C illustrate binding affinity of AAV2.N54.120 (AVMX-110) derived VEGF-Trap to VEGF-A₁₆₅.

FIG. 6 shows amino acid sequence (SEQ ID NO: 61) and nucleic sequence (SEQ ID NO: 62) of AMI068-pFB-scCMV-SV40-intron-kozak-Vh-VEGF-Trap-GC. The bold letters indicate the human antibody heavy chain signal peptide.

FIG. 7 shows amino acid sequence (SEQ ID NO: 63) and nucleic acid sequence (SEQ ID NO: 64) of AMI119-pFB-scCMV-SV40-intron-kozak-Af-VEGF-Trap-GCRP (CCT). The bold letters indicate the VEGF-Trap signal peptide, underlines indicate the 16 AGA to AGG changes and italics indicates the 30 CCC to CCT changes.

FIG. 8 shows amino acid sequence (SEQ ID NO: 65) and nucleic acid sequence (SEQ ID NO: 66) of AMI120-pFB-scCMV-SV40-intron-kozak-Af-VEGF-Trap-GCRS (TCC). The bold letters indicate the VEGF-Trap signal peptide, the underlines indicate the 16 AGA to AGG changes and italics indicate the 36 AGC to TCC changes.

FIG. 9 shows amino acid sequence (SEQ ID NO: 67) and nucleic acid sequence (SEQ ID NO: 68) of AMI130-pFB-scCMV-SV40-intron-kozak-Af-VEGF-Trap-GCRP (CCG). The bold letters indicate the VEGF-Trap signal peptide, the underlines indicate the 16 AGA to AGG changes and italics indicates the 29 CCC to CCG changes.

FIG. 10A illustrates determination of AVMX-110 DNA and protein concentration.

FIG. 10B shows a time course experiment of AAV2.VEGF-Trap expression level in HEK93 cell culture.

FIG. 11 shows durability of AAV2.N53-VEGF-Trap and AAV2.N54-VEGF-Trap expression in HEK293 cells (top panel) and human retina cells (ARPE-19, bottom panel).

FIG. 12 shows AAV2.054-AMI120 derived VEGF-Trap was glycosylated similarly to aflibercept on reducing or non-reducing gel (reducing Gel: with and without PNGase F treatment. non-reducing gel: with and without PNGase F treatment). Lane 1 and 3 show AAV2-VEGF-Trap, while lane 2 depicts commercial aflibercept. Multiple bands in the PNGase F treatment lane indicate that the deglycosylation was not complete.

FIG. 13 shows binding affinity of AAV2.054-AMI120 derived VEGF-Trap to human VEGF-A₁₆₅ detected by Biacore assay.

FIG. 14 shows binding affinity of vector expressed VEGF-Trap to rhVEGF-A₁₆₅.

FIG. 15 shows comparison of vector derived VEGF-Trap to VEGF-Trap in inhibition of HUVEC cell proliferation.

FIG. 16 illustrates day 7 GFP fundus images prior to angiography in Example 11.

FIG. 17 illustrates day 7 fluorescein angiography in Example 11. Representative images from each group are shown.

FIG. 18 illustrates quantification of fluorescein leakage on day 7 post-laser treatment plotted as averages (top) and individual values (bottom). * signifies p=0.0113 and *** signifies p=0.0002.

FIG. 19 illustrates representative lesion images from Flatmount on day 7 post-laser treatment.

FIG. 20 illustrates quantification of Flatmount lesion area on day 7 post-laser treatment plotted as averages (top) and individual values (bottom).

FIG. 21 illustrates fundus images of in Example 12.

FIG. 22 illustrates IHC images of mouse eyes administered with selected constructs in Example 12.

FIG. 23 illustrates fundus imaging of mouse eyes administered with selected AAV constructs performed on day 24 for the study.

FIG. 24 illustrates IHC images of pig eyes administered with selected constructs in Example 12.

FIG. 25 illustrates fundus imaging of pig eyes administered with selected constructs performed on day 24 after injection.

FIG. 26 illustrates immunohistochemistry images on day 28 of the pig study.

FIG. 27 illustrates confocal microscopic images comparing AMI054 and V226 capsid penetrability.

FIG. 28A illustrates AAV construct details for AVMX-110/116.

FIG. 28B illustrates an exemplary chromatogram showing the manufacturing of a vector described herein (AVMX-110) as indicated by a single and sharp fraction (arrow).

FIG. 28C illustrates an exemplary SDS-PAGE showing the expression of AAV VP1, VP2, and VP3.

FIG. 28D illustrates retention difference of empty and full AAV in separation column process.

FIG. 28E illustrates separation of empty capsid for the purified AVMX-110.

FIG. 28F illustrates exemplary SDS-PAGE detected with mouse anti-AAV2 antibody. Samples in lanes 1 and 2 were CsCl purified AVMX-110l; lanes 3 and 4 were samples from 2L shake; 5-8 were samples from 2L bioreactors; and lanes 9 and 10 were the final purified drug substance at 10 & 20 μL per lane. The Western blot with AAV2 specific antibody reaction demonstrates AVMX-110 as an AAV2 specific serotype.

FIG. 28G. illustrates exemplary silver stain image of AVMX-110 separated by 10% SDS-PAGE. Lanes 1 and 5: samples of capture eluate. Lanes 2 and 6: empty. Lanes 3 and 7: non-reducing samples from peak 2 of the column chromatography. The purity of AVMX-110 process intermediates was analyzed by SDS-PAGE stained with silver stain gel. AAV VP1, VP2 and VP3 were clearly visualized, and no single impurity high than 4% was found.

FIG. 29 illustrates representative images of the fluorescence angiograph (FA) data from different study groups and bar graph showing the efficacy. Statistical analysis: one-way ANOVA followed by Tukey's multiple comparison (*=0.0332; ***=0.0002).

FIG. 30 illustrates in vitro cell based assay for aflibercept expression in different serotypes.

FIG. 31 illustrates FA data for different AAV serotypes. ΔAflibercept was sham vector that did not generate protein. Statistics was performed by one-way ANOVA followed by Dunnett's multiple comparison (*=0.0332; **=0.0021; ***=0.0002).

FIG. 32 illustrates ELISA to quantify the aflibercept expression in ocular sample and serum samples. Statistics was performed using one-way ANOVA followed by Dunnett's multiple comparison (***=0.0002; ****=<0.0001).

FIG. 33 illustrates FA data for mouse CNV study with different AAV6.N54-Aflibercept vectors.

FIG. 34 illustrates comparison of AAV1, AAV2 and AAV6-GFP expression using IHC images.

FIG. 35 illustrates comparison of the expression of GFP in different serotypes injected in pig eyes.

FIG. 36 illustrates in vitro comparison of wild type (wt) AAV2 and N54-AAV2.

FIG. 37A illustrates comparison of GFP expression in HEK293 cells transduced with different lots of N54-GFP.

FIG. 37B illustrates GFP expression in HEK293 transduced with different lots of N54-GFP.

FIG. 38A illustrates FA measurement comparison and statistical analysis.

FIG. 38B illustrates FA representative images in Example 13. Arrows indicate the leaser lesions (bubbles).

FIG. 39A illustrates comparison of VEGF-Trap level in serum sample.

FIG. 39B illustrates comparison VEGF-Trap level in ocular sample.

FIG. 40A illustrates correlation between VEGF-Trap expression and lesion area.

FIG. 40B illustrates comparison of the AAV2 capsid protein level in ocular sample.

FIG. 41A illustrates that the aflibercept expression in the animals treated with the sham vector control and untreated animals was similar. In Group 3 animals that were injected with AAV6.N54-Aflibercept, about 2.3 μg aflibercept/mg of ocular homogenate was measured (FIG. 41A). AAV2 and the mid-range level for AAV6.N54-Aflibercept had about 8.8 and about 25 μg aflibercept/mg of ocular homogenate respectively, which was a threefold-elevated expression for the AAV6-treated animals. High dose-AAV6 treated animals showed the highest level of aflibercept, with about 400 μg aflibercept/mg of ocular homogenate. Serum samples followed a similar trend as the ocular tissue samples. Groups 1 (Sham), 2 (AAV2-Aflibercept) and 3 (AAV6-low dose) had no detectable aflibercept. Both the medium and high dose AAV6-Aflibercept treated animals showed 2-3 ng/mL aflibercept in the serum (FIG. 41B). The data were analyzed in GraphPad Prism software using a one-way ANOVA followed by Dunnett's multiple comparison (**=0.005 and ****=<0.0001).

FIG. 42 illustrates representative images from day 0 fundus imaging (Groups 6-8).

FIG. 43 illustrates representative images from day 7 fluorescein angiography.

FIG. 44 illustrates quantification of average fluorescein Leakage on day 7.

FIG. 45 illustrates representative images of Isolectin lesion area.

FIG. 46 illustrates Isolectin area measurements

FIG. 47 illustrates representative Images of Day 0 IHC (Groups 6-8 Only).

FIG. 48A illustrates Flatmount analysis of the images obtained from the dose response study (Example 14).

FIG. 48B illustrates FA analysis of the images obtained from the dose response study (Example 14).

FIG. 49A illustrates laser-caused lesion area and AVMX-110 dose responsive curve.

FIG. 49B illustrates FA analysis of the images from the AVMX-110 dosing study.

FIG. 50 illustrates fluorescent angiography images of AVMX-110 in mouse LCNV model.

FIG. 51A illustrates VEGF-Trap levels in sera of animals (n=6 per group).

FIG. 51B illustrates that VEGF-Trap levels in retina tissue were also measured in the retina homogenates using the ELISA. In the retina tissue suspension, the expression of VEGF-Trap was in a dose dependent manner to the AVMX-110 injected intravitreally. An average of 13 ng/mL of VEGF-Trap was detected for the high dose group of 1.6e10 vg/eye. The highest expression level reached over 40 ng/mL.

### DETAILED DESCRIPTION

Provided herein are compositions and methods for use in ocular therapies. In an aspect, provided herein is an isolated, non-naturally occurring nucleic acid. The nucleic acid can be utilized in a composition for use in ocular therapy of various ocular diseases and conditions. In some cases, nucleic acids can comprise one or more modifications that confer certain advantages over unmodified otherwise comparable nucleic acids.

In an aspect, an isolated, non-naturally occurring nucleic acid is provided herein that encodes a biologic. Biologics include a wide range of products such as vaccines, blood and blood components, allergenics, cells, gene therapies, tissues, and recombinant therapeutic proteins. Biologics can be composed of sugars, proteins, or nucleic acids or complex combinations of these substances, or may be living entities such as cells and tissues. Biologics are isolated from a variety of natural sources (e.g., human, animal, microorganism) and may be produced using various methods. Gene-based and cellular biologics, for example, often are at the forefront of biomedical research, and may be used to treat a variety of medical conditions for which no other treatments are available.

Biologics as disclosed herein can comprise an anti-angiogenic agent. Angiogenesis refers to the formation of new blood vessels and/or maintenance of existing vasculature. The process involves the migration, growth, and/or differentiation of endothelial cells, which line the inside wall of blood vessels. The process of angiogenesis is modulated at least in part by chemical signals in the body. Some of these signals, such as vascular endothelial growth factor (VEGF), bind to receptors on the surface of normal endothelial cells. When VEGF and other endothelial growth factors bind to their receptors on endothelial cells, signals within these cells are initiated that promote the growth and survival of new blood vessels. Other chemical signals, for example anti-angiogenics can interfere with blood vessel formation. Normally, the angiogenesis-stimulating and -inhibiting effects of these signals are balanced so that blood vessels form only when and where they are needed, such as during growth and healing. In cases of disease or various conditions, these signals can become unbalanced, causing increased or aberrant blood vessel growth that can lead to abnormal conditions or disease. For example, aberrant angiogenesis is a cause of wet age-related macular degeneration.

Anti-angiogenic agents can reduce or eliminate vascularization. Anti-angiogenic agents can reduce or eliminate neovascularization. Anti-angiogenic agents can also reduce or eliminate existing vasculature. Angiogenesis inhibitors interfere in several ways with various steps in blood vessel growth. Some are biologics such as monoclonal antibodies that specifically recognize and bind to VEGF and/or other anti-angiogenic agents. For example, when VEGF is bound to these agents, it is unable to activate the VEGF receptor. Other anti-angiogenic agents bind to VEGF and/or its receptor as well as to other receptors on the surface of endothelial cells or to other proteins in the downstream signaling pathways, blocking their activities. Some anti-angiogenic agents are immunomodulatory drugs (e.g., agents that stimulate or suppress the immune system) that also have anti-angiogenic properties.

In an embodiment, an anti-angiogenic agent binds to a component of the VEGF signaling pathway. Components of the VEGF signaling pathway include but are not limited to: PLC, VRAP, Sck, Src, PI3K, PIP3, Akt, Bad, Caspase, eNOS, FAK, paxillin, Cdc42, p38 MAPK, Hsp27, GRB2, SOS, SHC, Ras, Raf, MEK1/2, ERK1/2, PKC, cPLA2, PGI2, IP3, and combinations thereof.

In an embodiment, a biologic is selected from a macromolecule such as a protein, peptide, aptamer, and/or non-translated RNAs, such as an antisense RNA, a ribozyme, an RNAi and/or an siRNA.

Biologics provided herein can comprise an anti-angiogenic agent. In some cases, a biologic is a protein or polypeptide. In some cases, a biologic comprises a polypeptide. A polypeptide can enhance and/or reduce one or more functions of an ocular cell, e.g., a rod or cone photoreceptor cell, a retinal ganglion cell, a Müller cell, a bipolar cell, an amacrine cell, a horizontal cell, and/or a retinal pigmented epithelial cell.

Exemplary classes of polypeptides include but are not limited to: neuroprotective polypeptides (e.g., GDNF, CNTF, NT4, NGF, and NTN); anti-angiogenic polypeptides (e.g., a soluble vascular endothelial growth factor (VEGF) receptor; a VEGF-binding antibody; a VEGF-binding antibody fragment (e.g., a single chain anti-VEGF antibody); endostatin; tumstatin; angiostatin; a soluble Flt polypeptide (Lai et al. (2005) Mol. Ther. 12:659); an Fc fusion protein comprising a soluble Flt polypeptide (see, e.g., Pechan et al. (2009) Gene Ther. 16:10); pigment epithelium-derived factor (PEDF); a soluble Tie-2 receptor; etc.); tissue inhibitor of metalloproteinases-3 (TIMP-3); a light-responsive opsin, e.g., a rhodopsin; anti-apoptotic polypeptides (e.g., Bcl-2, Bcl-X1); and the like. Exemplary polypeptides include, but are not limited to, glial derived neurotrophic factor (GDNF); fibroblast growth factor 2; neurturin (NTN); ciliary neurotrophic factor (CNTF); nerve growth factor (NGF); neurotrophin-4 (NT4); brain derived neurotrophic factor (BDNF; epidermal growth factor; rhodopsin; X-linked inhibitor of apoptosis; and Sonic hedgehog.

In some cases, a polypeptide can comprise retinoschisin, retinitis pigmentosa GTPase regulator (RGPR)-interacting protein-1 (see, e.g., GenBank Accession Nos. Q96KN7, Q9EPQ2, and Q9GLM3, peripherin-2 (Prph2) (see, e.g., GenBank Accession No. NP_000313, peripherin, a retinal pigment epithelium-specific protein (RPE65), (see, e.g., GenBank AAC39660; and Morimura et al. (1998) Proc. Natl. Acad. Sci. USA 95:3088), CHM (choroidermia (Rab escort protein 1)), a polypeptide that, when defective or missing, causes choroideremia (see, e.g., Donnelly et al. (1994) Hum. Mol. Genet. 3:1017; and van Bokhoven et al. (1994) Hum. Mol. Genet. 3:1041); and Crumbs homolog 1 (CRB1), a polypeptide that, when defective or missing, causes Leber congenital amaurosis and retinitis pigmentosa (see, e.g., den Hollander et al. (1999) Nat. Genet. 23:217; and GenBank Accession No. CAM23328). Suitable polypeptides also include polypeptides that, when defective or missing, lead to achromotopsia, where such polypeptides include, e.g., cone photoreceptor cGMP-gated channel subunit alpha (CNGA3) (see, e.g., GenBank Accession No. NP_001289; and Booij et al. (2011) Ophthalmology 118:160-167); cone photoreceptor cGMP-gated cation channel beta-subunit (CNGB3) (see, e.g., Kohl et al. (2005) Eur J Hum Genet. 13(3):302); guanine nucleotide binding protein (G protein), alpha transducing activity polypeptide 2 (GNAT2) (ACHM4); and ACHM5; and polypeptides that, when defective or lacking, lead to various forms of color blindness.

In some cases, a biologic comprises a protein or polypeptide coding for a site-specific endonuclease that provides for site-specific knockdown or knockout of gene function, e.g., where the endonuclease knocks out an allele associated with a retinal disease. For example, where a dominant allele encodes a defective copy of a gene that, when wild-type, is a retinal structural protein and/or provides for normal retinal function, a site-specific endonuclease can be targeted to the defective allele and knock out the defective allele. In addition to knocking out a defective allele, a site-specific nuclease can also be used to stimulate homologous recombination with a donor DNA that encodes a functional copy of the protein encoded by the defective allele. Thus, a non-naturally occurring nucleic acid can be used to deliver both a site-specific endonuclease that knocks out a defective allele and can be used to deliver a functional copy of the defective allele, resulting in repair of the defective allele, thereby providing for production of a functional retinal protein (e.g., functional retinoschisin, functional RPE65, functional peripherin, etc.). See, e.g., Li et al. (2011) Nature 475:217. In some embodiments, a non-naturally occurring nucleic acid comprises a transgene that encodes a site-specific endonuclease; and a heterologous nucleotide sequence that encodes a functional copy of a defective allele, where the functional copy encodes a functional retinal protein.

Exemplary functional retinal proteins include, e.g., retinoschisin, RPE65, retinitis pigmentosa GTPase regulator (RGPR)-interacting protein-1, peripherin, peripherin-2, and the like. Site-specific endonucleases that are suitable for use include, e.g., CRISPR, zinc finger nucleases (ZFNs); and transcription activator-like effector nucleases (TALENs), where such site-specific endonucleases are non-naturally occurring and are modified to target a specific gene. Such site-specific nucleases can be engineered to cut specific locations within a genome, and non-homologous end joining can then repair the break while inserting or deleting several nucleotides. Such site-specific endonucleases (also referred to as “INDELs”) then throw the protein out of frame and effectively knock out the gene. See, e.g., U.S. Patent Publication No. 2011/0301073. In some cases, a protein or polypeptide biologic is selected from: Lipoprotein Lipase, Retinoid Isomerohydrolase RPE65, or complement H. In some cases, a biologic is a polypeptide such as a fusion protein, such as aflibercept.

In an aspect, a biologic is aflibercept. Aflibercept is also known as VEGF Trap-eye (VTE) and EYLEA® and can be used interchangeably herein. Aflibercept is a recombinant fusion protein comprising extracellular domains of human VEGF receptors 1 and 2 fused to the Fc portion of human IgG. In contrast to the antibody-based VEGF binding strategy used by ranibizumab and bevacizumab, aflibercept incorporates the second binding domain of the VEGFR-1 receptor and the third domain of the VEGFR-2 receptor. Aflibercept acts as a soluble decoy receptor that binds VEGF-A and PDGF with greater affinity than the native receptors. SEQ ID NO: 30 illustrates aflibercept amino acid sequence aligned with DNA coding sequence (SEQ ID NO: 31) for aflibercept. For SEQ ID NO: 31, the nucleic acid comprises: an Af signal peptide; and a high GC content coding sequence containing 16 Arg codon changes from AGA to AGG and 36 Ser codon changes from AGC to TCC as compared with SEQ ID NO: 70.

The approved dose of intravitreal aflibercept injection is 2.0 mg, the dosing of which varies according to indication. Aflibercept is indicated for the treatment of neovascular (wet) age-related macular degeneration, macular edema following retinal vein occlusion, diabetic macular edema, and diabetic retinopathy. In some cases, a signal peptide provided herein can be from aflibercept or derived from aflibercept. In an embodiment, a signal peptide comprises a percent homology from about 50%, about 60%, about 70%, about 80%, about 85%, about 90%, about 95%, about 97%, about 98%, about 99%, or about 100% identity to a sequence (e.g., a signal peptide sequence) from aflibercept. In an embodiment, a signal peptide is from the human antibody heavy chain from aflibercept. In an embodiment, a signal peptide is from the human antibody light chain from aflibercept.

In some cases, a biologic is an aptamer. An aptamer can be a DNA aptamer or RNA aptamer. Aptamers are oligonucleotides (single-stranded or double-stranded) that fold into defined architectures and bind to targets such as proteins. Unlike some protein-based biologics, aptamers do not elicit or elicit reduced antibodies as compared to a protein-based biologic because aptamers generally contain sugars modified (for example at their 2′-positions). Additionally, Toll-like receptor-mediated innate immune responses are also abrogated or reduced. Aptamer therapeutics can be developed for intracellular, extracellular, or cell-surface targets. In some aspects, a biologic therapeutic is an aptamer. Exemplary aptamers of interest include an aptamer against vascular endothelial growth factor (VEGF). See, e.g., Ng et al. (2006) Nat. Rev. Drug Discovery 5:123; and Lee et al. (2005) Proc. Natl. Acad. Sci. USA 102:18902. For example, a VEGF aptamer can comprise the nucleotide sequence 5′-cgcaaucagugaaugcuuauacauccg-3′ (SEQ ID NO: 69). Also suitable for use is a PDGF-specific aptamer, e.g., E10030; see, e.g., Ni and Hui (2009) Ophthalmologica 223:401; and Akiyama et al. (2006)J. Cell Physiol. 207:407). Exemplary aptamers include Pegaptanib. Pegaptanib is a 50 kDa aptamer that is a specific nucleic acid ligand binding to VEGF165. Exemplary aptamers include but are not limited to Pegaptanib or Fovista.

In an embodiment, a biologic is a nucleic acid. Nucleic acids can include but are not limited to: non-translated RNAs, such as an antisense RNA, a ribozyme, an RNAi and/or an siRNA. In an embodiment, a biologic therapeutic is an interfering RNA (RNAi). Suitable RNAi include RNAi that can reduce a level of an apoptotic or angiogenic factor in a cell. For example, an RNAi can be an shRNA or siRNA that reduces the level of a gene product that induces or promotes apoptosis in a cell. Genes whose gene products induce or promote apoptosis are referred to herein as “pro-apoptotic genes” and the products of those genes (mRNA; protein) are referred to as “pro-apoptotic gene products.” Pro-apoptotic gene products include, e.g., Bax, Bid, Bak, and Bad gene products. See, e.g., U.S. Pat. No. 7,846,730. Interfering RNAs could also be against an angiogenic product, for example VEGF (e.g., Cand5; see, e.g., U.S. Patent Publication No. 2011/0143400; U.S. Patent Publication No. 2008/0188437; and Reich et al. (2003) Mol. Vis. 9:210), VEGFR1 (e.g., Sirna-027; see, e.g., Kaiser et al. (2010) Am. J. Ophthalmol. 150:33; and Shen et al. (2006) Gene Ther. 13:225), or VEGFR2 (Kou et al. (2005) Biochem. 44:15064). See also, U.S. Pat. Nos. 6,649,596, 6,399,586, 5,661,135, 5,639,872, and 5,639,736; and 7,947,659 and 7,919,473.

In some cases, a biologic comprises a siRNA that targets VEGF-A, for example Bevasiranib.

In some embodiments, cell type-specific or a tissue-specific promoter can be operably linked to a transgene encoding for a subject therapeutic, such that the gene product is produced selectively or preferentially in a particular cell type(s) or tissue(s). In some embodiments, an inducible promoter may be operably linked to a transgene sequence. In some cases, a promoter can be operably linked to a photoreceptor-specific regulatory element (e.g., a photoreceptor-specific promoter), e.g., a regulatory element that confers selective expression of the operably linked gene in a photoreceptor cell. Suitable photoreceptor-specific regulatory elements include, e.g., a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225) and the like.

In some embodiments, a biologic delivered by a subject modified AAV can act to inhibit angiogenesis. In an embodiment, a biologic comprises an anti-angiogenic agent. Exemplary anti-angiogenic agents can comprise: a VEGF inhibitor, Multi-tyrosine kinase inhibitor, Receptor tyrosine kinase inhibitor, inhibitor of Akt phosphorylation, PDGF-1 inhibitor, PDGF-2 inhibitor, NP-1 inhibitor, NP-2 inhibitor, Del 1 inhibitor, or/or integrin inhibitor. In an embodiment, the anti-angiogenic agent comprises a VEGF inhibitor. A VEGF inhibitor can target VEGF or the VEGF receptor.

The VEGF family includes placental growth factor (PLGF), VEGF-A, VEGF-B, VEGF-C, VEGF-D and VEGF-E. These agents are important regulators of angiogenesis and vascular permeability; VEGF-A in particular, plays a pivotal role in pathologic ocular angiogenesis. The VEGF-A gene has been localized to chromosome 6p12.3 and comprises of 8 exons and 8 intermediate introns. VEGF-A has 9 isoforms including VEGF121, VEGF145, VEGF148, VEGF162, VEGF165, VEGF165b, VEGF183, VEGF189 and VEGF206. These isoforms differ from each other by the number of amino acids and heparin-binding affinity. Anyone of the aforementioned VEGF family members and/or their receptors can be targeted with an inhibitor.

In certain preferred embodiments, the biologic delivered by a subject modified AAV can act to inhibit the activity of one or more mammalian VEGF proteins selected from the group consisting of VEGF-A, VEGF-B, VEGF-C, VEGF-D, and/or PDGF. In particularly preferred embodiments, the biologic delivered by the subject AAV variants inhibit the activity of VEGF-A. VEGF-A has 9 isoforms generated by alternative splicing, the most physiologically relevant of which is VEGF 165. VEGF-A levels have been found to be elevated in the vitreous of patients with wet age-related macular degeneration, diabetic macular edema and retinal vein occlusion. Gene product(s) which inhibit the activity of VEGF-A in the eye and which are therefore effective to treat patients with elevated vitreous VEGF-A include, but are not limited to, aflibercept, Ranibizumab, Brolucizumab, Bevacizumab, and soluble fms-like tyrosine kinase 1 (sFLT1) (GenBank Acc. No. U01134). In some embodiments, an infectious AAV virion is provided comprising (i) a variant AAV capsid protein as herein described and (ii) a transgene comprising a VEGF inhibitor. In an embodiment, a transgene comprises multiple sequences, each of which encodes a distinct VEGF-A inhibitor. In an embodiment, an anti-angiogenic is Ranibizumab. Ranibizumab is a humanized monoclonal antibody Fab fragment that inhibits all human isoforms of VEGF-A. In an embodiment, the transgene can be aflibercept.

In yet another embodiment, an isolated, non-naturally occurring nucleic acid provided herein comprises a modification. Various modifications are contemplated and can be employed for improved introduction, expression, persistence, and/or functionality of biologics that comprise anti-angiogenic agents as compared to otherwise comparable biologics. In some cases, an otherwise comparable biologic can be aflibercept.

In an embodiment, an isolated, non-naturally occurring nucleic acid comprises a modification that confers enhanced expression of a biologic that comprises an anti-angiogenic agent. For example, some biologics known in the art are derived from natural gene sequences and contain unmodified sequences that are not optimized for introduction and expression in target cells. In an embodiment, an isolated, non-naturally occurring nucleic acid is codon optimized. Codon optimization can be specific for cell type-specific codon usage. Different organisms and cell types exhibit bias towards use of certain codons over others for the same amino acid. Some species are known to avoid certain codons almost entirely. Similarly, certain cell types are biased toward use of certain codons over others for the same amino acid. In an embodiment, a method of optimizing a codon of a non-naturally occurring nucleic acid can comprise reassigning codon usage based on the frequencies of each codon's usage in a target cell. In some cases, a target cell can be of a certain tissue or organ.

In some cases, a modification is performed to increase guanine and/or cytosine content in a sequence as provided in, Grzegorz Kudla, et al., High guanine and cytosine content increases mRNA levels in mammalian cells, PLoS Biol 4(6): e180. DOI: 10.1371/journal.pbio.0040180, herein incorporated by reference in its entirety.

TABLE 1
Non-limiting, exemplary codons that can be interchanged for modification of nucleic
acids. Thymine can be replaced with uracil in the below codons.

| AA | Codons | AA | Codons |
|---|---|---|---|
| Ala | GCT, GCC, GCA, GCG | Leu | TTA, TTG, CTT, CTC, CTA, CTG |
| Arg | CGT, CGC, CGA, CGG, AGA, AGG | Lys | AAA, AAG |
| Asn | AAT, AAC | Met | ATG |
| Asp | GAT, GAC | Phe | TTT, TTC |
| Cys | TGT, TGC | Pro | CCT, CCC, CCA, CCU |
| Gln | CAA, CAG | Ser | TCT, TCC, TCA, TCG, AGT, AGC |
| Glu | GAA, GAG | Thr | ACT, ACC, ACA, ACG |
| Gly | GGT, GGC, GGA, GGG | Trp | TGG |
| His | CAT, CAC | Tyr | TAT, TAC |
| Ile | ATT, ATC, ATA | Val | GTT, GTC, GTA, GTG |
| Start | ATG | Stop | TAA, TGA, TAG |

In an embodiment, a non-naturally occurring nucleic acid sequence can be modified to replace at least one codon with another codon coding for an identical amino acid. In some cases, a codon is modified within a coding region of a sequence. In some cases, a codon is modified within a non-coding region of a sequence. In some cases, a codon is modified within about 100, about 50, about 25, about 15, or about 5 bases from a termination codon. E-CAI can be utilized to estimate a value of a codon adaptation index as provided in: Puigbo, P., Bravo, I. G. & Garcia-Vallve, S. E-CAI: a novel server to estimate an expected value of Codon Adaptation Index (eCAI). BMC Bioinformatics 9, 65, doi:10.1186/1471-2105-9-65 (2008).

Various modifications are contemplated herein. In some cases, codons can be interchanged. For example, a sequence can be modified to replace AGA with AGG. In other cases, CCC is replaced with CCT. In other cases, AGC is replaced with TCC. In other cases, CCC is replaced with CCG. Any of the non-limiting replacements provided in Table 1 can be applied to modify a nucleic acid. Any number of codons can be interchanged in a nucleic acid. In some cases, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 32, at least 34, at least 36, at least 38, at least 40, at least 42, at least 44, at least 46, at least 48, or up to 50 codons can be replaced. In an embodiment, a non-naturally occurring nucleic acid comprises 3 codon modifications. In an embodiment, a non-naturally occurring nucleic acid comprises 16 codon modifications. In an embodiment, a non-naturally occurring nucleic acid comprises 3-5, 5-10, 5-15, 10-15, 10-20, 15-20, 1-20, 12-20, 12-25, 15-30, or 15-25 codon modifications. In an embodiment, a non-naturally occurring nucleic acid comprises two codon modifications that are: AGA to AGG and at least one of: CCT to CCC, AGC to TCC, or CCC to CCG. In an embodiment, a non-naturally occurring nucleic acid comprises three codon modifications that are: AGA to AGG and at least two of: CCT to CCC, AGC to TCC, or CCC to CCG. In an embodiment, a non-naturally occurring nucleic acid comprises four codon modifications that are: AGA to AGG, CCT to CCC, AGC to TCC, and CCC to CCG. Additional modifications can comprise any of the codon modifications provided in Table 1 in combination with any of the above codons and/or any additional modifications possible from Table 1. In an embodiment, a nucleic acid is modified such that AGA is replaced with AGG and CCT is replaced with CCC. In an embodiment, a nucleic acid is modified such that AGA is replaced with AGG and AGC is replaced with TCC. In an embodiment, a nucleic acid is modified such that AGA is replaced with AGG and CCC is replaced with CCG.

In an embodiment, a non-naturally occurring nucleic acid sequence is modified to replace AGA with AGG in 16 codons of a sequence. In an embodiment, a non-naturally occurring nucleic acid sequence is modified to replace AGA with AGG in 16 codons of a coding sequence. In some cases, a sequence is modified to replace CCT with CCC in at least 3, at least 6, at least 9, at least 12, at least 15, at least 18, at least 21, at least 24, at least 27, at least 29, or up to 30 codons of the coding region of a nucleic acid sequence. In some cases, a sequence is modified to replace CCT with CCC in at least 30 codons of the coding region of the sequence. In some cases, a sequence is modified to replace AGC with TCC in at least 3, at least 6, at least 9, at least 12, at least 15, at least 18, at least 21, at least 24, at least 27, at least 30, at least 33, or up to 36 codons of a region of a nucleic acid sequence. In some cases, a sequence is modified to replace AGC with TCC in 36 codons of the coding region of a nucleic acid sequence. In some cases, a non-naturally occurring nucleic acid sequence is modified to replace CCC with CCG in at least 3, at least 6, at least 9, at least 12, at least 15, at least 18, at least 21, at least 24, at least 27, at least 29, or up to 30 codons of the coding region of the sequence. In some cases, the non-naturally occurring nucleic acid sequence is modified to replace CCC with CCG in 29 codons of the coding region of the non-naturally occurring nucleic acid sequence.

In some embodiments, an isolated, non-naturally occurring nucleic acid described herein comprises a sequence encoding a biologic comprising an anti-angiogenic agent, said sequence comprises a modification in a coding region of the sequence as compared to an otherwise comparable sequence lacking the modification in the coding region, said modification comprises replacing at least one, at least two, at least three, or at least four non-AGG arginine codons with AGG. In some embodiments, the non-AGG arginine codon is AGA. In some embodiments, the sequence is modified to replace non-AGG arginine codon with AGG in at least 5, at least 6, at least 7, at least 8, at least 9, at least 10, at least 11, at least 12, at least 13, at least 14, at least 15, or at least 16 codons of the coding region of the sequence. In some embodiments, the sequence is modified to replace non-AGG arginine codon with AGG in 16 codons of the coding region of the sequence. In some embodiments, the sequence is modified to replace non-AGG arginine codon with AGG in any one of 16 codons of the coding region of the sequence. In some embodiments, the sequence is modified to replace non-AGG arginine codon with AGG in at least 5, at least 6, at least 7, at least 8, at least 9, at least 10, at least 11, at least 12, at least 13, at least 14, at least 15, or at least 16 codon positions as compared to SEQ ID NO: 70. In some embodiments, the sequence is modified to replace non-AGG arginine codon with AGG in 16 codon positions as compared to SEQ ID NO: 70. In some embodiments, the sequence is modified to replace non-AGG arginine codon with AGG in any one of 16 codon positions as compared to SEQ ID NO: 70. In some embodiments, the non-AGG arginine codon is AGA. In some embodiments, the sequence is modified to replace AGA with AGG in at least 5, at least 6, at least 7, at least 8, at least 9, at least 10, at least 11, at least 12, at least 13, at least 14, at least 15, or at least 16 codons of the coding region of the sequence. In some embodiments, the sequence is modified to replace AGA with AGG in 16 codons of the coding region of the sequence. In some embodiments, the sequence is modified to replace AGA with AGG in any one of 16 codons of the coding region of the sequence. In some embodiments, the sequence is modified to replace AGA with AGG in at least 5, at least 6, at least 7, at least 8, at least 9, at least 10, at least 11, at least 12, at least 13, at least 14, at least 15, or at least 16 codon positions as compared to SEQ ID NO: 70. In some embodiments, the sequence is modified to replace AGA with AGG in 16 codon positions as compared to SEQ ID NO: 70. In some embodiments, the sequence is modified to replace AGA with AGG in any one of 16 codon positions as compared to SEQ ID NO: 70.

In some embodiments, an isolated, non-naturally occurring nucleic acid described herein comprises a sequence encoding a biologic comprising an anti-angiogenic agent, said sequence comprises a modification in a coding region of the sequence as compared to an otherwise comparable sequence lacking the modification in the coding region, said modification comprises replacing at least one non-CCC proline codon with CCC. In some embodiments, the at least one non-CCC proline codon is CCT. the sequence is modified to replace non-CCC proline codon with CCC in at least 3, at least 6, at least 9, at least 12, at least 15, at least 18, at least 21, at least 24, at least 27, at least 29, or up to 30 codons of the coding region of the sequence. In some embodiments, the sequence is modified to replace non-CCC proline codon with CCC in 30 codons of the coding region of the sequence. In some embodiments, the sequence is modified to replace non-CCC proline codon with CCC in any one of 30 codons of the coding region of the sequence. In some embodiments, the sequence is modified to replace non-CCC proline codon with CCC in at least 3, at least 6, at least 9, at least 12, at least 15, at least 18, at least 21, at least 24, at least 27, at least 29, or up to 30 codon positions as compared to SEQ ID NO: 70. In some embodiments, the sequence is modified to replace non-CCC proline codon with CCC in 30 codon positions as compared to SEQ ID NO: 70. In some embodiments, the sequence is modified to replace non-CCC proline codon with CCC in any one of 30 codon positions as compared to SEQ ID NO: 70. In some embodiments, the non-CCC proline codon is CCT. In some embodiments, the sequence is modified to replace CCT with CCC in at least 3, at least 6, at least 9, at least 12, at least 15, at least 18, at least 21, at least 24, at least 27, at least 29, or up to 30 codons of the coding region of the sequence. In some embodiments, the sequence is modified to replace CCT with CCC in 30 codons of the coding region of the sequence. In some embodiments, the sequence is modified to replace CCT with CCC in any one of 30 codons of the coding region of the sequence. In some embodiments, the sequence is modified to replace CCT with CCC in at least 3, at least 6, at least 9, at least 12, at least 15, at least 18, at least 21, at least 24, at least 27, at least 29, or up to 30 codon positions as compared to SEQ ID NO: 70. In some embodiments, the sequence is modified to replace CCT with CCC in 30 codon positions as compared to SEQ ID NO: 70. In some embodiments, the sequence is modified to replace CCT with CCC in any one of 30 codon positions as compared to SEQ ID NO: 70.

In some embodiments, an isolated, non-naturally occurring nucleic acid described herein comprises a sequence encoding a biologic comprising an anti-angiogenic agent, said sequence comprises a modification in a coding region of the sequence as compared to an otherwise comparable sequence lacking the modification in the coding region, said modification comprises replacing at least one non-TCC serine codon with TCC. In some embodiments, the sequence is modified to replace non-TCC serine codon with TCC in at least 3, at least 6, at least 9, at least 12, at least 15, at least 18, at least 21, at least 24, at least 27, at least 30, at least 33, or up to 36 codons of the coding region of the sequence. In some embodiments, the sequence is modified to replace non-TCC serine codon with TCC in 36 codons of the coding region of the sequence. In some embodiments, the sequence is modified to replace non-TCC serine codon with TCC in any one of 36 codons of the coding region of the sequence. In some embodiments, the sequence is modified to replace non-TCC serine codon with TCC in at least 3, at least 6, at least 9, at least 12, at least 15, at least 18, at least 21, at least 24, at least 27, at least 30, at least 33, or up to 36 codon positions as compared to SEQ ID NO: 70. In some embodiments, the sequence is modified to replace non-TCC serine codon with TCC in 36 codon positions as compared to SEQ ID NO: 70. In some embodiments, the sequence is modified to replace non-TCC serine codon with TCC in any one of 36 codon positions as compared to SEQ ID NO: 70. In some embodiments, the non-TCC serine codon is AGC. In some embodiments, the sequence is modified to replace AGC with TCC in at least 3, at least 6, at least 9, at least 12, at least 15, at least 18, at least 21, at least 24, at least 27, at least 30, at least 33, or up to 36 codons of the coding region of the sequence. In some embodiments, the sequence is modified to replace AGC with TCC in 36 codons of the coding region of the sequence. In some embodiments, the sequence is modified to replace AGC with TCC in any one of 36 codons of the coding region of the sequence. In some embodiments, the sequence is modified to replace AGC with TCC in at least 3, at least 6, at least 9, at least 12, at least 15, at least 18, at least 21, at least 24, at least 27, at least 30, at least 33, or up to 36 codon positions as compared to SEQ ID NO: 70. In some embodiments, the sequence is modified to replace AGC with TCC in 36 codon positions as compared to SEQ ID NO: 70. In some embodiments, the sequence is modified to replace AGC with TCC in any one of 36 codon positions as compared to SEQ ID NO: 70.

The isolated, non-naturally occurring nucleic acid of any one of claims 3-38, wherein the sequence is modified to replace non-CCG proline codon with CCG in at least 3, at least 6, at least 9, at least 12, at least 15, at least 18, at least 21, at least 24, at least 27, or up to 30 codons of the coding region of the sequence.

In some embodiments, an isolated, non-naturally occurring nucleic acid described herein comprises a sequence encoding a biologic comprising an anti-angiogenic agent, said sequence comprises a modification in a coding region of the sequence as compared to an otherwise comparable sequence lacking the modification in the coding region, said modification comprises replacing at least one non-CCG proline codon with CCG. In some embodiments, the sequence is modified to replace non-CCG proline codon with CCG in 30 codons of the coding region of the sequence. In some embodiments, the sequence is modified to replace non-CCG proline codon with CCG in any one of 30 codons of the coding region of the sequence. In some embodiments, the sequence is modified to replace non-CCG proline codon with CCG in at least 3, at least 6, at least 9, at least 12, at least 15, at least 18, at least 21, at least 24, at least 27, or up to 30 codons position as compared to SEQ ID NO: 70. In some embodiments, the sequence is modified to replace non-CCG proline codon with CCG in 30 codon positions as compared to SEQ ID NO: 70. In some embodiments, the sequence is modified to replace non-CCG proline codon with CCG in any one of 30 codon positions as compared to SEQ ID NO: 70. In some embodiments, the non-CCG proline codon is CCC. In some embodiments, the sequence is modified to replace CCC with CCG in in at least 3, at least 6, at least 9, at least 12, at least 15, at least 18, at least 21, at least 24, at least 27, or up to 30 codons of the coding region of the sequence. In some embodiments, the sequence is modified to replace CCC with CCG in 30 codons of the coding region of the sequence. In some embodiments, the sequence is modified to replace CCC with CCG in any one of 30 codons of the coding region of the sequence. In some embodiments, the sequence is modified to replace CCC with CCG in at least 3, at least 6, at least 9, at least 12, at least 15, at least 18, at least 21, at least 24, at least 27, or up to 30 codon positions as compared to SEQ ID NO: 70. In some embodiments, the sequence is modified to replace CCC with CCG in 30 codon positions as compared to SEQ ID NO: 70. In some embodiments, the sequence is modified to replace CCC with CCG in any one of 30 codon positions as compared to SEQ ID NO: 70.

In some embodiments, a non-naturally occurring nucleic acid sequence described herein comprises a nucleic acid sequence that is at least about 60% sequence identity or similarity with any one of SEQ ID NOS: 13-19, 21-27, 31, 62, 64, 66, or 68. In some embodiments, the sequence identity is from about 70%, about 80%, about 85%, about 90%, about 95%, about 96%, about 97%, about 98%, about 99%, and up to about 100%. In some embodiments, the isolated, non-naturally occurring nucleic acid comprises the sequence identity is from about 70%, about 80%, about 85%, about 90%, about 95%, about 96%, about 97%, about 98%, about 99%, and up to about 100% to SEQ ID NO: 31. In some embodiments, the isolated, non-naturally occurring nucleic acid comprises a nucleic acid sequence of SEQ ID NO: 31. In some embodiments, the isolated, non-naturally occurring nucleic acid is 100% identical to a nucleic acid sequence of SEQ ID NO: 31. In some embodiments, the isolated, non-naturally occurring nucleic acid comprises the sequence identity is from about 70%, about 80%, about 85%, about 90%, about 95%, about 96%, about 97%, about 98%, about 99%, and up to about 100% to SEQ ID NO: 66. In some embodiments, the isolated, non-naturally occurring nucleic acid comprises a nucleic acid sequence of SEQ ID NO: 66. In some embodiments, the isolated, non-naturally occurring nucleic acid is 100% identical to a nucleic acid sequence of SEQ ID NO: 66. In some embodiments, the isolated, non-naturally occurring nucleic acid is single stranded. In some embodiments, the isolated, non-naturally occurring nucleic acid is double stranded.

In some embodiments, a non-naturally occurring nucleic acid sequence can be modified to replace AGA with AGG at positions: X1-X16 as compared to SEQ ID NO: 70. In some embodiments, a non-naturally occurring nucleic acid sequence can be modified to replace CCT with CCC in at least 3, at least 6, at least 9, at least 12, at least 15, at least 18, at least 21, at least 24, at least 27, at least 29, or up to 30 codons of the coding region of the non-naturally occurring nucleic acid sequence. In some embodiments, a non-naturally occurring nucleic acid sequence can be modified to replace CCT with CCC in 30 codons of the coding region of the non-naturally occurring nucleic acid sequence. In some embodiments, a non-naturally occurring nucleic acid sequence can be modified to replace CCT with CCC at positions: X1-X30 as compared to SEQ ID NO: 70. In some embodiments, a non-naturally occurring nucleic acid sequence can be modified to replace AGC with TCC in at least 3, at least 6, at least 9, at least 12, at least 15, at least 18, at least 21, at least 24, at least 27, at least 30, at least 33, or up to 36 codons of the coding region of the non-naturally occurring nucleic acid sequence. In some embodiments, a non-naturally occurring nucleic acid sequence can be modified to replace AGC with TCC in 36 codons of the coding region of the non-naturally occurring nucleic acid sequence. In some embodiments, a non-naturally occurring nucleic acid sequence can be modified to replace AGC with TCC at positions: X1-X36 as compared to SEQ ID NO: 70. In some embodiments, a non-naturally occurring nucleic acid sequence can be modified to replace CCC with CCG in at least 3, at least 6, at least 9, at least 12, at least 15, at least 18, at least 21, at least 24, at least 27, at least 29, or up to 30 codons of the coding region of the non-naturally occurring nucleic acid sequence. In some embodiments, a non-naturally occurring nucleic acid sequence can be modified to replace CCC with CCG in 29 codons of the coding region of the non-naturally occurring nucleic acid sequence. In some embodiments, a non-naturally occurring nucleic acid sequence can be modified to replace CCC with CCG at positions: X1-X29 as compared to SEQ ID NO: 70. In some embodiments, a non-naturally occurring nucleic acid sequence can be modified to replace AGA with AGG at positions: X1-X16 as compared to SEQ ID NO: 28. In some embodiments, a non-naturally occurring nucleic acid sequence can be modified to replace CCT with CCC in at least 3, at least 6, at least 9, at least 12, at least 15, at least 18, at least 21, at least 24, at least 27, at least 29, or up to 30 codons of the coding region of the non-naturally occurring nucleic acid sequence. In some embodiments, a non-naturally occurring nucleic acid sequence can be modified to replace CCT with CCC in 30 codons of the coding region of the non-naturally occurring nucleic acid sequence. In some embodiments, a non-naturally occurring nucleic acid sequence can be modified to replace CCT with CCC at positions: X1-X30 as compared to SEQ ID NO: 28. In some embodiments, a non-naturally occurring nucleic acid sequence can be modified to replace AGC with TCC in at least 3, at least 6, at least 9, at least 12, at least 15, at least 18, at least 21, at least 24, at least 27, at least 30, at least 33, or up to 36 codons of the coding region of the non-naturally occurring nucleic acid sequence. In some embodiments, a non-naturally occurring nucleic acid sequence can be modified to replace AGC with TCC in 36 codons of the coding region of the non-naturally occurring nucleic acid sequence. In some embodiments, a non-naturally occurring nucleic acid sequence can be modified to replace AGC with TCC at positions: X1-X36 as compared to SEQ ID NO: 28. In some embodiments, a non-naturally occurring nucleic acid sequence can be modified to replace CCC with CCG in at least 3, at least 6, at least 9, at least 12, at least 15, at least 18, at least 21, at least 24, at least 27, at least 29, or up to 30 codons of the coding region of the non-naturally occurring nucleic acid sequence. In some embodiments, a non-naturally occurring nucleic acid sequence can be modified to replace CCC with CCG in 29 codons of the coding region of the non-naturally occurring nucleic acid sequence. In some embodiments, a non-naturally occurring nucleic acid sequence can be modified to replace CCC with CCG at positions: X1-X29 as compared to SEQ ID NO: 28. In some embodiments, a non-naturally occurring nucleic acid sequence can comprise a viral vector sequence. In some embodiments, a viral vector sequence can be a scAAV vector sequence. In some embodiments, a AAV vector sequence can be of serotype AAV1, AAV2, AAV3, AAV4, AAV5, AAV6, AAV7, AAV8, AAV9, AAV10, AAV11, AAV12, or any combination thereof. In some embodiments, an AAV vector sequence can be of the AAV2 serotype. In some embodiments, a viral vector sequence can comprise sequences of at least 2 AAV serotypes. In some embodiments, at least two serotypes can be selected from AAV1, AAV2, AAV3, AAV4, AAV5, AAV6, AAV8, AAV9, AAV11, and AAV12. In some embodiments, an isolated non-naturally occurring nucleic acid sequence can comprise a sequence having at least about 60% sequence identity or similarity with any one of SEQ ID NOS: 13-19 or 21-27. In some embodiments, the AAV vector sequence is single stranded. In some embodiments, the AAV vector sequence is double stranded.

In some cases, a modification can also comprise a chemical modification. Modified nucleic acids can comprise modifications of their backbones, sugars, or nucleobases, and even novel bases or base pairs. Modified nucleic acids can have improved chemical and/or biological stability. Decoration with diverse chemical substituents (e.g., hydrophobic groups) can also yield improved properties and functionalities such as new structural motifs and enhanced target binding.

Exemplary chemical modification include but are not limited to: 2′F, 2′-fluoro; 2′OMe, 2′-O-methyl; LNA, locked nucleic acid; FANA, 2′-fluoro arabinose nucleic acid; HNA, hexitol nucleic acid; 2′MOE, 2′-O-methoxyethyl; ribuloNA, (1′-3′)-β-L-ribulo nucleic acid; TNA, α-L-threose nucleic acid; tPhoNA, 3′-2′ phosphonomethyl-threosyl nucleic acid; dXNA, 2′-deoxyxylonucleic acid; PS, phosphorothioate; phNA, alkyl phosphonate nucleic acid; PNA, and peptide nucleic acid.

In some cases, a nucleic acid comprises additional features. Additional features can comprise sequences such as tags, signal peptides, intronic sequences, promoters, stuffer sequences, and the like.

In some cases, a nucleic acid comprises a signal peptide. A signal peptide is sometimes referred to as signal sequence, targeting signal, localization signal, localization sequence, transit peptide, leader sequence or leader peptide, is a short peptide present at the N-terminus of the majority of newly synthesized proteins that are destined toward the secretory pathway. These proteins include those that reside either inside certain organelles (the endoplasmic reticulum, Golgi or endosomes), secreted from the cell, or inserted into most cellular membranes. In some cases, nucleic acids provided herein can comprise signal peptides. A signal peptide can be of any length but typically from 15-30 amino acids long. A signal peptide can be from about: 10-15, 10-20, 10-30, 15-20, 15-25, 15-30, 20-30, or 25-30 amino acids long. Various signal peptides can be utilized and include but are not limited to: human antibody heavy chain (Vh), human antibody light chain (Vl), and aflibercept.

In some cases, a nucleic acid comprises an intronic sequence. An intron is any nucleotide sequence within a sequence that can be removed by RNA splicing during maturation of the final RNA product. In other words, introns are non-coding regions of an RNA transcript, or the DNA encoding it, that are eliminated by splicing before translation. While introns do not encode protein products, they are players in gene expression regulation. Some introns themselves encode functional RNAs through further processing after splicing to generate noncoding RNA molecules. Alternative splicing is widely used to generate multiple proteins from a single gene. Furthermore, some introns play essential roles in a wide range of gene expression regulatory functions such as nonsense-mediated decay and mRNA export. In an embodiment, an intronic sequence is included in a nucleic acid of the disclosure and can be selected from: hCMV intron A, adenovirus tripartite leader sequence intron, SV40 intron, hamster EF-1 alpha gene intron 1, intervening sequence intron, human growth hormone intron, and/or human beta globin intron. Any number of intronic sequences are contemplated. In an embodiment, the intronic sequence is SV40. In some cases, at least 1, at least 2, at least 3, at least 4, at least 5, at least 6, at least 7, at least 8, at least 9, or up to 10 intronic sequences can be included in a nucleic acid.

In an embodiment, an additional feature includes a promoter. Promoter is a sequence of DNA to which proteins bind that initiate transcription of a single RNA from the DNA downstream of it. This RNA may encode a protein, or can have a function in and of itself, such as tRNA, mRNA, or rRNA. Promoters are located near the transcription start sites of genes, upstream on the DNA (towards the 5′ region of the sense strand). Promoters can be about 100-1000 base pairs long. Various promoters are contemplated and can be employed in the non-naturally occurring nucleic acids of the disclosure. In an embodiment, a promoter is: a cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, a Ca2+/calmodulin-dependent protein kinase II (CaMKIIa) promoter, a GAL1 promoter, a GAL 10 promoter, a TEF1 promoter, a glyceraldehyde 3-phosphage dehydrogenase (GDS) promoter, an ADH1 promoter, a CaMV35S promoter, a Ubi promoter, a human polymerase III RNA (H1) promoter, a U6 promoter, a polyadenylated construct thereof, and any combination thereof. In some cases, the promoter is the CMV promoter.

Any of the provided nucleic acid sequences can comprise viral vector sequences. A viral vector can be, without limitation, a lentivirus, a retrovirus, or an adeno-associated virus. A viral vector can be an adeno-associated viral (AAV) vector. In some cases, a viral vector is an adeno-associated viral vector. Many serotypes of AAV vectors are contemplated and include but are not limited to: AAV1, AAV2, AAV3, AAV4, AAV5, AAV6, AAV7, AAV8, AAV9, AAV10, AAV11, and/or AAV12. Based on these initial serotypes, AAV capsid of each serotype can be engineered to make them better suited for biological functions, tissue or cell selection. In some embodiments, an AAV vector is AAV2 and variants AAV2.N53 and AAV2.N54 which are used in the examples of the invention. Chimeric AAV vectors are also contemplated that may contain at least 2 AAV serotypes. In some cases, at least 3, at least 4, at least 5, at least 6, at least 7, or up to 8 different serotypes are combined in a chimeric AAV vector. In some cases, only a portion of the AAV is chimeric. For example, suitable portions can include the capsid, VP1, VP2, or VP3 domains and/or Rep. In some cases, at least one of VP1, VP2, and VP3 has at least one amino acid substitution compared to an otherwise comparable wild-type AAV capsid protein. In some cases, a mutation can occur in VP1 and VP2, in VP1 and VP3, in VP2 and VP3, or in VP1, VP2, and VP3. In some embodiments, at least one of VP1, VP2, and VP3 has from one to about 25 amino acid substitutions compared to wild-type AAV VP1, VP2, and VP3, e.g., from about one to about 5, from about 5 to about 10, from about 10 to about 15, from about 15 to about 20, or from about 20 to about 25 amino acid substitutions compared to wild-type AAV VP1, VP2, and VP3. In some cases, a VP can be removed. For example, in some embodiments a mutant AAV does not comprise at least one of VP1, VP2, or VP3.

In some cases, an AAV vector can be modified. For example, an AAV vector can comprise a modification such as an insertion, deletion, chemical alteration, or synthetic modification. In some cases, a single nucleotide is inserted into an AAV vector. In other cases, multiple nucleotides are inserted into a vector. Nucleotides that can be inserted can range from about 1 nucleotide to about 5 kb.

In some cases, a mutation can occur at any of the previously mentioned AAV capsid positions and can include any number of mutations. In some cases, a mutation can be from one amino acid to another amino acid. Any combination or permutation of the canonical amino acids can be performed. Any of the following amino acid modifications can be made at any of VP1, VP2, and VP3: A to R, A to N, A to D, A to C, A to Q, A to E, A to G, A to H, A to I, A to L, A to K, A to M, A to F, A to P, A to S, A to T, A to W, A to Y, A to V, R to N, R to D, R to C, R to Q, R to E, R to G, R to H, R to I, R to L, R to K, R to M, R to F, R to P, R to S, R to T, R to W, R to Y, R to V, N to D, N to C, N to Q, N to E, N to G, N to H, N to I, N to L, N to K, N to M, N to F, N to P, N to S, N to T, N to W, N to Y, N to V, D to C, D to Q, D to E, D to G, D to H, D to I, D to L, D to K, D to M, D to F, D to P, D to S, D to T, D to W, D to Y, D to V, C to Q, C to E, C to G, C to H, C to I, C to L, C to K, C to M, C to F, C to P, C to S, C to T, C to W, C to Y, C to V, Q to E, Q to G, Q to H, Q to I, Q to L, Q to K, Q to M, Q to F, Q to P, Q to S, Q to T, Q to W, Q to Y, Q to V, E to G, E to H, E to I, E to L, E to K, E to M, E to F, E to P, E to S, E to T, E to W, E to Y, E to V, G to H, G to I, G to L, G to K, G to M, G to F, G to P, G to S, G to T, G to W, G to Y, G to V, H to I, H to L, H to K, H to M, H to F, H to P, H to S, H to T, H to W, H to Y, H to V, I to L, I to K, I to M, I to F, I to P, I to S, I to T, I to W, I to Y, I to V, L to K, L to M, L to F, L to P, L to S, L to T, L to W, L to Y, L to V, K to M, K to F, K to P, K to S, K to T, K to W, K to Y, K to V, M to F, M to P, M to S, M to T, M to W, M to Y, M to V, F to P, F to S, F to T, F to W, F to Y, F to V, P to S, P to T, P to W, P to Y, P to V, S to T, S to W, S to Y, S to V, T to W, T to Y, T to V, W to Y, W to V, Y to V, and any of the previously described mutations in reverse.

A mutation can be a conservative mutation or replacement. For example, 20 naturally occurring amino acids can share similar characteristics. Aliphatic amino acids can be glycine, alanine, valine, leucine, or isoleucine. Hydroxyl or sulfur/selenium-containing amino acids can be: Serine, cysteine, selenocysteine, threonine, or methionine. A cyclic amino acid can be proline. An aromatic amino acid can be phenylalanine, tyrosine, or tryptophan. A basic amino acid can be histidine, lysine, and arginine. An acidic amino acid can be aspartate, glutamate, asparagine, or glutamine. A conservative mutation can be, serine to glycine, serine to alanine, serine to serine, serine to threonine, serine to proline. A conservative mutation can be arginine to asparagine, arginine to lysine, arginine to glutamine, arginine to arginine, arginine to histidine. A conservative mutation can be Leucine to phenylalanine, leucine to isoleucine, leucine to valine, leucine to leucine, leucine to methionine. A conservative mutation can be proline to glycine, proline to alanine, proline to serine, proline to threonine, proline to proline. A conservative mutation can be threonine to glycine, threonine to alanine, threonine to serine, threonine to threonine, threonine to proline. A conservative mutation can be alanine to glycine, alanine to threonine, alanine to proline, alanine to alanine, alanine to serine. A conservative mutation can be valine to methionine, valine to phenylalanine, valine to isoleucine, valine to leucine, valine to valine. A conservative mutation can be glycine to alanine, glycine to threonine, glycine to proline, glycine to serine, glycine to glycine. A conservative mutation can be Isoleucine to phenylalanine, isoleucine to isoleucine, isoleucine to valine, isoleucine to leucine, isoleucine to methionine. A conservative mutation can be phenylalanine to tryptophan, phenylalanine to phenylalanine, phenylalanine to tyrosine. A conservative mutation can be tyrosine to tryptophan, tyrosine to phenylalanine, tyrosine to tyrosine. A conservative mutation can be cysteine to serine, cysteine to threonine, cysteine to cysteine. A conservative mutation can be histidine to asparagine, histidine to lysine, histidine to glutamine, histidine to arginine, histidine to histidine. A conservative mutation can be glutamine to glutamic acid, glutamine to asparagine, glutamine to aspartic acid, glutamine to glutamine. A conservative mutation can be asparagine to glutamic acid, asparagine to asparagine, asparagine to aspartic acid, asparagine to glutamine. A conservative mutation can be lysine to asparagine, lysine to lysine, lysine to glutamine, lysine to arginine, lysine to histidine. A conservative mutation can be aspartic acid to glutamic acid, aspartic acid to asparagine, aspartic acid to aspartic acid, aspartic acid to glutamine. A conservative mutation can be glutamine to glutamine, glutamine to asparagine, glutamine to aspartic acid, glutamine to glutamine. A conservative mutation can be methionine to phenylalanine, methionine to isoleucine, methionine to valine, methionine to leucine, methionine to methionine. A conservative mutation can be tryptophan to tryptophan, tryptophan to phenylalanine, tryptophan to tyrosine.

In some embodiments, the modified AAV vector comprises modified AAV2 serotype vector. In some embodiments, the modified AAV vector comprises modified VP1, VP2, VP3, or a combination thereof. In some embodiments, the modified AAV2 serotype vector comprises modified VP1, VP2, VP3, or a combination thereof.

In an aspect, the isolated non-naturally occurring nucleic acid comprises at least about 60%, at least about 70%, at least about 80%, at least about 85%, at least about 90%, at least about 95%, at least about 96%, at least about 97%, at least about 98%, at least about 99%, and up to about 100% sequence identity or similarity with any one of SEQ ID NOS: 13-19. In an embodiment, the isolated non-naturally occurring nucleic acid comprises at least about 60% sequence identity or similarity with any one of SEQ ID NOS: 13-19. In some cases, the isolated non-naturally occurring nucleic acid is any one of SEQ ID NOS: 13-19. In an aspect, the isolated non-naturally occurring nucleic acid comprises at least about 60%, at least about 70%, at least about 80%, at least about 85%, at least about 90%, at least about 95%, at least about 96%, at least about 97%, at least about 98%, at least about 99%, and up to about 100% sequence identity or similarity with any one of SEQ ID NO: 21-SEQ ID NO: 27. In an embodiment, the isolated non-naturally occurring nucleic acid comprises at least 60% sequence identity or similarity with any one of SEQ ID NO: 21-SEQ ID NO: 27. In some cases, the isolated non-naturally occurring nucleic acid is any one of SEQ ID NO: 21-SEQ ID NO: 27. In some cases, a nucleic acid encodes for a polypeptide having 60%, 70%, 75%, 80%, 85%, 90%, 95%, 98%, 99% or 100% identity with SEQ ID NO: 12. In some cases, a nucleic acid encodes for the polypeptide of SEQ ID NO: 12.

In an aspect, provided herein are also methods of modifying cells to thereby generate engineered cells. Cells can refer to primary cells, recombinant cells, or cell lines. In some cases, a cell is a packaging cell. A packaging cell can be any one of: HEK 293 cells, HeLa cells, and Vero cells to name a few. An engineered cell can be a primary cell. In some cases, an engineered cell can be an ocular cell. Suitable ocular cells include but are not limited to a: photoreceptor, ganglion cell, RPE cell, amacrine cell, horizontal cell, muller cell, and the like.

In some cases, a cell is a packaging cell utilized to generate viral particles. To general AAV virions or viral particles, an AAV expression vector is introduced into a suitable host cell using known techniques, such as by transfection. In some cases, transfection techniques are used, e.g., CaPO₄ transfection or electroporation, and/or infection by hybrid adenovirus/AAV vectors into cell lines such as the human embryonic kidney cell line HEK 293 (a human kidney cell line containing functional adenovirus E1 genes which provides trans-acting E1 proteins). Transfection techniques are known in the art. See, e.g., Graham et al. (1973) Virology, 52:456, Sambrook et al. (1989) Molecular Cloning, A Laboratory Manual, Cold Spring Harbor Laboratories, New York, Davis et al. (1986) Basic Methods in Molecular Biology, Elsevier, and Chu et al. (1981) Gene 13:197. Suitable transfection methods include calcium phosphate co-precipitation, direct micro-injection, electroporation, liposome mediated gene transfer, and nucleic acid delivery using high-velocity microprojectiles, which are known in the art.

To engineer a cell, a plurality of cells may be contacted with an isolated non-naturally occurring nucleic acid. A contacting can comprise any length of time and may include from about 5 min to about 5 days. Contacting can last from about 5, about 10, about 15, about 20, about 25, about 30, about 35, about 40, about 45, about 50, about 55, or about 60 minutes. In some cases, the contacting can last from 1 hour, 3 hours, 5 hours, 10 hours, 15 hours, 20 hours, 1 day, 2 days, 3 days, 4 days or up to about 5 days.

In some cases, supernatant of the packaging cell line is treated by PEG precipitation for concentrating the virus. In other cases, a centrifugation step can be used to concentrate a virus. For example, a column can be used to concentration a virus during a centrifugation. In some embodiments, a precipitation occurs at no more than about 4° C. (for example about 3° C., about 2° C., about 1° C., or about 1° C.) for at least about 2 hours, at least about 3 hours, at least about 4 hours, at least about 6 hours, at least about 9 hours, at least about 12 hours, or at least about 24 hours. In some embodiments, the recombinant AAV is isolated from the PEG-precipitated supernatant by low-speed centrifugation followed by CsCl gradient. The low-speed centrifugation can be to can be about 4000 rpm, about 4500 rpm, about 5000 rpm, or about 6000 rpm for about 20 minutes, about 30 minutes, about 40 minutes, about 50 minutes or about 60 minutes. In some cases, recombinant AAV is isolated from the PEG-precipitated supernatant by centrifugation at about 5000 rpm for about 30 minutes followed by CsCl gradient. In some cases, CsCl purification can be replaced with IDX gradient ultracentrifugation. Supernatant can be collected at about 12 hours, about 24 hours, about 36 hours, about 48 hours, about 72 hours, about 96 hours, about 120 hours, or a time between any of these two time points after a transfection. Supernatant can also be purified, concentrated, or a combination thereof. For example, a concentration or viral titer can be determined by qPCR or silver stain.

In an aspect, provided is also a plurality of AAV particles isolated from an engineered cell. A viral titer can be from about 10² vp/mL, about 10³ vp/mL, about 10⁴ vp/mL, about 10⁵ vp/mL, about 10⁶ vp/mL, about 10⁷ vp/mL, about 10⁸ vp/mL, or up to about 10⁹ vp/mL. A viral titer can be from about 10² GC/mL, about 10³ GC/mL, about 10⁴ GC/mL, about 10⁵ GC/mL, about 10⁶ GC/mL, about 10⁷ GC/mL, about 10⁸ GC/mL, or up to about 10⁹ GC/mL. In some cases, a viral titer can be from about 10² TU/mL, about 10³ TU/mL, about 10⁴ TU/mL, about 10⁵ TU/mL, about 10⁶ TU/mL, about 10⁷ TU/mL, 10⁸ TU/mL, or up to about 10⁹ TU/mL. An optimal viral titer can vary depending on cell type to be transduced. A range of virus can be from about 1000 MOI to about 2000 MOI, from about 1500 MOI to about 2500 MOI, from about 2000 MOI to about 3000 MOI, from about 3000 MOI to about 4000 MOI, from about 4000 MOI to about 5000 MOI, from about 5000 MOI to about 6000 MOI, from about 6000 MOI to about 7000 MOI, from about 7000 MOI to about 8000 MOI, from about 8000 MOI to about 9000 MOI, from about 9000 MOI to about 10,000 MOI. For example, to infect 1 million cells using a MOI of 10,000, one will need 10,000×1,000,000=10¹⁰ GC.

In some cases, a plurality of AAV particles can be formulated into unit dose form. Various formulations are contemplated for adult or pediatric delivery and include but are not limited to: 0.5×10⁹ vg, 1.0×10⁹ vg, 1.0×10¹⁰, 1.0×10¹¹ vg, 3.0×10¹¹ vg, 6×10¹¹ vg, 8.0×10¹¹ vg, 1.0×10¹² vg, 1.0×10¹³ vg, 1.0×10¹⁴ vg, 1.0×10¹⁵ vg, or up to 1.5×10¹⁵ vg. Compositions of viral particles can be cryopreserved or otherwise stored in suitable containers.

Provided compositions and methods herein can be sufficient to enhance delivery and/or expression of subject biologic by at least about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or up to 100% more than an otherwise comparable unmodified nucleic acid. In some cases, the otherwise comparable unmodified nucleic acid is one that encodes VEGF-Trap. In some cases, modifications can be sufficient to enhance delivery and/or expression of subject biologics by at least about 1-fold, about 6-fold, about 11-fold, about 16-fold, about 21-fold, about 26-fold, about 31-fold, about 36-fold, about 41-fold, about 46-fold, about 51-fold, about 56-fold, about 61-fold, about 66-fold, about 71-fold, about 76-fold, about 81-fold, about 86-fold, about 91-fold, about 96-fold, about 101-fold, about 106-fold, about 111-fold, about 116-fold, about 121-fold, about 126-fold, about 131-fold, about 136-fold, about 141-fold, about 146-fold, about 151-fold, about 156-fold, about 161-fold, about 166-fold, about 171-fold, about 176-fold, about 181-fold, about 186-fold, about 191-fold, about 196-fold, about 201-fold, about 206-fold, about 211-fold, about 216-fold, about 221-fold, about 226-fold, about 231-fold, about 236-fold, about 241-fold, about 246-fold, about 251-fold, about 256-fold, about 261-fold, about 266-fold, about 271-fold, about 276-fold, about 281-fold, about 286-fold, about 291-fold, about 296-fold, about 301-fold, about 306-fold, about 311-fold, about 316-fold, about 321-fold, about 326-fold, about 331-fold, about 336-fold, about 341-fold, about 346-fold, or about 350-fold more than an otherwise comparable unmodified nucleic acid. In an embodiment, increased expression comprises at least a 5-fold, at least a 10-fold, at least a 20-fold, at least a 50-fold, at least a 100-fold, at least a 200-fold, or at least a 500-fold increase as determined by in in vitro assay. Suitable in vitro assays include ELISA, Western blot, Luminex, microscopy, imaging, and/or flow cytometry.

A subject AAV virion can exhibit at least 1-fold, at least 6-fold, at least 10-fold, at least 15-fold, at least 20-fold, at least 25-fold, at least 50-fold, or more than 50-fold, increased infectivity of a retinal cell, compared to the infectivity of the retinal cell (photoreceptor, ganglion cell, RPE cell, amacrine cell, horizontal cell, muller cell, and the like) by an AAV virion comprising an otherwise comparable WT AAV capsid protein.

Provided herein are also methods of treating a disease or condition. A method of treatment can comprise introducing to a subject in need a virion or vector coding for a biologic provided herein. In some cases, a method of treatment comprises introducing a plurality of virions or vectors that code for the biologic that comprises an anti-angiogenic agent. Also provided is a method of treating disease that comprises administering a pharmaceutical composition to a subject in need thereof. A pharmaceutical composition can comprise a sequence that encodes a biologic that comprises an anti-angiogenic agent and/or virions that code for the anti-angiogenic agent.

In an embodiment, a method of treatment comprises administering a therapeutically effective amount of a pharmaceutical composition that comprises an isolated non-naturally occurring nucleic acid that comprises a sequence that encodes a biologic that comprises an anti-angiogenic agent. A sequence can be or can comprise any of the nucleic acids provided herein. For example, the sequence can comprises a nucleic acid sequence that is at least about 60% sequence identity or similarity with any one SEQ ID NOS: 13-19, 21-27, 31, 62, 64, 66, or 68. In some embodiments, the sequence identity is from about 70%, about 80%, about 85%, about 90%, about 95%, about 96%, about 97%, about 98%, about 99%, and up to about 100%. In some embodiments, the sequence comprises the sequence identity is from about 70%, about 80%, about 85%, about 90%, about 95%, about 96%, about 97%, about 98%, about 99%, and up to about 100% to SEQ ID NO: 31. In some embodiments, the sequence comprises a nucleic acid sequence of SEQ ID NO: 31. In some embodiments, the sequence is 100% identical to a nucleic acid sequence of SEQ ID NO: 31. In some embodiments, the sequence comprises the sequence identity is from about 70%, about 80%, about 85%, about 90%, about 95%, about 96%, about 97%, about 98%, about 99%, and up to about 100% to SEQ ID NO: 66. In some embodiments, the iso sequence comprises a nucleic acid sequence of SEQ ID NO: 66. In some embodiments, the sequence is 100% identical to a nucleic acid sequence of SEQ ID NO: 66. In some embodiments, the sequence is single stranded. In some embodiments, the sequence is double stranded.

In some cases, a sequence is modified according to the disclosure, for example the sequence is modified to replace AGA with AGG in at least one codon of a coding region of the sequence as compared to an otherwise comparable sequence lacking the modification in the coding region. As provided herein, the modifications of the disclosure can have certain benefits such as increasing a level of the biologic in a subject as compared to an otherwise comparable subject administered an otherwise comparable isolated non-naturally occurring nucleic acid lacking a modification. Increasing levels of biologics in subjects can have therapeutic effects and can reduce or eliminate any of the diseases or conditions provided herein.

In some cases, an increased level of a biologic in a subject is at least a 5-fold, a 10-fold, a 20-fold, a 50-fold, a 100-fold, a 200-fold, or a 500-fold increased, as determined by a diagnostic assay.

Suitable diagnostic assays can include ocular diagnostic assays. Ocular diagnostic assays can include ophthalmic testing such as refraction testing, ocular scans, Ocular coherence tomography, Farnworth-Munsell 100 Hue Test, Computerized Optic Disc Imaging and Nerve Fiber Layer Analysis (GDX, HRT, OCT), Corneal Topography, Electroretinography (ERG), electro-oculography (EOG), visual evoked potentials (VEP), visual evoked response (VER), Fluorescein Angiography, Ocular Coherence Tomography (OCT), retinal photography, fundus photography, Specular Microscopy, Goldmann, Humphrey, FDT, Octopus, Biometry/IOL calculation, A-Scan, B-Scan, and combinations thereof.

In some cases, a retinal test can be utilized. Nonlimiting methods for assessing retinal function and changes thereof include assessing visual acuity (e.g. best-corrected visual acuity [BCVA], ambulation, navigation, object detection and discrimination), assessing visual field (e.g. static and kinetic visual field perimetry), performing a clinical examination (e.g. slit lamp examination of the anterior and posterior segments of the eye), assessing electrophysiological responsiveness to all wavelengths of light and dark (e.g. all forms of electroretinography (ERG) [full-field, multifocal and pattern], all forms of visual evoked potential (VEP), electrooculography (EOG), color vision, dark adaptation and/or contrast sensitivity). Nonlimiting methods for assessing anatomy and retinal health and changes thereof include Optical Coherence Tomography (OCT), fundus photography, adaptive optics scanning laser ophthalmoscopy (AO-SLO), fluorescence and/or autofluorescence; measuring ocular motility and eye movements (e.g. nystagmus, fixation preference, and stability), measuring reported outcomes (patient-reported changes in visual and non-visually-guided behaviors and activities, patient-reported outcomes [PRO], questionnaire-based assessments of quality-of-life, daily activities and measures of neurological function (e.g. functional Magnetic Resonance Imaging (MRI)).

Relevant ocular diseases and conditions can include but are not limited to: blindness, Achromatopsia, Age-related macular degeneration (AMD), Diabetic retinopathy (DR), Glaucoma, Bardet-Biedl Syndrome, Best Disease, Choroideremia, Leber Congenital Amaurosis, Macular degeneration, Polypoidal choroidal vasculopathy (PCV), Retinitis pigmentosa, Refsum disease, Stargardt disease, Usher syndrome, X-linked retinoschisis (XLRS), Rod-cone dystrophy, Cone-rod dystrophy, Oguchi disease, Malattia Leventinese (Familial Dominant Drusen), and Blue-cone monochromacy. In an embodiment, the ocular disease or condition is AMD. AMD can be wet AMD or dry AMD.

In some cases, an administration of a pharmaceutical is sufficient to reduce at least a symptom of a disease or condition, treat the disease or condition, and/or eliminate the disease or condition. In some cases, improvements of diseases or conditions can be ascertained by any of the provided diagnostic assays. In other cases, an improvement can be obtained via an interview with the treated subject. For example, a subject may be able to communicate to an attending physician that their vision is improved as compared to their vision prior to administration of a subject pharmaceutical. In other cases, an in vivo animal model may be used to ascertain reduction of a disease or condition after treatment. Suitable animal models include mouse models, primate models, rat models, canine models, and the like.

Pharmaceutical compositions can be administered to a subject using various techniques, such as: intravitreally, intramuscular, intravenous, subcutaneous, and/or intraperitoneal injection.

For in vivo delivery, subject nucleic acids and/or AAV virions can be formulated into pharmaceutical compositions and will generally be administered intravitreally or parenterally (e.g., administered via an intramuscular, subcutaneous, intratumoral, transdermal, intrathecal, etc., route of administration). In some aspects, a pharmaceutical composition can be used to treat a subject such as a human or mammal, in need thereof. In some cases, a subject can be diagnosed with a disease, e.g., ocular disease. In some aspects, subject pharmaceutical compositions are co-administered with secondary therapies. A secondary therapy can comprise any therapy for ocular use. In some cases, a secondary therapy comprises nutritional therapy, vitamins, laser treatment, such as laser photocoagulation, photodynamic therapy, Visudyne, anti-VEGF therapy, eye-wear, eye drops, numbing agents, Orthoptic vision therapy, Behavioral/perceptual vision therapy, and the like. In some aspects, any of the previously described biologics can be considered a secondary therapy.

Any of the pharmaceutical compositions can also comprise an excipient. Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

In some embodiments, an effective amount of the subject rAAV virion results in a decrease in the rate of loss of retinal function, anatomical integrity, or retinal health, e.g. a 2-fold, 3-fold, 4-fold, or 5-fold or more decrease in the rate of loss and hence progression of disease, for example, a 10-fold decrease or more in the rate of loss and hence progression of disease. In some embodiments, the effective amount of the subject rAAV virion results in a gain in visual function, retinal function, an improvement in retinal anatomy or health, and/or an improvement in ocular motility and/or improvement in neurological function, e.g. a 2-fold, 3-fold, 4-fold or 5-fold improvement or more in retinal function, retinal anatomy or health, and/or improvement in ocular motility, e.g. a 10-fold improvement or more in retinal function, retinal anatomy or health, and/or improvement in ocular motility. As will be readily appreciated by the ordinarily skilled artisan, the dose required to achieve the desired treatment effect will typically be in the range of 1×10⁸ to about 1×10¹⁵ recombinant virions, typically referred to by the ordinarily skilled artisan as 1×10⁸ to about 1×10¹⁵ “vector genomes”.

In some aspects, compositions provided herein, such as pharmaceutical compositions are administered to a subject in need thereof. In some cases, an administration comprises delivering a dosage of an AAV vector of about vector 0.5×10⁹ vg, 1.0×10⁹ vg, 1.0×10¹⁰, 1.0×10¹¹ vg, 3.0×10¹¹ vg, 6×10¹¹ vg, 8.0×10¹¹ vg, 1.0×10¹² vg, 1.0×10¹³ vg, 1.0×10¹⁴ vg, 1.0×10¹⁵ vg, 1.5×10¹⁵ vg. For example, for in vivo injection, i.e., injection directly into the eye, a therapeutically effective dose can be on the order of from about 10⁶ to about 10¹⁵ of subject AAV virions, e.g., from about 10⁸ to 10¹² engineered AAV virions. For in vitro transduction, an effective amount of engineered AAV virions to be delivered to cells will be on the order of from about 10⁸ to about 10¹³ of the engineered AAV virions. Other effective dosages can be readily established by one of ordinary skill in the art through routine trials establishing dose response curves.

Administrations can be repeated for any amount of time. In some aspects, administering is performed: twice daily, every other day, twice a week, bimonthly, trimonthly, once a month, every other month, semiannually, annually, or biannually.

Dosage treatment may be a single dose schedule or a multiple dose schedule. Moreover, the subject may be administered as many doses as appropriate. One of skill in the art can readily determine an appropriate number of doses. In some aspects, a pharmaceutical composition is administered via intravitreal injection, subretinal injection, microinjection, or supraocular injection.

In some aspects, a subject can be screened via genetic testing for a mutation before, during, and/or after administration of a pharmaceutical composition provided herein. Relevant genes that can be screened for mutations comprise: RPE65, CRB1, AIPL1, CFH, or RPGRIP.

Also provided are kits comprising any of the compositions provided herein. Provided is also a container that comprises a) a subject modified adeno-associated virus (AAV) capsid; b) a subject vector; or c) a subject engineered virion. In an aspect, the container is a vial, syringe, or needle. In some cases, the container is configured for ocular delivery.

Kits may comprise a suitably aliquoted composition. The components of the kits may be packaged either in aqueous media or in lyophilized form. The container means of the kits will generally include at least one vial, test tube, flask, bottle, syringe, or another container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow-molded plastic containers into which the desired vials are retained.

In some instances, a packaged product comprising a composition described herein can be properly labeled. In some instances, the pharmaceutical composition described herein can be manufactured according to good manufacturing practice (cGMP) and labeling regulations. In some cases, a pharmaceutical composition disclosed herein can be aseptic.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1: Codon Optimization and Cloning of Nucleic Acid Constructs

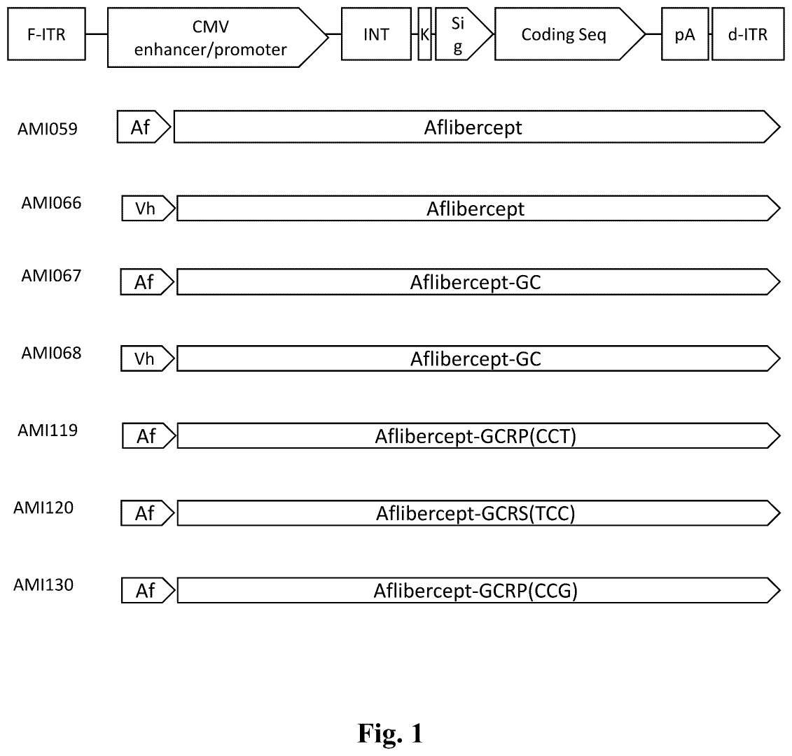

A DNA sequence containing SV40 intron-VEGF-Trap open reading frame (ORF)-synthetic poly A signal was synthesized by a service company (Twist Bioscience, South San Francisco, CA) to contain 40% GC-content in the VEGF-Trap ORF. The DNA sequence was PCR amplified with forward primer A024 and reverse primer A025 and cloned into the XbaI and SphI sites of pFB-scCMV-MIF using the NEBuilder HiFi DNA Assembly kit (New England Biolabs, Ipswich, MA) to create AMI059-pFB-scCMV-SV40-intron-Af-VEGF-Trap as shown in FIG. 1. The pFB-scCMV-MIF plasmid that contains one full AAV ITR and one truncated ITR for self-complementary AAV (scAAV) is a derivative of pFastBac shuttle plasmid from the Bac-to-Bac Baculovirus Expression System (Invitrogen). In the cloning process, MIF was replaced with VEGF-Trap gene. The VEGF-Trap gene contains its original signal peptide (Af). To replace the Af with the human antibody heavy chain signal peptide (Vh), the SV40-intron-Vh-fragment was PCR amplified with forward primer A024 and reverse primer A082, and the plasmid AMI060 that contains the SV40-intron-Vh sequence as template. The VEGF-Trap ORF was amplified with forward primer A085 and reverse primer A025 and plasmid AMI059-pFB-scCMV-SV40-intron-kozak-VEGF-Trap as template. These two PCR fragments were cloned into the XbaI and SphI sites of AMI059 using the NEBuilder HiFi DNA Assembly kit to create AMI066-pFB-scCMV-SV40-intron-Vh-VEGF-Trap as shown in FIG. 1.

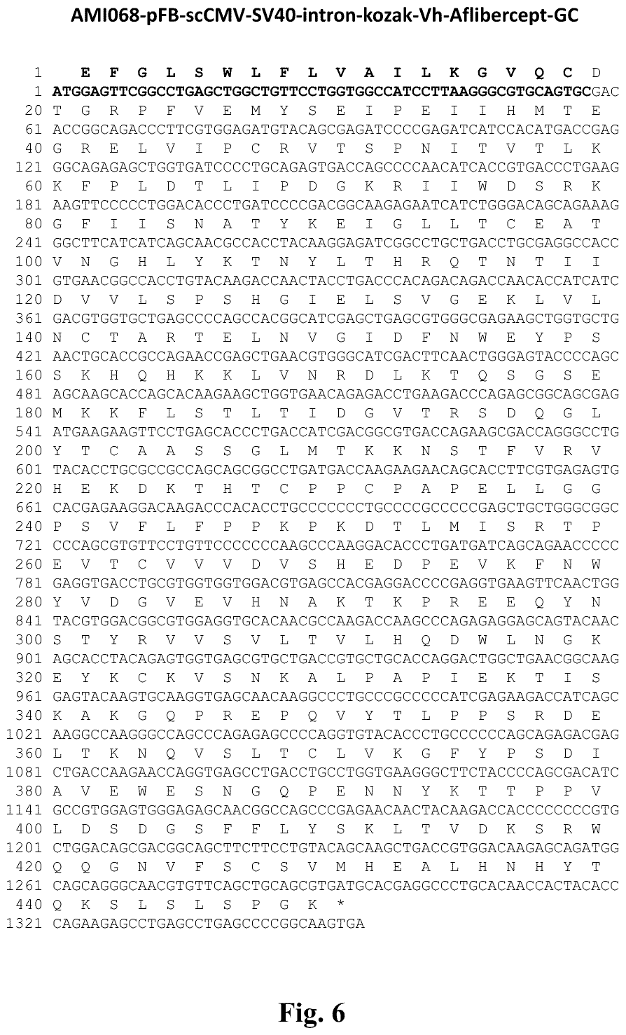

To optimize the VEGF-Trap ORF, reverse translation of the VEGF-Trap protein sequence into DNA was performed using the SnapGene Software (GSL Biotech, San Diego, CA) by selecting the Homo sapiens preferred codon output. This sequence was synthesized by Twist Bioscience and PCR amplified with forward primer A086 and reverse primer A087. The PCR fragment was cloned into the StuI and BstBI sites of AMI059 via HiFi reaction to create AMI067-pFB-scCMV-SV40-intron-kozak-Af-VEGF-Trap-GC as shown in FIG. 1. To replace the signal peptide Af in AMI067 with Vh, the SV40-intron-Vh-fragment was PCR amplified with forward primer A024 and reverse primer A089, and the plasmid AMI060 that contains the SV40-intron-Vh sequence as template. The Twist synthesized VEGF-Trap ORF was PCR amplified with forward primer A088 and reverse primer A090. These two PCR fragments were cloned into the XbaI and BstBI sites of AMI059 using the NEBuilder HiFi DNA Assembly kit to create AMI068-pFB-scCMV-SV40-intron-kozak-Vh-VEGF-Trap-GC as shown in FIG. 1 and FIG. 6.

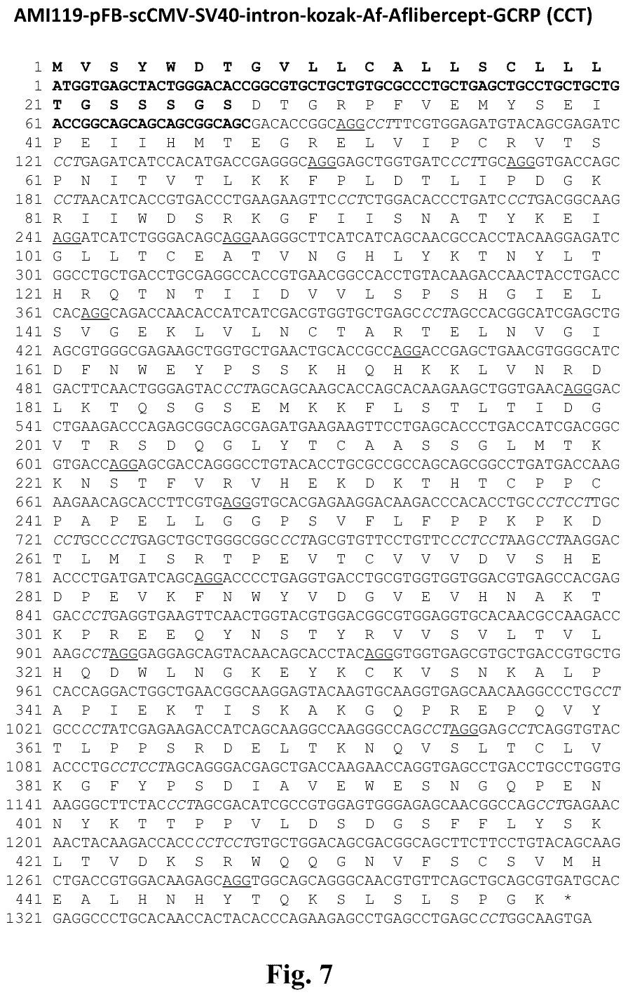

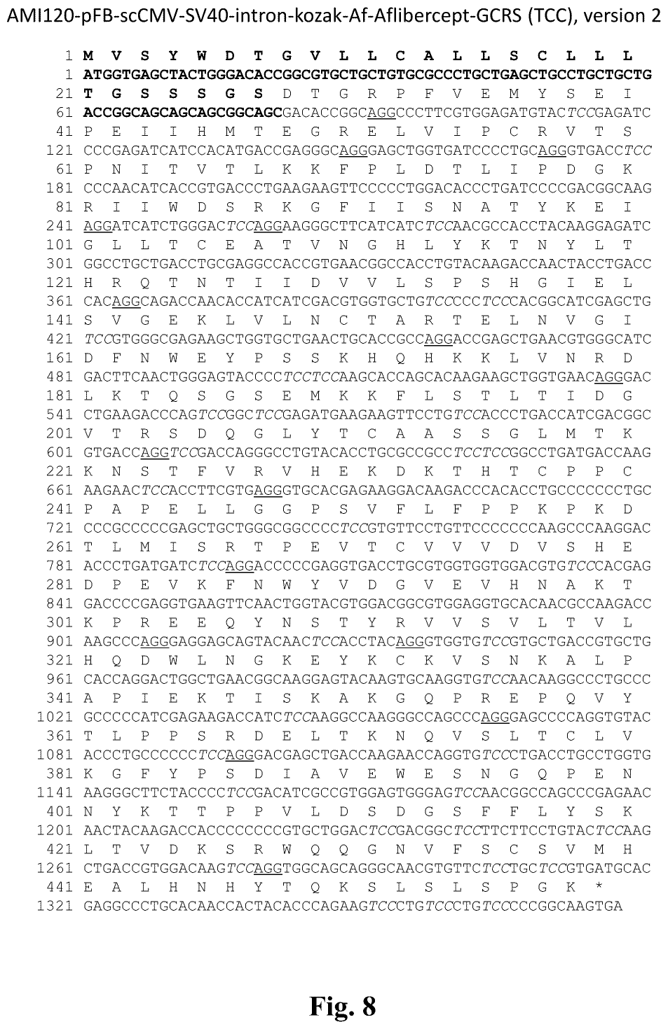

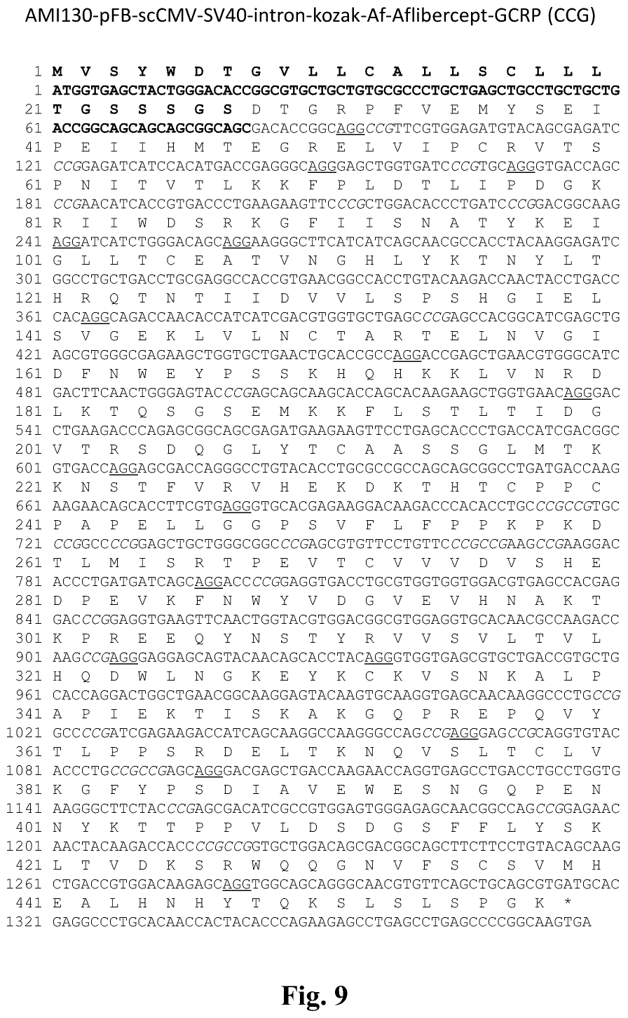

To perform further optimization of the VEGF-Trap DNA codons, the DNA sequence was manually adjusted to make three more versions of variant VEGF-Trap DNA molecules. In Version 1, 16 Arg codons were changed from AGA to AGG and 29 Pro codons were changed from CCC to CCT to create AMI119-pFB-scCMV-SV40-intron-kozak-Af-VEGF-Trap-GCRP (CCT), see FIG. 1 and FIG. 7. In Version 2, in addition to the 16 Arg codon changes, 36 Ser codons were further changed from AGC to TCC to create AMI120-pFB-scCMV-SV40-intron-kozak-Af-VEGF-Trap-GCRS (TCC), see FIG. 1 and FIG. 8. In Version 3, in addition to the 16 Arg codon changes, 29 Pro codons were further changed from CCC to CCG to create AMI130-pFB-scCMV-SV40-intron-kozak-Af-VEGF-Trap-GCRP (CCG), see FIG. 1 and FIG. 9. All the variant versions of VEGF-Trap DNA sequences were first synthesized by Twist Bioscience, then PCR amplified with forward and reverse primers, and finally cloned into the BstBI and StuI sites of AMI067 plasmid using the NEBuilder HiFi DNA Assembly kit. The plasmid constructs are listed in Table 2 and the information of the primers used for the PCR amplifications are listed in Table 3 and Table 4.

TABLE 2
List of pFB shuttle plasmids containing the
VEGF-Trap gene with different codons

| Clone | | |
|---|---|---|
| No. | Clone Name | Codon changes |
| AMI059 | pFB-scCMV-SV40-intron-kozak-Af- | Low GC |
| | VEGF-Trap | |
| AMI066 | pFB-scCMV-SV40-intron-kozak-Vh- | Low GC |
| | VEGF-Trap | |
| AMI067 | pFB-scCMV-SV40-intron-kozak-Af- | High GC |
| | VEGF-Trap-GC | |
| AMI068 | pFB-scCMV-SV40-intron-kozak-Vh- | High GC |
| | VEGF-Trap-GC | |
| AMI119 | pFB-scCMV-SV40-intron-kozak-Af- | 16 Arg codons from AGA |
| | VEGF-Trap-GCRP(CCT) | to AGG and 29 Pro codons |
| | | from CCC to CCT |
| AMI120 | pFB-scCMV-SV40-intron-kozak-Af- | 16 Arg codons from AGA |
| | VEGF-Trap-GCRS(TCC) | to AGG and 36 Ser codons |
| | | from AGC to TCC |
| AMI130 | pFB-scCMV-SV40-intron-kozak-Af- | 16 Arg codons from AGA |
| | VEGF-Trap-GCRP | to AGG and 29 Pro codons |
| | | from CCC to CCG |

TABLE 3
List of clone number and corresponding PCR primers used
to generate the PCR fragments for the cloning process

| | 5′ PCR Fragment | | 3′ PCR Fragment | |
|---|---|---|---|---|
| Clone No. | F-primer | R-primer | F-primer | R-primer |
| AMI059 | A024 | A025 | — | — |
| AMI066 | A024 | A082 | A085 | A025 |
| AMI067 | A086 | A087 | — | — |
| AMI068 | A024 | A089 | A088 | A090 |
| AMI119 | A086 | A381 | — | — |
| AMI120 | A086 | A382 | — | — |
| AMI130 | A086 | A087 | — | — |

TABLE 4
Sequences of primers used for PCR reactions
and DNA sequencing analysis

| Primer | SEQ ID | |
|---|---|---|
| ID | NO | DNA sequence |
| A024 | 1 | 5′-ATCCAGCCTCCGGACTC |
| | | TAGAGTTAACTGGTAAGTTT |
| | | AGT-3′ |
| A025 | 2 | 5′-TGGGGTTGATCTCTCCC |
| | | CAGCATGCCACACAAAAAAC |
| | | CAA-3′ |
| A082 | 3 | 5′-TACATTTCTACAAAAGG |
| | | TCTACCAGTATCGCACTGCA |
| | | CGCCCTTAAGGA-3′ |
| A085 | 4 | 5′-GATACTGGTAGACCTTT |
| | | TGTAG-3′ |
| A086 | 5 | 5′-GTTGCCTTTACTTCTAG |
| | | GCCTGCCGCCACCATGGTGA |
| | | GCTACTGGGAC-3′ |
| A087 | 6 | 5′-TAATGAAAATAAAGATA |
| | | TTTTATTTTCGAATCACTTG |
| | | CCGGGGCTCAGG-3′ |
| A088 | 7 | 5′-GACACCGGCAGACCCTT |
| | | CGTGG-3′ |
| A089 | 8 | 5′-ACGAAGGGTCTGCCGGT |
| | | GTCGCACTGCACGCCCTTAA |
| | | GGA-3′ |
| A090 | 9 | 5′-TAATGAAAATAAAGATA |
| | | TTTTATTTTCGAATCACTTG |
| | | CCGGGGCTCAGG-3′ |
| A381 | 10 | 5′-TAATGAAAATAAAGATA |
| | | TTTTATTTTCGAATCACTTG |
| | | CCaggGCTCAGG-3′ |
| A382 | 11 | 5′-TAATGAAAATAAAGATA |
| | | TTTTATTTTCGAATCACTTG |
| | | CCGGGggaCAGg-3′ |

To determine the relationship between codon optimization and protein expression levels in the human cells, four plasmids were constructed containing the same expression cassette but with variations of VEGF-Trap DNA coding sequences. Two of the coding sequences have low GC-contents and two high GC-content. In one of each the low and high GC-content coding sequence, a human antibody heavy chain (Vh) secretion signal peptide was used to replace the VEGF-Trap secretion signal peptide (Af). All four plasmids have the identical DNA sequence except the VEGF-Trap open reading frame (ORF). The expression cassette contains a CMV promoter, a SV40 intron, a Kozak sequence located upstream of the start codon of the VEGF-Trap ORF and a synthetic polyadenylation signal located downstream the stop codon. In order to make more variations of the VEGF-Trap codons, the VEGF-Trap codons were changed and AMI119, AMI120, and AMI130 plasmids were cloned. The diagrams of these expression cassettes flanked by a full AAV2 ITR and a truncated AAV2 ITR are shown in FIG. 1. The full DNA sequences of each expression cassettes are SEQ ID NO: 13-SEQ ID NO: 19 or SEQ ID NO: 21-SEQ ID NO: 27 (with SEQ ID NO: 20 being control plasmid sequence; and SEQ ID NO: 28 or SEQ ID NO: 70 being control coding sequence for reference VEGF-Trap).

The estimated eCAI values of each ORF for human cells and its relationship to the GC content were determined. The Expected Codon Adaptation Index (eCAI) values were estimated based on the web-based free E-CAI server. Briefly, the target DNA coding sequences were respectively pasted on the dialog box. The human codon usage table was selected from the “Codon Usage Databases” in the same server and pasted on the next dialog box. The Poissen Method was chosen (The Markov Method gave very similar results) and “Standard” genetic code was selected. The eCAI value was estimated by pressing the “Accept” button. The results are shown in Table 5. The GC contents are positively correlated with the eCAI values, the higher the GC contents, the higher the eCAI values. AMI059 and AMI066 both contained low GC content of 40% and eCAI of 0.723 and 0.722, respectively. AMI067 and AMI068 both contained high GC of 62% and eCAI values of 0.867 and 0.864, respectively. A natural cDNA sequence of VEGF-Trap was compiled using the secretion signal peptide sequence and FLT1 domain from natural occurring mRNA sequence of NM_001160031, the natural occurring mRNA KDR domain from NM_002252.3, and the human antibody IgG1 Fc domain of natural occurring mRNA AK129809.1. This compiled natural occurring VEGF-Trap was estimated to contain 52% GC and have an eCAI of 0.790, well below the optimized codons of AMI067 and AMI068. Further changes of the VEGF-Trap codons slightly changed the GC contents and eCAI values as shown in Table 5. Since higher eCAI values indicate more commonly used codons are in the ORF, that can suggest higher protein expression levels in those expression cassettes.

TABLE 5
The correlation of GC content with Expected Codon
Adaptation Index (eCAI) in human cells

| Clone | | GC | Expected Codon Adaptation |
|---|---|---|---|
| No. | Description | Content | Index (eCAI) |
| N/A | Natural cDNA sequence | 52% | 0.790 |
| AMI059 | Af-VEGF-Trap | 40% | 0.723 |
| AMI066 | Vh-VEGF-Trap | 40% | 0.722 |
| AMI067 | Af-VEGF-Trap-GC | 62% | 0.867 |
| AMI068 | Vh-VEGF-Trap-GC | 62% | 0.864 |
| AMI119 | Af-VEGF-Trap- | 61% | 0.857 |
| | GCRP(CCT) | | |
| AMI120 | Af-VEGF-Trap- | 63% | 0.874 |
| | GCRS(TCC) | | |
| AMI130 | Af-VEGF-Trap-GCRP | 63% | 0.873 |

### Example 2: Increase of GC-Content Level Enhances VEGF-Trap Expression in Transient Transfected Mammalian Cells

To determine the impact of GC-content on the expression of VEGF-Trap in mammalian cells, the four plasmids (AMI059, AMI066, AMI067, and AMI068) were purified and transfected into HEK 293 cells. For the transfection, cells were seeded on 100 mm cell culture dish (Corning, NY) at about 2×10⁶ cells/dish in 10 mL media overnight. Fourteen μg plasmid DNA and 22 μL of Lipofectamine 3000 (Thermo fisher) were each diluted in 0.5 mL of Opti-medium (Thermo fisher) and mixed together. The mixture was added to the cells drop-wise and cells were incubated at 37° C. in the CO₂ incubator for 48 hours. Media were harvested.

Forty-eight hours after transfection, media were harvested and loaded directly (non-reducing) or mixed with loading buffer and heated at 90° C. for 5 minutes (reducing) before loading to the SDS-gel. After blotting onto a PVDF membrane, the VEGF-Trap protein was detected with anti-human IgG Fc antibody.

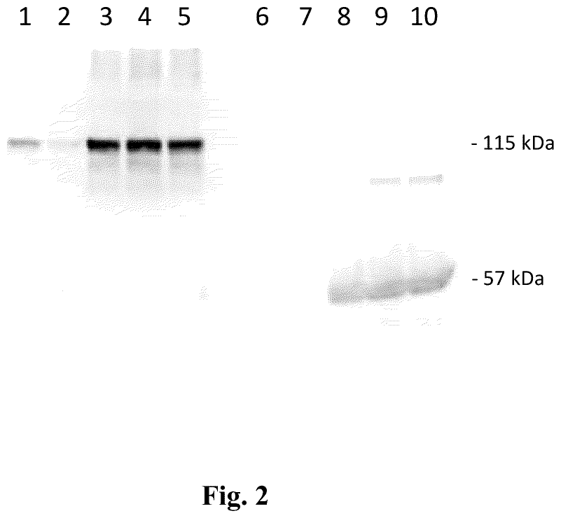

As shown in FIG. 2, a high GC-content of a coding sequence had higher protein expression than a low GC-content coding sequence. Lanes 1 through 5 are shown on a non-reducing gel and lanes 6 through 10 are shown on a reducing gel. Lanes 3, 4, 5, 8, 9 and 10 show a high GC-content (plasmids AMI067, lanes 3 and 8 and AMI068, lanes 4, 5, 9 and 10) and lanes 1, 2, 6, and 7 show a low GC-content (plasmids AMI059, lanes 1 and 6, and AMI066 lanes 2 and 7). Based on the intensity of the Western blot image measured with ImageJ (free software, NIH), high GC-content ORF displayed a 9 to 11-fold increase in protein expression over the low GC-content ORF. Different secretion signal peptides seemed to have a minor impact on the secretion of protein into the media. In the low GC-content ORF, the original VEGF-Trap secretion peptide showed slightly higher expression than the human antibody heavy chain secretion signal peptide, whereas there was no difference in protein secretion between these two secretion peptides in the high GC-content ORF.

### Example 3: GC-Content Level has No Impact on AAV2.N53 and AAV2.N54 Vector Production

The expression cassettes were cloned into the scAAV shuttle plasmid backbone and rBVs were generated to produce AAV2, AAV2.53, and AAV2.N54 vectors by co-infection of Sf9 cells.

Sf9 cells (Expression Systems) were cultured in ESF AF media (Expression Systems) containing 100 units/mL penicillin and 100 μg/mL streptomycin (Thermo Fisher Scientific, Pleasanton, CA) in Corning bottle with gentle shaking at 150 rpm and 28° C. Once cells grew to ˜1e+7cells/mL, they were split 1:4 in fresh media into a new bottle and continuously cultured for maintenance purpose.

Recombinant baculoviruses (rBV) were generated using the Bac-to-Bac Baculovirus Expression System according to the manufacturer's instruction (Invitrogen, Carlsbad, CA). Briefly, the pFB shuttle plasmids containing the target genes were each diluted into 1 ng/uL in TE buffer, and 2 ng of each DNA was mixed with 20 μL of Δcath-DH10Bac competent bacteria containing a bacmid DNA molecule with the cathepsin gene deleted (Virovek, Hayward, CA) and incubated on ice for 30 minutes followed by heat-shock at 42° C. for 30 seconds. After incubating on ice for 2 minutes, the bacteria were cultured at 37° C. for 4 hours to recover and then plated on agar plates containing 50 μg/mL of kanamycin, 7 μg/mL of gentamycin, 10 μg/mL of tetracycline, 40 μg/mL of IPTG, and 100 μg/mL of X-gal. After 48 hours of incubation at 37° C., white colonies containing the recombinant bacmid DNAs were picked and miniprep bacmid DNAs purified under sterile condition. About 5 μg of each bacmid DNA and 10 μL of GeneJet Reagent (SignaGen Laboratories, Fredrick, MD) were respectively diluted in 100 μL ESFAF media (Expression Systems, Davis, CA) and then mixed together for about 30 min to form the transfection mixture. Sf9 cells were plated in the 6-well plate at 1.5e+6 cells/well in 2 mL ESFAF media at 28° C. for about 30 min. After removing the old media from the Sf9 cells, each transfection mixture was diluted in 800 uL ESFAF media and then added to the Sf9 cells. After incubation at 28° C. overnight, each well was added with additional 1 mL ESFAF media. After a total incubation time of 4 days, media containing the rBVs were collected and amplified at 1:200 ratio to generate sufficient quantity of rBVs ready for use in the AAV production process.

To produce and purify the AAV vectors comprising the non-naturally occurring nucleic acid comprising a modification described herein (e.g., at least one codon modification), the rBV carrying the AAV2 Rep and capsid genes (rBV-Cap2-Rep, rBV-Cap2.N53-Rep, rBV-Cap2.N54-Rep), and the rBV carrying the VEGF-Trap expression cassettes (rBV-VEGF-Trap) were used to co-infect Sf9 cells for AAV production. Briefly, 10 multiplicity of infection (moi) of rBV-Cap2-Rep (or rBV-Cap2.N53-Rep or rBV-Cap2.N54-Rep) and 5 moi of rBV-VEGF-Trap were used to co-infect the Sf9 cell line at density of about 5e+6 cells/mL with 50% fresh ESFAF media for 3 days at 28° C. with shaking speed of 180 revolutions per minute (rpm) in a shaker incubator. At the end of infection, cell pellets were collected by centrifugation at 3,000 rpm for 10 minutes. The cells were lysed in Sf9 lysis buffer containing 50 mM Tris-HCl, pH 8.0, 2 mM MgCl2, 1% sarkosyl, 1% Triton X-100, and 125 units/mL benzonase with vigorous vortex followed by shaking at 350 rpm, 37° C. for 1 hour. At the end of shaking, salt concentration was increased to 500 millimolar (mM) by vortexing, and the lysates were cleared by centrifugation at 8,000 rpm for 20 min at 4° C. The cleared lysates were transferred to ultraclear centrifuge tubes for SW28 swing bucket rotor which contain 5 mL of 1.50 g/cc and 10 mL of 1.30 g/cc cesium chloride solutions. After centrifugation at 28,000 rpm, 15° C. for about 18 hours, the AAV bands were collected with syringes and transferred to ultraclear centrifuge tubes for the 70 Ti centrifuge rotor. The centrifuge tubes were filled with 1.38 g/cc cesium chloride solution and heat-sealed. The AAV samples were subjected to a second round of ultracentrifugation at 65,000 rpm, 15° C. for about 18 hours and AAV bands were collected with syringes. The purified AAV samples were buffer-exchanged into PBS buffer containing 0.001% Pluronic F-68 and filter-sterilized with 0.22 μm syringe filters. The sterilized AAV samples were stored at 4° C. within a month and then transferred to −80° C. for long term storage. AAV titer was determined with real-time PCR method using the QuantStudio 7 Flex Real-Time PCR System (Invitrogen).

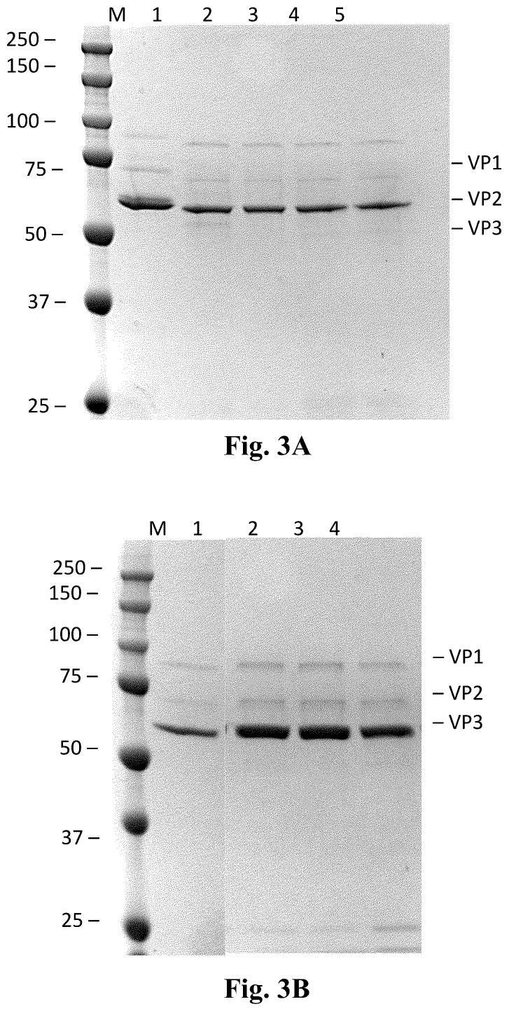

The AAV2, AAV2.53, and AAV2.N54 vector titers are listed below in the Table 6. The purity of AAV2.53 vectors is shown in FIG. 3, which shows an SDS-PAGE with Simply Blue Staining of low-GC and high-GC AAV vector production in Sf9 cells. M indicates a size marker for the gel. FIG. 3A shows the AAV5 vector loaded as the control (1e+11 viral genome (vg)/lane loaded) compared to AMI059 loaded into lane 2 (5e+10 vg/lane loaded), AMI066 loaded into lane 3 (5e+10 vg/lane loaded), AMI067 loaded into lane 4 (5e+10 vg/lane loaded), and AMI068 loaded into lane 5 (5e+10 vg/lane loaded). FIG. 3B shows the AAV2 vector loaded as the control (1e+11 vg/lane loaded) compared to AMI119 loaded into lane 2 (1e+11 vg/lane loaded), AMI120 loaded into lane 3 (1e+11 vg/lane loaded) and AMI130 loaded into lane 4 (1e+11 vg/lane loaded). All AAV vectors were produced at normal range and no GC-content related difference was observed.

TABLE 6
Comparison of AAV2.N53 production yields between
constructs containing low and high-GC DNA content

| Clone | | AAV Titer | AAV Production |
|---|---|---|---|
| No. | AAV name (production Lot no.) | (vg/mL) | Yield (vg/L) |
| AMI059 | AAV2-CMV-VEGF-Trap (20-118) | 1.32e+12 | 3.29e+13 |
| AMI066 | AAV2-CMV-VEGF-Trap (20-119) | 2.02e+12 | 4.29e+13 |
| AMI067 | AAV2-CMV-VEGF-Trap (20-120) | 0.99e+12 | 2.38e+13 |
| AMI068 | AAV2-CMV-VEGF-Trap (20-121) | 1.85e+12 | 3.88e+13 |
| AMI119 | AAV2-CMV-VEGF-Trap (20-113) | 4.59e+12 | 3.52e+13 |
| AMI120 | AAV2-CMV-VEGF-Trap (20-114) | 5.01e+12 | 3.50e+13 |
| AMI130 | AAV2-CMV-VEGF-Trap (20-115) | 2.96e+12 | 1.97e+13 |
| AMI120 | AAV2.N53-CMV-VEGF-Trap | 1.92e+13 | 1.92e+14 |
| | (20-140) | | |
| AMI120 | AAV2.N54-CMV-VEGF-Trap | 1.62e+13 | 1.45e+14 |
| | (21-004) | | |

### Example 4: Increase of GC-Content Level Enhances VEGF-Trap Expression in scAAV2.N53 Transduced Mammalian Cells

scAAV2.N53 vectors harboring VEGF-Trap expression cassettes were produced and purified. They were used to transduce HEK293 cells and the expression levels of VEGF-Trap were determined with Western blot analysis and ELISA assay using the goat anti-human IgG-Fc antibody.

For cell culture, Human HEK293 cells were cultured in DMEM medium (Thermo fisher) with 5% FBS (ATCC, Manassas, VA) in a Series II Water Jacketed CO₂ incubator (Thermo Forma) at 37° C. For maintenance, cells were split 1:10 once a week.

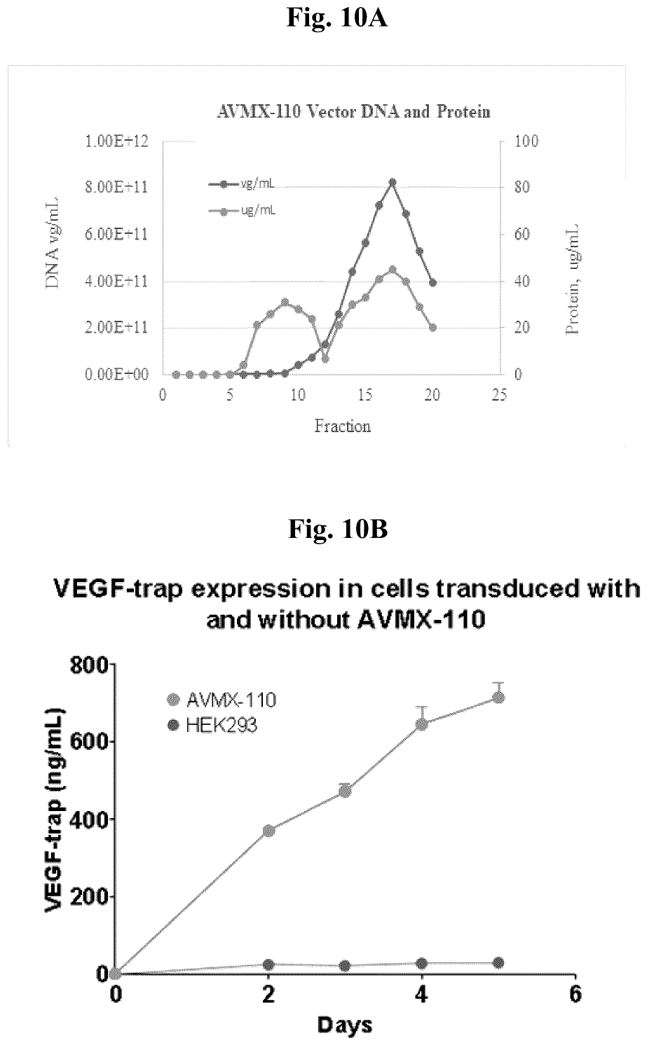

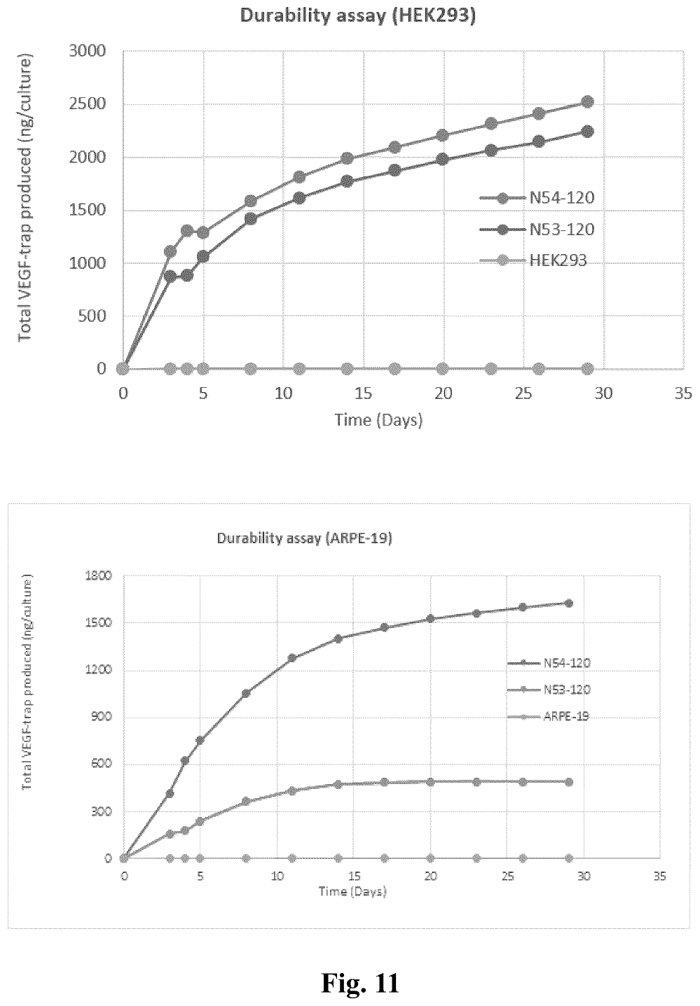

For AAV vector transduction, HEK293 cells (FIG. 10) or hARPE-19 cells (FIG. 11) were seeded at a density of 2×10⁶ cells in 100 mm dish and grown overnight to about 80% confluency in the CO₂ incubator at 37° C. The next morning AAV vectors were added to the cells at MOI of 1.0×10⁴ vg/cell and transduction was carried out for 48 hours at 37° C. Supernatants were collected without the cells and analyzed for protein expression by Western Blot or protein levels by ELISA. All transductions were performed at least three independent experiments.

For the ELISA, media harvested from transient transfection or AAV transduction were centrifuged at 10,000 rpm for 5 minutes to remove any particulates and used for the assay. A 96-well plate was first coated with recombinant human endothelial growth factor-A (rhVEGF-A) at 100 μL/well in coating buffer at room temperature overnight. After removing the coating buffer, the plate was washed 3 times with 200 μL/well of washing buffer (0.05% Tween-20 in PBS, pH 7.3±0.1), and blocked with 200 μL/well of Blocking buffer (1% BSA in PBS, pH 7.3±0.1) for a minimum 1 hour at room temperature. After removing the Blocking buffer, the plate was washed 3 times with the washing buffer. After removing the residue of washing buffer, 100 μL/well of testing samples diluted in the Blocking buffer was added to the plate for 2 hours at room temperature. After removing the testing samples, the plate was washed again 3 times with the washing buffer. After removing the residue of washing buffer, 100 μL/well of the Biotinylated Mouse monoclonal (H2) anti-Human IgG Fc (Biotin) Detection Antibody (Abcam, Cambridge, MA) was added to the plate and incubated at room temperature for one hour protected from direct light. At the end of incubation, the plate was washed 3 times, and 100 μL/well of 1× HRP-Streptavidin solution was added to the plate and incubated at room temperature for 45 min. At the end of incubation, the plate was washed 3 times and 200 μL/well of Substrate Solution (Tetramethylbenzidine) was added to the plate. After incubating at room temperature for about 10 min to about 15 min to develop the color, 50 μL/well of the Stop solution (2 N sulfuric acid) was added to the plate and optical density values were determined at wavelength 450 nm with the Multimode Plate Reader EnVision (PerkinElmer, Santa Clara, CA).

For the Western blot, HEK293 cell supernatants at 48 hours post-transfection were collected. A total of 20 μl of the supernatant was mixed with loading buffer, heated at 95° C. for 5 minutes, and loaded onto NuPAGE 10% Tris-Glycine gels (Invitrogen) for electrophoresis. For non-reducing gel, the supernatant was mixed with a non-reducing loading buffer and then loaded on the gel. Proteins on the gels were transferred onto PVDF membranes using the X Cell II™ Blot Module (Invitrogen, Carlsbad, CA, USA). The membranes were treated with casein blocker in PBS (Thermo Scientific, Waltham, MA, USA) for at least one hour at room temperature and probed with the appropriate primary antibody, followed by incubation with the appropriate anti-rabbit or anti-mouse IgG conjugated to horseradish peroxidase (Amersham Biosciences, Uppsala, Sweden). Proteins were detected using ECL™ Western blotting reagents (Amersham).

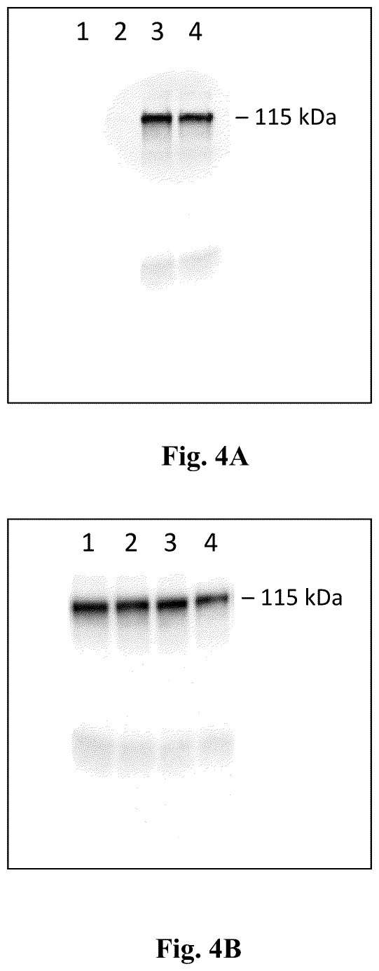

The results are shown in FIG. 4 and Table 7. In FIG. 4A, lanes 1-4 were loaded with AMI059, AMI066, AMI067, and AMI068, respectively. In FIG. 4B, lanes 1-4 were loaded with AMI120, AMI119, AMI067, and AMI130, respectively. As shown, in FIG. 4A in lanes 3 and 4 corresponding to AMI067, and AMI068, respectively, the increase of GC content in the VEGF-Trap ORF increased the expression of the VEGF-Trap proteins. In the low-GC content of VEGF-Trap ORFs, the Af signal peptide yielded higher expression than the human Vh signal peptide (7 folds difference) as indicated in Table 6. However, in the high-GC content of VEGF-Trap ORFs, there is no difference between the Af and the human Vh signal peptide as shown in Table 7 and by comparing FIG. 4A lane 3 (Af signal peptide) with FIG. 4A lane 4 and FIG. 4B lanes 1, 2, 3, and 4 (Vh signal peptide).

TABLE 7
ELISA measurement of VEGF-Trap expression level in media of
AAV2 vector transduced HEK293 cells

| Clone | scAAV2.N53 | VEGF-Trap | |
|---|---|---|---|
| No. | vectors | (ng/mL) | Fold |
| AMI059 | scCMV-SV40-intron-kozak-Af- | 456 | 7 |
| | VEGF-Trap | | |
| AMI066 | scCMV-SV40-intron-kozak-Vh- | 64 | 1 |
| | VEGF-Trap | | |
| AMI067 | scCMV-SV40-intron-kozak-Af- | 19993 | 312 |
| | VEGF-Trap-GC | | |
| AMI068 | scCMV-SV40-intron-kozak-Vh- | 19383 | 303 |
| | VEGF-Trap-GC | | |
| AMI119 | scCMV-SV40-intron-kozak-Vh- | 19019 | 297 |
| | VEGF-Trap-GC | | |
| AMI120 | scCMV-SV40-intron-kozak-Vh- | 18995 | 297 |
| | VEGF-Trap-GC | | |
| AMI130 | scCMV-SV40-intron-kozak-Vh- | 18507 | 289 |
| | VEGF-Trap-GC | | |

### Example 5: High Purity of VEGF-Trap Purified from HEK293 Cell Media

A 500 mL culture supernatant was harvested from an AAV2-VEGF-Trap (AMI067) transduced HEK293 cells and filtered through 0.2 μm OptiScale filter (Millipore) by peristaltic pump. The HiTrap™ 1 mL MabSelect™ PrismA Protein A column (GE Health Care Lifesciences) was first equilibrated with equilibration buffer (Tris-HCl pH 7.2, 150 mM NaCl) at flow rate of 1 mL/min with 5 column volume (CV). Afterwards the filtered supernatant was loaded into the column at the same flow rate using the AKTA explorer 100 system according to instruction manual. The column was washed with equilibration buffer for 10 CV at same flow rate before elution. AAV-Eylea protein was eluted with elution buffer (0.1 M Sodium Citrate Dihydrate pH 3.0) (Fisher Chemical) 6CV at 0.5 mL/min flow rate. Fractions were collected into tubes containing 0.1 volume of neutralization buffer (1 M Tris/HCl buffer pH 9.0). The eluted collection was desalted with 1×PBS using centrifugal filter (Millipore, Amico Ultra-4).

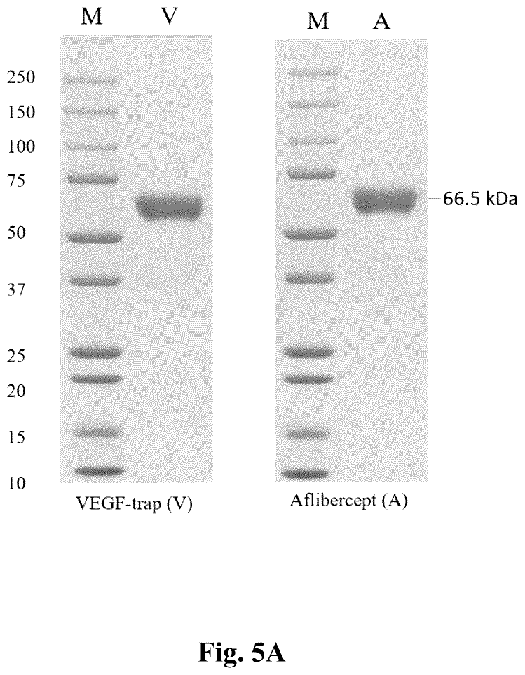

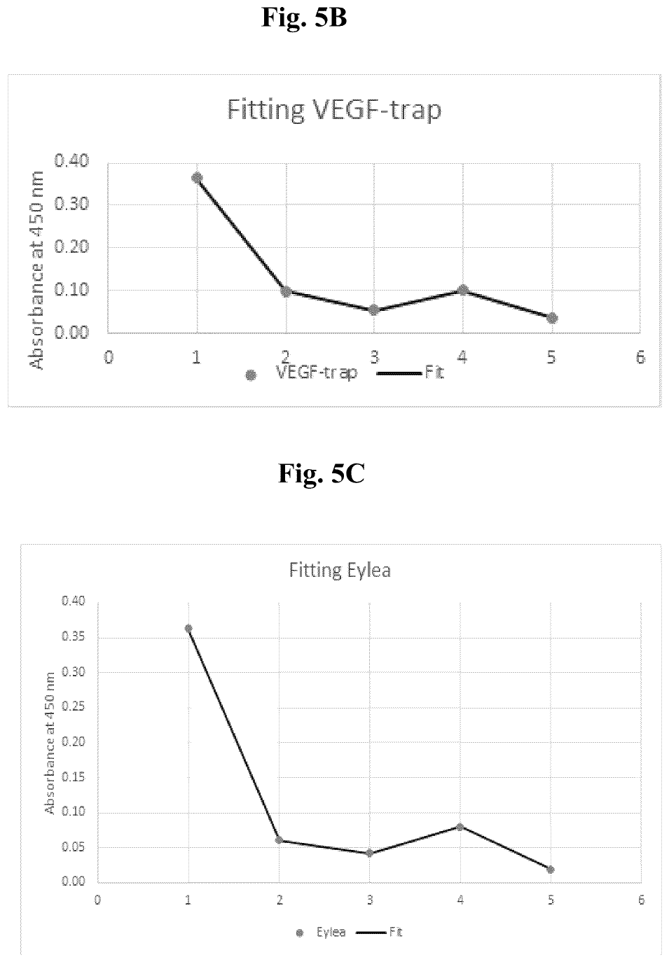

Media from HEK293 cells transduced with AAV2.53-AMI067 were subjected to column chromatography purification. A total of about 4.3 mg pure VEGF-Trap was obtained from about 500 mL of media harvested from scAAV2.53-AMI067 transduced HEK293 cells. SDS-PAGE and SimplyBlue staining show that the protein was pure and had the correct molecular weight of 66.5 kDa as the same size in gel as the commercial product (Regeneron Tarrytown, NY) shown in FIG. 5.

Purified AAV2.VEGF-Trap vector was used to transduce HEK293 cells at MOI of 500,000 vector genome (vg) per cells. VEGF-Trap produced and released into cell culture supernatant was analyzed daily using an operational protocol using commercial VEGF-A₁₆₅ as a coating antigen. VEGF-A₁₆₅ was expressed in HEK293 cells and purified to homogeneity (GeneScript, Cat #Z03073) and reconstituted into sterile Milli-Q water at 100 μg/mL. The ELISA procedures were: coating 96-well plate with 0.1 μg/mL VEGF final concentration per well in the coating buffer with a 50 μL volume; covering the plate with sealing; and putting the plate at 4° C. overnight. Next day, the ELISA procedures were: removing the plate; discarding the solution; tapping the plate on the paper towel to remove excess solution; and washing the plate thrice with wash buffer (300 μL). After last wash, the plate was tapped again on a fresh paper towel to remove as much buffer as possible. 300 μL of blocking buffer was added to each well using multichannel pipette. The plate was covered with sealing cover into 37° C. incubator for 2 hours. After incubation, the blocking buffer was discarded, and the plate was tapped on paper towel to remove excess buffer. Sample dilutions were prepared in blocking buffer by adding 50 μL into each well according to the scheme of the experiment. The plate was covered with sealing cover and placed back into the incubator for 1 hour (hr). After 1 hour, the solution was discarded, and the plate was washed with 300 μL of wash buffer 6 times. Excess solution was removed by tapping on paper towel as described above. Capture antibody was added at 1:40,000 dilution in blocking buffer and incubate at 37° C. for 1 hour. Solution was discarded, and washing procedure was repeated as necessary. Detection antibody was added at 1:40,000 dilution in blocking buffer at 37° C. for 1 hour. The solution was discarded, and the plate was washed with additional incubation (5 minutes in wash buffer) for last wash. The wash buffer was then discarded, and one additional wash was performed with PBS. 50 μL TMB was added. The plate was kept from direct light source for 15-20 min (it could be for fewer than 15 minutes) or until saturation of the signal was observed at highest concentration wells. 50 μL stop solution was then added, and the plate was read at 450 nm with 620 nm as reference within 15 minutes.

As seen from FIG. 10, the cell culture supernatant harvested from HEK293 cells transduced with AAV2.054-AMI120 secreted VEGF-Trap to approximately 800 ng/mL, while the HEK93 cells without transduction did not secrete VEGF-Trap. The production continues with increase of date of cultivation before the cells aged (FIG. 11).

VEGF-Trap expressed by transduction of HEK293 cells with AAV2.N53-VEGF-Trap vector was purified by the protein A affinity column chromatography. The column eluate was neutralized and buffer changed into 10 mM phosphate buffer (pH 7.0; 150 mM NaCl). The purity of the vector derived VEGF-Trap was determined by loading 1.5 μg/lane into the SDS-PAGE gel (10%) and stained with Coomassie blue R-250 and a single band at 66.5 kDa is seen in gel. The size was identical to that of commercial VEGF-Trap (FIG. 5A).

AVMX-110 derived VEGF-Trap binding affinity was measured by two methods for analysis of AVMX-110 derived VEGF-Trap binding to VEGF-A₁₆₅: ELISA and surface plasmon resonance (SPR). For the ELISA, binding of AVMX-110 derived VEGF-Trap and commercial aflibercept (Regeneron, Tarrytown, NY) to VEGF-A₁₆₅ was performed in parallel under the same conditions. The results showed both were the same in binding, with IC₅₀ of 159.1 ng/mL and 158.8 ng/mL respectively for AVMX-110 derived VEGF-Trap (A) and aflibercept (B) as shown in FIG. 5B and FIG. 5C. The binding constant (Kd) was calculated in nanomolar concentration using molecular weight of 115000 Da. The affinity of AVMX-110 derived VEGF-Trap was identical to that of commercial aflibercept (1.38 nM for both AVMX-110 derived VEGF-Trap and commercial aflibercept). Accordingly, the purified product functioned as the same as those from AAV2.VEGF-Trap.

Based on the biochemical and biological characterization, AVMX-110 is a qualified AAV vector carrying a VEGF-Trap gene for transduction of human cells including HEK293 and retina cell ARPE-19. The product of AAV2.N54-VEGF-Trap can produce VEGF-Trap in vivo similar to that of currently commercial aflibercept. Therefore, AVMX-110 (AAV2.N54-VEGF-Trap) can be used for long term expression for the treatment of neovasculogenic retina disorders in vivo.

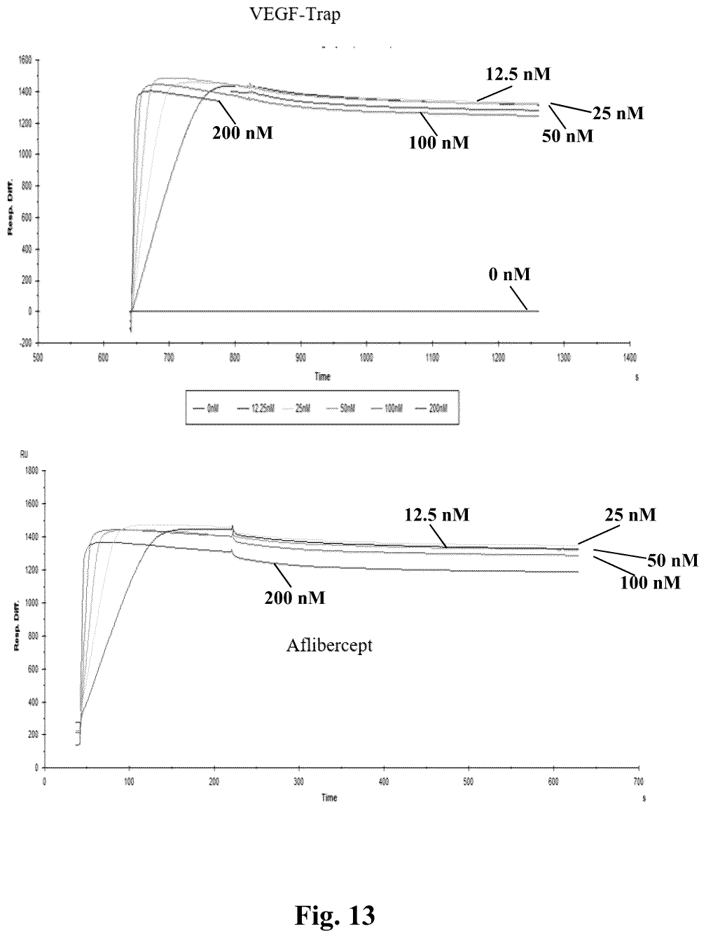

Interaction between AVMX-110 derived VEGF-Trap and human VEGF-A₁₆₅ was analyzed by the surface plasmon resonance (SPR) method. 15 μl of 200 nM of AVMX-110 derived VEGF-Trap or aflibercept in phosphate buffered saline with 0.01% Tween 20 (PBS-T) was bound to a sensor chip protein A. AVMX-110 VEGF-Trap and aflibercept were bound to HEK293 expressed human VEGF-A₁₆₅ tightly with a Kd at 33 pM and 55 pM respectively (FIG. 13 and Table 8).

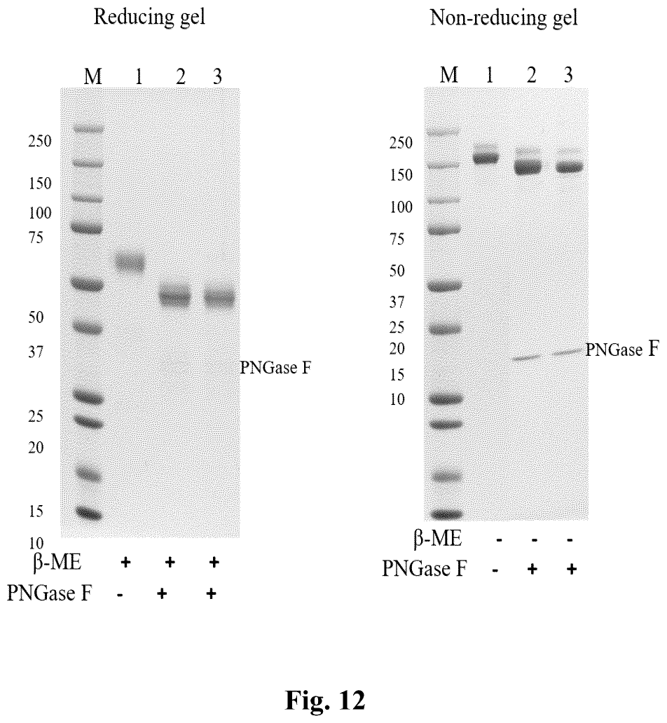

Proliferation inhibition of HUVEC cells was used to determine the potency AVMX-110 in vitro. Briefly, AVMX-110 was firstly used to transduce HEK293 cells, which were cultivated at 37° C. for 4 days. The cell culture supernatant was harvested and purified via affinity column chromatography. The purified VEGF-Trap was compared to the commercial aflibercept (Eylea, Regeneron Pharmaceuticals, Tarrytown, NY) for blocking human VEGF-A₁₆₅ induced HUVEC cell proliferation. The assay was repeated in duplicates. Briefly, in a 96-well microplate, HUVEC cells (ATCC, Cat #, CRL-1730, Manassas, VA) was seeded at 5000 cells/well in 100 μL cell culture medium and incubated in a CO₂ incubator with 5% CO₂ and 90% humidity. Cells were allowed to adhere for 24 hours, and cell culture medium was removed and replaced with 100 mL/well fresh assay medium (vascular cell basal medium with 1% dFBS) containing 20 ng/mL human VEGF-A₁₆₅ and followed by the purified VEGF-Trap at molar ratio (VEGF-A₁₆₅/VEGF-Trap) of I/O, 1:2, 1:5, 1:10 and 1:100 individually or 0, 101, 253, 506 and 5060 ng/mL respectively. Both the commercial aflibercept (40 mg/mL) and AAV2.VEGF-Trap were diluted to the same ratio. The plate was incubated for 72 hours followed by measurement procedures. The assay media in each well were removed and fresh assay medium (90 μL) along with 10 μL of WST-8 (Dojindo Molecular Technologies, Inc., Cat. #CK04-11, Rockville, MD) was added. The plate was read every hour post addition. Data demonstrated that AAV2.VEGF-Trap inhibition of HUVEC cell proliferation was VEGF-Trap concentration dependent. The potency is equally effective as aflibercept (FIG. 12).

### Example 6: Determination of Glycosylation of AAV2 Derived VEGF-Trap

AAV2.N54-AMI120 derived VEGF-Trap protein purified from cell culture supernatant of HEK293 transduced with AVMX-110 at a MOI of 100,000 was purified via affinity column chromatography. The purified VEGF-Trap was treated with recombinant F. meningosepticum PNGase F enzyme which was ideal for removing all N-linked carbohydrates from glycoproteins. The treatment was carried out with the commercial aflibercept. The treatment mix was separated by SDS-PAGE gel shift analysis. In the presence of NPGase F, the carbohydrates were removed from the glycoprotein via N-linkage, then the deglycosylated protein migrated faster because of decreased molecular weight (FIG. 12).

### Example 7: Biacore Binding Assay for Binding Affinity (KD) of VEGF-A₁₆₅ with Vector Expressed VEGF-Trap and Afflibercept (Elyea)

VEGF-Trap protein was expressed by HEK293 cell transfected with expression vector, AAV2.N54-AMI120, and purified by a protein A affinity column chromatography. The eluate was neutralized and concentrated to the concentration of 4.31 mg/ml. Aflibercept was commercial product, which was diluted into 4 mg/ml. VEGF-A₁₆₅ was obtained from Genescript (GeneScript, Cat #Z03073, Nanjing China), which was reconstituted into 1 mg/ml. Working solution of each stock protein was prepared at 200 nM prior to experimentation. Molarity concentrations were calculated using MW: 115 kDa for VEGF-Trap and aflibercept, 45 kDa for VEGF-A₁₆₅. Biacore operation was started with coating a protein A sensor chip with VEGF-Trap or aflibercept at 200 nM by injecting 15 μL at 5 μL/min, followed by injection of 90 μL of serially diluted VEGF-A₁₆₅ solutions of 200, 100, 50, 25, 12.5, and 0 nM at a flow rate of 30 μL/min. Binding kinetic simulation was analyzed with Biacore evaluation software. The KD of AAV2.N54-AMI120 derived VEGF-Trap is 33 pM for its binding to VEGF-A₁₆₅. The KD of aflibercept was 55 pM for its binding to VEGF-A₁₆₅. Binding kinetic parameters shown in Table 8 and FIG. 13 demonstrate that the AAV2.N54-AMI120 derived VEGF-Trap are similar to those of aflibercept.

TABLE 8
Kinetic binding affinity determined by Biacore assay

| | VEGF-Trap | Aflibercept |
|---|---|---|
| | AAV2.N54-AMI120 | Commercial Product |
| | expressed in | (Regeneron |
| Sources | HEK293 cells | Pharmaceuticals) |
| Ligand | VEGF-Trap | aflibercept |
| Analyte | VEGF-A₁₆₅ | VEGF-A₁₆₅ |
| KD(M) | 3.3e−11 | 5.45e−11 |
| KA(1/M) | 3e+10 | 1.83e+10 |
| Kd(1/s) | 2.02e+04 | 1.88e−04 |
| Ka(1/Ms) | 6.07E+06 | 3.45E+06 |
| Rmax(RU) | 1.43E+03 | 1.41E+03 |

### Example 8. Binding Affinity of Vector Expressed VEGF-Trap with rhVEGF-A₁₆₅

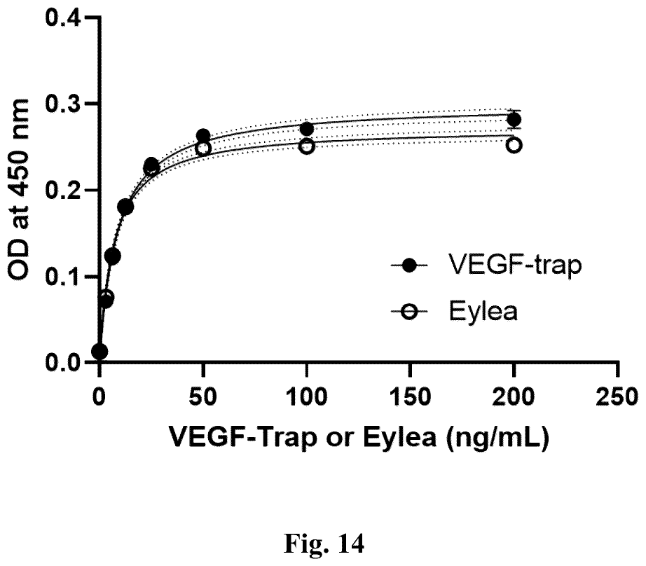

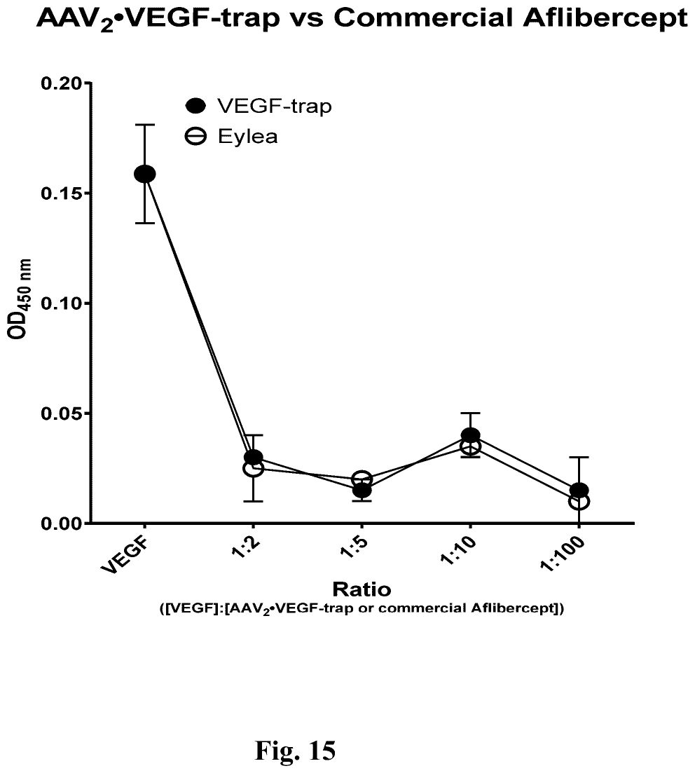

The assay for determining binding affinity was performed. Briefly, purified from AAV2.N54-VEGF-Trap expressed VEGF-Trap was assayed for its binding affinity to rhVEGF. A 96-well plate was coated with rhVEGF-A₁₆₅ at 0.1 μg/mL at 2-8° C. overnight. The plate was washed 3×300 μL with washing buffer (0.05% Tween−20 in PBS, pH 7.3±0.1) and blocked with blocking buffer containing 1% casein for 120 min. Serially diluted VEGF-Trap or aflibercept (the same lot as mentioned above) at 200, 100, 50, 25, 12.5, 6.25, and 3.1 ng/mL, was added individually to each well at 100 μL/well and incubated at 37±1° C. for 60 minutes. Diluted (1:40,000) biotinylated rabbit anti-human IgG1 Fc antibody solution was added at 100 μL/well after 3×300 μL/well of washing buffer. Further incubation at 37±1° C. for 60 minutes and washing with 3×300 μL/well of washing buffer were performed. Finally, to each well, streptavidin-HRP, 1:40,000, was added at 100 μL/well and incubated at 37±1° C. for 60 minutes. The substrate TMB solution after 3×300 μL/well washing, was added and incubated at 37±1° C. for 15 minutes followed by stopping with stop solution 100 μL/well. The plate was read at 600 nm wavelength in a microplate reader (Molecular Device, Sunnyvale, CA). Results from assay showed that the binding affinity of VEGF-Trap and aflibercept to rhVEGF-A₁₆₅ was 75 pM and 60 pM separately, demonstrating similar both proteins binding to rhVEGF-A₁₆₅ (FIG. 14 and Table 9).

TABLE 9
Binding affinity analysis using Biacore assay

| | | |
|---|---|---|
| Vmax | 0.2999 | 0.2724 |
| Kd (ng/mL) | 8.633 | 6.856 |
| Kd (nM) | 0.075 | 0.060 |
| pM | 75 | 60 |

### Example 9. Inhibition of HUVEC Cell Proliferation Stimulated by VEGF-A₁₆₅

The purified VEGF-Trap was compared to the commercial aflibercept (Eylea) (Regeneron Pharmaceuticals, Tarrytown, NY) for blocking human VEGF-A induced HUVEC cell proliferation. The assay was repeated in duplicates. Briefly, in a 96-well microplate, HUVEC cells (ATCC, Cat #. CRL-1730, Manassas, VA) were seeded at 5000 cells/well in 100 μL cell culture medium and incubated in a CO₂ incubator with 5% CO₂ and 90% humidity. After cultivation for 24 hours, cell culture medium was removed and replaced with 100 mL/well fresh assay medium containing 20 ng/mL human VEGF-A₁₆₅ and following by the purified VEGF-Trap at molar ratio (VEGF-A₁₆₅/VEGF-Trap) of 1:0, 1:2, 1:5, 1:10, and 1:100 individually or 0, 101, 253, 506 and 5060 ng/mL respectively. The commercial aflibercept (40 mg/mL) and AAV.VEGF-Trap were diluted in the same ratio. The assay medium was vascular basal medium with 1% dFBS, and the plate was incubated for 72 hours followed by measurement procedures. The assay media in each well was removed, and fresh assay medium (90 μL) along with 10 μL of WST-8 (Dojindo Molecular Technologies, Inc., Cat. #CK04-11, Rockville, MD) was added. The plate was read every hour post addition. Data demonstrated that AAV.VEGF-Trap inhibited HUVEC cell proliferation a VEGF-Trap concentration dependent. The potency was equally effective as aflibercept (FIG. 15).

### Example 10. In Vivo Evaluation of AAV2.N54-AMI120VEGF-Trap Protection of Choroidal Damage Caused by Laser Induced Neovascularization

Efficacy and pharmacokinetics of AAV2.N54-AMI120-VEGF-Trap were tested using mouse LCNV model. The study of the cohorts is shown in Table 10.

TABLE 10
Experimental design of AAV2.N54-AMI120-VEGF-Trap
in mouse laser choroidal neovascularization

| | | OU | | | | |
|---|---|---|---|---|---|---|
| | No. of | Treatment/ | Volume/ | Treatment | CNV | Experimental |
| Group | Animals | Dose | Route | Day | Induction | Endpoints |
| 1 | 8 | AAV2-GFP | 1.0 μL | Day −3 | OU: CNV | DAY 7: |
| | | 4e+8 vg/eye | IVT | | Laser Day | Fluorescein |
| 2 | 8 | Aflibercept | | | 0 | angiography |
| | | (Eylea ®) 200 μg | | | | Flatmount |
| 3 | 8 | AVMX110 | | Day −28 | | analysis and |
| | | Low Dose | | | | necropsy (n = 6 |
| | | 2e+7 vg/eye | | | | mice/group or 12 |
| 4 | 8 | AVMX110 | | | | eyes per group) |
| | | Medium Dose | | | | Dissect eye cups |
| | | 4e+8 vg/eye | | | | and homogenize |
| 5 | 8 | AVMX110 | | | | for ELISA (n = 2 |
| | | High Dose | | | | mice/group for |
| | | 1.6e+10 vg/eye | | | | Groups 1-5). |
| | | | | | | Collect serum of |
| | | | | | | each animal for |
| | | | | | | ELISA |
| 6 | 2 | NA | NA | NA | NA | DAY 7: |
| | | | | | | Dissect eye cups and |
| | | | | | | homogenize for ELISA |
| | | | | | | (n = 2 mice/4 eyes) |

Mouse (Mus Musculus)/C57BL/6 (Charles River or Tacomic Farms) of 8-12 weeks old, 15-25 grams each, are used in the study. The animals are randomly divided into groups, 5 animals per group. Each animal is injected bilaterally, 10 eyes per group, with the test article intravitreally (IVT) 28 days prior to lasering. The test article of AAV2.N54AMI120 is tested at three doses, a high (1.6e+10), medium (4e+8) and low dose (2e+7) per microliter (μL). As a control, AAV2.N54-GFP is used as a vector transmission control. AAV2.N54-AMI120 formulation buffer (vehicle) is used as a negative control. The mice are dosed (IVT administration) with test articles and controls 28 days prior to laser injury control animals are dosed 3 days prior to lasering for aflibercept and vehicle. Seven (7) days post lasering, all groups are analyzed for fluorescent angiography, VEGF-Trap level serum and eye cups. VEGF-Trap concentration are detected using ELISA. According to in vitro potency results mentioned above, the animals dosed with AAV2.N54-AMI120 can gain protection from inflammation and wound healing responsive neovascularization, while the AAV2.N54-GFP and vehicle controls may not have any protection.

### Example 11. Efficacy of Modified Adeno-Associated Virus (AAV2) Encoding Aflibercept (AVMX-110) in a Laser-Induced Choroidal Neovascularization (CNV) Model in Mice

The objective of this study was to evaluate the inhibition of neovascularization in a laser-induced model of choroidal neovascularization (CNV) in the mouse using the adeno-associated viral (AAV2) vectors. Table 11 illustrates the experimental design for this study.

TABLE 11
Experimental design for examining CNV in mice

| | No. of | OU Treatment/ | Volume/ | Treatment | | |
|---|---|---|---|---|---|---|
| Group | Animals | Dose | Route | Day | CNV Induction | Experimental Endpoints |
| 1 | 8 | Formulation | 1.0 μL | Day −3 | OU: CNV | DAY 7: |
| | | Buffer (Vehicle) | IVT | | Laser Day | Color/Cobalt |
| | | N/A | | | 0 | Blue Fundus |
| 2 | 8 | Aflibercept | | | | Imaging (Group 3 only): Day 7, |
| | | (Eylea)) 40 μg | | | | Fluorescein angiography: |
| 3 | 8 | AAV2-GFP | | Day −28 | | n = 8 mice/group |
| | | 1.6e10 vg/eye | | | | Terminal blood collection |
| 4 | 8 | AVMX110 | | | | (serum): n = 8 mice/group |
| | | 2e7 vg/eye | | | | Flatmount analysis: n = 6 |
| 5 | 8 | AVMX110 | | | | mice (12 eyes)/group |
| | | 4e8 vg/eye | | | | Dissect and homogenize eye |
| 6 | 8 | AVMX110 | | | | cups for ELISA: n = 2 |
| | | 1.6e10 vg/eye | | | | mice (4 eyes)/group |
| 7 | 2 | N/A | N/A | N/A | N/A | DAY 7: |
| | | | | | | Terminal blood collection |
| | | | | | | (serum): n = 2 mice |
| | | | | | | Dissect and homogenize eye |
| | | | | | | cups for |

Abbreviations: CNV—choroidal neovascularization; IVT—Intravitreal; OU - both eyes; N/A—not applicable.

This laser choroidal neovascularization (CNV) study was conducted in mice (Mus Musculus) C57BL/6 strain. The study met the acceptance criteria for a valid, non-GLP status study, because average CNV lesion size evaluated via fluorescein angiography (FA) and Flatmount immunofluorescence was greatly reduced in aflibercept treated animals when compared to formulation buffer or a control vector (GFP-AAV2) following IVT injections.

Test articles were provided in a ready-to-inject format and stored at <−70° C. until use. Aflibercept, which was stored refrigerated (2-8° C.) and injected neat without dilution. Thirty minutes prior to injection, each test article was thawed in a 37° C. water bath for 20 minutes and vortexed to reduce virus aggregation.

On Days −28 (Groups 3-6) and −3 (Groups 1 and 2) prior to injection, mice were given buprenorphine 0.01-0.05 mg/kg subcutaneously (SQ). Animals were then tranquilized for the intravitreal injections with inhaled isoflurane and one drop of 0.5% proparacaine HCL was applied to both eyes. The conjunctiva was gently grasped with Dumont #4 forceps, and the injection was made using a 33 G needle and a Hamilton Syringe. After dispensing the syringe contents, the syringe needle was slowly withdrawn. Following the injection procedure, 1 drop of Ofloxacin ophthalmic solution was applied topically to the ocular surface with eye lube.

On day 0, mice were given buprenorphine 0.01-0.05 mg/kg SQ. A topical mydriatic (1.0% Tropicamide HCL, and 2.5% phenylephrine HCL) was applied at least 15 minutes prior to the laser procedure. The mice were tranquilized with an intraperitoneal (IP) injection of ketamine/xylazine. The cornea was kept moistened using topical eyewash, and body temperature was maintained using hot pads. A 532 nm diode laser delivered through a slit-lamp was used to create 4 single laser spots surrounding the optic nerve. Both mouse eyes received laser treatment at predetermined time intervals. Eye lube was placed (OU) after laser.

Morbidity and mortality were observed daily along with cage-side observations, with particular attention paid to both eyes. Four (4) animals died post-dosing but before lasering in Groups 2 (#'s 210, 212, and 216) and 4 (#432). Two (2) additional animals died prior to final tissue collections but after lasering in Groups 4 (#430) and 6 (#644).

### Fundus Imaging

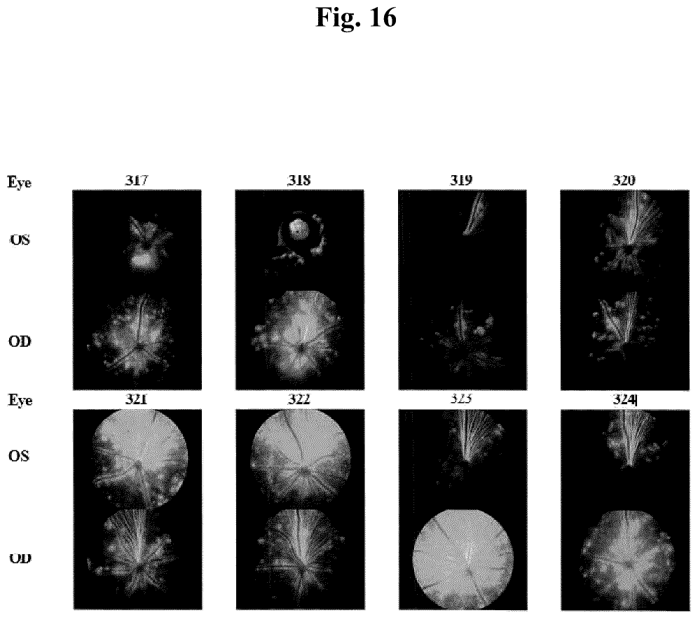

Both color and cobalt blue (EGFP expression) fundus imaging were performed on both eyes of Group 3 (AAV2-GFP; 1.6e10 vg/eye) animals on Day 7 after laser and prior to fluorescein angiography (FA). Animals were given a cocktail of tropicamide (1.0%) and phenylephrine (2.5%) topically to dilate and proptose the eyes, and topical eye anesthetic was applied to the eyes (proparacaine 0.5% or similar). Color fundus photography was followed by cobalt blue photography. Intensity settings were kept constant between animals during acquisition. As shown in FIG. 16, GFP was observed in all eyes, with the exception of 318 OS, which had a cataract that prevented imaging of the fundus. The observed cataract may have been caused by trauma to the lens during the IVT injection.

### Fluorescein Angiography (FA)

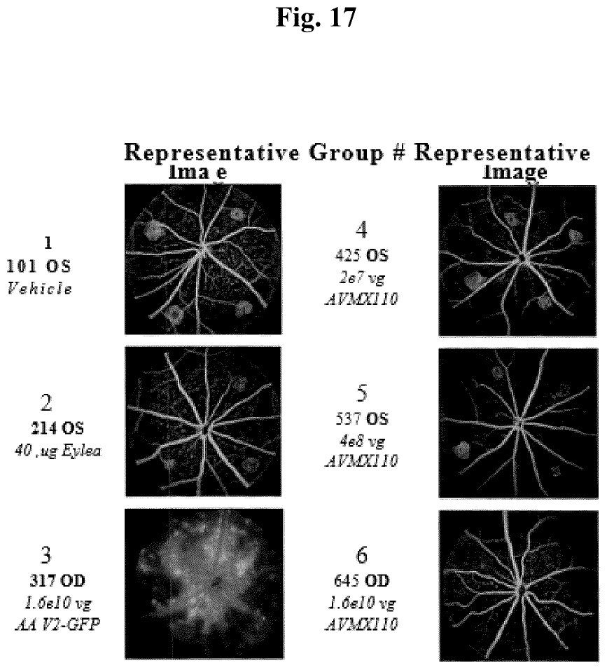

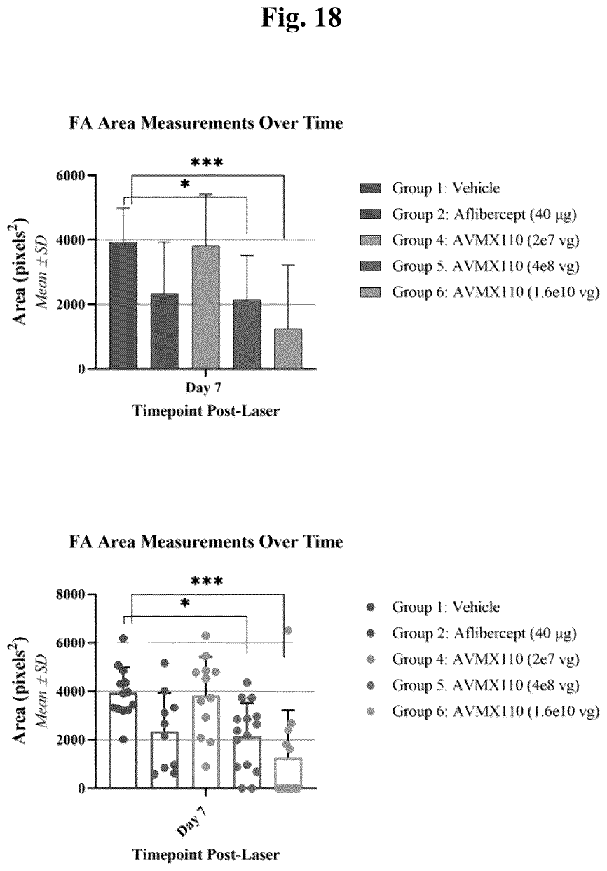

FA was performed on both eyes on Day 7 post-laser. Mydriasis for FA was achieved using a topical drop in each eye 15 minutes prior to examination (1.0% tropicamide HCL, and 2.5% phenylephrine HCL). The mice were tranquilized with an IP injection of ketamine/xylazine. Retinal photography was performed approximately 1 minute after intravenous sodium fluorescein injection (1% stock solution; 12 mg/kg). Fluorescein leakage was analyzed with ImageJ software; representative images are shown in FIG. 17, which illustrates day 7 fluorescein angiography. Representative images from each group are shown. Measurement of lesions in Group 3 was not feasible as the GFP expressed by the AAV precluded analysis. Angiograms were used to quantify lesion area as pixels ±standard deviation (SD) and the results are displayed in FIG. 18. On study day 7 post-laser, Group 6 (AVMX110, 1.6e10 vg/eye) had the smallest average lesion area while the vehicle group had the largest lesion area.

### Ocular Tissue Collection and Processing

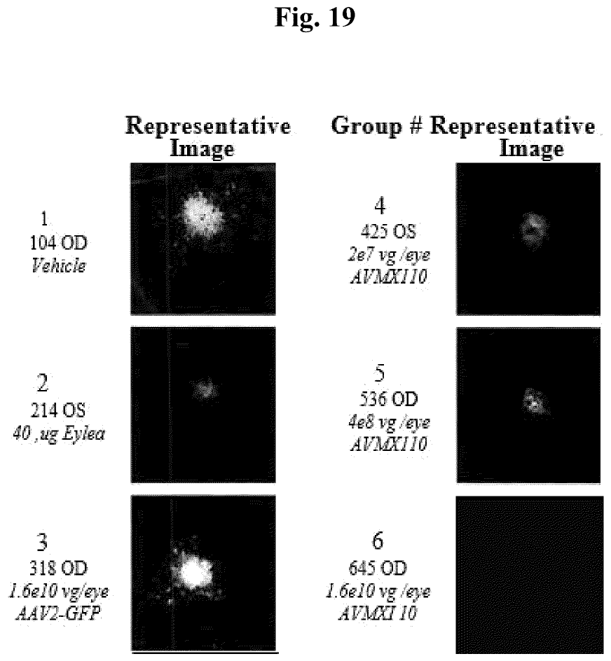

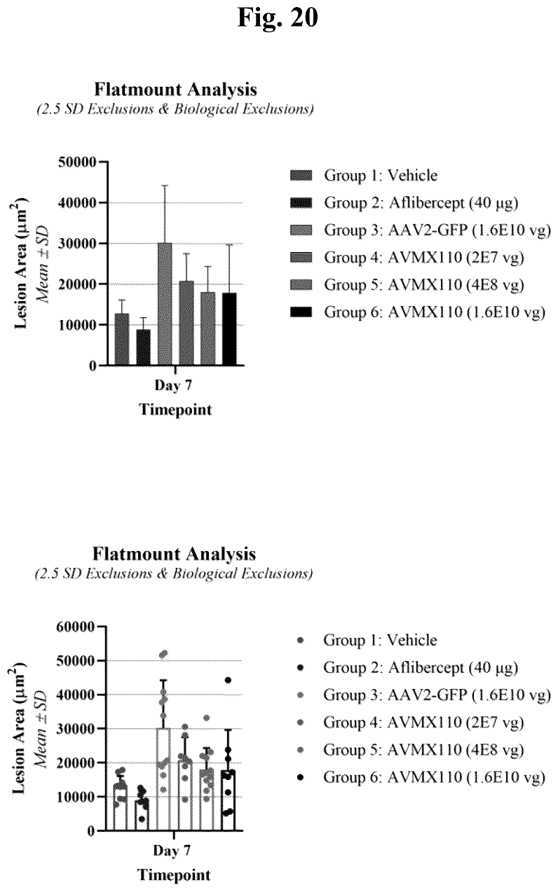

On Day 7, animals were humanely euthanized, and eyes were enucleated and immediately fixed in 4% paraformaldehyde (PFA) in phosphate-buffered saline (PBS) overnight at 4° C. Using a dissecting microscope, each eye was trimmed of extraneous tissue and the anterior segment and lens were removed. The retina was detached and removed from the optic nerve head with fine curved scissors. Eye cups were rinsed with cold immunocytochemistry (ICC) buffer (PBS+0.5% BSA and 0.2% Tween 20) and blocked for at least 6 hours in ICC buffer. Eye cups were then placed in cold ICC buffer containing the 4′,6-diamidino-2-phenylindole (DAPI; nucleus), Phalloidin DyLight 550 (actin cytoskeleton) and Isolectin IB4-DyLight 649 (blood vessels) and incubated at 4° C. with gentle rotation for 2.5 hours. Tissues were washed, and radial cuts were made toward the optic nerve head, avoiding lesions, and tissue was flat-mounted. Two-dimensional (2D) fluorescent microscopy images were acquired on an Olympus Bx63 fluorescent scope and neoformed vessels were quantified using the Isolectin IB4 signal area (μm2) and CellSens software. Representative images are shown in FIG. 19. Quantification of isolectin area is shown in FIG. 20. Injection of aflibercept reduced the lesion area. Group 3 eyes had the largest lesion area, though this may have been impacted in part by the GFP driven by the AAV2 construct. When compared to the AAV2-GFP from Group 3, AVMX-110 reduced lesion size at all doses tested (Groups 4-6).

This study was conducted to determine the inhibition of retinal vascular leakage after IVT administration of test articles (Groups 3-6) and a positive control (aflibercept, Group 2) in mice. AAV2 vectors (Groups 3-6) were injected on day 28 to ensure adequate expression prior to induction of laser CNV; vehicle and aflibercept (Groups 1 and 2) were injected on day 3. Based on the tests performed and the objectives of this study, the aflibercept control resulted in decreased lesion size at Day 7 post-laser when compared to vehicle. In comparison to Aflibercept (positive control; 40.5% reduction in fluorescein area), the test articles in Groups 5 and 6 demonstrated equivalence or superiority by at least one endpoint analysis, with the highest dose of AVMX-110 (1.6e10 vg/eye) having a 68% reduction in vascular leakage for the angiography endpoint. Analysis of lesion size in Flatmount revealed that all AAV2-preparation injected eyes had larger lesions than vehicle or aflibercept-injected eyes. However, when AVMX-110 was compared to AAV2-GFP, lesion sized was reduced at all three AVMX-110 dose levels, with the highest dose of AVMX-100 leading to a 40% reduction in isolectin area.

### Example 12. Analysis of AVMX-110 and AVMX-116

Example 12 illustrates a comprehensive analysis of data that lead to the candidate selection for AVMX-110 and AVMX-116. The strategy involved construct design, cloning, sequencing, production, purification and bioanalytics. The data was analyzed using one-way ANOVA method. Candidates that had best expression, potency and penetrability (in vivo) were selected on following criteria: Gene of Interest (GOI); capsid; and AAV serotype.

### Capsid Engineering and Selection

Wild type AAV2 capsid had been engineered methodologically to introduce mutations at specific sites to increase the penetrability or expression of GOI. The following list of mutations were introduced and screened in mouse and/or pig models to check for expression and penetrability (Table 12). The sequences and positions were inserted with a short peptide fragment in the loop 4 of AAV2-VP1 (Table 12), and the plasmid names are listed in Table 13.

TABLE 12
AAV construct with modified capsid region

| Construct | | SEQ ID | HEK293 | ARPE19 |
|---|---|---|---|---|
| ID | Modified Capsid Region | NO: | Cell | Cell |
| Wild type | No insertion | N/A | ++++ | ++++ |
| (Wt) | | | | |
| V466 | TPSG(453): LALGETTRPA: TTTQ | 40 | ++ | ++ |
| V467 | QRGN(587): LALGETTKPA: RQAA | 41 | ++++ | ++++ |
| V468 | NTPS(452):- ALGETTKP: GTTT | 42 | ++ | ++ |
| V471 | NTPS(452):- ALGETTKP: GTTT; | 43 | ++ | ++ |
| | QRGN(587): LALGETTKPA: RQAA | 41 | | |
| AMI051 | QRGN(587): LKLGQTTKPK: RQAA | 44 | + | ND |
| AMI052 | QRGN(587): LALGQTTKPK: RQAA | 45 | | |
| AMI053 | QRGN(587): LKLGQTTKPA: RQAA | 46 | ++++ | ++++ |
| AMI054 | QRGN(587): LALGQTTKPA: RQAA | 47 | ++++ | ++++ |
| AMI055 | R585_588A | N/A | + | ND |
| AMI056 | R585A | N/A | ++ | ND |
| AMI057 | R588A | N/A | ++ | ND |
| AMI097 | TPSG(453): LALGQTTKPA: TTTQ | 48 | ND | ND |
| AMI098 | QRGN(587): LALGQTTEPA: RQAA | 49 | ND | ND |
| AMI099 | RGNR(588): LALGQTTKPA: QAAT | 50 | ND | ND |
| AMI100 | NLQR(585): LALGQTTKPA: GNRQ | 51 | ND | ND |
| AMI101 | QRGN(587): VALGQTTKPA: RQAA | 52 | ND | ND |
| AMI102 | LQRG(586): LALGESTARG: NRQA | 53 | ND | ND |
| AMI103 | LQRG(586): LALGETSKRA: NRQA | 54 | ND | ND |
| AMI104 | LQRG(586): LALGQSTKPA: NRQA | 55 | ND | ND |
| AMI105 | NTPS(452): LALGQTTKPA: GTTT | 56 | ND | ND |
| AMI106 | TPSG(453): LALGQTTKPA: TTTQ | 57 | ND | ND |
| | QRGN(587): LALGQTTKPA: RQAA | 47 | | |
| AMI107 | QRGN(587): LALGQTTKPA | 58 | ND | ND |
| | LALGQTTKPA: RQAA | | | |
| AMI110 | QRGN(587): VKLGQTTKPA: RQAA | 59 | ND | ND |

TABLE 13
List of different AAV2 capsid mutations screened in animal models

| Construct | Description | Study/Animal |
|---|---|---|
| AMI051 | pFB-inCap2-587-KQKK-inRep- | 20-AVI-002/Mouse |
| | kozak-hr2 | |
| AMI052 | pFB-inCap2-587-QKK-inRep- | 20-AVI-002/Mouse |
| | kozak-hr2 | |
| AMI053 | pFB-inCap2-587-KQK-inRep- | 20-AVI-002 & 005/ |
| | kozak-hr2 | Mouse & Pig |
| AMI054 | pFB-inCap2-587-QK-inRep- | 20-AVI-002 & 005/ |
| | kozak-hr2 | Mouse & Pig |
| AMI055 | pFB-inCap2-R585_588A-inRep- | 20-AVI-002/ |
| | kozak-hr2 | Mouse |
| AMI056 | pFB-inCap2-R585A-inRep- | 20-AVI-002/ |
| | kozak-hr2 | Mouse |
| AMI057 | pFB-inCap2-R588A-inRep- | 20-AVI-002/ |
| | kozak-hr2 | Mouse |
| AMI097 | pFB-inCap2-453QK-inRep-hr2 | 20-AVI-003/Mouse |
| AMI099 | pFB-inCap2-588QK-inRep-hr2 | 20-AVI-003/Mouse |
| AMI101 | pFB-inCap2-587VQK-inRep-hr2 | 20-AVI-003 & -005/ |
| | | Mouse & Pig |
| AMI102 | pFB-inCap2-586SARG-inRep-hr2 | 20-AVI-003/Mouse |
| AMI103 | pFB-inCap2-586SKR-inRep-hr2 | 20-AVI-003/Mouse |
| AMI104 | pFB-inCap2-586QSK-inRep-hr2 | 20-AVI-003 & -005/ |
| | | Mouse |
| AMI105 | pFB-inCap2-452QK-inRep-hr2 | 20-AVI-003 & -005/ |
| | | Mouse & Pig |
| AMI106 | pFB-inCap2-453_587QK-inRep-hr2 | 20-AVI-003/Mouse |
| AMI107 | pFB-inCap2-587QKx2-inRep- | 20-AVI-003/Mouse |
| | kozak-hr2 | |
| AMI110 | pFB-inCap2-587VKQK-inRep-hr2 | 20-AVI-003/Mouse |
| V104 (wt) | pFB-inCap2-inRepOpt-Kan | 20-AVI-002 & -003/ |
| | | Mouse |
| V226 (+) | pFB-inCap2-7m8-GFP WT | 20-AVI-002, 003 & 005/ |
| | | Mouse & Pig |
| V467 | pFB-inCap2-587RtoK-GFP WT | 20-AVI-002/Mouse |

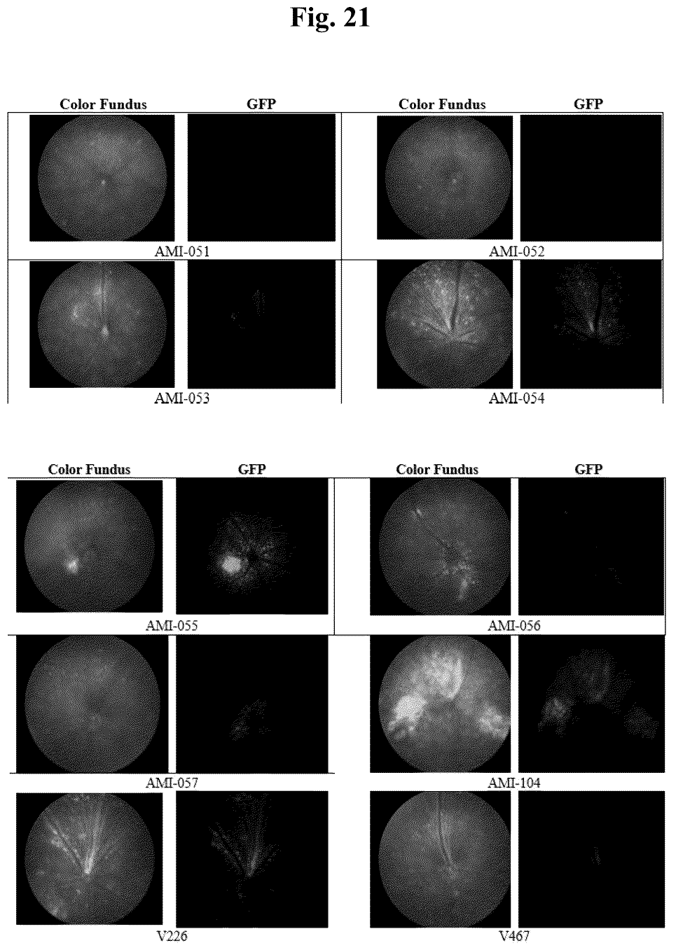

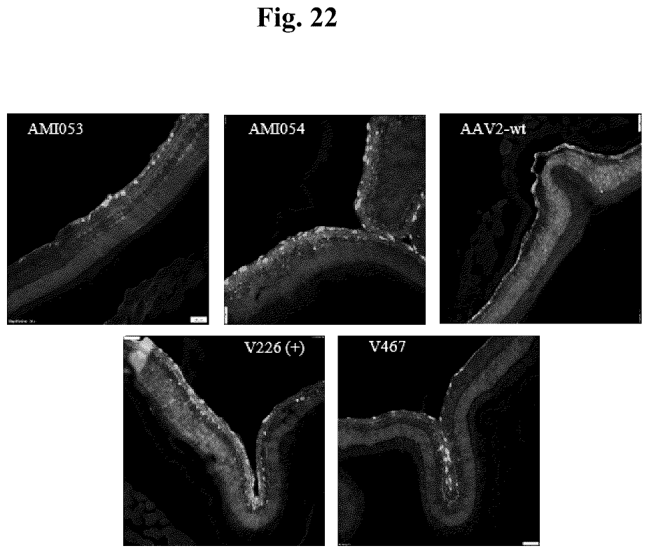

2E+10 vg/eye of different constructs were injected in the mouse model. FIG. 21 shows the fundus images all the groups. These images were noted on day 23 after injection of constructs. V226 was the positive control and showed good expression. One eye from each group was further analyzed by immunohistochemistry (FIG. 22), and eyes injected with construct AMI-053, AMI-054, AMI104 and V226 showed bright GFP expression. AMI054 particularly showed uniform expression in Ganglion cell layer (GCL), inner plexiform layer (IPL), and outer nuclear layer (ONL). In the other mouse study, the rest of the constructs mentioned previously were analyzed. Similar to the 20-AVI-002, 2E+10 vg/eye construct was injected into the mouse eyes. The fundus imaging was done on day 24 (FIG. 23).

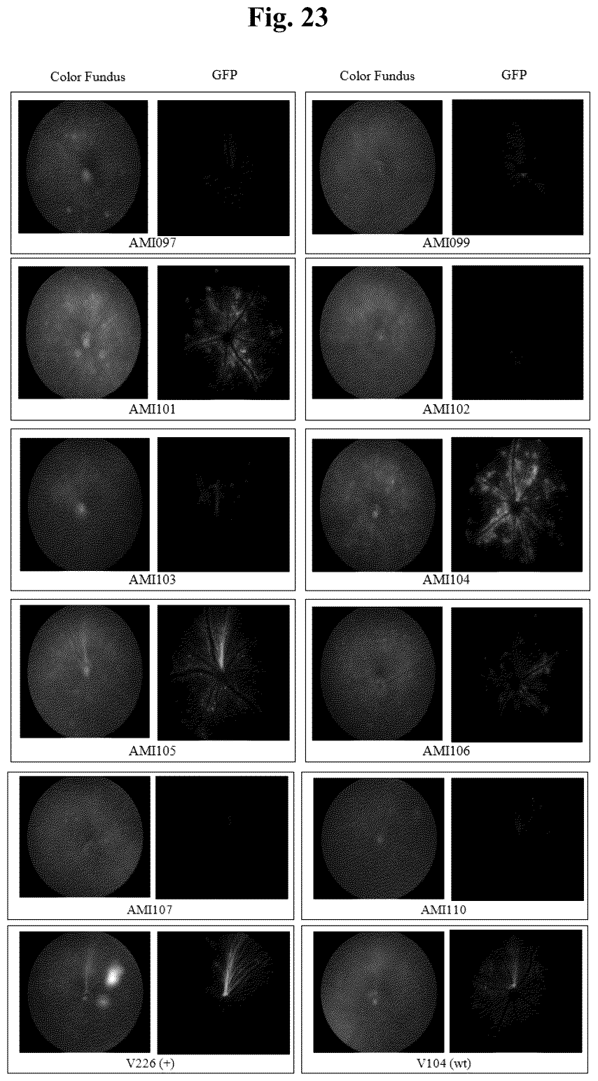

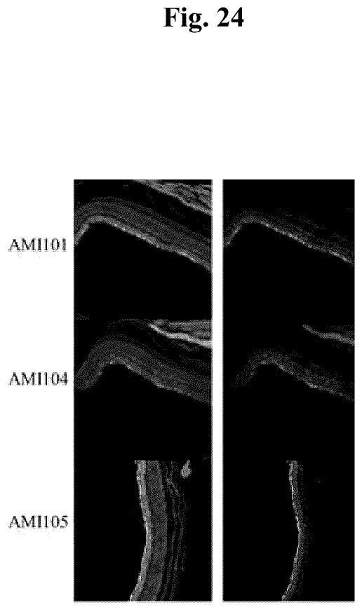

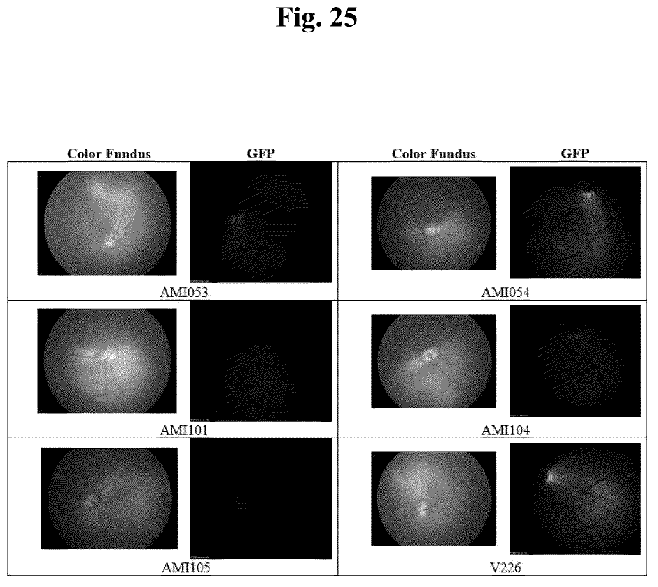

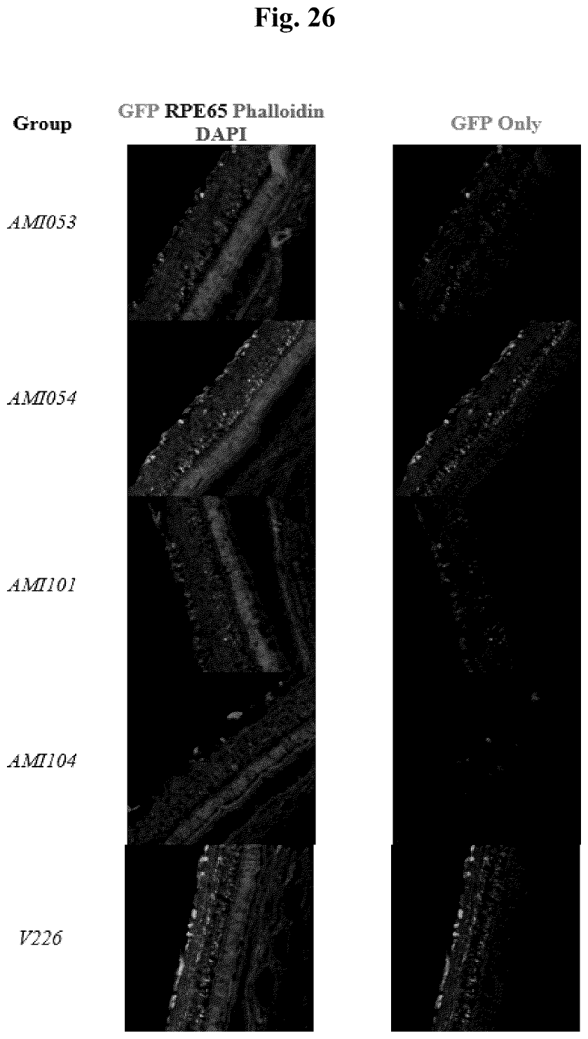

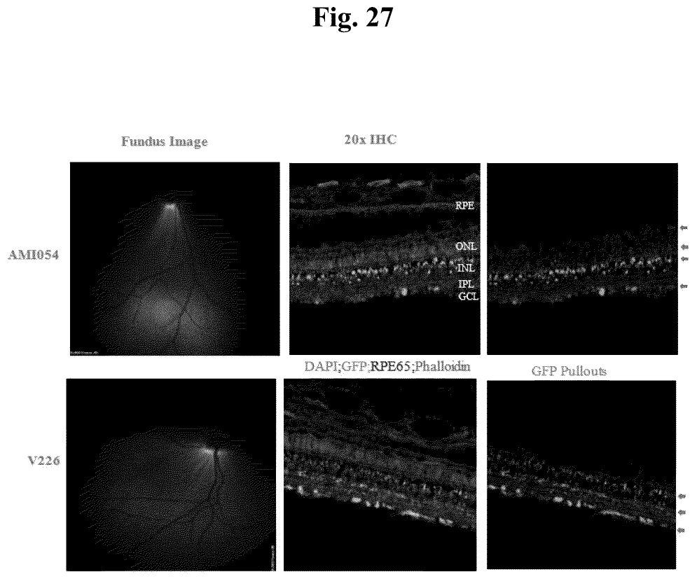

AMI101, AMI104 and AMI105 were subjected to the immunohistochemistry (FIG. 24) on day 28, and AMI104 showed uniform distribution of GFP expression in GCL, IPL, inner nuclear layer (INL), outer plexiform layer (OPL), ONL, inner segment (IS), and outer segment (OS). From the mouse studies, several candidates (AMI053, AMI054, AMI101, AMI104, AMI105 and V226) were picked up for additional pig study. Each pig eye was injected with 1E+11 vg/eye. FIG. 25 shows the fundus images of the animals in the pig study. AMI053 showed faint GFP expression similar to the results observed in the mouse study. AMI054 showed uniform and bright expression similar to V226. To further confirm the expression and penetrability of the constructs, immunohistochemistry was done on the cryosections generated on day 28 after injection (FIG. 6) similar to the mouse studies. According to the IHC data, AMI054 and V226 showed brightest, broadest and consistent GFP expression. V226 showed intense GFP expression mainly in the GCL and AMI054 showed intense GFP signal at INL. From the mouse and pig studies it can be concluded that AMI054 had brightest, broadest and consistent results. Therefore, AMI054 was selected and finalized as the AAV capsid for inserting and screening for AVMX-110. Confocal microscopic images were also documented for better visualization of different layers of eye. These images illustrated penetrability of capsid into different ocular layers (FIG. 27). AMI054, even though expressed lower GFP, showed deeper penetrability into INL. V226 showed more expression in RGC layer.

### GOI Screening

Several constructs were designed and screened before finalizing the candidate. Expression profile of all the constructs was checked in human embryonic kidney cells (HEK293) cells. Some of the constructs were also checked in ARPE-19 cells (Human retinal pigment epithelial cells) and hTERT-rpe 1 (human Telomerase reverse transcriptase-retinal pigmental cells). hTERT-rpe 1 cells were derived by transfecting the RPE-340 cell line (primary human RPE cell line) with the pGRN145 hTERT-expressing plasmid. This was used to mimic primary RPE cells. Table 14 shows the expression of these constructs in different cell lines. For example, AAV2.N54.120 viral particle (also named AVMX-110) comprises the non-naturally occurring nucleic acid packaged inside the AAV2.N54 capsid comprising: high GC content (denoted by “GC” in the “GCRS”); codon AGG for encoding arginine (denoted by “R” in the “GCRS”); and codon TCC for encoding serine (denoted by “S” in the “GCRS”). For another example, AAV6.N54.120 virus particle (also named AVMX-116) comprises the non-naturally occurring nucleic acid packaged inside the AAV6.N54 capsids comprising: high GC content (denoted by “GC” in the “GCRS”); codon AGG for encoding arginine (denoted by “R” in the “GCRS”); and codon TCC for encoding serine (denoted by “S” in the “GCRS”).

TABLE 14
Constructs screened for the AVMX-110/116 project

| | | | | | | Aflibercept | |
|---|---|---|---|---|---|---|---|
| | | | | | | Expression | |
| | | | | | | (μg/mL) | |
| | | | Self- | | Production | HEK2 | ARP |
| Construct | Lot# | Description | Complimentary | Promoter | date | 93 | E-19 |
| N54.120 | 21-004 | scCMV- | ds | CMV | Feb. 1, 2021 | 20.9 ± 3.1 | 1.4 ± 0.2 |
| | | Aflibercept- | | | | | |
| | | GCRS(TCC) | | | | | |
| N54.120 | 21-077 | scCBA- | ds | CBA | Aug. 9, 2021 | 9.7 ± 2.2 | ND |
| | | Aflibercept- | | | | | |
| | | GCRS(TCC) | | | | | |
| N54.164 | 21-088 | pFB-CMV- | ss | CMV | Sep. 10, 2021 | 1.0 | 0.07 |
| | | SV40in- | | | | | |
| | | Aflibercept- | | | | | |
| | | pA-Stuffer- | | | | | |
| | | GFP | | | | | |
| N54-190 | 21-097 | pFB-scCMV- | ds | CMV | Oct. 6, 2021 | 19.6 ± 6.3 | 2.1 |
| | | SV40-intron- | | | | | |
| | | kozak- | | | | | |
| | | Aflibercept-rev | | | | | |
| N54-191 | 21-093 | pFB-scAd- | ds | CMV | Sep. 27, 2021 | 9.8 | 1.4 |
| | | enhancer- | | | | | |
| | | CMV-SV40in- | | | | | |
| | | Aflibercept-rev | | | | | |
| N54.192 | 21-094 | pFB-scCBA- | ds | CBA | Oct. 6, 2021 | 3.3 ± 1.4 | 1.1 |
| | | SV40-intron- | | | | | |
| | | kozak- | | | | | |
| | | Aflibercept-rev | | | | | |
| N54.221 | 21-114 | scCMV-SV40- | ds | CMV | Oct. 25, 2021 | 22.9 ± 2.0 | 1.6 |
| | | intron-kozak- | | | | | |
| | | VEGF-Trap- | | | | | |
| | | GCRS(TCC)- | | | | | |
| | | WPREa | | | | | |
| N54.222 | 21-115 | scCMV-TPL- | ds | CMV | Oct. 25, 2021 | 11.1 | 0.8 |
| | | kozak-VEGF- | | | | | |
| | | Trap- | | | | | |
| | | GCRS(TCC) | | | | | |
| N54.223 | 21-116 | scCMV-SV40- | ds | CMV | Oct. 25, 2021 | 7.5 | 0.5 |
| | | intron-kozak- | | | | | |
| | | VEGF-Trap- | | | | | |
| | | geneious opt | | | | | |
| N54.224 | 21-117 | scCMV-SV40- | ds | CMV | Oct. 25, 2021 | 5.0 | 0.7 |
| | | intron-kozak- | | | | | |
| | | VEGF-Trap- | | | | | |
| | | GenScript | | | | | |
| | | optimized | | | | | |
| N54.225 | 21-118 | scmCMV- | ds | Murine | Oct. 25, 2021 | 1.7 | 1.1 |
| | | SV40-intron- | | CMV | | | |
| | | kozak-VEGF- | | | | | |
| | | Trap- | | | | | |
| | | GCRS(TCC) | | | | | |
| N54.226 | 21-119 | scmCMV- | ds | Murine | Oct. 25, 2021 | 3.1 | 1.3 |
| | | SV40-intron- | | CMV | | | |
| | | kozak-VEGF- | | | | | |
| | | Trap- | | | | | |
| | | GCRS(TCC)- | | | | | |
| | | WPREa | | | | | |
| N54.228 | 21-126 | scmCMV- | ds | Murine | Nov. 10, 2021 | 0.6 | |
| | | SV40-intron- | | CMV | | | |
| | | kozak- | | | | | |
| | | Aflibercept- | | | | | |
| | | geneious | | | | | |
| | | opt-WPREa | | | | | |
| N54.229 | 21-127 | scmCMV- | ds | Murine | Nov. 10, 2021 | 1.5 | |
| | | SV40-intron- | | CMV | | | |
| | | kozak- | | | | | |
| | | Aflibercept-GS | | | | | |
| | | opt-WPREa | | | | | |
| N54.230 | 21-128 | scmCMV- | ds | Murine | Nov. 10, 2021 | 1.6 | |
| | | SV40-intron- | | CMV | | | |
| | | kozak-VEGF- | | | | | |
| | | Trap- | | | | | |
| | | GCRS(TCC)- | | | | | |
| | | rev | | | | | |
| N54.231 | 21-129 | scmCMV- | ds | Murine | Nov. 10, 2021 | 1.4 | |
| | | SV40-intron- | | CMV | | | |
| | | kozak-VEGF- | | | | | |
| | | Trap- | | | | | |
| | | GCRS(TCC)- | | | | | |
| | | WPREa-rev | | | | | |
| N54.232 | 21-130 | scmCMV- | ds | Murine | Nov. 10, 2021 | 0.6 | |
| | | SV40-intron- | | CMV | | | |
| | | kozak- | | | | | |
| | | Geneious opt- | | | | | |
| | | Aflibercept- | | | | | |
| | | WPREa-rev | | | | | |
| N54.233 | 21-131 | scmCMV- | ds | Murine | Nov. 10, 2021 | 1.4 | |
| | | SV40-intron- | | CMV | | | |
| | | kozak- | | | | | |
| | | GenScript opt- | | | | | |
| | | Aflibercept- | | | | | |
| | | WPREa-rev | | | | | |
| N54.200 | 21-155 | pFB-CMV- | ss | CMV | Jan. 5, 2022 | 27.4 ± 7.2 | 2.3 ± 0.4 |
| | | TPL-enh- | | | | | |
| | | Intron- | | | | | |
| | | Aflibercept- | | | | | |
| | | structure-pA | | | | | |
| N54.204 | 21-156 | pFB-scCMV- | ds | CMV | Jan. 5, 2022 | 8.4 | 0.1 |
| | | Vh-Leader-Fc- | | | | | |
| | | 4xGGGGS- | | | | | |
| | | VEGF-Trap | | | | | |
| N54.296 | 22-019 | pFB-CMV- | ss | CMV | Feb. 23, 2022 | 2.0 | 0.1 |
| | | TPL-Full-enh- | | | | | |
| | | Aflibercept- | | | | | |
| | | structure- | | | | | |
| | | hGHpA | | | | | |
| | | (fusion) | | | | | |
| N54.298 | 22-020 | pFB-CMV- | ss | CMV | Mar. 2, 2022 | 5.2 | 0.5 |
| | | TPL-Full-enh- | | | | | |
| | | intron- | | | | | |
| | | Aflibercept- | | | | | |
| | | structure- | | | | | |
| | | hGHpA | | | | | |
| N54.261 | 22-017 | pFB-scCMV- | ds | CMV | Feb. 23, 2022 | 3.5 | 0.1 |
| | | VEGF- | | | | | |
| | | TrapGCRS(TC | | | | | |
| | | C)-1xGGGGS- | | | | | |
| | | Endostatin- | | | | | |
| | | IgG4Fc- | | | | | |
| | | WPREa | | | | | |
| N54.268 | 22-018 | pFB-scCMV- | ds | CMV | Feb. 23, 2022 | 3.9 | 0.1 |
| | | SV40in-kozak- | | | | | |
| | | VEGF-Trap- | | | | | |
| | | Fc4- | | | | | |
| | | 1xGGGGS- | | | | | |
| | | Endostatin- | | | | | |
| | | WPREa | | | | | |
| N54.243 | 22-026 | pFB-scCMV- | ds | CMV | Mar. 10, 2022 | | |
| | | SV40-intron- | | | | | |
| | | kozak-FLT1- | | | | | |
| | | KDR-Fc4- | | | | | |
| | | WPREa | | | | | |
| N54.244 | 22-027 | pFB-scCBA- | ds | CBA | Mar. 10, 2022 | | |
| | | SV40-intron- | | | | | |
| | | kozak-FLT1- | | | | | |
| | | KDR-Fc4- | | | | | |
| | | WPREa | | | | | |
| N54.248 | 22-036 | pFB-scCMV- | ss | CMV | Mar. 17, 2022 | | |
| | | TPL-AdEnh- | | | | | |
| | | Intron- | | | | | |
| | | AfliberceptGC | | | | | |
| | | RS- | | | | | |
| | | WPREmini-pA | | | | | |
| N54.290 | 22-028 | pFB-scCMV- | ds | CMV | Mar. 10, 2022 | | |
| | | SV40in-kozak- | | | | | |
| | | VEGF-Trap- | | | | | |
| | | GCRS(TCC)- | | | | | |
| | | Albumin 3′- | | | | | |
| | | UTR-pA | | | | | |
| N54 | HEK030920 | Jcat optmized- | ds | CMV | Mar. 17, 2022 | | |
| (184)-311 | 22-17 | VEGF-Trap | | | | | |
| | | hGHPA | | | | | |

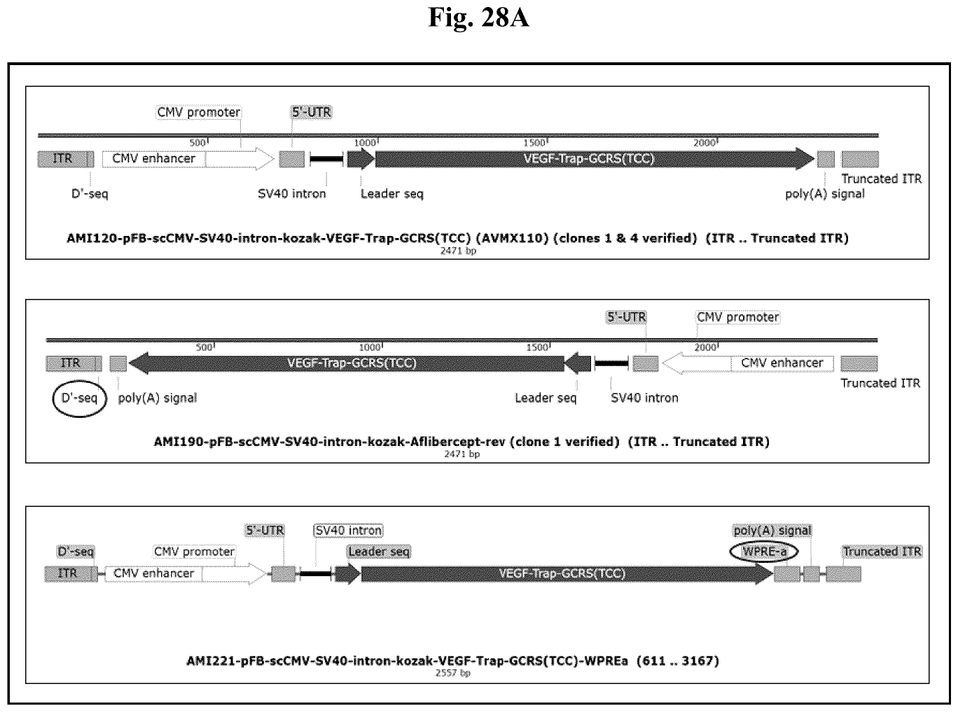

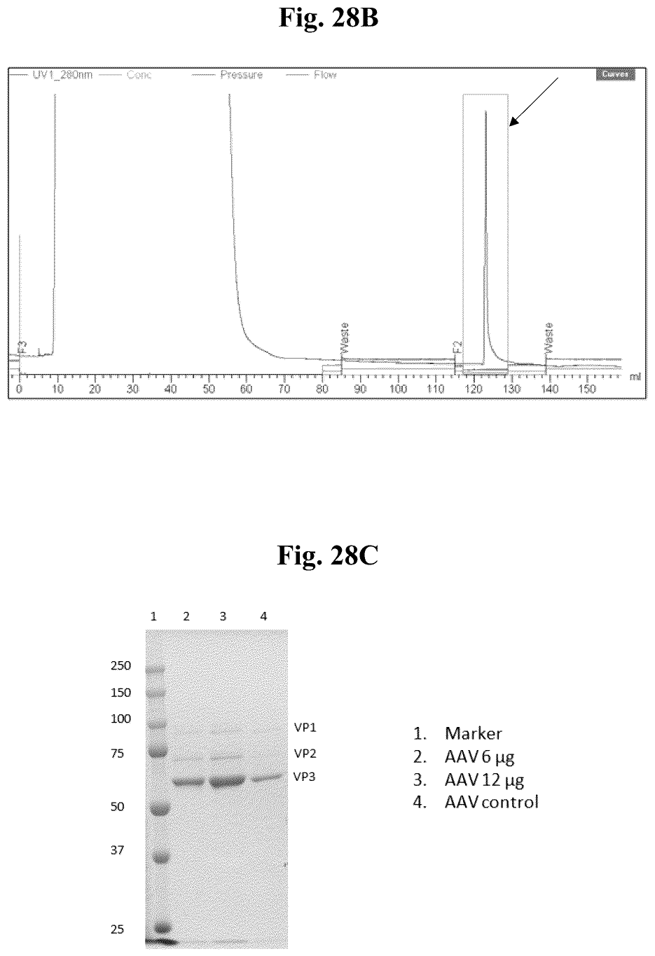

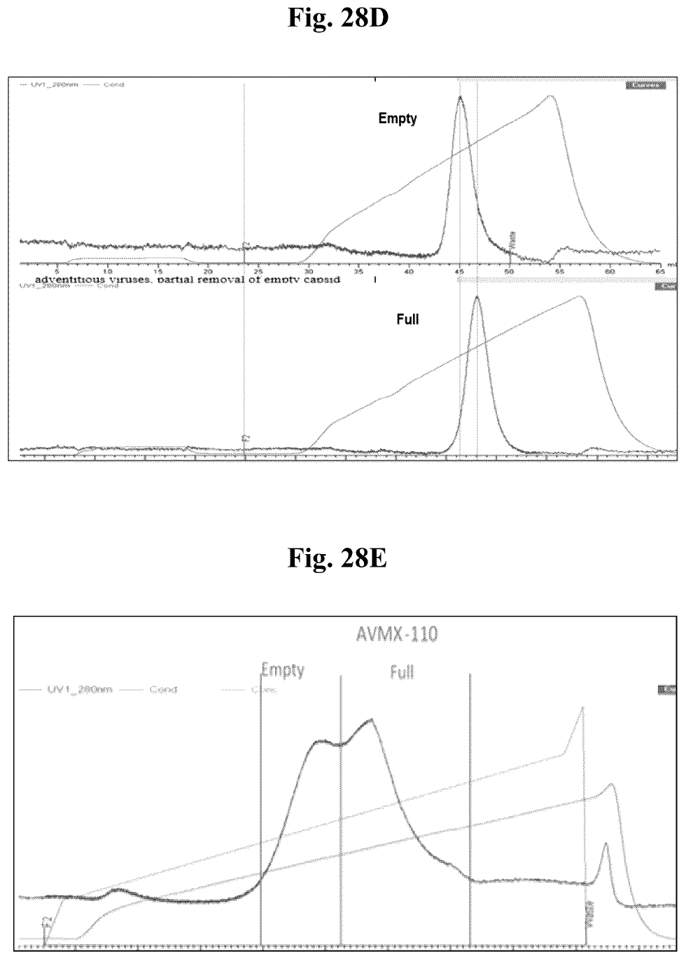

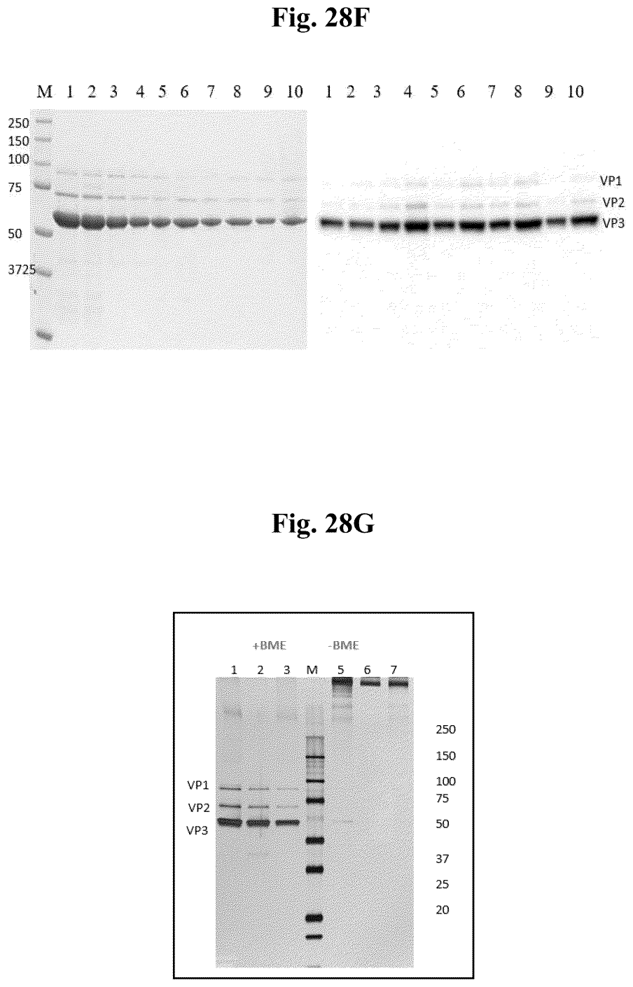

Three constructs listed in Table 14 (N54.120, N54.190 and N54.221) exhibited higher aflibercept expression compared to others. The AAV construct back bone for these three candidates are shown in the FIG. 28A. During the manufacturing process, samples were taken at various points of process steps for various purposes of testing in order to achieve accurate control of processes. Exemplary sample process chromatograms for two manufacturing runs are shown in FIG. 28B. Through the capture step, a single and sharp fraction of the AVMX-110 vectors was obtained during the process (arrow). Almost 100% particles of AVMX-110 were captured by our capturing process and with more than 80% eluted in process intermediate pool. FIG. 28C analysis of the eluted AVMX-110 process intermediate purity by SDS-PAGE. AAV VP1, VP2 and VP3 were clearly visualized, and no other major impurity protein was visible. From the capsid charge difference, the performance profile of empty and full AAV was established in the column process. Under this condition, empty capsid ran inside column faster than the full capsid. The differential retention time was sufficient for separation of empty from the full capsid (FIG. 28D). The eluate pool for the capture step was processed for separation of a portion of empty capsid. The separation curves are shown in FIG. 28E. Using this separation model, AVMX-110 was purified and enriched in full capsid content. In the separation chromatograms, two separate peaks were obtained. The full capacity enriched fraction post-column chromatography was succeeded with concentration and buffer exchange to make a drug substance for further evaluation. AVMX-110 manufacturing intermediate and drug substance samples were analyzed for specify detected with AAV2 antibody Western blot, for each development lot. The antibody to AAV2 VP1, VP2, and VP3 were the only protein bands seen in gel and the Western blot (FIG. 28F). AVMX-110 purity was analyzed by gel electrophoresis stained by silver stain method. The first peak was empty, and the second peak was full capsid. The product was AAV particles containing VP1, VP2, and VP3. The purity of AVMX-110 process intermediates was analyzed by SDS-PAGE stained with silver stain gel (FIG. 28G). AAV VP1, VP2, and VP3 were clearly visualized, and no single impurity high than 4% was found.

### Host Cell Contaminants

Sf9 host cell protein contaminants in AVMX-110 developmental product was measured by ELISA. Exemplary measurements of the host cell protein contaminant is shown in Table 15. The host protein level was very low (≤170 ng/10¹² vg). The purification process was extremely specific for GOI as it removed 1.5+10-fold of host protein from starting cell culture lysate. The level of host protein had exceeded the requirement of ≤100 ng/dose for product release. For this lot, if a dose was at 2e+11 vg, the host cell protein level was 4 ng/dose. The PCNA of host cell DNA detection the limits of another batch was ≤46.12 pg/1e+12 vg. The host cell protein and DNA testing indicated that AVMX-110 was highly purified and that exceeds regulatory requirements.

TABLE 15
Host cell protein and host cell DNA quantification

| | AAV2.N54- | | | | | |
|---|---|---|---|---|---|---|
| Sample | VEGF-Trap | Host Cell Protein | | | Host Cell DNA | |
| Capture | 3.06e+10 | 204 | 67e+3 | 6.1E−09 | ≤10 | ≤10 is the |
| eluate | | | | | | LoD at |
| Polish | 6.82e+10 | 11.6 | 170 | 1.5E−10 | ≤10 | pg/reaction |
| column | | | | | | |

Sf9 cell genomic DNA was purified with the Qiagen genomic DNA purification kit, and the DNA was diluted to 1000, 250, 63, 16, 4, and 1 ng/mL and used as standards. AVMX-110 process samples were diluted 2 folds, and 10 folds in QPCR dilution buffer and used for qPCR primers and probes to Sf9 cells. All tests performed with AVMX-110 process samples were non-detectable with Sf9 cell DNA at ≤10 pg/reaction, which was equivalent to 1 ng/mL≤300 μg/dose.

### Determination of Baculoviral and Other Plasmid DNA Contaminants

Baculoviral DNA was purified according to the protocol provided by Qiagen genomic DNA purification kit with few modifications. Briefly, 7.5 mL baculovirus in medium was mixed with equal volume of cold 20% PEG 8000 in 1 N NaCl and let stand at room temperature for 30 minutes. The baculoviral particles were pelleted by centrifugation at 10,000 rpm for 10 minutes and dissolved in 5 mL G2 buffer. 95 μL proteinase K was added to digest the protein capsids at 50° C. for 60 minutes. The column was equilibrated, and the sample was loaded. After washing, the baculoviral DNA was eluted and qPCR tested with primers and probe complementary to GP64 gene of baculovirus genome. The DNA was diluted to 1000, 250, 63, 16, 4, and 1 ng/mL and used as standards. AVMX-110 samples were diluted 2 and 10 folds in qPCR dilution buffer (without DNase digestion) and used for qPCR assay. The baculovirus DNA level was 8.58e+5 per 10¹² vg of AVMX-110 vector as shown in Table 16.

TABLE 16
Level of Baculovirus and plasmid DNA in copies/per 1e+12 vg of plasmid genes

| Vector | | | | | | |
|---|---|---|---|---|---|---|
| copy* (vg) | Gentamicin | Ampicillin | PCNA (pg) | Bac IE1 | GP64 | Rep |
| 1E+12 | 5.48e+09 | 4.17e+09 | ≤46.12 | 4.45e+07 | 8.58e+05 | 1.57e+07 |

AVMX-110 vector copy number. PCNA was the house keeping gene of SF9 cells. Bac IE1 was the DNA fragment cloned in the SF9 cell. GP64 was a gene of baculovirus DNA. Rep was DNA fragment in helper baculovirus.

The other plasmid selection marker genes such as gentlemen, ampicillin (AMP), Rep, host cell DNA marker, PCNA, BacIE1 DNA contaminants were below the regulatory requirements, indicating AVMX-110 was pure and met regulatory safety requirements. The levels of these evaluated DNA markers were approximately 10-fold lower than regulatory requirements.

### Endotoxin Level

Endotoxin level in AVMX-110 sample was measured by a chromogenic limulus amebocyte lysate (LAL) release assay using commercial kit. In the presence of endotoxin, proteolytic cleavage of the colorless chromogenic substrate in Pyrochrome LAL releases pNA, which produces yellow color solution and has absorption at 405 nm. The result of endotoxin level in AVMX-110 preparation are shown in the Table 17. The endotoxin level in AVMX-110 sample was 0.0154 EU/mL or 0.005 EU/dose.

TABLE 17
Endotoxin level of AVMX-110 sample

| I.D. | undiluted | 1:2 | 1:4 | 1:8 | 1:16 | 1:32 |
|---|---|---|---|---|---|---|
| Unspiked | 0.0132 | 0.0077 | 0.0059 | 0.0051 | 0.0048 | 0.0045 |
| sample | | | | | | |
| (21-004) | | | | | | |
| Spiked | n/a | 0.0295 | 0.0275 | 0.0370 | 0.0436 | 0.0408 |
| sample | | | | | | |
| (0.04 EU/ml) | | | | | | |
| Recovery | n/a | 0.0219 | 0.0216 | 0.0318 | 0.0388 | 0.0363 |
| (spike- | | | | | | |
| unspike) | | | | | | |
| % Recovery | n/a | 55 | 54 | 80 | 97 | 91 |

### Sterility

Each batch of AVMX-110 was assayed for bioburden, and no microbiological contamination was detected for drug substance used for animal test.

### Replication Competent AAV Level

Traditional methods of using mammalian cells to produce AAV vectors with plasmid transfection or virus infection generally produces some replication competent AAV (rcAAV) virus particles due to the non-homologous recombination between AAV genome and AAV rep-cap plasmids. Contamination of rcAAV is not desirable in GMP manufacturing and the amount of contamination is required to be reported to the FDA. The AAV system described herein removed and disrupted the AAV promoters and replaced them with insect cell promoters which were not active in mammalian cells. No rcAAV is able to be generated in the Bac-to-AAV system during the manufacturing process. To establish the assay to monitor the rcAAV, 100 wild type (wt) AAV particles were spiked into 1e+12 vg AAV sample. The rcAAV assay was performed. Briefly, 1e+12 vg AAV sample was mixed with 100 vg of wtAAV2 particles in the presence of adenovirus type helper and transduced into HEK293 cells for 3 days. The cells were harvested, and lysate was prepared. After heat inactivation at 56° C. for 30 minutes to inactivate the adenovirus, 50% of the lysate was used to transduce the HEK293 cells in the presence of adenovirus type 5 to amplify the rcAAV or spiked wtAAV for 3 days. The cells were harvested, and lysate was prepared for qPCR detection for the rcAAV signal. The results showed that, without spike in wtAAV2, there was no positive signal of rcAAV. When spiked with 100 vg wtAAV in 1e+12 vg AAV sample, positive signal was detected (Table 18).

TABLE 18
Detection of replication competent AAV in AVMX-110 drug substance

| | | | | rcAAV |
|---|---|---|---|---|
| Virus | Description | Lot # | vg/ml | (vg) |
| AVMX-110 | AAV2.N54-VEGF-Trap | 20-140 | 1.92E+13 | <1E−10 |

### Empty/Full Capsid Estimation

Ratio of full and empty capsid of each lot was estimated using vector specific qPCR and AAV2 specific antibody ELISA. The product fractions of the column chromatography were analyzed for vector copy numbers and protein level. The empty capsid had no or very low vector DNA level but the capsid protein only, while the full vectors had both vector DNA and protein contents. The data showed that the elution 1 of the polish step had no or extremely low gene of interests. The eluate of peak 1 was extremely low or even to non-detectable for AAV2.N54-VEGF-Trap DNA but the protein. The second peak (Peak 2) in the profile showed both vector DNA and protein. The results demonstrated that the purified AVMX-110 and UFDF pool were all full particles (FIG. 10A).

### AVMX-110 Infectivity

AVMX-110 infectivity was determined by TCID₅₀ assay (Tissue Culture Infectious Dose 50%/mL), which was the concentration of infectious organisms in the inoculum determined from the dilution at which the inoculum infects 50% of the target cultures (i.e., when the starting sample was diluted by an amount equal to the TCID₅₀/mL, 1 mL aliquots added to multiple target cultures for infection, on average, 50% of the cultures). The TCID₅₀ assay was used to determine the potency of AAV vectors based on manufacturer's protocol (ATCC). Briefly, a series of diluted AAV samples were used to transduce the HelaRC32 cells in the presence of adenovirus 5 helper for 3 days in a 96-well plate. Cell lysates were prepared and positive signals were detected with qPCR method. TCID₅₀ value was calculated based on the Spearman-Karber formula. The result is shown in Table 19.

TABLE 19
Detection of AVMX-110 TCID₅₀

| | | | | | vg/ |
|---|---|---|---|---|---|
| Virus | Description | Lot # | vg/ml | TCID₅₀ | TCID₅₀ |
| AVMX-110 | AAV2.N54- | 20-140 | 1.92E+13 | 2.00E+11 | 96.23 |
| | VEGF-Trap | | | | |
| ATCC | AAV2-GFP | | | | |
| reference | | | | | |

### AVMX-110 Expression of VEGF-Trap (AAV2.VEGF-Trap) in HEK293 Cells

Purified AAV2-VEGF-Trap vector was used to transduce HEK293 cells at MOI of 100,000 vector genome (vg) per cell. VEGF-Trap produced and released into cell culture supernatant was analyzed daily using an internal standard operational protocol (21-AD-VEGF-ELISA.01) using commercial VEGF-A₁₆₅ as a coating antigen. VEGF-A₁₆₅ was expressed in HEK293 cells, purified to homogeneity (GeneScript, Cat #Z03073), and reconstituted with sterile Milli-Q water at 100 μg/mL. The ELISA procedures were as followed. VGEF-A₁₆₅ was coated onto the 96-well ELISA plate and incubated 0/N at 4° C. Next day the plate was washed with wash buffer thrice, and blocking buffer was added to each well. The plate was covered and incubated at 37° C. for 2 hours. Plate was covered and put back into the incubator for 1 hour. Thereafter, the plate was washed, and anti-human Fc antibody was added to the plate and again incubated for 1 hour at 37° C. Plate was washed, and streptavidin-HRP was added for 45 minutes of incubation. Plate was washed, and TMB substrate was added. After about 15 minutes, stop solution was added. The wells were read at 450 nm in a microplate reader. As seen from FIG. 10B, the cell culture supernatant harvested from HEK293 cells transduced with AMVX-110 (AAV2.N53-VEGF-Trap) secreted VEGF-Trap to approximately 800 ng/mL, while the HEK293 cells without transduction did not secrete VEGF-Trap. The production continued with daily increase during cultivation before the cells aged.

### Durability of AAV2.VEGF-Trap Expression In Vitro

HEK293 and ARPE-19 (human retina pigment epithelium) cells were cultured with 10% FBS and transduced with AAV2.N53-VEGF-Trap and AAV2.N54-VEGF-Trap at MOI of 100,000 per cell. Samples were taken for ELISA quantification of VEGE-trap expression. AAV2.N54-VEGF-Trap showed better expression when compared to AAV2.N53-VEGF-Trap (FIG. 11). This variation was observed significantly lower in ARPE19 cells than in HEK293 cells. In ARPE19 cells, after day 20 the VEGF-Trap expression stemmed from transduction of N53-VEGF-Trap construct was negligible. In contrary, VEGF-Trap expression stemmed from transduction of N54-VEGF-Trap construct was still present in quantifiable amounts. VEGF-Trap expression was higher in HEK293 cells compared to ARPE19 cells.

### Mouse Efficacy Study

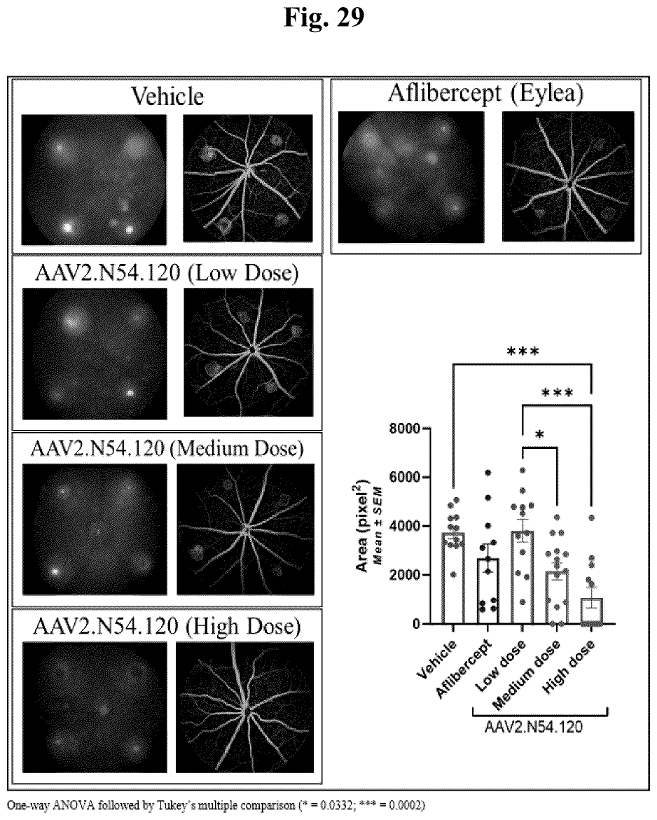

Construct AAV2.N54.120 (AVMX-110) was selected to be administered into the mouse at three different doses to check for the efficacy. Briefly, the study had 6 groups, i.e., vehicle (formulation buffer), commercial Eylea (protein), AAV2.N54-GFP (−) dosed at 1.6E+10 vg/eye, AVMX-110 low dose (2.0E+07 vg/eye), medium dose (4.8E+08 vg/eye) and high dose (1.6E+10 vg/eye). FIG. 29 shows the fluorescence angiography (FA) data. The medium dose of the AAV2.N54-120 had similar efficacy compared to the commercial aflibercept (Eylea) protein administered at 40 μg/eye.

Aflibercept ELISA was used to quantify the expression level of aflibercept in the ocular samples (local expression) and serum samples (systemic level). Aflibercept expression in ocular samples injected with high dose of construct showed detectable expression. However, the other groups showed very low or no detectable aflibercept expression.

### Serotype Selection

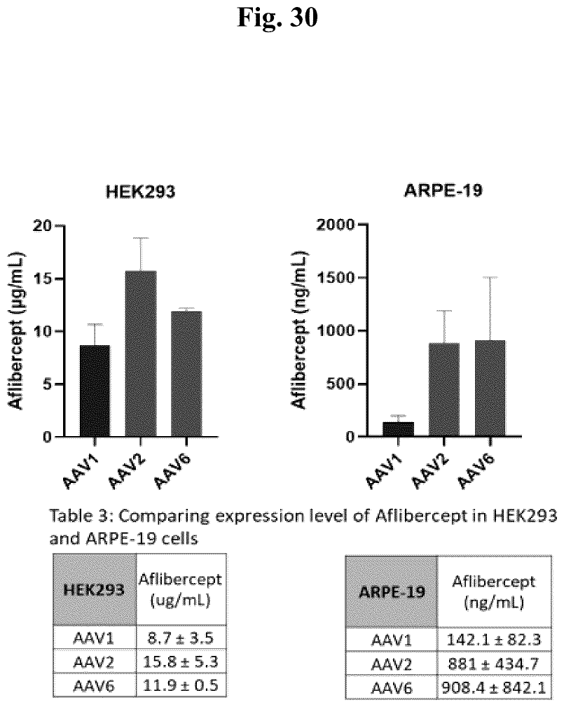

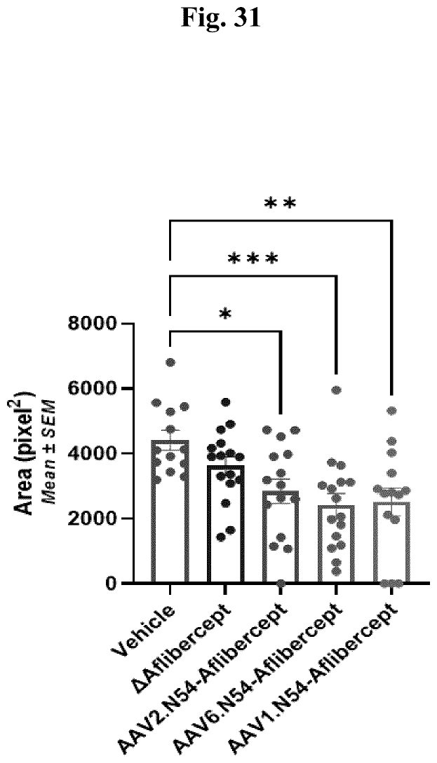

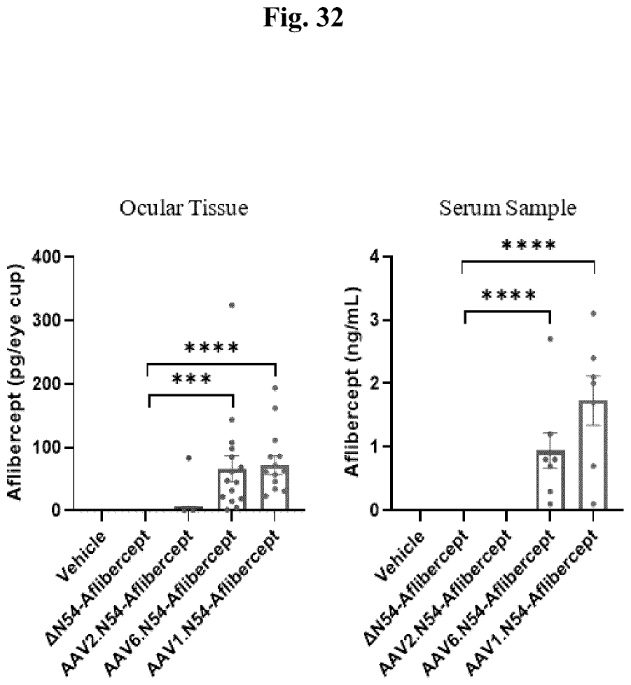

It is well known that AAV (especially AAV2) neutralizing antibodies are widely present in humans. Another challenge in using AAV2 is lack of tropism. Therefore, AAV1 and AAV6 serotypes were screened in vitro (FIG. 30 and Table 30) for expression level and in vivo for efficacy (FA data, FIG. 31) and aflibercept expression in ocular tissue (FIG. 32 and Table 20).

TABLE 20
Tabular representation of aflibercept expression
in ocular and serum samples

| | | Eye Cup (Retina) | | Serum | |
|---|---|---|---|---|---|
| | Dose/eye | | ng/ | | ng/ |
| Serotype | (IVT) | ng/mL | eye cup | ng/mL | animal |
| AAV1.N54- | 4.8e+8 vg | 7.2±5.3 | ≤LoQ | ≤LoQ | ≤LoQ |
| Aflibercept | | | | | |
| AAV2.N54- | 4.8e+8 vg | ≤LoQ | ≤LoQ | ≤LoQ | ≤LoQ |
| Aflibercept | | | | | |
| AAV6.N54- | 4.8e+8 vg | 6.6±8.1 | ≤LoQ | ≤LoQ | ≤LoQ |
| Aflibercept | | | | | |

LoQ = Limits of Quantification

Aflibercept ELISA method had been performed, and LoQ for that assay was determined to be 2 ng/mL. The values that were under 2 ng/mL were denoted as >LoQ. From Table 20, it could be concluded that serum samples did not have any detectable aflibercept. However, AAV6 and AAV1 had detectable level of aflibercept in ocular samples.

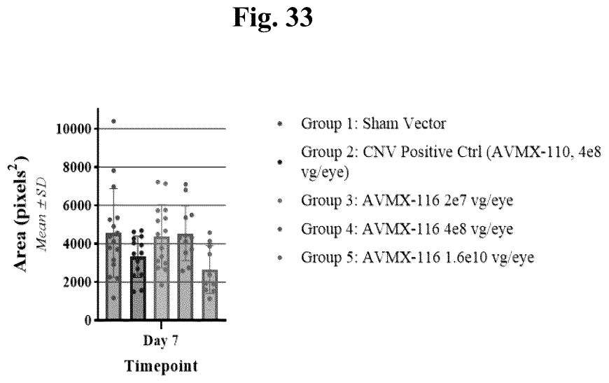

A dose response study was designed and conducted, where AAV6.N54-Aflibercept was dosed in mice at 2.0E+07 (low dose), 4.8E+08 (medium dose), and 1.6E+10 high dose. FIG. 33 shows the FA data as a bar graph for comparing different groups.

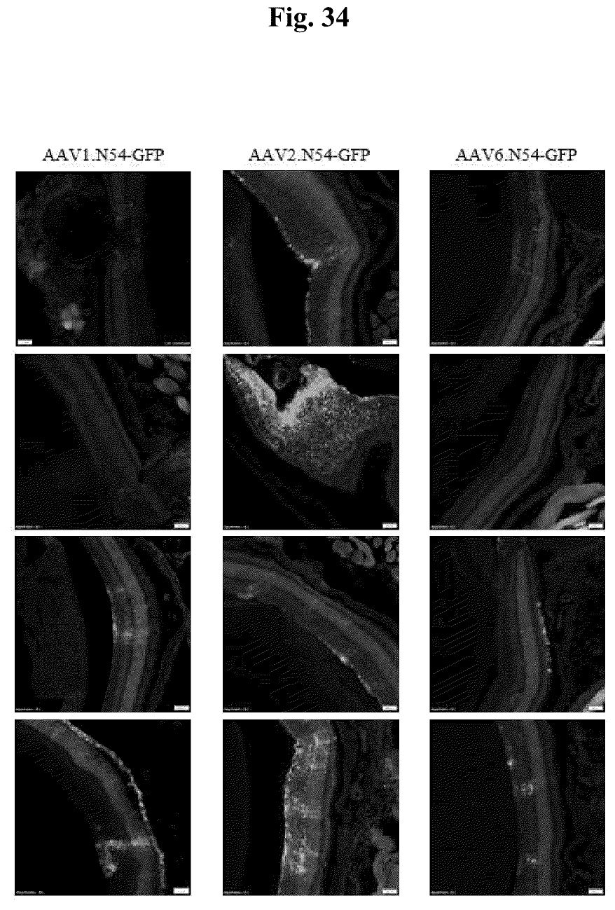

Animals were injected with 5E+09 vg/eye of AAV2, AAV1, and AAV6-GFP to check for the expression and penetrability. Those animals didn't show any supremacy over AAV2.N54-GFP (FIG. 34).

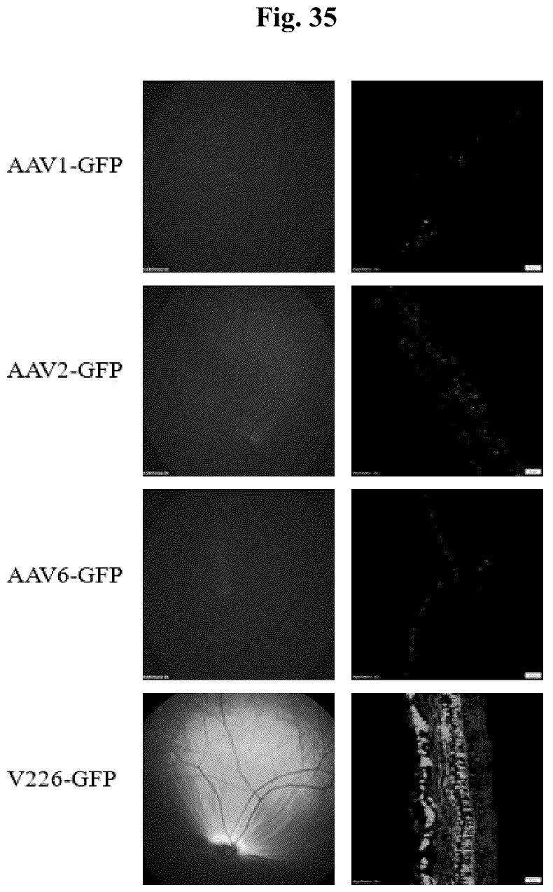

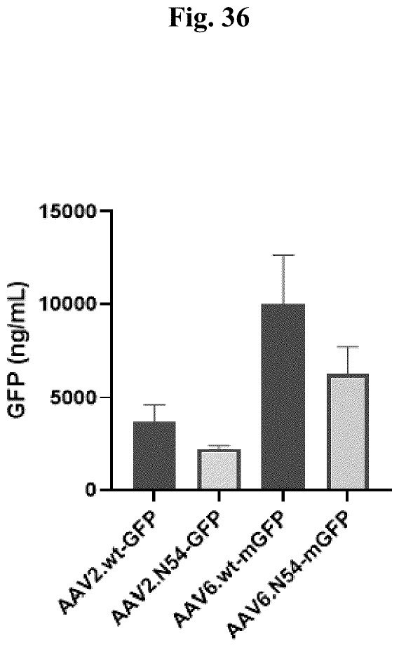

In order to compare different serotypes, pig study was performed where pigs were injected with 1E+11 vg/eye with AAV1, AAV2, and AAV6-GFP. Similar to the previous pig study, V226 (+control) had also been included. FIG. 35 shows the representative fundus and IHC images. The fundus imaging was done on day 21, 28, and 34 due relatively low GFP signal in AAV1, AAV2, and AAV6-GFP injected animals. Finally, on day 34 the animals were sacrificed, and IHC was performed on the selected eyes. The in vitro ELISA assays for GFP did not show higher expression of GFP in AAV2.N54-GFP when compared to AAV2.wt-GFP (FIG. 36).

During the GOI screening, lot to lot variability in the AAV constructs was detected. For example, N54.120 construct was purified multiple times and stored at −80° C. The purification process varied from using different volumes of starting cell culture, purifying using either 2 cycles of ultracentrifugation (UC) or AAVx affinity column, cell viability and other culture condition variation. Table 21 shows the summary of few lots of N54.120.

TABLE 21
Comparison of lot to lot variability in N54.120 construct

| | | | | Aflibercept expression | | |
|---|---|---|---|---|---|---|
| | Culture | | Purification | | | hTERT- |
| Lot# | Volume | | method | HEK293 | ARPE-19 | rpe1 |
| 21-004 | 200 | mL | UC | 20.9 ± 3.1 | 1.4 ± 0.2 | ND |
| 21-030 | 200 | mL | UC | 10.4 ± 0.6 | 0.6 ± 0.04 | 0.4 |
| 21-038 | 2 | L | AAVx + UC | 31.9 ± 2.1 | ND | ND |
| 21-045 | 2 | L | AAVx + UC | 33.5 ± 2.6 | 1.2 | 1.2 |
| 21-050 | 2 | L | AAVx + UC | 31.6 | ND | ND |
| 21-096 | 50 | L | AAVx + UC | 16.6 | 1.3 | 0.8 |

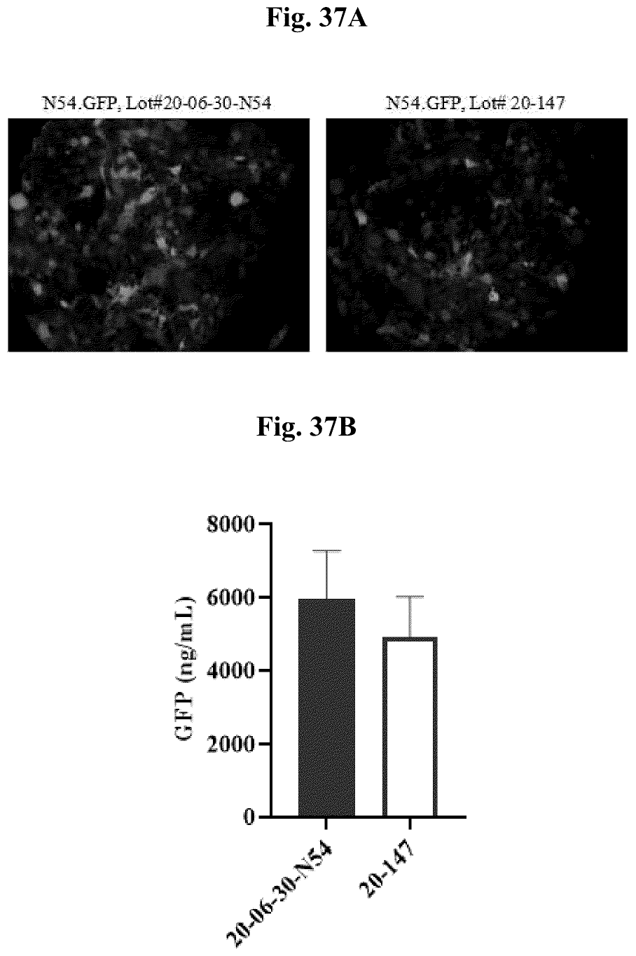

Below is exemplary GFP expression captured in fluorescence microscope images (FIG. 37A) and also analyzed using GFP quantification ELISA (FIG. 37B and Table 22) showing lot to lot variability. Although intensity of GFP lot #20-147 was lower than 20-06-30-N54 lot, ELISA did not show significant difference in GFP expression in vitro.

TABLE 22
GFP concentration determined by
GFP ELISA for different N54-GFP lots

| N54.GFP | GFP |
|---|---|
| lot# | (μg/mL) |
| 20-06-30-N54 | 6.0 ± 2.6 |
| 20-147 | 4.9 ± 2.2 |

After mouse and pig study capsid N54 was selected and AMI120 optimized GOI was selected. Animal efficacy study showed significant laser injury recovery stemmed from the AAV vector treatment that was comparable to commercial aflibercept. AAV2 serotype appeared to be more consistent in mouse efficacy CNV model than AAV6 serotype.

### Example 13. Bioanalytical Comparison Results for Animal Studies

Example 13 illustrates the bioanalytical comparison of mouse ocular Choroidal Neovascularization (CNV) or macular neovascularization (MNV) efficacy model resulted from two different studies to check for the reproducibility of the model. Both studies used the same titer and volume of non-GLP, AAV2 construct, for intravitreal (IVT) injection. The study protocols were also similar (Table 23). AAV2 capsid quantification was performed using commercial AAV2 capsid protein kit (Progen, Cat #: PRAAV2R and Lot #: A20008). For the statistical analysis GraphPad Prism (v9.0.1) was used.

TABLE 23
Protocol comparison for Example 13

| Study | No. of | OU | | Treatment | MNV |
|---|---|---|---|---|---|
| code | animals | treatment/dose | Vol./Route | day | induction |
| 21-AVI- | 8 | Vehicle¹ | 1.0 μL/eye | Day −3 | OU: CNV |
| AX110.01 | 8 | AVMX-110 | IVT | Day −28 | Laser |
| | | 4E+08 | | | Day 0 |
| | | vg/eye | | | |
| 21-AVI- | 8 | Vehicle² | 1.0 μL/eye | Day −28 | OU: CNV |
| AX112.01 | 8 | AVMX-110 | IVT | | Laser |
| | | 4E+08 | | | Day 0 |
| | | vg/eye | | | |

Vehicle = AAV formulation buffer;
AVMX-110 = AAV2.N54-120.
¹1X PBS pH 8.0, 0.1 mM Sodium citrate, 0.001% Pluronic F-68
²150 mM NaCl, 20 mM Sodium phosphate, pH 7.3, 0.01% Pluronic F-68

### Material and Methods

Table 24 shows the number of serum and ocular samples obtained from the mouse studies.

TABLE 24
Summary of number of serum and ocular
samples obtained after euthanasia

| Study Code | Treatment | Serum samples (no.) | Ocular samples (no.) |
|---|---|---|---|
| 21-AVI- | Vehicle | 8 | 4 |
| AX110.01 | AVMX-110 | 8 | 4 |
| 21-AVI- | Vehicle | 7 | 14 |
| AX112.01 | AVMX-110 | 8 | 16 |

A 532 nm diode laser was used to create 4 single laser spots surrounding the optic nerve. If the construct injection or vehicle caused any inflammation reaction, then the lasering would not make a lesion (bubble). If there was no bubble, then this spot would not be used for analysis. For every eye, 4 laser spots were induced, and the size of the lesions were estimated after 7 days post laser treatment. The lesions corresponded to the wound healing due to the effects of the gene product of various constructs. Eight animals were used in every group, corresponding to 16 eyes with 4 laser spots per each eye resulting in a total of 64 laser spots. Table 25 summarizes the number of no bubbles formed by each group injected with AVMX-110 or vehicle control.

TABLE 25
Number of no bubbles formed out of total 64 laser spots created

| | | Number of no |
|---|---|---|
| Study Code | Treatment | bubbles/total bubbles |
| 21-AVI-AX110.01 | Vehicle | 13/64 |
| | AVMX-110 | 4/64 |
| 21-AVI-AX112.01 | Vehicle | 4/56 |
| | AVMX-110 | 8/64 |

Eye cup samples consisting of Retina/RPE/Choroid/Sclera were homogenized, as described in the study protocol for both the studies, using 1×PBS and BSA without protease inhibitors. Each ocular sample was further homogenized using a sonicator with a pulse of 21 seconds and an interval of 30 second with 5-6 repeats. When a clear supernatant, visible to naked eye, was obtained, the samples were centrifuged at 13,000 rpm for 3 minutes. The supernatant was then collected and used to determine the VEGF-Trap levels.

### FA Analysis Comparison

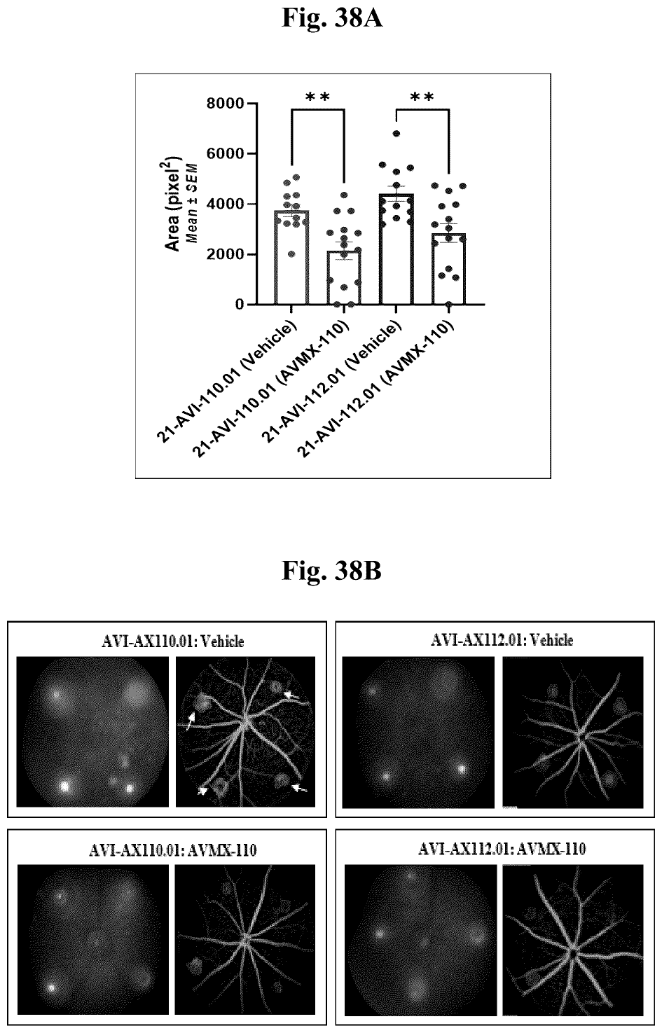

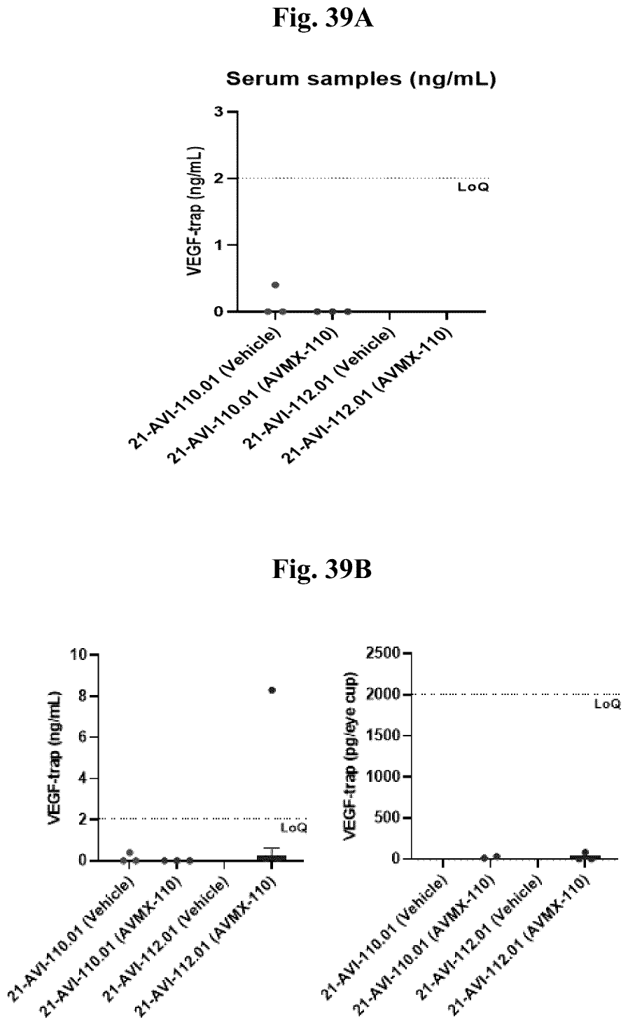

Seven days after laser injury, both eyes for each mouse were analyzed by FA for lesion area. The gene product of AVMX-110 in treated mice was expected to reduce the laser injury as compared to the vehicle control treated mice. FIG. 38 and Table 26 show the comparison of efficacy of AVMX-110 in laser lesion recovery. In both studies, AVMX-110 treated mice showed equal laser wound recovery. There was a variation in the baseline of area for the two studies, but the AVMX-110 treated animals showed almost identical lesion recovery. Representative images from FA image analysis also shows similar reduction in the lesion area FIG. 39.

TABLE 26
Tabular comparison of the lesion area

| | | Lesion Area |
|---|---|---|
| Study Code | Treatment | (pixel²) ± S.D. |
| 21-AVI-AX110.01 | Vehicle | 3930 ± 1052 |
| | AVMX-110 | 2145 ± 1368 |
| 21-AVI-AX112.01 | Vehicle | 4411 ± 1083 |
| | AVMX-110 | 2852 ± 1444 |

### Comparing VEGF-Trap Levels in Serum Samples

Serum samples were directly placed into 96-well plates without dilution. FIG. 39A shows the VEGF-Trap levels in serum samples. VEGF-Trap levels in the serum samples were below the limit of quantification of the qualified VEGF-Trap ELISA.

### VEGF-Trap Levels in Ocular Samples

VEGF-Trap in ocular samples was quantified by ELISA and directly plotted as ng/mL. VEGF-Trap concentrations per mg of tissue weight were calculated by determining the absolute amount of VEGF-Trap determined with ELISA, divided by the volume in which the ocular tissue was homogenized. The amount calculated was then divided by the tissue weight to determine the pg of VEGF-trap per eye cup. The VEGF-trap data was plotted (FIG. 39B), and the VEGF-Trap levels for serum and ocular samples were documented in Table 31 (vehicle samples were excluded from the table since the values were below the LoQ). Overall, the VEGF-Trap expression in all serum and ocular samples were very low, and most of the animals were below limits of quantification of VEGF-Trap ELISA.

TABLE 31
VEGF-Trap levels in Ocular samples

| | | Eye Cup | | | |
|---|---|---|---|---|---|
| | | (Retina/RPE/ | | | |
| | | Choroid/Sclera) | | Serum | |
| | Dose/eye | | ng/ | | ng/ |
| Study code | (IVT) | ng/mL | eye cup | ng/mL | animal |
| 21-AVI- | 4.8e + 8 vg | ≤LoQ | ≤LoQ | ≤LoQ | ≤LoQ |
| AX110.01 | | | | | |
| 21-AVI- | 4.8e + 8 vg | 0.1 ± 2.2 | 0.01 ± 0.1 | ≤LoQ | ≤LoQ |
| AX112.01 | | | | | |

Correlation Analysis Between VEGF-Trap Concentration and Area Recovered after Laser Treatment

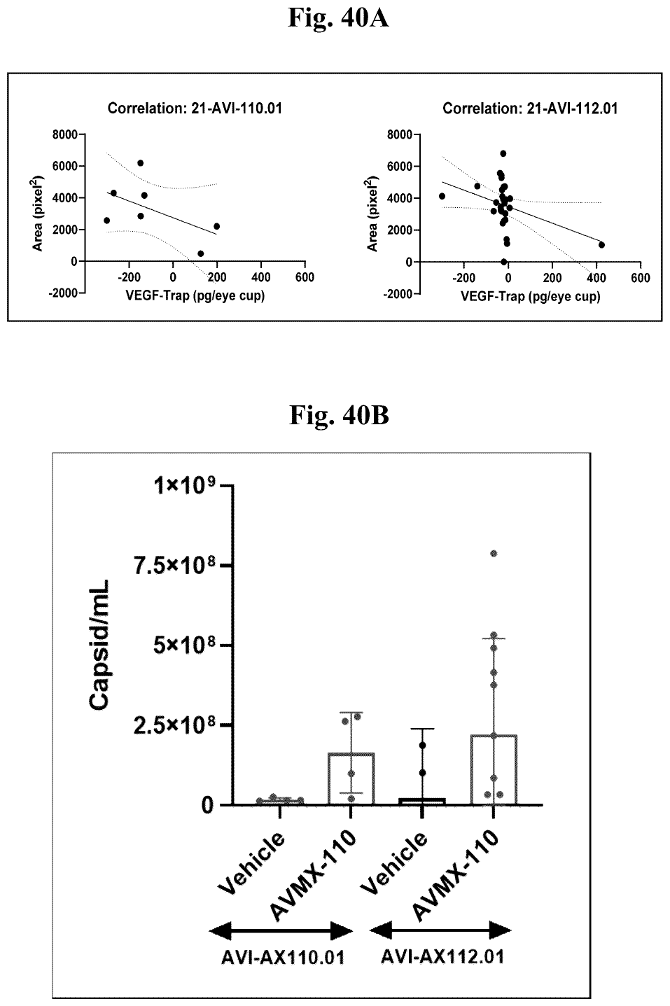

Presence of lesion was measured as area in pixels. The lesion pixel data was plotted against pg of VEGF-Trap per eye cup, and the correlation was calculated using Pearson correlation with a two-tailed p value estimation at 95% confidence interval using Graphpad Prism software. FIG. 40A shows a comparison between the two experiments. Both analysis showed negative correlation of VEGF-Trap concentration with the lesion pixel area. This data indicates that lower lesion areas (pixels) are associated with higher VEGF-Trap concentrations. Correlation coefficients and p values for both the studies are documented in Table 32.

TABLE 32
Correlation coefficients and p values

| Study Code | Correlation coefficient | p value |
|---|---|---|
| 21-AVI-AX110.01 | −0.55 | 0.21 |
| 21-AVI-AX112.01 | −0.36 | 0.057 |

### Comparing the AAV2 Capsid Protein Levels in Both Studies

The AAV2 capsid assay was performed using the AAV2 capsid ELISA kit. The AAV2 capsid protein levels also in both studies were comparable (FIG. 40B). However, it was important to note that the volume of ocular samples (in both studies) varied significantly from sample to sample, impacted the overall capsid protein estimation.

FA images and lesion area analysis results showed consistency between the two studies. There were more “no bubbles” in the vehicle group. For VEGF-Trap levels in ocular samples, many data points were below the limit of quantification. The assay had a LoQ of ˜2 ng/mL. This LoQ value was lower than the commercially available kit (˜5-6 ng/mL). In these studies, the ocular samples consisted of an eye cup and not the entire eye, which might be affecting the volume and level of VEGF-Trap in the samples. Hence, it was decided to include whole eyes in future studies. Similar to VEGF-Trap, AAV2 capsid protein ELISA was also affected by the volume of the samples. Commercial aflibercept (40 μg/eye) was injected as a reference and positive control. 4.0E+08 vg/eye of AVMX-110 showed similar lesion recovery as commercial aflibercept. Since the results of these two studies were similar, it could be concluded that this AVMX-110 dose showed similar results as commercial aflibercept. Overall, the mouse MNV model was a relevant model for initial construct screening before proceeding to larger animal models, such as NHP.

### Example 14. Efficacy of Modified Adeno-Associated Virus (AAV6.N54-120) Encoding Aflibercept (AVMX-116) in a Laser-Induced Choroidal Neovascularization (CNV) Model

Example 14 illustrates evaluation of inhibition of neovascularization in a laser-induced model of choroidal neovascularization (LCNV) in the mouse using different serotypes of adeno-associated viral vectors in Groups 1-5 and to evaluate the distribution of AAV6-N54-GFP, AAV1-N54-GFP, and AAV2.N54-GFP in mouse retina tissue in Groups 6-8; two naïve animals (Group 9) were included for the method development and bioanalysis efforts. Table 33 illustrates the experimental design.

TABLE 33
Experimental design

| | | OU | | | | |
|---|---|---|---|---|---|---|
| | No. of | Treatment/ | Volume/ | Treatment | CNV | |
| Gp | Animals | Dose | Route | Day | Induction | Experimental Endpoints |
| 1 | 10 | AAV6.N54- | 1.0 μL | Day −28 | OU: CNV | DAY 7: |
| | CNV: 8 | Δ120 | IVT | | Laser Day | Fluorescein |
| | PK: 2 | 1.6e+10 | | | 0 | angiography (CNV |
| | | vg/eye | | | (n = 8/group) | animals only): n = 8 |
| | | *Sham Vector | | | | mice/group |
| | | | | | | Terminal blood |
| | | | | | | collection (serum): |
| 2 | 10 | AVMX-110 | | | | n = 10 mice/group |
| | CNV: 8 | 4e+8 vg/eye | | | | Flatmount analysis |
| | PK: 2 | CNV | | | | (CNV animals only): |
| | | positive | | | | n = 8 mice/group |
| | | Control | | | | Non-CNV animals |
| 3 | 10 | AVMX-116 | | | | (n = 2 animals/group). |
| | CNV: 8 | 2e7 vg/eye | | | | |
| | PK: 2 | | | | | |
| 4 | 10 | AVMX-116 | | | | |
| | CNV: 8 | 4e8 vg/eye | | | | |
| | PK: 2 | | | | | |
| 5 | 10 | AVMX-116 | | | | |
| | CNV: 8 | 1.6e10 | | | | |
| | PK: 2 | vg/eye | | | | |
| 6 | 2 IHC | AAV1.N54- | | | N/A | DAY 0: |
| | | GFP | | | | Color/Cobalt Blue |
| | | 5e9 vg/eye | | | | Fundus Imaging |
| 7 | 2 IHC | AAV2.N54- | | | | IHC for GFP: n = 2 |
| | | GFP | | | | mice/group |
| | | 5e9 vg/eye | | | | |
| | | GFP | | | | |
| | | Positive | | | | |
| | | Control | | | | |
| 8 | 2 IHC | AAV6.N54- | | | | |
| | | GFP | | | | |
| | | 5e9 vg/eye | | | | |
| 9 | 2 PK | N/A | N/A | N/A | N/A | DAY 7: |
| | | | | | | Terminal blood |
| | | | | | | collection (serum): |
| | | | | | | n = 2 mice |

Abbreviations: CNV—choroidal neovascularization; IVT—Intravitreal; OU - both eyes. For Groups 1-5, n = 8 mice/group will receive an IVT injection of test material on Day −28 and then undergo laser-induced choroidal neovascularization; n = 2 animals/group will only receive the IVT injection and will be processed for pharmacokinetic ELISA analyses. Sham vector is the non-expression vector of AVMX-110 whose open reading frame (ORF) of GOI (aflibercept) was disrupted.

### Methods

On day 28, mice were given buprenorphine 0.01-0.05 mg/kg subcutaneously (SQ). Animals were then tranquilized for the intravitreal injections with inhaled isoflurane and one drop of 0.5% proparacaine HCl was applied to both eyes. The conjunctiva was gently grasped with Dumont #4 forceps, and the injection was made using a 33 G needle and a Hamilton Syringe. After dispensing the syringe contents, the syringe needle was slowly withdrawn. Following the injection procedure, 1 drop of Ofloxacin ophthalmic solution was applied topically to the ocular surface with eye lube.

On day 0, mice were given buprenorphine 0.01-0.05 mg/kg SQ. A topical mydriatic (1.0% tropicamide HCl, and 2.5% phenylephrine HCl) was applied at least 15 minutes prior to the laser procedure. The mice were tranquilized with an intraperitoneal (IP) injection of ketamine/xylazine. The cornea was kept moistened using topical eyewash, and body temperature was maintained using hot pads. A 532 nm diode laser delivered through a slit-lamp was used to create 4 single laser spots surrounding the optic nerve. Both mouse eyes received laser treatment according to the schedule in the Experimental Design. Eye lube was placed (OU) after laser. Morbidity and mortality were observed daily along with cage-side observations, with particular attention paid to both eyes. Animal 436 (Group 4) died on day 7 prior to imaging procedures. The baseline body weight of all animals was 25.1±1.5 g. Animals in all groups gained a normal amount of body weight over the course of the study.

Samples from Groups 1-5 and 9 were shipped or stored on dry ice. Animals allocated for pharmacokinetic analysis (n=2/group) did not undergo LCNV induction for aflibercept quantification. Ocular samples (i.e., whole eye globes) were enucleated, frozen after isolation, and weighed. Table 34 shows the detailed documentation of tissue weight and the volume of 1×PBS with protease inhibitor (PI) added to each sample ahead of homogenization. Samples were placed on ice and homogenized using a sonicator with a 20 second pulse followed by a 20 second rest for three cycles. Following sonication, the samples were rested on ice.

TABLE 34
Tissue Weight and PBS Volume

| | | | Pre- | Post- | Tissue | μL of |
|---|---|---|---|---|---|---|
| Animal | Group | | weight | weight | Weight | PBS + |
| # | # | Eye | (g) | (g) | (g) | PI |
| 109 | 1 | OS | 1.5858 | 1.6089 | 0.0231 | 231 |
| | | OD | 1.6161 | 1.6385 | 0.0224 | 224 |
| 110 | 1 | OS | 1.5831 | 1.6083 | 0.0252 | 252 |
| | | OD | 1.5922 | 1.6177 | 0.0255 | 255 |
| 219 | 2 | OS | 1.6117 | 1.6355 | 0.0238 | 238 |
| | | OD | 1.5975 | 1.6209 | 0.0234 | 234 |
| 220 | 2 | OS | 1.5967 | 1.6213 | 0.0246 | 246 |
| | | OD | 1.5802 | 1.6062 | 0.0260 | 260 |
| 329 | 3 | OS | 1.5930 | 1.6194 | 0.0264 | 264 |
| | | OD | 1.6011 | 1.6278 | 0.0267 | 267 |
| 330 | 3 | OS | 1.5784 | 1.6018 | 0.0234 | 234 |
| | | OD | 1.5927 | 1.6201 | 0.0274 | 274 |
| 439 | 4 | OS | 1.5940 | 1.6188 | 0.0248 | 248 |
| | | OD | 1.5802 | 1.6038 | 0.0236 | 236 |
| 440 | 4 | OS | 1.5970 | 1.6204 | 0.0234 | 234 |
| | | OD | 1.5973 | 1.6226 | 0.0253 | 253 |
| 549 | 5 | OS | 1.5916 | 1.6147 | 0.0231 | 231 |
| | | OD | 1.5819 | 1.6036 | 0.0217 | 217 |
| 550 | 5 | OS | 1.6051 | 1.6302 | 0.0251 | 251 |
| | | OD | 1.6137 | 1.6371 | 0.0234 | 234 |
| 957 | 9 | OS | 1.5959 | 1.6194 | 0.0235 | 235 |
| | | OD | 1.5962 | 1.6200 | 0.0238 | 238 |
| 958 | 9 | OS | 1.5772 | 1.6027 | 0.0255 | 255 |
| | | OD | 1.5960 | 1.6207 | 0.0247 | 247 |

The aflibercept expression in ocular (whole eye), and serum samples was quantified using the aflibercept ELISA. Briefly, 0.1 μg/mL VEGF was coated onto the 96-well plate and incubated 0/N at 4° C. The plate was washed with wash buffer and blocked with blocking buffer. Ocular and serum samples were delivered to the specified wells directly without any dilution with the exception of Group 5 samples (1:5, 1:10, and 1:100 dilution). Samples were incubated for 1 hour. Plates were washed and the detection antibody was added into each well at a 1:40,000 dilution. After a 1 hour incubation, plates were washed and Streptavidin-horse radish peroxidase (HRP) was added at a 1:40,000 dilution. After a 45 minutes of incubation, plates were washed, and aflibercept was detected by the addition of 3,3′,5,5′-tetramethylbenzidine (TMB) substrate. The reaction was stopped by the addition of stop solution, and the plate was read immediately at 450 nm along with the reference wavelength of 600 nm.

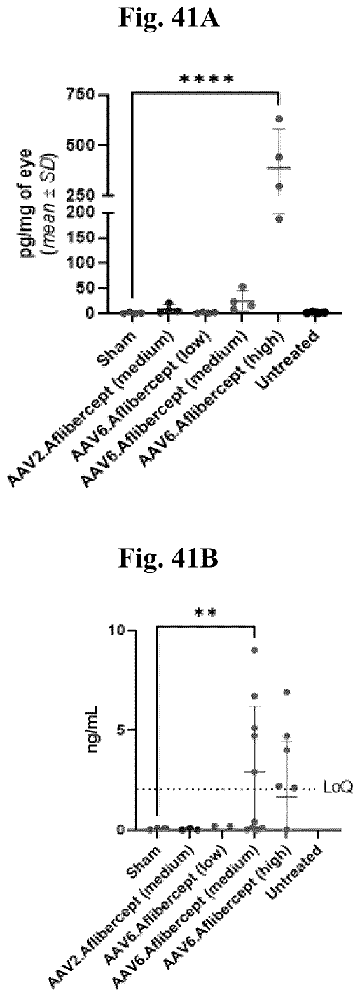

Aflibercept expression in the animals treated with the sham vector control and untreated animals was similar. In Group 3, animals that were injected with AAV6.N54-Aflibercept, about 2.3 pg aflibercept/mg of ocular homogenate was measured (FIG. 41A). AAV2 and the mid-range level for AAV6.N54-Aflibercept had about 8.8 and about 25 pg aflibercept/mg of ocular homogenate respectively, which was a threefold-elevated expression for the AAV6-treated animals. High dose-AAV6 treated animals showed the highest level of aflibercept, with about 400 pg aflibercept/mg of ocular homogenate. Serum samples followed a similar trend as the ocular tissue samples. Group 1 (Sham), 2 (AAV2-Aflibercept) and 3 (AAV6-low dose) had no detectable aflibercept. Both the medium and high dose AAV6-Aflibercept treated animals showed 2-3 ng/mL aflibercept in the serum (FIG. 41B). The data were analyzed in GraphPad Prism software using a one-way ANOVA followed by Dunnett's multiple comparison (**=0.005 and ****=<0.0001). Aflibercept expression is located in Table 35 (showing that AAV1.N54-Aflibercept and AAV6.N54-Aflibercept expressed 30-fold higher Aflibercept than AAV2.N54-Aflibercept) and Table 36.

TABLE 35
Aflibercept Expression Level in Mice by 3 AAV Vectors

| | Dose/eye | Eye Cup (Retina) | | | Serum | | |
|---|---|---|---|---|---|---|---|
| Serotype | (IVT) | ng/mL | pg/eye cup | -fold | ng/mL | pg/animal | -fold |
| AAV2.N54-A120 | 1.6e+10 | <0.01 | <0.01 | NA | <0.01 | <0.01 | NA |
| Sham vector | vg/eye | | | | | | |
| AAV1.N54-Aflibercept | 4.8e+8 | 7.2 ± 5.3 | 0.3 ± 0.3 | 30 | 2.4 ± 0.5 | 0.6 ± 0.1 | 60 |
| | vg | | | | | | |
| AAV2.N54-Aflibercept | 4.8e+8 | 0.1 ± 2.2 | 0.01 ± 0.1 | 1.0 | 0.0 ± 0.12 | 0.0 ± 0.03 | 1.0 |
| | vg | | | | | | |
| AAV6.N54-Aflibercept | 4.8e+8 | 6.6 ± 8.1 | 0.31 ± 0.3 | 31 | 1.3 ± 0.95 | 0.3 ± 0.2 | 30 |
| | vg | | | | | | |

TABLE 36
Aflibercept Expression in Ocular Homogenate and Serum Samples

| | | | Eye Cup (Retina)/Eyeball | | | | |
|---|---|---|---|---|---|---|---|
| | | Dose/eye | | pg/eye | pg/eye | Serum | |
| Product | Entity | (IVT) | ng/mL | cup | ball | ng/mL | ng/animal |
| Vehicle | | | ≤LoQ* | ≤LoQ | ND | ≤LoQ | −0.01 ± 0.02 |
| (FB-01) | | | | | | | |
| Sham | AAV2.N54.Δ120 | 4.8E+08 | ≤LoQ | ≤LoQ | ND | ≤LoQ | −0.1 ± 0.0 |
| vector | | vg/eye | | | | | |
| control | | 1.6E+10 | 0.2 ± 0.1 | | 1.8 ± 1.2 | ≤LoQ | −0.01 ± 0.01 |
| | | vg/eye | ≤LoQ | | ≤LoQ | | |
| Aflibercept | Commercial | 40 | ≤LoQ | ≤LoQ | ND | 48.5 ± 10.8 | 9.71 ± 2.2 |
| (Protein) | Eylea | μg/eye | | | | | |
| AVMX-110 | AAV2.N54- | 2E+07 | ≤LoQ | ≤LoQ | ND | 0.4 ± 0.5 | 0.06 ± 0.08 |
| Study | Aflibercept | vg/eye | | | | ≤LoQ | |
| Number | | | | | | | |
| 21-AVI- | | 4.8E+08 | 0.03 ± 2.3 | 2.4 ± 20 | 8.8 ± 8.6** | 0.1 ± 0.4 | 0.005 ± 0.02 |
| 002 | | vg/eye | (eye cup); | | | ≤LoQ | |
| | | | 0.9 ± 0.9 | | | | |
| | | | (globe) | | | | |
| | | | ≤LoQ | | | | |
| | | 1.6E+10 | 4 ± 2.7 | 36.4 ± 24 | ND | 1.3 ± 1.6 | 0.191 ± 0.22 |
| | | vg/eye | | | | ≤LoQ | |
| AVMX-116 | AAV6.N54- | 2E+07 | 0.2 ± 0.1 | ND | 2.3 ± 1.0 | ≤LoQ | ≤LoQ |
| Study | Aflibercept | vg/eye | ≤LoQ | | | | |
| Number | | | | | | | |
| 21-AVI- | | 4.8E+08 | 5.1 ± 5.2 | 51.3 ± 51.5** | 25.2 ± 19.6 | 2.4 ± 2.9 | 0.17 ± 0.2 |
| 004/−007 | | vg/eye | (eye cup); | | | | |
| | | | 2.5 ± 2.0 | | | | |
| | | | (globe) | | | | |
| | | 1.6E+10 | 39 ± 19.2 | | 389.7 ± 192.4 | 1.7 ± 2.8 | 0.17 ± 0.3 |
| | | vg/eye | | | | | |

*≤LoQ = 2 ng/mL

### Fundus Imaging (Groups 6-8, Day 0)

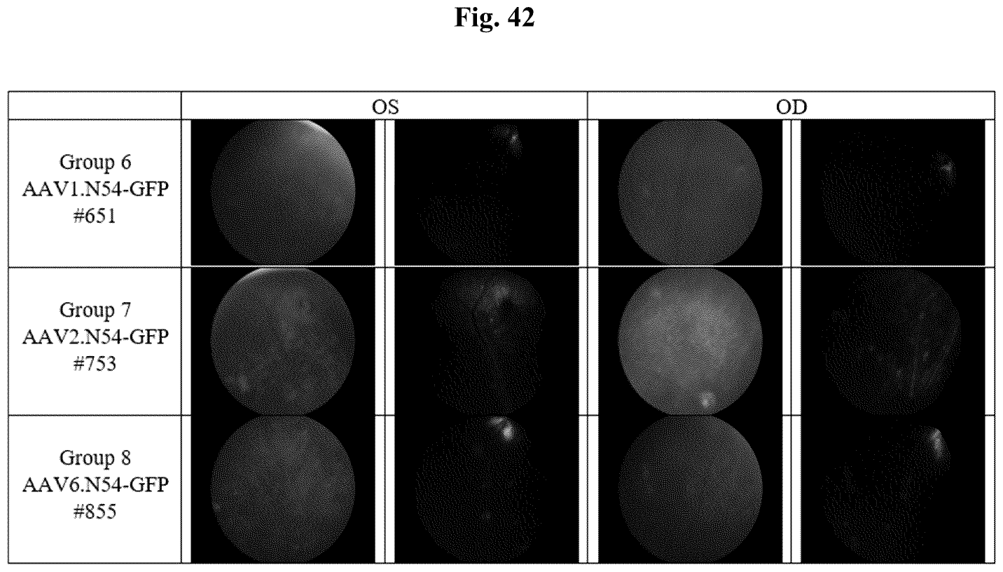

Color and cobalt blue fundus imaging for EGFP was performed on both eyes from the animals enrolled in Groups 6-8. Animals were given topical mydriatic (1.0% tropicamide HCl and 2.5% phenylephrine HCl) to dilate and proptose the eyes, and topical anesthetic was applied to the eyes (proparacaine 0.5%). Color fundus photography was followed by cobalt blue photography. Acquisition settings were set to Group 7 (positive control) and held consistent across Groups 6 and 8. Representative images are located in FIG. 42. GFP signal was observed in all three groups. It was strongest and brightest in Group 7 (positive control), then Group 8, then Group 6. Expression was more restricted than observed in previous studies despite holding the exposure time consistent across all studies.

### Fluorescein Angiography (FA)

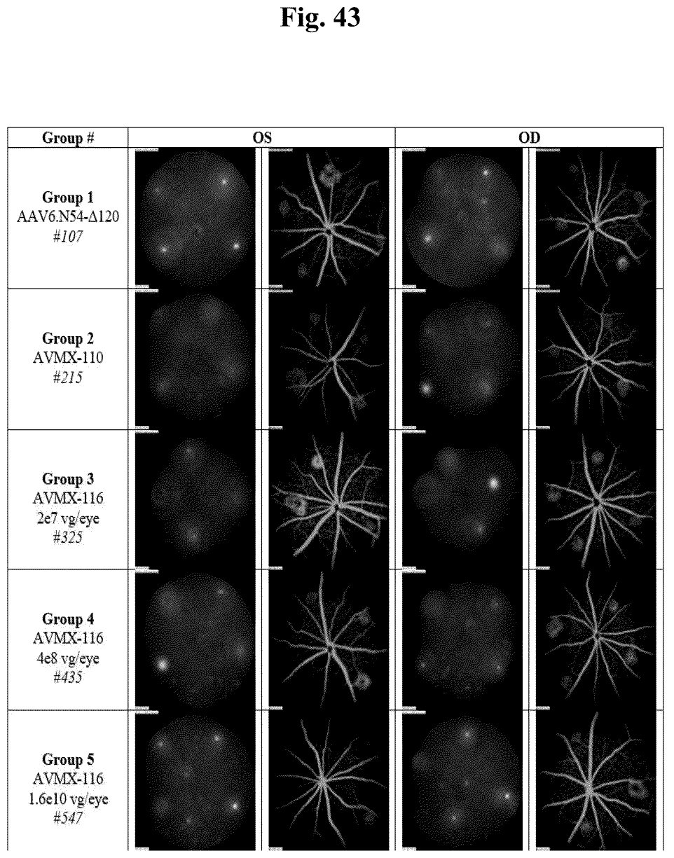

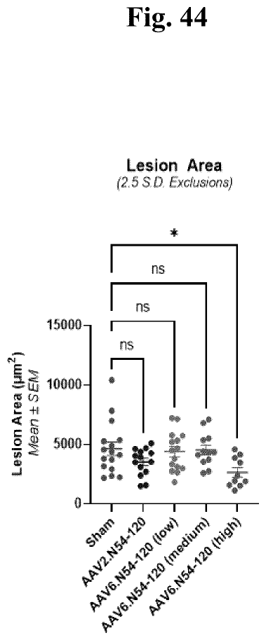

FA was performed on both eyes on day 7 post-laser. Mydriasis for FA was achieved using a topical drop in each eye 15 minutes prior to examination (1.0% tropicamide HCl and 2.5% phenylephrine HCl). The mice were tranquilized with an IP injection of ketamine/xylazine. Retinal photography was performed approximately 1 minute after intravenous sodium fluorescein injection (1% stock solution; 12 mg/kg). Fluorescein leakage was analyzed with ImageJ software; representative images showing the lesions are shown below in FIG. 43. Angiograms were used to quantify lesion area as pixels, 2.5 standard deviations (SD), and the results are displayed in FIG. 44. When analyzing Isolectin area size, lesions where Bruch's membrane did not rupture, where vitreal hemorrhages were noted, or where data points more than 2.5 standard deviations from the mean were excluded. The Group 5 (highest dose of AVMX-116) test material outperformed the Group 2 AVMX-110 (positive control), having the lowest lesion area of all groups. Statistical analysis was performed by one-way ANOVA followed by Dunnett multiple comparison on GraphPad Prism software (*=0.0138).

When the data from this study were compared to a previous study, homogenization of whole eyes was superior for the estimation of aflibercept expression when compared to eye cups only, increasing the accuracy of quantification. Ocular samples from AAV6.Aflibercept showed about 10-fold increase in protein expression with a 20-fold increase in viral vector dose, and when compared to AAV2, the AAV6 constructs showed a 3-fold enhancement in aflibercept expression. Animals receiving the medium- or high-dose AAV6.Aflibercept demonstrated considerable aflibercept expression in serum samples. The study showed the average CNV lesion size evaluated via fluorescein angiography (FA) was greatly reduced in animals receiving IVT injections of their positive control, AVMX-110, consistent with a other study utilizing the same construct.

### Ocular Tissue Collection and Processing

On Day 7, animals were humanely euthanized via carbon dioxide asphyxiation and death was confirmed by cervical dislocation.

Tissue Collection for Flatmount Analysis (Groups 1-5 only)

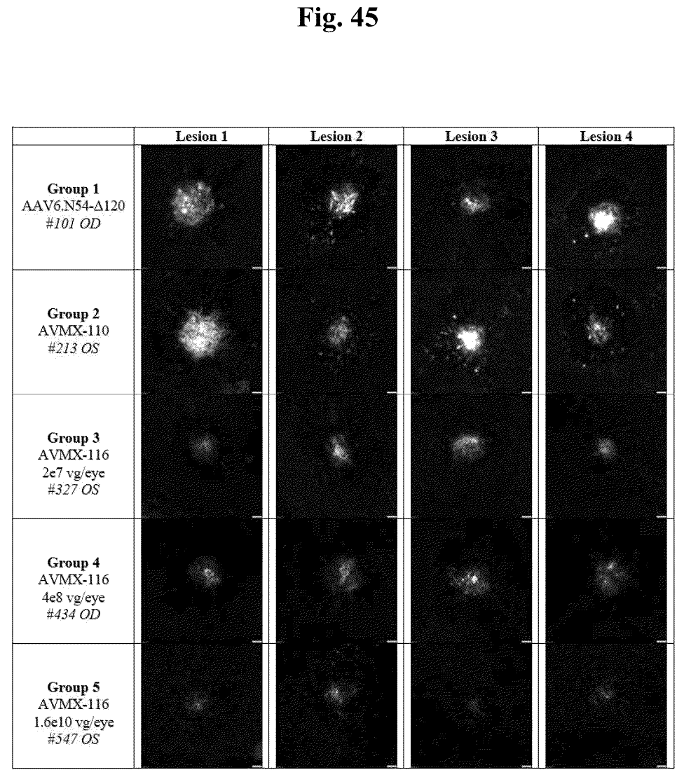

Eyes were enucleated and immediately fixed in 4% paraformaldehyde in phosphate-buffered saline (PBS) and stored overnight at 4° C. The following day, the eyes were transferred to cold immunocytochemistry (ICC) buffer (PBS containing 0.5% BSA and 0.2% Tween 20) until processing. Using a dissecting microscope, the eye was carefully trimmed of extraneous tissue and the anterior segment and lens were removed. The retina was detached and removed from the optic nerve head with fine curved scissors. Eye cups were rinsed with cold ICC buffer. Eye cups were placed in cold ICC containing 4′,6-diamidino-2-phenylindole (DAPI; nuclear stain) isolectin IB4 AlexaFluor 649 (blood vessels; Vector Labs cat #DL-1208-.5), and phalloidin AlexaFluor 555 (f-actin; ThermoFisher A34055). Eye cups were incubated at 4° C. with gentle rotation for 4 hours and washed with cold ICC buffer. Radial cuts were then made toward the optic nerve head avoiding lesions, and the sclera-choroid/RPE complexes were, flat mounted, covered, and sealed. Two-dimensional (2D) fluorescent microscopy images were acquired, digitized, and analyzed using an Olympus Bx63 upright fluorescent microscope and Cell Sens (Olympus) software. Post-acquisition analysis was performed with Cell Sens software. To quantify neoformed vessels, the isolectin IB4 area was measured in μm2. Representative images from the isolectin lesion area measurements are located in FIG. 45.

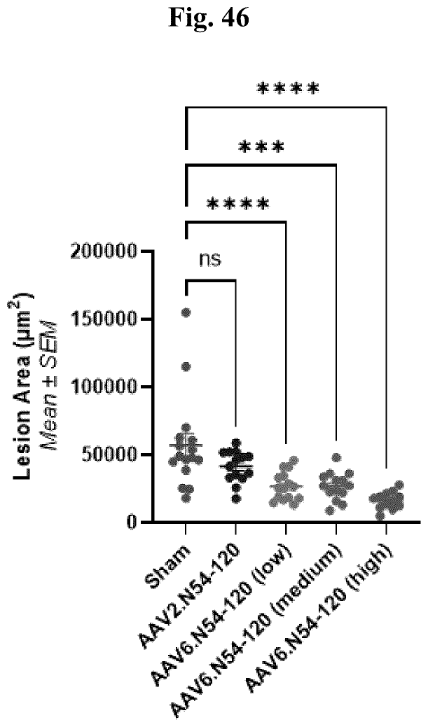

When analyzing isolectin area size (FIG. 46), lesions where Bruch's membrane did not rupture, where vitreal hemorrhages were noted, or where data points more than 2.5 standard deviations from the mean were excluded. Group 1 animals had the largest lesion area at 60,900±38,500 μm², while Group 5 had the smallest lesion area at 18,700±7,200 μm², a 70% reduction in lesion area. The positive control performed similarly to other studies, with a lesion area 30% smaller than sham vector controls. Statistical analysis was done using one-way ANOVA followed by Dunnett multiple comparison (***=0.0001 and ****=<0.0001).

### Tissue Collection for Immunohistochemistry (Groups 6-8 Only)

All eyes designated for immunohistochemistry (IHC) were enucleated, the approximate site of injection was marked, and the tissue was then placed into 1×PBS. Eyes were fixed in 4% paraformaldehyde in separately labeled vials overnight at 4° C. Eyes were then transferred into 0.1 M phosphate buffer (PB), brought through a sequential sucrose gradient (10-30%, 1 hour each) followed by embedding in OCT medium and freezing on dry ice. The entire eye was cryosectioned (14 μm sections) and stained with the following antibodies: 1/250 chicken anti-GFP, 1/250 rabbit anti-RPE65, and followed by 1/200 anti-chicken Cy2, 1/200 anti-rabbit Cy3, 1/100 PNA Lectin Cy5, and 1/1,000 DAPI.

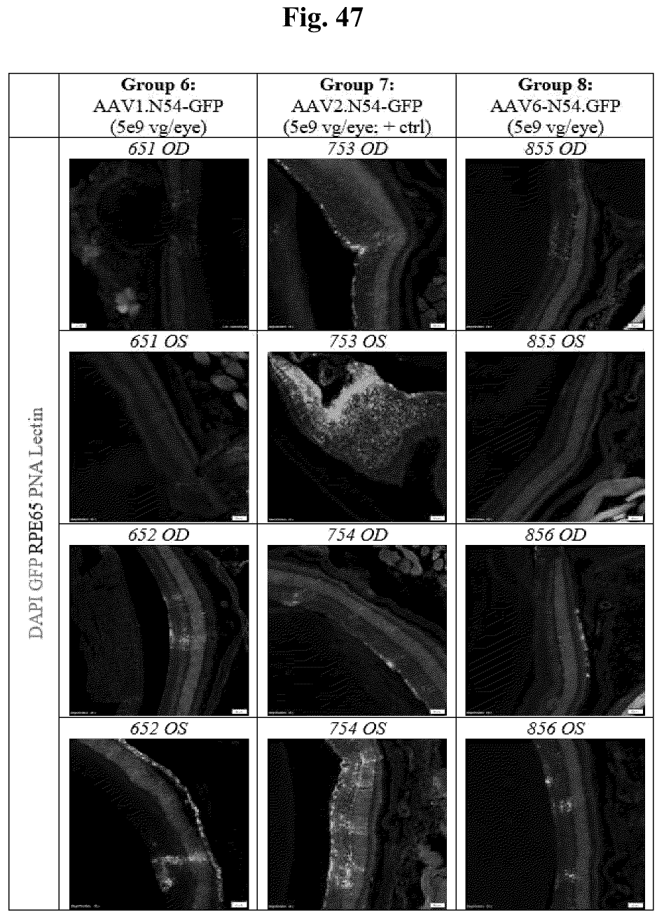

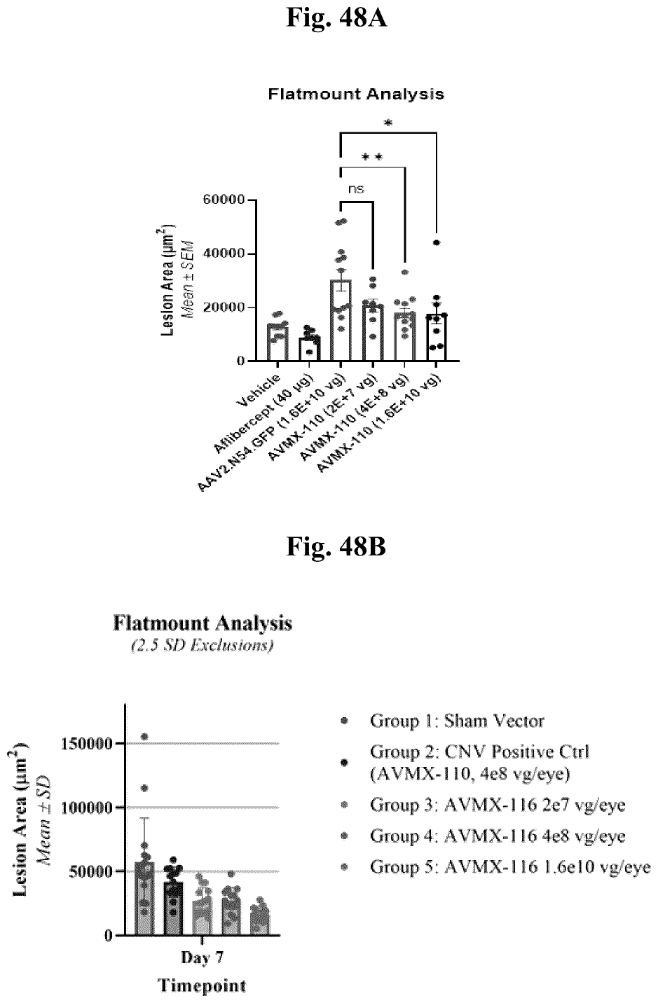

Groups 6 and 7 had the brightest and broadest GFP signal, but Group 8 had GFP signal as well. In all groups, GFP expression tended to be localized in the central part of the retina near the optic nerve, but there was some geographic variability. Expression was consistently observed from the ganglion cell layer through the inner segment and was less consistently seen in the outer segment and retinal pigment epithelium. The GFP signal seen in the stained cryosections matched the GFP signal seen in the fundus imaging. The RPE staining seen in 856 OD was due to transduction of the AAV into the RPE following a standard IVT injection while the GFP expression observed in the RPE of mouse 652 OS may have been enhanced due to the unintentional delivery of the AAV to the subretinal space, rather than the intravitreal space. Representative images of day 0 IHC are located in FIG. 47. FIG. 48A illustrates Flatmount analysis of the images obtained from the dose response study of Example 14. FIG. 48B illustrates fluorescence angiograph (FA) analysis of the images obtained from the dose response study of Example 14.

### Example 15. AVMX Formulation and Efficacy Study

AVMX-110 was formulated into a liquid compotation sterile vials. AVMX-110 drug product concentrate for solution for IVT injection was manufactured as a sterile frozen liquid formulation stored in a 1 mL of 13 mm cyclic olefin copolymer (COC) vials with a 13 mm gray bromobutyl stopper and a 13 mm flip-off aluminum seal. Each vial was filled to a target weight of 100 μL to allow for the withdrawal volume of at least 50 μL (density 1.01 g/mL). The product was supplied at a concentration of ≥4.0×1012 vg/mL. For clinical use, drug product was thawed and aseptically withdraw with provided filter needle and delivered to patient via IVT by retina specialist.

The efficacy studying with AVMX-110: AAV2.N54-VEGF-Trap was performed based on the protocol shown in Table 37. Efficacy of AVMX-110 in mice was evaluated by using a Laser-Induced Choroidal Neovascularization (LCNV) Model.

TABLE 37
Experimental design of mouse LCNV

| | | OU | | | |
|---|---|---|---|---|---|
| | No. of | Treatment/ | Volume/ | Treatment | CNV |
| Group | Animals | Dose | Route | Day | Induction |
| | 8 | Vehicle | 1.0 μL | Day-3 | OU: CNV |
| | | Formulation | IVT | | Laser Day 0 |
| | | buffer | | | |
| 2 | 8 | AAV2.N54- | | Day-28 | |
| | | GFP | | | |
| | | 1.6e+10 | | | |
| | | vg/eye | | | |
| 3 | 8 | Aflibercept | | Day-3 | |
| | | (Eylea ®) 40 | | | |
| | | μg | | | |
| 4 | 8 | AVMX110 | | Day-28 | |
| | | Low Dose | | | |
| | | 2e+7 vg/eye | | | |
| 5 | 8 | AVMX110 | | | |
| | | Medium Dose | | | |
| | | 4e+8 vg/eye | | | |
| 6 | 8 | AVMX110 | | | |
| | | High Dose | | | |
| | | 1.6e+10 | | | |
| | | vg/eye | | | |
| 7 | 2 | NA | NA | NA | NA |

Animals were acclimated to the study environment for a minimum of 3 days. At the completion of the acclimation period, each animal was physically examined by a laboratory animal technician for determination of suitability for study participation. Examinations included, but not be limited to, the skin and external ears, eyes, abdomen, behavior, and general body condition. Animals determined to be in good health were released to the study. The animal randomization, intravitreal injection, laser-induced CNV procedure, animal examination, fluorescein angiography (FA), ocular tissue collection and flat mount (n=6 mice/group) were described in the protocol. In addition, all mice were terminally bled to collect serum and retina tissues after observation by fluorescent angiography (FA) and collection of flat mounts.

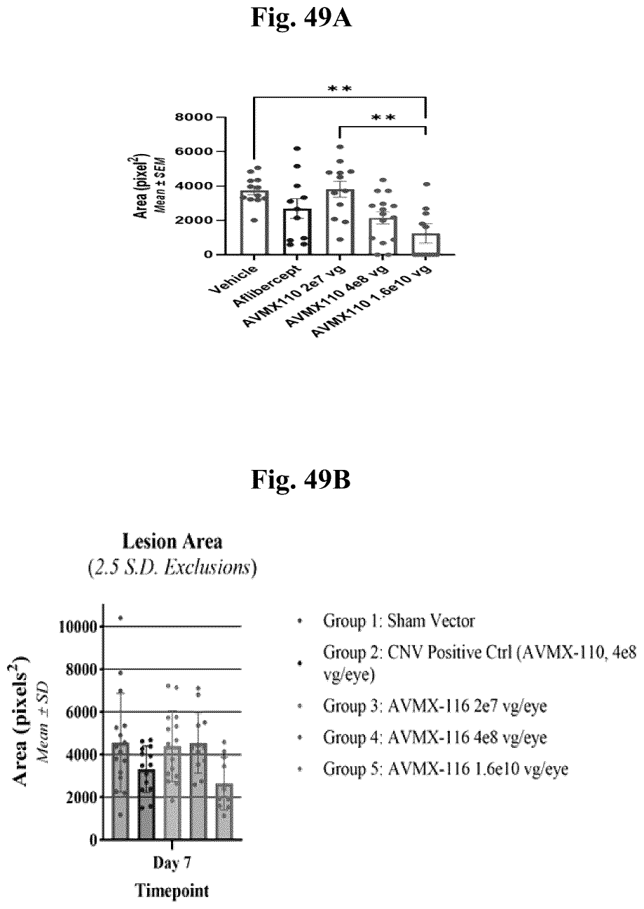

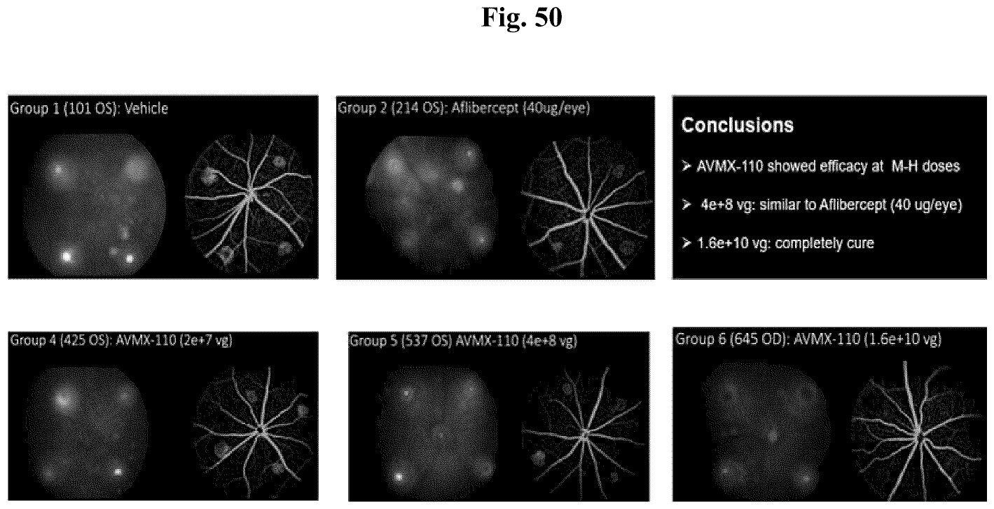

In FA analysis, animals received aflibercept at 40 μg/eye or AVMX-110 IVT-injection showed significant reduction of lesion area (FIG. 49A). For AVMX-110 groups, the lesion area reduction values were correlated inversely with dose of vector genome copies (vg) (P<0.01 for 4e+8 vg/eye and P<0.001 at 1.6e+10 vg/eye), the higher vector injected, the smaller damaged area observed 7 days post laser shinning (Table 38). In the case of 1.6e+10 vg/eye, complete cure of laser injury was achieved. FIG. 49B illustrates FA analysis of the images from the AVMX-110 dosing study.

TABLE 38
Statistical analysis of AVMX-110 efficacy in LCNM

| Dunnett's multiple comparisons test | Adjusted P Value |
|---|---|
| Group 1: Vehicle vs. Group 2: Aflibercept | 0.055 |
| Group 1: Vehicle vs. Group 4: AVMX110 2e7 vg | 0.999 |
| Group 1: Vehicle vs. Group 5. AVMX110 4e8 vg | 0.0113 |
| Group 1: Vehicle vs. Group 6: AVMX110 1.6e10 vg | 0.0002 |

Following euthanasia, animals in Groups 1-5 and Group 9 had blood collected for serum isolation and eyes were enucleated and snap frozen. The eyes were placed into appropriate pre-weighed labeled analytical vials, immediately reweighed to determine sample weight, and placed on dry ice until being transferred to a freezer. Samples were weighed on a balance capable of measuring out to 4 decimal places. Following snap freezing and weighing, samples were returned to the −80° C. freezer. VEFG-Trap concentration in serum and retina tissues were tested by ELISA assay. The eyes were enucleated and the retina and RPE/choroid segments were dissected from fresh eyes and snap frozen. The tissues were placed into appropriate pre-weighed labeled analytical vials, immediately reweighed to determine sample weight, and placed on dry ice until being transferred to a freezer. Samples were weighed on a balance capable of measuring out to 4 decimal places.

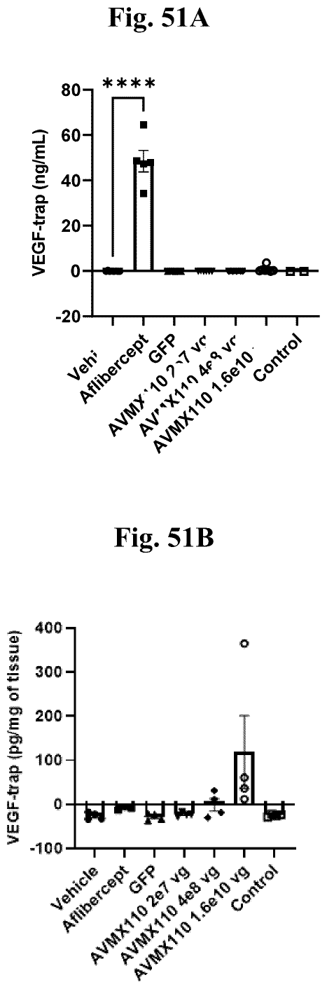

The samples were assayed using ELISA. VEGF-Trap was ˜50 ng/mL for the aflibercept control group, low level of VEGF-Trap at ˜2 ng/mL was detected in sera of high dose group, 1.6e10 vg/eye and was not detectable in the low and medium dose groups (FIG. 51A). VEGF-Trap level in retina tissue was also measured in the retina homogenates using ELISA (FIG. 51B). In the retina tissue suspension, the expression of VEGF-Trap is in good dose dependent manner to the AVMX-110 injected intravitreally. On average of 13 ng/mL of VEGF-Trap was detected for the high dose group of 1.6e10 vg/eye. The highest expression level reached over 40 ng/mL.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

Sequence Table

| SEQ | | |
|---|---|---|
| ID | | |
| NO: | Annotation | Sequence |
| 12 | VEGF-Trap | MVSYWDTGVLLCALLSCLLLTGSSSGSDTG |
| | plasmid | RPFVEMYSEIPEIIHMTEGRELVIPCRVTS |
| | | PNITVTLKKFPLDTLIPDGKRIIWDSRKGF |
| | | IISNATYKEIGLLTCEATVNGHLYKTNYLT |
| | | HRQTNTIIDVVLSPSHGIELSVGEKLVLNC |
| | | TARTELNVGIDFNWEYPSSKHQHKKLVNRD |
| | | LKTQSGSEMKKFLSTLTIDGVTRSDQGLYT |
| | | CAASSGLMTKKNSTFVRVHEKDKTHTCPPC |
| | | PAPELLGGPSVFLFPPKPKDTLMISRTPEV |
| | | TCVVVDVSHEDPEVKFNWYVDGVEVHNAKT |
| | | KPREEQYNSTYRVVSVLTVLHQDWLNGKEY |
| | | KCKVSNKALPAPIEKTISKAKGQPREPQVY |
| | | TLPPSRDELTKNQVSLTCLVKGFYPSDIAV |
| | | EWESNGQPENNYKTTPPVLDSDGSFFLYSK |
| | | LTVDKSRWQQGNVFSCSVMHEALHNHYTQK |
| | | SLSLSPGK |
| 13 | AMI059 | CATTCGCCATTCAGGCTGCAAATAAGCGTT |
| | | GATATTCAGTCAATTACAAACATTAATAAC |
| | | GAAGAGATGACAGAAAAATTTTCATTCTGT |
| | | GACAGAGAAAAAGTAGCCGAAGATGACGGT |
| | | TTGTCACATGGAGTTGGCAGGATGTTTGAT |
| | | TAAAAACATAACAGGAAGAAAAATGCCCCG |
| | | CTGTGGGCGGACAAAATAGTTGGGAACTGG |
| | | GAGGGGTGGAAATGGAGTTTTTAAGGATTA |
| | | TTTAGGGAAGAGTGACAAAATAGATGGGAA |
| | | CTGGGTGTAGCGTCGTAAGCTAATACGAAA |
| | | ATTAAAAATGACAAAATAGTTTGGAACTAG |
| | | ATTTCACTTATCTGGTTCGGATCTCCTAGG |
| | | CTCAAGCAGTGATCAGATCCAGACATGATA |
| | | AGATACATTGATGAGTTTGGACAAACCACA |
| | | ACTAGAATGCAGTGAAAAAAATGCTTTATT |
| | | TGTGAAATTTGTGATGCTATTGCTTTATTT |
| | | GTAACCATTATAAGCTGCAATAAACAAGTT |
| | | AACAACAACAATTGCATTCATTTTATGTTT |
| | | CAGGTTCAGGGGGAGGTGTGGGAGGTTTTT |
| | | TAAAGCAAGTAAAACCTCTACAAATGTGGT |
| | | ATGGCTGATTATGATCCTCTAGTACTTCTC |
| | | GACAAGCTCGGATCCTGGCGCGCTCGCTCG |
| | | CTCACTGAGGCCGCCCGGGCAAAGCCCGGG |
| | | CGTCGGGCGACCTTTGGTCGCCCGGCCTCA |
| | | GTGAGCGAGCGAGCGCGCAGAGAGGGAGTG |
| | | GCCAACTCCATCACTAGGGGTTCCTAGGAA |
| | | GCTGATCTGAATTCGGTACCCGTTACATAA |
| | | CTTACGGTAAATGGCCCGCCTGGCTGACCG |
| | | CCCAACGACCCCCGCCCATTGACGTCAATA |
| | | ATGACGTATGTTCCCATAGTAACGCCAATA |
| | | GGGACTTTCCATTGACGTCAATGGGTGGAG |
| | | TATTTACGGTAAACTGCCCACTTGGCAGTA |
| | | CATCAAGTGTATCATATGCCAAGTACGCCC |
| | | CCTATTGACGTCAATGACGGTAAATGGCCC |
| | | GCCTGGCATTATGCCCAGTACATGACCTTA |
| | | TGGGACTTTCCTACTTGGCAGTACATCTAC |
| | | GTATTAGTCATCGCTATTACCATGGTGATG |
| | | CGGTTTTGGCAGTACATCAATGGGCGTGGA |
| | | TAGCGGTTTGACTCACGGGGATTTCCAAGT |
| | | CTCCACCCCATTGACGTCAATGGGAGTTTG |
| | | TTTTGGCACCAAAATCAACGGGACTTTCCA |
| | | AAATGTCGTAACAACTCCGCCCCATTGACG |
| | | CAAATGGGCGGTAGGCGTGTACGGTGGGAG |
| | | GTCTATATAAGCAGAGCTCGTTTAGTGAAC |
| | | CGTCAGATCGCCTGGAGACGCCATCCACGC |
| | | TGTTTTGACCTCCATAGAAGACACCGGGAC |
| | | CGATCCAGCCTCCGGACTCTAGAGTTAACT |
| | | GGTAAGTTTAGTCTTTTTGTCTTTTATTTC |
| | | AGGTCCCGGATCCGGTGGTGGTGCAAATCA |
| | | AAGAACTGCTCCTCAGTGGATGTTGCCTTT |
| | | ACTTCTAGGCCTGCCGCCACCATGGTATCC |
| | | TATTGGGATACGGGTGTTCTCTTGTGTGCA |
| | | CTCCTTTCCTGTCTCCTGCTCACTGGATCT |
| | | TCTTCTGGGTCTGATACTGGTAGACCTTTT |
| | | GTAGAAATGTATTCAGAAATTCCGGAAATA |
| | | ATTCATATGACAGAAGGACGAGAACTCGTT |
| | | ATACCATGTCGCGTCACGTCCCCTAATATT |
| | | ACTGTTACGCTCAAGAAGTTTCCTCTCGAT |
| | | ACACTTATTCCAGATGGGAAACGCATAATT |
| | | TGGGATTCACGCAAAGGGTTTATTATTAGT |
| | | AACGCAACGTATAAAGAAATTGGGCTGCTC |
| | | ACATGTGAAGCTACGGTAAATGGGCATCTT |
| | | TATAAAACAAATTATTTGACTCATCGGCAA |
| | | ACTAATACTATTATCGATGTAGTACTCTCC |
| | | CCATCCCATGGTATTGAATTGTCAGTTGGG |
| | | GAGAAGTTGGTATTGAATTGTACTGCACGG |
| | | ACAGAACTCAACGTTGGTATTGATTTTAAT |
| | | TGGGAATATCCATCATCTAAACATCAGCAT |
| | | AAGAAGTTGGTAAATCGTGATCTCAAAACT |
| | | CAAAGTGGGTCCGAAATGAAGAAGTTTCTG |
| | | TCCACACTTACGATTGATGGGGTCACTAGA |
| | | AGTGATCAAGGGCTCTATACGTGTGCAGCA |
| | | TCTAGTGGGTTGATGACAAAGAAGAATTCA |
| | | ACTTTTGTTCGTGTCCATGAAAAGGATAAA |
| | | ACACATACTTGTCCACCGTGTCCTGCGCCA |
| | | GAACTTCTCGGTGGTCCATCCGTCTTTCTC |
| | | TTTCCACCTAAACCAAAAGATACTTTGATG |
| | | ATTTCACGGACTCCAGAAGTAACATGTGTT |
| | | GTCGTTGATGTATCACACGAAGATCCAGAA |
| | | GTCAAATTTAATTGGTATGTTGATGGTGTA |
| | | GAAGTTCATAATGCGAAGACAAAACCACGA |
| | | GAAGAACAATACAATAGTACATATCGGGTA |
| | | GTATCCGTCTTGACTGTACTTCACCAAGAT |
| | | TGGCTTAATGGGAAAGAATACAAATGTAAA |
| | | GTTTCTAATAAAGCTCTTCCTGCGCCGATC |
| | | GAAAAGACAATTTCCAAAGCAAAAGGTCAA |
| | | CCTCGGGAACCTCAAGTTTATACGCTCCCA |
| | | CCATCACGGGATGAACTCACTAAGAATCAA |
| | | GTATCCTTGACTTGTCTCGTAAAAGGGTTT |
| | | TATCCTTCAGATATTGCTGTAGAATGGGAA |
| | | TCCAATGGGCAACCAGAAAATAATTATAAA |
| | | ACAACACCACCTGTTCTTGATTCAGATGGT |
| | | TCATTCTTTCTCTATTCCAAACTTACTGTC |
| | | GATAAATCACGCTGGCAACAAGGTAATGTT |
| | | TTCTCTTGTTCCGTCATGCATGAAGCACTC |
| | | CATAATCACTATACGCAAAAGTCTCTCTCT |
| | | CTCTCACCAGGTAAATAATAATTCGAAAAT |
| | | AAAATATCTTTATTTTCATTACATCTGTGT |
| | | GTTGGTTTTTTGTGTGGCATGCTGGGGAGA |
| | | GATCAACCCCACTCCCTCTCTGCGCGCTCG |
| | | CTCGCTCACTGAGGCCGGGCGACCAAAGGT |
| | | CGCCCGACGCCCGGGCTTTGCCCGGGCGGC |
| | | CTCAGTGAGCGAGCGAGCGCGCAGCAAGCT |
| | | GTAGCCAACCACTAGAACTATAGCTAGAGT |
| | | CCTGGGCGAACAAACGATGCTCGCCTTCCA |
| | | GAAAACCGAGGATGCGAACCACTTCATCCG |
| | | GGGTCAGCACCACCGGCAAGCGCCGCGACG |
| | | GCCGAGGTCTTCCGATCTCCTGAAGCCAGG |
| | | GCAGATCCGTGCACAGCACCTTGCCGTAGA |
| | | AGAACAGCAAGGCCGCCAATGCCTGACGAT |
| | | GCGTGGAGACCGAAACCTTGCGCTCGTTCG |
| | | CCAGCCAGGACAGAAATGCCTCGACTTCGC |
| | | TGCTGCCCAAGGTTGCCGGGTGACGCACAC |
| | | CGTGGAAACGGATGAAGGCACGAACCCAGT |
| | | TGACATAAGCCTGTTCGGTTCGTAAACTGT |
| | | AATGCAAGTAGCGTATGCGCTCACGCAACT |
| | | GGTCCAGAACCTTGACCGAACGCAGCGGTG |
| | | GTAACGGCGCAGTGGCGGTTTTCATGGCTT |
| | | GTTATGACTGTTTTTTTGTACAGTCTATGC |
| | | CTCGGGCATCCAAGCAGCAAGCGCGTTACG |
| | | CCGTGGGTCGATGTTTGATGTTATGGAGCA |
| | | GCAACGATGTTACGCAGCAGCAACGATGTT |
| | | ACGCAGCAGGGCAGTCGCCCTAAAACAAAG |
| | | TTAGGTGGCTCAAGTATGGGCATCATTCGC |
| | | ACATGTAGGCTCGGCCCTGACCAAGTCAAA |
| | | TCCATGCGGGCTGCTCTTGATCTTTTCGGT |
| | | CGTGAGTTCGGAGACGTAGCCACCTACTCC |
| | | CAACATCAGCCGGACTCCGATTACCTCGGG |
| | | AACTTGCTCCGTAGTAAGACATTCATCGCG |
| | | CTTGCTGCCTTCGACCAAGAAGCGGTTGTT |
| | | GGCGCTCTCGCGGCTTACGTTCTGCCCAGG |
| | | TTTGAGCAGCCGCGTAGTGAGATCTATATC |
| | | TATGATCTCGCAGTCTCCGGCGAGCACCGG |
| | | AGGCAGGGCATTGCCACCGCGCTCATCAAT |
| | | CTCCTCAAGCATGAGGCCAACGCGCTTGGT |
| | | GCTTATGTGATCTACGTGCAAGCAGATTAC |
| | | GGTGACGATCCCGCAGTGGCTCTCTATACA |
| | | AAGTTGGGCATACGGGAAGAAGTGATGCAC |
| | | TTTGATATCGACCCAAGTACCGCCACCTAA |
| | | CAATTCGTTCAAGCCGAGATCGGCTTCCCG |
| | | GCCGCGGAGTTGTTCGGTAAATTGTCACAA |
| | | CGCCGCGAATATAGTCTTTACCATGCCCTT |
| | | GGCCACGCCCCTCTTTAATACGACGGGCAA |
| | | TTTGCACTTCAGAAAATGAAGAGTTTGCTT |
| | | TAGCCATAACAAAAGTCCAGTATGCTTTTT |
| | | CACAGCATAACTGGACTGATTTCAGTTTAC |
| | | AACTATTCTGTCTAGTTTAAGACTTTATTG |
| | | TCATAGTTTAGATCTATTTTGTTCAGTTTA |
| | | AGACTTTATTGTCCGCCCACACCCGCTTAC |
| | | GCAGGGCATCCATTTATTACTCAACCGTAA |
| | | CCGATTTTGCCAGGTTACGCGGCTGGTCTG |
| | | CGGTGTGAAATACCGCACAGATGCGTAAGG |
| | | AGAAAATACCGCATCAGGCGCTCTTCCGCT |
| | | TCCTCGCTCACTGACTCGCTGCGCTCGGTC |
| | | GTTCGGCTGCGGCGAGCGGTATCAGCTCAC |
| | | TCAAAGGCGGTAATACGGTTATCCACAGAA |
| | | TCAGGGGATAACGCAGGAAAGAACATGTGA |
| | | GCAAAAGGCCAGCAAAAGGCCAGGAACCGT |
| | | AAAAAGGCCGCGTTGCTGGCGTTTTTCCAT |
| | | AGGCTCCGCCCCCCTGACGAGCATCACAAA |
| | | AATCGACGCTCAAGTCAGAGGTGGCGAAAC |
| | | CCGACAGGACTATAAAGATACCAGGCGTTT |
| | | CCCCCTGGAAGCTCCCTCGTGCGCTCTCCT |
| | | GTTCCGACCCTGCCGCTTACCGGATACCTG |
| | | TCCGCCTTTCTCCCTTCGGGAAGCGTGGCG |
| | | CTTTCTCAATGCTCACGCTGTAGGTATCTC |
| | | AGTTCGGTGTAGGTCGTTCGCTCCAAGCTG |
| | | GGCTGTGTGCACGAACCCCCCGTTCAGCCC |
| | | GACCGCTGCGCCTTATCCGGTAACTATCGT |
| | | CTTGAGTCCAACCCGGTAAGACACGACTTA |
| | | TCGCCACTGGCAGCAGCCACTGGTAACAGG |
| | | ATTAGCAGAGCGAGGTATGTAGGCGGTGCT |
| | | ACAGAGTTCTTGAAGTGGTGGCCTAACTAC |
| | | GGCTACACTAGAAGGACAGTATTTGGTATC |
| | | TGCGCTCTGCTGAAGCCAGTTACCTTCGGA |
| | | AAAAGAGTTGGTAGCTCTTGATCCGGCAAA |
| | | CAAACCACCGCTGGTAGCGGTGGTTTTTTT |
| | | GTTTGCAAGCAGCAGATTACGCGCAGAAAA |
| | | AAAGGATCTCAAGAAGATCCTTTGATCTTT |
| | | TCTACGGGGTCTGACGCTCAGTGGAACGAA |
| | | AACTCACGTTAAGGGATTTTGGTCATGAGA |
| | | TTATCAAAAAGGATCTTCACCTAGATCCTT |
| | | TTAAATTAAAAATGAAGTTTTAAATCAATC |
| | | TAAAGTATATATGAGTAAACTTGGTCTGAC |
| | | AGTTACCAATGCTTAATCAGTGAGGCACCT |
| | | ATCTCAGCGATCTGTCTATTTCGTTCATCC |
| | | ATAGTTGCCTGACTCCCCGTCGTGTAGATA |
| | | ACTACGATACGGGAGGGCTTACCATCTGGC |
| | | CCCAGTGCTGCAATGATACCGCGAGACCCA |
| | | CGCTCACCGGCTCCAGATTTATCAGCAATA |
| | | AACCAGCCAGCCGGAAGGGCCGAGCGCAGA |
| | | AGTGGTCCTGCAACTTTATCCGCCTCCATC |
| | | CAGTCTATTAATTGTTGCCGGGAAGCTAGA |
| | | GTAAGTAGTTCGCCAGTTAATAGTTTGCGC |
| | | AACGTTGTTGCCATTGCTACAGGCATCGTG |
| | | GTGTCACGCTCGTCGTTTGGTATGGCTTCA |
| | | TTCAGCTCCGGTTCCCAACGATCAAGGCGA |
| | | GTTACATGATCCCCCATGTTGTGCAAAAAA |
| | | GCGGTTAGCTCCTTCGGTCCTCCGATCGTT |
| | | GTCAGAAGTAAGTTGGCCGCAGTGTTATCA |
| | | CTCATGGTTATGGCAGCACTGCATAATTCT |
| | | CTTACTGTCATGCCATCCGTAAGATGCTTT |
| | | TCTGTGACTGGTGAGTACTCAACCAAGTCA |
| | | TTCTGAGAATAGTGTATGCGGCGACCGAGT |
| | | TGCTCTTGCCCGGCGTCAATACGGGATAAT |
| | | ACCGCGCCACATAGCAGAACTTTAAAAGTG |
| | | CTCATCATTGGAAAACGTTCTTCGGGGCGA |
| | | AAACTCTCAAGGATCTTACCGCTGTTGAGA |
| | | TCCAGTTCGATGTAACCCACTCGTGCACCC |
| | | AACTGATCTTCAGCATCTTTTACTTTCACC |
| | | AGCGTTTCTGGGTGAGCAAAAACAGGAAGG |
| | | CAAAATGCCGCAAAAAAGGGAATAAGGGCG |
| | | ACACGGAAATGTTGAATACTCATACTCTTC |
| | | CTTTTTCAATATTATTGAAGCATTTATCAG |
| | | GGTTATTGTCTCATGAGCGGATACATATTT |
| | | GAATGTATTTAGAAAAATAAACAAATAGGG |
| | | GTTCCGCGCACATTTCCCCGAAAAGTGCCA |
| | | CCTGAAATTGTAAACGTTAATATTTTGTTA |
| | | AAATTCGCGTTAAATTTTTGTTAAATCAGC |
| | | TCATTTTTTAACCAATAGGCCGAAATCGGC |
| | | AAAATCCCTTATAAATCAAAAGAATAGACC |
| | | GAGATAGGGTTGAGTGTTGTTCCAGTTTGG |
| | | AACAAGAGTCCACTATTAAAGAACGTGGAC |
| | | TCCAACGTCAAAGGGCGAAAAACCGTCTAT |
| | | CAGGGCGATGGCCCACTACGTGAACCATCA |
| | | CCCTAATCAAGTTTTTTGGGGTCGAGGTGC |
| | | CGTAAAGCACTAAATCGGAACCCTAAAGGG |
| | | AGCCCCCGATTTAGAGCTTGACGGGGAAAG |
| | | CCGGCGAACGTGGCGAGAAAGGAAGGGAAG |
| | | AAAGCGAAAGGAGCGGGCGCTAGGGCGCTG |
| | | GCAAGTGTAGCGGTCACGCTGCGCGTAACC |
| | | ACCACACCCGCCGCGCTTAATGCGCCGCTA |
| | | CAGGGCGCGTC |
| 14 | AMI066 | CATTCGCCATTCAGGCTGCAAATAAGCGTT |
| | plasmid | GATATTCAGTCAATTACAAACATTAATAAC |
| | | GAAGAGATGACAGAAAAATTTTCATTCTGT |
| | | GACAGAGAAAAAGTAGCCGAAGATGACGGT |
| | | TTGTCACATGGAGTTGGCAGGATGTTTGAT |
| | | TAAAAACATAACAGGAAGAAAAATGCCCCG |
| | | CTGTGGGCGGACAAAATAGTTGGGAACTGG |
| | | GAGGGGTGGAAATGGAGTTTTTAAGGATTA |
| | | TTTAGGGAAGAGTGACAAAATAGATGGGAA |
| | | CTGGGTGTAGCGTCGTAAGCTAATACGAAA |
| | | ATTAAAAATGACAAAATAGTTTGGAACTAG |
| | | ATTTCACTTATCTGGTTCGGATCTCCTAGG |
| | | CTCAAGCAGTGATCAGATCCAGACATGATA |
| | | AGATACATTGATGAGTTTGGACAAACCACA |
| | | ACTAGAATGCAGTGAAAAAAATGCTTTATT |
| | | TGTGAAATTTGTGATGCTATTGCTTTATTT |
| | | GTAACCATTATAAGCTGCAATAAACAAGTT |
| | | AACAACAACAATTGCATTCATTTTATGTTT |
| | | CAGGTTCAGGGGGAGGTGTGGGAGGTTTTT |
| | | TAAAGCAAGTAAAACCTCTACAAATGTGGT |
| | | ATGGCTGATTATGATCCTCTAGTACTTCTC |
| | | GACAAGCTCGGATCCTGGCGCGCTCGCTCG |
| | | CTCACTGAGGCCGCCCGGGCAAAGCCCGGG |
| | | CGTCGGGCGACCTTTGGTCGCCCGGCCTCA |
| | | GTGAGCGAGCGAGCGCGCAGAGAGGGAGTG |
| | | GCCAACTCCATCACTAGGGGTTCCTAGGAA |
| | | GCTGATCTGAATTCGGTACCCGTTACATAA |
| | | CTTACGGTAAATGGCCCGCCTGGCTGACCG |
| | | CCCAACGACCCCCGCCCATTGACGTCAATA |
| | | ATGACGTATGTTCCCATAGTAACGCCAATA |
| | | GGGACTTTCCATTGACGTCAATGGGTGGAG |
| | | TATTTACGGTAAACTGCCCACTTGGCAGTA |
| | | CATCAAGTGTATCATATGCCAAGTACGCCC |
| | | CCTATTGACGTCAATGACGGTAAATGGCCC |
| | | GCCTGGCATTATGCCCAGTACATGACCTTA |
| | | TGGGACTTTCCTACTTGGCAGTACATCTAC |
| | | GTATTAGTCATCGCTATTACCATGGTGATG |
| | | CGGTTTTGGCAGTACATCAATGGGCGTGGA |
| | | TAGCGGTTTGACTCACGGGGATTTCCAAGT |
| | | CTCCACCCCATTGACGTCAATGGGAGTTTG |
| | | TTTTGGCACCAAAATCAACGGGACTTTCCA |
| | | AAATGTCGTAACAACTCCGCCCCATTGACG |
| | | CAAATGGGCGGTAGGCGTGTACGGTGGGAG |
| | | GTCTATATAAGCAGAGCTCGTTTAGTGAAC |
| | | CGTCAGATCGCCTGGAGACGCCATCCACGC |
| | | TGTTTTGACCTCCATAGAAGACACCGGGAC |
| | | CGATCCAGCCTCCGGACTCTAGAGTTAACT |
| | | GGTAAGTTTAGTCTTTTTGTCTTTTATTTC |
| | | AGGTCCCGGATCCGGTGGTGGTGCAAATCA |
| | | AAGAACTGCTCCTCAGTGGATGTTGCCTTT |
| | | ACTTCTAGGCCTGCCGCCACCATGGAGTTC |
| | | GGCCTGAGCTGGCTGTTCCTGGTGGCCATC |
| | | CTTAAGGGCGTGCAGTGCGATACTGGTAGA |
| | | CCTTTTGTAGAAATGTATTCAGAAATTCCG |
| | | GAAATAATTCATATGACAGAAGGACGAGAA |
| | | CTCGTTATACCATGTCGCGTCACGTCCCCT |
| | | AATATTACTGTTACGCTCAAGAAGTTTCCT |
| | | CTCGATACACTTATTCCAGATGGGAAACGC |
| | | ATAATTTGGGATTCACGCAAAGGGTTTATT |
| | | ATTAGTAACGCAACGTATAAAGAAATTGGG |
| | | CTGCTCACATGTGAAGCTACGGTAAATGGG |
| | | CATCTTTATAAAACAAATTATTTGACTCAT |
| | | CGGCAAACTAATACTATTATCGATGTAGTA |
| | | CTCTCCCCATCCCATGGTATTGAATTGTCA |
| | | GTTGGGGAGAAGTTGGTATTGAATTGTACT |
| | | GCACGGACAGAACTCAACGTTGGTATTGAT |
| | | TTTAATTGGGAATATCCATCATCTAAACAT |
| | | CAGCATAAGAAGTTGGTAAATCGTGATCTC |
| | | AAAACTCAAAGTGGGTCCGAAATGAAGAAG |
| | | TTTCTGTCCACACTTACGATTGATGGGGTC |
| | | ACTAGAAGTGATCAAGGGCTCTATACGTGT |
| | | GCAGCATCTAGTGGGTTGATGACAAAGAAG |
| | | AATTCAACTTTTGTTCGTGTCCATGAAAAG |
| | | GATAAAACACATACTTGTCCACCGTGTCCT |
| | | GCGCCAGAACTTCTCGGTGGTCCATCCGTC |
| | | TTTCTCTTTCCACCTAAACCAAAAGATACT |
| | | TTGATGATTTCACGGACTCCAGAAGTAACA |
| | | TGTGTTGTCGTTGATGTATCACACGAAGAT |
| | | CCAGAAGTCAAATTTAATTGGTATGTTGAT |
| | | GGTGTAGAAGTTCATAATGCGAAGACAAAA |
| | | CCACGAGAAGAACAATACAATAGTACATAT |
| | | CGGGTAGTATCCGTCTTGACTGTACTTCAC |
| | | CAAGATTGGCTTAATGGGAAAGAATACAAA |
| | | TGTAAAGTTTCTAATAAAGCTCTTCCTGCG |
| | | CCGATCGAAAAGACAATTTCCAAAGCAAAA |
| | | GGTCAACCTCGGGAACCTCAAGTTTATACG |
| | | CTCCCACCATCACGGGATGAACTCACTAAG |
| | | AATCAAGTATCCTTGACTTGTCTCGTAAAA |
| | | GGGTTTTATCCTTCAGATATTGCTGTAGAA |
| | | TGGGAATCCAATGGGCAACCAGAAAATAAT |
| | | TATAAAACAACACCACCTGTTCTTGATTCA |
| | | GATGGTTCATTCTTTCTCTATTCCAAACTT |
| | | ACTGTCGATAAATCACGCTGGCAACAAGGT |
| | | AATGTTTTCTCTTGTTCCGTCATGCATGAA |
| | | GCACTCCATAATCACTATACGCAAAAGTCT |
| | | CTCTCTCTCTCACCAGGTAAATAATAATTC |
| | | GAAAATAAAATATCTTTATTTTCATTACAT |
| | | CTGTGTGTTGGTTTTTTGTGTGGCATGCTG |
| | | GGGAGAGATCAACCCCACTCCCTCTCTGCG |
| | | CGCTCGCTCGCTCACTGAGGCCGGGCGACC |
| | | AAAGGTCGCCCGACGCCCGGGCTTTGCCCG |
| | | GGCGGCCTCAGTGAGCGAGCGAGCGCGCAG |
| | | CAAGCTGTAGCCAACCACTAGAACTATAGC |
| | | TAGAGTCCTGGGCGAACAAACGATGCTCGC |
| | | CTTCCAGAAAACCGAGGATGCGAACCACTT |
| | | CATCCGGGGTCAGCACCACCGGCAAGCGCC |
| | | GCGACGGCCGAGGTCTTCCGATCTCCTGAA |
| | | GCCAGGGCAGATCCGTGCACAGCACCTTGC |
| | | CGTAGAAGAACAGCAAGGCCGCCAATGCCT |
| | | GACGATGCGTGGAGACCGAAACCTTGCGCT |
| | | CGTTCGCCAGCCAGGACAGAAATGCCTCGA |
| | | CTTCGCTGCTGCCCAAGGTTGCCGGGTGAC |
| | | GCACACCGTGGAAACGGATGAAGGCACGAA |
| | | CCCAGTTGACATAAGCCTGTTCGGTTCGTA |
| | | AACTGTAATGCAAGTAGCGTATGCGCTCAC |
| | | GCAACTGGTCCAGAACCTTGACCGAACGCA |
| | | GCGGTGGTAACGGCGCAGTGGCGGTTTTCA |
| | | TGGCTTGTTATGACTGTTTTTTTGTACAGT |
| | | CTATGCCTCGGGCATCCAAGCAGCAAGCGC |
| | | GTTACGCCGTGGGTCGATGTTTGATGTTAT |
| | | GGAGCAGCAACGATGTTACGCAGCAGCAAC |
| | | GATGTTACGCAGCAGGGCAGTCGCCCTAAA |
| | | ACAAAGTTAGGTGGCTCAAGTATGGGCATC |
| | | ATTCGCACATGTAGGCTCGGCCCTGACCAA |
| | | GTCAAATCCATGCGGGCTGCTCTTGATCTT |
| | | TTCGGTCGTGAGTTCGGAGACGTAGCCACC |
| | | TACTCCCAACATCAGCCGGACTCCGATTAC |
| | | CTCGGGAACTTGCTCCGTAGTAAGACATTC |
| | | ATCGCGCTTGCTGCCTTCGACCAAGAAGCG |
| | | GTTGTTGGCGCTCTCGCGGCTTACGTTCTG |
| | | CCCAGGTTTGAGCAGCCGCGTAGTGAGATC |
| | | TATATCTATGATCTCGCAGTCTCCGGCGAG |
| | | CACCGGAGGCAGGGCATTGCCACCGCGCTC |
| | | ATCAATCTCCTCAAGCATGAGGCCAACGCG |
| | | CTTGGTGCTTATGTGATCTACGTGCAAGCA |
| | | GATTACGGTGACGATCCCGCAGTGGCTCTC |
| | | TATACAAAGTTGGGCATACGGGAAGAAGTG |
| | | ATGCACTTTGATATCGACCCAAGTACCGCC |
| | | ACCTAACAATTCGTTCAAGCCGAGATCGGC |
| | | TTCCCGGCCGCGGAGTTGTTCGGTAAATTG |
| | | TCACAACGCCGCGAATATAGTCTTTACCAT |
| | | GCCCTTGGCCACGCCCCTCTTTAATACGAC |
| | | GGGCAATTTGCACTTCAGAAAATGAAGAGT |
| | | TTGCTTTAGCCATAACAAAAGTCCAGTATG |
| | | CTTTTTCACAGCATAACTGGACTGATTTCA |
| | | GTTTACAACTATTCTGTCTAGTTTAAGACT |
| | | TTATTGTCATAGTTTAGATCTATTTTGTTC |
| | | AGTTTAAGACTTTATTGTCCGCCCACACCC |
| | | GCTTACGCAGGGCATCCATTTATTACTCAA |
| | | CCGTAACCGATTTTGCCAGGTTACGCGGCT |
| | | GGTCTGCGGTGTGAAATACCGCACAGATGC |
| | | GTAAGGAGAAAATACCGCATCAGGCGCTCT |
| | | TCCGCTTCCTCGCTCACTGACTCGCTGCGC |
| | | TCGGTCGTTCGGCTGCGGCGAGCGGTATCA |
| | | GCTCACTCAAAGGCGGTAATACGGTTATCC |
| | | ACAGAATCAGGGGATAACGCAGGAAAGAAC |
| | | ATGTGAGCAAAAGGCCAGCAAAAGGCCAGG |
| | | AACCGTAAAAAGGCCGCGTTGCTGGCGTTT |
| | | TTCCATAGGCTCCGCCCCCCTGACGAGCAT |
| | | CACAAAAATCGACGCTCAAGTCAGAGGTGG |
| | | CGAAACCCGACAGGACTATAAAGATACCAG |
| | | GCGTTTCCCCCTGGAAGCTCCCTCGTGCGC |
| | | TCTCCTGTTCCGACCCTGCCGCTTACCGGA |
| | | TACCTGTCCGCCTTTCTCCCTTCGGGAAGC |
| | | GTGGCGCTTTCTCAATGCTCACGCTGTAGG |
| | | TATCTCAGTTCGGTGTAGGTCGTTCGCTCC |
| | | AAGCTGGGCTGTGTGCACGAACCCCCCGTT |
| | | CAGCCCGACCGCTGCGCCTTATCCGGTAAC |
| | | TATCGTCTTGAGTCCAACCCGGTAAGACAC |
| | | GACTTATCGCCACTGGCAGCAGCCACTGGT |
| | | AACAGGATTAGCAGAGCGAGGTATGTAGGC |
| | | GGTGCTACAGAGTTCTTGAAGTGGTGGCCT |
| | | AACTACGGCTACACTAGAAGGACAGTATTT |
| | | GGTATCTGCGCTCTGCTGAAGCCAGTTACC |
| | | TTCGGAAAAAGAGTTGGTAGCTCTTGATCC |
| | | GGCAAACAAACCACCGCTGGTAGCGGTGGT |
| | | TTTTTTGTTTGCAAGCAGCAGATTACGCGC |
| | | AGAAAAAAAGGATCTCAAGAAGATCCTTTG |
| | | ATCTTTTCTACGGGGTCTGACGCTCAGTGG |
| | | AACGAAAACTCACGTTAAGGGATTTTGGTC |
| | | ATGAGATTATCAAAAAGGATCTTCACCTAG |
| | | ATCCTTTTAAATTAAAAATGAAGTTTTAAA |
| | | TCAATCTAAAGTATATATGAGTAAACTTGG |
| | | TCTGACAGTTACCAATGCTTAATCAGTGAG |
| | | GCACCTATCTCAGCGATCTGTCTATTTCGT |
| | | TCATCCATAGTTGCCTGACTCCCCGTCGTG |
| | | TAGATAACTACGATACGGGAGGGCTTACCA |
| | | TCTGGCCCCAGTGCTGCAATGATACCGCGA |
| | | GACCCACGCTCACCGGCTCCAGATTTATCA |
| | | GCAATAAACCAGCCAGCCGGAAGGGCCGAG |
| | | CGCAGAAGTGGTCCTGCAACTTTATCCGCC |
| | | TCCATCCAGTCTATTAATTGTTGCCGGGAA |
| | | GCTAGAGTAAGTAGTTCGCCAGTTAATAGT |
| | | TTGCGCAACGTTGTTGCCATTGCTACAGGC |
| | | ATCGTGGTGTCACGCTCGTCGTTTGGTATG |
| | | GCTTCATTCAGCTCCGGTTCCCAACGATCA |
| | | AGGCGAGTTACATGATCCCCCATGTTGTGC |
| | | AAAAAAGCGGTTAGCTCCTTCGGTCCTCCG |
| | | ATCGTTGTCAGAAGTAAGTTGGCCGCAGTG |
| | | TTATCACTCATGGTTATGGCAGCACTGCAT |
| | | AATTCTCTTACTGTCATGCCATCCGTAAGA |
| | | TGCTTTTCTGTGACTGGTGAGTACTCAACC |
| | | AAGTCATTCTGAGAATAGTGTATGCGGCGA |
| | | CCGAGTTGCTCTTGCCCGGCGTCAATACGG |
| | | GATAATACCGCGCCACATAGCAGAACTTTA |
| | | AAAGTGCTCATCATTGGAAAACGTTCTTCG |
| | | GGGCGAAAACTCTCAAGGATCTTACCGCTG |
| | | TTGAGATCCAGTTCGATGTAACCCACTCGT |
| | | GCACCCAACTGATCTTCAGCATCTTTTACT |
| | | TTCACCAGCGTTTCTGGGTGAGCAAAAACA |
| | | GGAAGGCAAAATGCCGCAAAAAAGGGAATA |
| | | AGGGCGACACGGAAATGTTGAATACTCATA |
| | | CTCTTCCTTTTTCAATATTATTGAAGCATT |
| | | TATCAGGGTTATTGTCTCATGAGCGGATAC |
| | | ATATTTGAATGTATTTAGAAAAATAAACAA |
| | | ATAGGGGTTCCGCGCACATTTCCCCGAAAA |
| | | GTGCCACCTGAAATTGTAAACGTTAATATT |
| | | TTGTTAAAATTCGCGTTAAATTTTTGTTAA |
| | | ATCAGCTCATTTTTTAACCAATAGGCCGAA |
| | | ATCGGCAAAATCCCTTATAAATCAAAAGAA |
| | | TAGACCGAGATAGGGTTGAGTGTTGTTCCA |
| | | GTTTGGAACAAGAGTCCACTATTAAAGAAC |
| | | GTGGACTCCAACGTCAAAGGGCGAAAAACC |
| | | GTCTATCAGGGCGATGGCCCACTACGTGAA |
| | | CCATCACCCTAATCAAGTTTTTTGGGGTCG |
| | | AGGTGCCGTAAAGCACTAAATCGGAACCCT |
| | | AAAGGGAGCCCCCGATTTAGAGCTTGACGG |
| | | GGAAAGCCGGCGAACGTGGCGAGAAAGGAA |
| | | GGGAAGAAAGCGAAAGGAGCGGGCGCTAGG |
| | | GCGCTGGCAAGTGTAGCGGTCACGCTGCGC |
| | | GTAACCACCACACCCGCCGCGCTTAATGCG |
| | | CCGCTACAGGGCGCGTC |
| 15 | AMI067 | CATTCGCCATTCAGGCTGCAAATAAGCGTT |
| | plasmid | GATATTCAGTCAATTACAAACATTAATAAC |
| | | GAAGAGATGACAGAAAAATTTTCATTCTGT |
| | | GACAGAGAAAAAGTAGCCGAAGATGACGGT |
| | | TTGTCACATGGAGTTGGCAGGATGTTTGAT |
| | | TAAAAACATAACAGGAAGAAAAATGCCCCG |
| | | CTGTGGGCGGACAAAATAGTTGGGAACTGG |
| | | GAGGGGTGGAAATGGAGTTTTTAAGGATTA |
| | | TTTAGGGAAGAGTGACAAAATAGATGGGAA |
| | | CTGGGTGTAGCGTCGTAAGCTAATACGAAA |
| | | ATTAAAAATGACAAAATAGTTTGGAACTAG |
| | | ATTTCACTTATCTGGTTCGGATCTCCTAGG |
| | | CTCAAGCAGTGATCAGATCCAGACATGATA |
| | | AGATACATTGATGAGTTTGGACAAACCACA |
| | | ACTAGAATGCAGTGAAAAAAATGCTTTATT |
| | | TGTGAAATTTGTGATGCTATTGCTTTATTT |
| | | GTAACCATTATAAGCTGCAATAAACAAGTT |
| | | AACAACAACAATTGCATTCATTTTATGTTT |
| | | CAGGTTCAGGGGGAGGTGTGGGAGGTTTTT |
| | | TAAAGCAAGTAAAACCTCTACAAATGTGGT |
| | | ATGGCTGATTATGATCCTCTAGTACTTCTC |
| | | GACAAGCTCGGATCCTGGCGCGCTCGCTCG |
| | | CTCACTGAGGCCGCCCGGGCAAAGCCCGGG |
| | | CGTCGGGCGACCTTTGGTCGCCCGGCCTCA |
| | | GTGAGCGAGCGAGCGCGCAGAGAGGGAGTG |
| | | GCCAACTCCATCACTAGGGGTTCCTAGGAA |
| | | GCTGATCTGAATTCGGTACCCGTTACATAA |
| | | CTTACGGTAAATGGCCCGCCTGGCTGACCG |
| | | CCCAACGACCCCCGCCCATTGACGTCAATA |
| | | ATGACGTATGTTCCCATAGTAACGCCAATA |
| | | GGGACTTTCCATTGACGTCAATGGGTGGAG |
| | | TATTTACGGTAAACTGCCCACTTGGCAGTA |
| | | CATCAAGTGTATCATATGCCAAGTACGCCC |
| | | CCTATTGACGTCAATGACGGTAAATGGCCC |
| | | GCCTGGCATTATGCCCAGTACATGACCTTA |
| | | TGGGACTTTCCTACTTGGCAGTACATCTAC |
| | | GTATTAGTCATCGCTATTACCATGGTGATG |
| | | CGGTTTTGGCAGTACATCAATGGGCGTGGA |
| | | TAGCGGTTTGACTCACGGGGATTTCCAAGT |
| | | CTCCACCCCATTGACGTCAATGGGAGTTTG |
| | | TTTTGGCACCAAAATCAACGGGACTTTCCA |
| | | AAATGTCGTAACAACTCCGCCCCATTGACG |
| | | CAAATGGGCGGTAGGCGTGTACGGTGGGAG |
| | | GTCTATATAAGCAGAGCTCGTTTAGTGAAC |
| | | CGTCAGATCGCCTGGAGACGCCATCCACGC |
| | | TGTTTTGACCTCCATAGAAGACACCGGGAC |
| | | CGATCCAGCCTCCGGACTCTAGAGTTAACT |
| | | GGTAAGTTTAGTCTTTTTGTCTTTTATTTC |
| | | AGGTCCCGGATCCGGTGGTGGTGCAAATCA |
| | | AAGAACTGCTCCTCAGTGGATGTTGCCTTT |
| | | ACTTCTAGGCCTGCCGCCACCATGGTGAGC |
| | | TACTGGGACACCGGCGTGCTGCTGTGCGCC |
| | | CTGCTGAGCTGCCTGCTGCTGACCGGCAGC |
| | | AGCAGCGGCAGCGACACCGGCAGACCCTTC |
| | | GTGGAGATGTACAGCGAGATCCCCGAGATC |
| | | ATCCACATGACCGAGGGCAGAGAGCTGGTG |
| | | ATCCCCTGCAGAGTGACCAGCCCCAACATC |
| | | ACCGTGACCCTGAAGAAGTTCCCCCTGGAC |
| | | ACCCTGATCCCCGACGGCAAGAGAATCATC |
| | | TGGGACAGCAGAAAGGGCTTCATCATCAGC |
| | | AACGCCACCTACAAGGAGATCGGCCTGCTG |
| | | ACCTGCGAGGCCACCGTGAACGGCCACCTG |
| | | TACAAGACCAACTACCTGACCCACAGACAG |
| | | ACCAACACCATCATCGACGTGGTGCTGAGC |
| | | CCCAGCCACGGCATCGAGCTGAGCGTGGGC |
| | | GAGAAGCTGGTGCTGAACTGCACCGCCAGA |
| | | ACCGAGCTGAACGTGGGCATCGACTTCAAC |
| | | TGGGAGTACCCCAGCAGCAAGCACCAGCAC |
| | | AAGAAGCTGGTGAACAGAGACCTGAAGACC |
| | | CAGAGCGGCAGCGAGATGAAGAAGTTCCTG |
| | | AGCACCCTGACCATCGACGGCGTGACCAGA |
| | | AGCGACCAGGGCCTGTACACCTGCGCCGCC |
| | | AGCAGCGGCCTGATGACCAAGAAGAACAGC |
| | | ACCTTCGTGAGAGTGCACGAGAAGGACAAG |
| | | ACCCACACCTGCCCCCCCTGCCCCGCCCCC |
| | | GAGCTGCTGGGCGGCCCCAGCGTGTTCCTG |
| | | TTCCCCCCCAAGCCCAAGGACACCCTGATG |
| | | ATCAGCAGAACCCCCGAGGTGACCTGCGTG |
| | | GTGGTGGACGTGAGCCACGAGGACCCCGAG |
| | | GTGAAGTTCAACTGGTACGTGGACGGCGTG |
| | | GAGGTGCACAACGCCAAGACCAAGCCCAGA |
| | | GAGGAGCAGTACAACAGCACCTACAGAGTG |
| | | GTGAGCGTGCTGACCGTGCTGCACCAGGAC |
| | | TGGCTGAACGGCAAGGAGTACAAGTGCAAG |
| | | GTGAGCAACAAGGCCCTGCCCGCCCCCATC |
| | | GAGAAGACCATCAGCAAGGCCAAGGGCCAG |
| | | CCCAGAGAGCCCCAGGTGTACACCCTGCCC |
| | | CCCAGCAGAGACGAGCTGACCAAGAACCAG |
| | | GTGAGCCTGACCTGCCTGGTGAAGGGCTTC |
| | | TACCCCAGCGACATCGCCGTGGAGTGGGAG |
| | | AGCAACGGCCAGCCCGAGAACAACTACAAG |
| | | ACCACCCCCCCCGTGCTGGACAGCGACGGC |
| | | AGCTTCTTCCTGTACAGCAAGCTGACCGTG |
| | | GACAAGAGCAGATGGCAGCAGGGCAACGTG |
| | | TTCAGCTGCAGCGTGATGCACGAGGCCCTG |
| | | CACAACCACTACACCCAGAAGAGCCTGAGC |
| | | CTGAGCCCCGGCAAGTGATTCGAAAATAAA |
| | | ATATCTTTATTTTCATTACATCTGTGTGTT |
| | | GGTTTTTTGTGTGGCATGCTGGGGAGAGAT |
| | | CAACCCCACTCCCTCTCTGCGCGCTCGCTC |
| | | GCTCACTGAGGCCGGGCGACCAAAGGTCGC |
| | | CCGACGCCCGGGCTTTGCCCGGGCGGCCTC |
| | | AGTGAGCGAGCGAGCGCGCAGCAAGCTGTA |
| | | GCCAACCACTAGAACTATAGCTAGAGTCCT |
| | | GGGCGAACAAACGATGCTCGCCTTCCAGAA |
| | | AACCGAGGATGCGAACCACTTCATCCGGGG |
| | | TCAGCACCACCGGCAAGCGCCGCGACGGCC |
| | | GAGGTCTTCCGATCTCCTGAAGCCAGGGCA |
| | | GATCCGTGCACAGCACCTTGCCGTAGAAGA |
| | | ACAGCAAGGCCGCCAATGCCTGACGATGCG |
| | | TGGAGACCGAAACCTTGCGCTCGTTCGCCA |
| | | GCCAGGACAGAAATGCCTCGACTTCGCTGC |
| | | TGCCCAAGGTTGCCGGGTGACGCACACCGT |
| | | GGAAACGGATGAAGGCACGAACCCAGTTGA |
| | | CATAAGCCTGTTCGGTTCGTAAACTGTAAT |
| | | GCAAGTAGCGTATGCGCTCACGCAACTGGT |
| | | CCAGAACCTTGACCGAACGCAGCGGTGGTA |
| | | ACGGCGCAGTGGCGGTTTTCATGGCTTGTT |
| | | ATGACTGTTTTTTTGTACAGTCTATGCCTC |
| | | GGGCATCCAAGCAGCAAGCGCGTTACGCCG |
| | | TGGGTCGATGTTTGATGTTATGGAGCAGCA |
| | | ACGATGTTACGCAGCAGCAACGATGTTACG |
| | | CAGCAGGGCAGTCGCCCTAAAACAAAGTTA |
| | | GGTGGCTCAAGTATGGGCATCATTCGCACA |
| | | TGTAGGCTCGGCCCTGACCAAGTCAAATCC |
| | | ATGCGGGCTGCTCTTGATCTTTTCGGTCGT |
| | | GAGTTCGGAGACGTAGCCACCTACTCCCAA |
| | | CATCAGCCGGACTCCGATTACCTCGGGAAC |
| | | TTGCTCCGTAGTAAGACATTCATCGCGCTT |
| | | GCTGCCTTCGACCAAGAAGCGGTTGTTGGC |
| | | GCTCTCGCGGCTTACGTTCTGCCCAGGTTT |
| | | GAGCAGCCGCGTAGTGAGATCTATATCTAT |
| | | GATCTCGCAGTCTCCGGCGAGCACCGGAGG |
| | | CAGGGCATTGCCACCGCGCTCATCAATCTC |
| | | CTCAAGCATGAGGCCAACGCGCTTGGTGCT |
| | | TATGTGATCTACGTGCAAGCAGATTACGGT |
| | | GACGATCCCGCAGTGGCTCTCTATACAAAG |
| | | TTGGGCATACGGGAAGAAGTGATGCACTTT |
| | | GATATCGACCCAAGTACCGCCACCTAACAA |
| | | TTCGTTCAAGCCGAGATCGGCTTCCCGGCC |
| | | GCGGAGTTGTTCGGTAAATTGTCACAACGC |
| | | CGCGAATATAGTCTTTACCATGCCCTTGGC |
| | | CACGCCCCTCTTTAATACGACGGGCAATTT |
| | | GCACTTCAGAAAATGAAGAGTTTGCTTTAG |
| | | CCATAACAAAAGTCCAGTATGCTTTTTCAC |
| | | AGCATAACTGGACTGATTTCAGTTTACAAC |
| | | TATTCTGTCTAGTTTAAGACTTTATTGTCA |
| | | TAGTTTAGATCTATTTTGTTCAGTTTAAGA |
| | | CTTTATTGTCCGCCCACACCCGCTTACGCA |
| | | GGGCATCCATTTATTACTCAACCGTAACCG |
| | | ATTTTGCCAGGTTACGCGGCTGGTCTGCGG |
| | | TGTGAAATACCGCACAGATGCGTAAGGAGA |
| | | AAATACCGCATCAGGCGCTCTTCCGCTTCC |
| | | TCGCTCACTGACTCGCTGCGCTCGGTCGTT |
| | | CGGCTGCGGCGAGCGGTATCAGCTCACTCA |
| | | AAGGCGGTAATACGGTTATCCACAGAATCA |
| | | GGGGATAACGCAGGAAAGAACATGTGAGCA |
| | | AAAGGCCAGCAAAAGGCCAGGAACCGTAAA |
| | | AAGGCCGCGTTGCTGGCGTTTTTCCATAGG |
| | | CTCCGCCCCCCTGACGAGCATCACAAAAAT |
| | | CGACGCTCAAGTCAGAGGTGGCGAAACCCG |
| | | ACAGGACTATAAAGATACCAGGCGTTTCCC |
| | | CCTGGAAGCTCCCTCGTGCGCTCTCCTGTT |
| | | CCGACCCTGCCGCTTACCGGATACCTGTCC |
| | | GCCTTTCTCCCTTCGGGAAGCGTGGCGCTT |
| | | TCTCAATGCTCACGCTGTAGGTATCTCAGT |
| | | TCGGTGTAGGTCGTTCGCTCCAAGCTGGGC |
| | | TGTGTGCACGAACCCCCCGTTCAGCCCGAC |
| | | CGCTGCGCCTTATCCGGTAACTATCGTCTT |
| | | GAGTCCAACCCGGTAAGACACGACTTATCG |
| | | CCACTGGCAGCAGCCACTGGTAACAGGATT |
| | | AGCAGAGCGAGGTATGTAGGCGGTGCTACA |
| | | GAGTTCTTGAAGTGGTGGCCTAACTACGGC |
| | | TACACTAGAAGGACAGTATTTGGTATCTGC |
| | | GCTCTGCTGAAGCCAGTTACCTTCGGAAAA |
| | | AGAGTTGGTAGCTCTTGATCCGGCAAACAA |
| | | ACCACCGCTGGTAGCGGTGGTTTTTTTGTT |
| | | TGCAAGCAGCAGATTACGCGCAGAAAAAAA |
| | | GGATCTCAAGAAGATCCTTTGATCTTTTCT |
| | | ACGGGGTCTGACGCTCAGTGGAACGAAAAC |
| | | TCACGTTAAGGGATTTTGGTCATGAGATTA |
| | | TCAAAAAGGATCTTCACCTAGATCCTTTTA |
| | | AATTAAAAATGAAGTTTTAAATCAATCTAA |
| | | AGTATATATGAGTAAACTTGGTCTGACAGT |
| | | TACCAATGCTTAATCAGTGAGGCACCTATC |
| | | TCAGCGATCTGTCTATTTCGTTCATCCATA |
| | | GTTGCCTGACTCCCCGTCGTGTAGATAACT |
| | | ACGATACGGGAGGGCTTACCATCTGGCCCC |
| | | AGTGCTGCAATGATACCGCGAGACCCACGC |
| | | TCACCGGCTCCAGATTTATCAGCAATAAAC |
| | | CAGCCAGCCGGAAGGGCCGAGCGCAGAAGT |
| | | GGTCCTGCAACTTTATCCGCCTCCATCCAG |
| | | TCTATTAATTGTTGCCGGGAAGCTAGAGTA |
| | | AGTAGTTCGCCAGTTAATAGTTTGCGCAAC |
| | | GTTGTTGCCATTGCTACAGGCATCGTGGTG |
| | | TCACGCTCGTCGTTTGGTATGGCTTCATTC |
| | | AGCTCCGGTTCCCAACGATCAAGGCGAGTT |
| | | ACATGATCCCCCATGTTGTGCAAAAAAGCG |
| | | GTTAGCTCCTTCGGTCCTCCGATCGTTGTC |
| | | AGAAGTAAGTTGGCCGCAGTGTTATCACTC |
| | | ATGGTTATGGCAGCACTGCATAATTCTCTT |
| | | ACTGTCATGCCATCCGTAAGATGCTTTTCT |
| | | GTGACTGGTGAGTACTCAACCAAGTCATTC |
| | | TGAGAATAGTGTATGCGGCGACCGAGTTGC |
| | | TCTTGCCCGGCGTCAATACGGGATAATACC |
| | | GCGCCACATAGCAGAACTTTAAAAGTGCTC |
| | | ATCATTGGAAAACGTTCTTCGGGGCGAAAA |
| | | CTCTCAAGGATCTTACCGCTGTTGAGATCC |
| | | AGTTCGATGTAACCCACTCGTGCACCCAAC |
| | | TGATCTTCAGCATCTTTTACTTTCACCAGC |
| | | GTTTCTGGGTGAGCAAAAACAGGAAGGCAA |
| | | AATGCCGCAAAAAAGGGAATAAGGGCGACA |
| | | CGGAAATGTTGAATACTCATACTCTTCCTT |
| | | TTTCAATATTATTGAAGCATTTATCAGGGT |
| | | TATTGTCTCATGAGCGGATACATATTTGAA |
| | | TGTATTTAGAAAAATAAACAAATAGGGGTT |
| | | CCGCGCACATTTCCCCGAAAAGTGCCACCT |
| | | GAAATTGTAAACGTTAATATTTTGTTAAAA |
| | | TTCGCGTTAAATTTTTGTTAAATCAGCTCA |
| | | TTTTTTAACCAATAGGCCGAAATCGGCAAA |
| | | ATCCCTTATAAATCAAAAGAATAGACCGAG |
| | | ATAGGGTTGAGTGTTGTTCCAGTTTGGAAC |
| | | AAGAGTCCACTATTAAAGAACGTGGACTCC |
| | | AACGTCAAAGGGCGAAAAACCGTCTATCAG |
| | | GGCGATGGCCCACTACGTGAACCATCACCC |
| | | TAATCAAGTTTTTTGGGGTCGAGGTGCCGT |
| | | AAAGCACTAAATCGGAACCCTAAAGGGAGC |
| | | CCCCGATTTAGAGCTTGACGGGGAAAGCCG |
| | | GCGAACGTGGCGAGAAAGGAAGGGAAGAAA |
| | | GCGAAAGGAGCGGGCGCTAGGGCGCTGGCA |
| | | AGTGTAGCGGTCACGCTGCGCGTAACCACC |
| | | ACACCCGCCGCGCTTAATGCGCCGCTACAG |
| | | GGCGCGTC |
| 16 | AMI068 | CATTCGCCATTCAGGCTGCAAATAAGCGTT |
| | plasmid | GATATTCAGTCAATTACAAACATTAATAAC |
| | | GAAGAGATGACAGAAAAATTTTCATTCTGT |
| | | GACAGAGAAAAAGTAGCCGAAGATGACGGT |
| | | TTGTCACATGGAGTTGGCAGGATGTTTGAT |
| | | TAAAAACATAACAGGAAGAAAAATGCCCCG |
| | | CTGTGGGCGGACAAAATAGTTGGGAACTGG |
| | | GAGGGGTGGAAATGGAGTTTTTAAGGATTA |
| | | TTTAGGGAAGAGTGACAAAATAGATGGGAA |
| | | CTGGGTGTAGCGTCGTAAGCTAATACGAAA |
| | | ATTAAAAATGACAAAATAGTTTGGAACTAG |
| | | ATTTCACTTATCTGGTTCGGATCTCCTAGG |
| | | CTCAAGCAGTGATCAGATCCAGACATGATA |
| | | AGATACATTGATGAGTTTGGACAAACCACA |
| | | ACTAGAATGCAGTGAAAAAAATGCTTTATT |
| | | TGTGAAATTTGTGATGCTATTGCTTTATTT |
| | | GTAACCATTATAAGCTGCAATAAACAAGTT |
| | | AACAACAACAATTGCATTCATTTTATGTTT |
| | | CAGGTTCAGGGGGAGGTGTGGGAGGTTTTT |
| | | TAAAGCAAGTAAAACCTCTACAAATGTGGT |
| | | ATGGCTGATTATGATCCTCTAGTACTTCTC |
| | | GACAAGCTCGGATCCTGGCGCGCTCGCTCG |
| | | CTCACTGAGGCCGCCCGGGCAAAGCCCGGG |
| | | CGTCGGGCGACCTTTGGTCGCCCGGCCTCA |
| | | GTGAGCGAGCGAGCGCGCAGAGAGGGAGTG |
| | | GCCAACTCCATCACTAGGGGTTCCTAGGAA |
| | | GCTGATCTGAATTCGGTACCCGTTACATAA |
| | | CTTACGGTAAATGGCCCGCCTGGCTGACCG |
| | | CCCAACGACCCCCGCCCATTGACGTCAATA |
| | | ATGACGTATGTTCCCATAGTAACGCCAATA |
| | | GGGACTTTCCATTGACGTCAATGGGTGGAG |
| | | TATTTACGGTAAACTGCCCACTTGGCAGTA |
| | | CATCAAGTGTATCATATGCCAAGTACGCCC |
| | | CCTATTGACGTCAATGACGGTAAATGGCCC |
| | | GCCTGGCATTATGCCCAGTACATGACCTTA |
| | | TGGGACTTTCCTACTTGGCAGTACATCTAC |
| | | GTATTAGTCATCGCTATTACCATGGTGATG |
| | | CGGTTTTGGCAGTACATCAATGGGCGTGGA |
| | | TAGCGGTTTGACTCACGGGGATTTCCAAGT |
| | | CTCCACCCCATTGACGTCAATGGGAGTTTG |
| | | TTTTGGCACCAAAATCAACGGGACTTTCCA |
| | | AAATGTCGTAACAACTCCGCCCCATTGACG |
| | | CAAATGGGCGGTAGGCGTGTACGGTGGGAG |
| | | GTCTATATAAGCAGAGCTCGTTTAGTGAAC |
| | | CGTCAGATCGCCTGGAGACGCCATCCACGC |
| | | TGTTTTGACCTCCATAGAAGACACCGGGAC |
| | | CGATCCAGCCTCCGGACTCTAGAGTTAACT |
| | | GGTAAGTTTAGTCTTTTTGTCTTTTATTTC |
| | | AGGTCCCGGATCCGGTGGTGGTGCAAATCA |
| | | AAGAACTGCTCCTCAGTGGATGTTGCCTTT |
| | | ACTTCTAGGCCTGCCGCCACCATGGAGTTC |
| | | GGCCTGAGCTGGCTGTTCCTGGTGGCCATC |
| | | CTTAAGGGCGTGCAGTGCGACACCGGCAGA |
| | | CCCTTCGTGGAGATGTACAGCGAGATCCCC |
| | | GAGATCATCCACATGACCGAGGGCAGAGAG |
| | | CTGGTGATCCCCTGCAGAGTGACCAGCCCC |
| | | AACATCACCGTGACCCTGAAGAAGTTCCCC |
| | | CTGGACACCCTGATCCCCGACGGCAAGAGA |
| | | ATCATCTGGGACAGCAGAAAGGGCTTCATC |
| | | ATCAGCAACGCCACCTACAAGGAGATCGGC |
| | | CTGCTGACCTGCGAGGCCACCGTGAACGGC |
| | | CACCTGTACAAGACCAACTACCTGACCCAC |
| | | AGACAGACCAACACCATCATCGACGTGGTG |
| | | CTGAGCCCCAGCCACGGCATCGAGCTGAGC |
| | | GTGGGCGAGAAGCTGGTGCTGAACTGCACC |
| | | GCCAGAACCGAGCTGAACGTGGGCATCGAC |
| | | TTCAACTGGGAGTACCCCAGCAGCAAGCAC |
| | | CAGCACAAGAAGCTGGTGAACAGAGACCTG |
| | | AAGACCCAGAGCGGCAGCGAGATGAAGAAG |
| | | TTCCTGAGCACCCTGACCATCGACGGCGTG |
| | | ACCAGAAGCGACCAGGGCCTGTACACCTGC |
| | | GCCGCCAGCAGCGGCCTGATGACCAAGAAG |
| | | AACAGCACCTTCGTGAGAGTGCACGAGAAG |
| | | GACAAGACCCACACCTGCCCCCCCTGCCCC |
| | | GCCCCCGAGCTGCTGGGCGGCCCCAGCGTG |
| | | TTCCTGTTCCCCCCCAAGCCCAAGGACACC |
| | | CTGATGATCAGCAGAACCCCCGAGGTGACC |
| | | TGCGTGGTGGTGGACGTGAGCCACGAGGAC |
| | | CCCGAGGTGAAGTTCAACTGGTACGTGGAC |
| | | GGCGTGGAGGTGCACAACGCCAAGACCAAG |
| | | CCCAGAGAGGAGCAGTACAACAGCACCTAC |
| | | AGAGTGGTGAGCGTGCTGACCGTGCTGCAC |
| | | CAGGACTGGCTGAACGGCAAGGAGTACAAG |
| | | TGCAAGGTGAGCAACAAGGCCCTGCCCGCC |
| | | CCCATCGAGAAGACCATCAGCAAGGCCAAG |
| | | GGCCAGCCCAGAGAGCCCCAGGTGTACACC |
| | | CTGCCCCCCAGCAGAGACGAGCTGACCAAG |
| | | AACCAGGTGAGCCTGACCTGCCTGGTGAAG |
| | | GGCTTCTACCCCAGCGACATCGCCGTGGAG |
| | | TGGGAGAGCAACGGCCAGCCCGAGAACAAC |
| | | TACAAGACCACCCCCCCCGTGCTGGACAGC |
| | | GACGGCAGCTTCTTCCTGTACAGCAAGCTG |
| | | ACCGTGGACAAGAGCAGATGGCAGCAGGGC |
| | | AACGTGTTCAGCTGCAGCGTGATGCACGAG |
| | | GCCCTGCACAACCACTACACCCAGAAGAGC |
| | | CTGAGCCTGAGCCCCGGCAAGTGATTCGAA |
| | | AATAAAATATCTTTATTTTCATTACATCTG |
| | | TGTGTTGGTTTTTTGTGTGGCATGCTGGGG |
| | | AGAGATCAACCCCACTCCCTCTCTGCGCGC |
| | | TCGCTCGCTCACTGAGGCCGGGCGACCAAA |
| | | GGTCGCCCGACGCCCGGGCTTTGCCCGGGC |
| | | GGCCTCAGTGAGCGAGCGAGCGCGCAGCAA |
| | | GCTGTAGCCAACCACTAGAACTATAGCTAG |
| | | AGTCCTGGGCGAACAAACGATGCTCGCCTT |
| | | CCAGAAAACCGAGGATGCGAACCACTTCAT |
| | | CCGGGGTCAGCACCACCGGCAAGCGCCGCG |
| | | ACGGCCGAGGTCTTCCGATCTCCTGAAGCC |
| | | AGGGCAGATCCGTGCACAGCACCTTGCCGT |
| | | AGAAGAACAGCAAGGCCGCCAATGCCTGAC |
| | | GATGCGTGGAGACCGAAACCTTGCGCTCGT |
| | | TCGCCAGCCAGGACAGAAATGCCTCGACTT |
| | | CGCTGCTGCCCAAGGTTGCCGGGTGACGCA |
| | | CACCGTGGAAACGGATGAAGGCACGAACCC |
| | | AGTTGACATAAGCCTGTTCGGTTCGTAAAC |
| | | TGTAATGCAAGTAGCGTATGCGCTCACGCA |
| | | ACTGGTCCAGAACCTTGACCGAACGCAGCG |
| | | GTGGTAACGGCGCAGTGGCGGTTTTCATGG |
| | | CTTGTTATGACTGTTTTTTTGTACAGTCTA |
| | | TGCCTCGGGCATCCAAGCAGCAAGCGCGTT |
| | | ACGCCGTGGGTCGATGTTTGATGTTATGGA |
| | | GCAGCAACGATGTTACGCAGCAGCAACGAT |
| | | GTTACGCAGCAGGGCAGTCGCCCTAAAACA |
| | | AAGTTAGGTGGCTCAAGTATGGGCATCATT |
| | | CGCACATGTAGGCTCGGCCCTGACCAAGTC |
| | | AAATCCATGCGGGCTGCTCTTGATCTTTTC |
| | | GGTCGTGAGTTCGGAGACGTAGCCACCTAC |
| | | TCCCAACATCAGCCGGACTCCGATTACCTC |
| | | GGGAACTTGCTCCGTAGTAAGACATTCATC |
| | | GCGCTTGCTGCCTTCGACCAAGAAGCGGTT |
| | | GTTGGCGCTCTCGCGGCTTACGTTCTGCCC |
| | | AGGTTTGAGCAGCCGCGTAGTGAGATCTAT |
| | | ATCTATGATCTCGCAGTCTCCGGCGAGCAC |
| | | CGGAGGCAGGGCATTGCCACCGCGCTCATC |
| | | AATCTCCTCAAGCATGAGGCCAACGCGCTT |
| | | GGTGCTTATGTGATCTACGTGCAAGCAGAT |
| | | TACGGTGACGATCCCGCAGTGGCTCTCTAT |
| | | ACAAAGTTGGGCATACGGGAAGAAGTGATG |
| | | CACTTTGATATCGACCCAAGTACCGCCACC |
| | | TAACAATTCGTTCAAGCCGAGATCGGCTTC |
| | | CCGGCCGCGGAGTTGTTCGGTAAATTGTCA |
| | | CAACGCCGCGAATATAGTCTTTACCATGCC |
| | | CTTGGCCACGCCCCTCTTTAATACGACGGG |
| | | CAATTTGCACTTCAGAAAATGAAGAGTTTG |
| | | CTTTAGCCATAACAAAAGTCCAGTATGCTT |
| | | TTTCACAGCATAACTGGACTGATTTCAGTT |
| | | TACAACTATTCTGTCTAGTTTAAGACTTTA |
| | | TTGTCATAGTTTAGATCTATTTTGTTCAGT |
| | | TTAAGACTTTATTGTCCGCCCACACCCGCT |
| | | TACGCAGGGCATCCATTTATTACTCAACCG |
| | | TAACCGATTTTGCCAGGTTACGCGGCTGGT |
| | | CTGCGGTGTGAAATACCGCACAGATGCGTA |
| | | AGGAGAAAATACCGCATCAGGCGCTCTTCC |
| | | GCTTCCTCGCTCACTGACTCGCTGCGCTCG |
| | | GTCGTTCGGCTGCGGCGAGCGGTATCAGCT |
| | | CACTCAAAGGCGGTAATACGGTTATCCACA |
| | | GAATCAGGGGATAACGCAGGAAAGAACATG |
| | | TGAGCAAAAGGCCAGCAAAAGGCCAGGAAC |
| | | CGTAAAAAGGCCGCGTTGCTGGCGTTTTTC |
| | | CATAGGCTCCGCCCCCCTGACGAGCATCAC |
| | | AAAAATCGACGCTCAAGTCAGAGGTGGCGA |
| | | AACCCGACAGGACTATAAAGATACCAGGCG |
| | | TTTCCCCCTGGAAGCTCCCTCGTGCGCTCT |
| | | CCTGTTCCGACCCTGCCGCTTACCGGATAC |
| | | CTGTCCGCCTTTCTCCCTTCGGGAAGCGTG |
| | | GCGCTTTCTCAATGCTCACGCTGTAGGTAT |
| | | CTCAGTTCGGTGTAGGTCGTTCGCTCCAAG |
| | | CTGGGCTGTGTGCACGAACCCCCCGTTCAG |
| | | CCCGACCGCTGCGCCTTATCCGGTAACTAT |
| | | CGTCTTGAGTCCAACCCGGTAAGACACGAC |
| | | TTATCGCCACTGGCAGCAGCCACTGGTAAC |
| | | AGGATTAGCAGAGCGAGGTATGTAGGCGGT |
| | | GCTACAGAGTTCTTGAAGTGGTGGCCTAAC |
| | | TACGGCTACACTAGAAGGACAGTATTTGGT |
| | | ATCTGCGCTCTGCTGAAGCCAGTTACCTTC |
| | | GGAAAAAGAGTTGGTAGCTCTTGATCCGGC |
| | | AAACAAACCACCGCTGGTAGCGGTGGTTTT |
| | | TTTGTTTGCAAGCAGCAGATTACGCGCAGA |
| | | AAAAAAGGATCTCAAGAAGATCCTTTGATC |
| | | TTTTCTACGGGGTCTGACGCTCAGTGGAAC |
| | | GAAAACTCACGTTAAGGGATTTTGGTCATG |
| | | AGATTATCAAAAAGGATCTTCACCTAGATC |
| | | CTTTTAAATTAAAAATGAAGTTTTAAATCA |
| | | ATCTAAAGTATATATGAGTAAACTTGGTCT |
| | | GACAGTTACCAATGCTTAATCAGTGAGGCA |
| | | CCTATCTCAGCGATCTGTCTATTTCGTTCA |
| | | TCCATAGTTGCCTGACTCCCCGTCGTGTAG |
| | | ATAACTACGATACGGGAGGGCTTACCATCT |
| | | GGCCCCAGTGCTGCAATGATACCGCGAGAC |
| | | CCACGCTCACCGGCTCCAGATTTATCAGCA |
| | | ATAAACCAGCCAGCCGGAAGGGCCGAGCGC |
| | | AGAAGTGGTCCTGCAACTTTATCCGCCTCC |
| | | ATCCAGTCTATTAATTGTTGCCGGGAAGCT |
| | | AGAGTAAGTAGTTCGCCAGTTAATAGTTTG |
| | | CGCAACGTTGTTGCCATTGCTACAGGCATC |
| | | GTGGTGTCACGCTCGTCGTTTGGTATGGCT |
| | | TCATTCAGCTCCGGTTCCCAACGATCAAGG |
| | | CGAGTTACATGATCCCCCATGTTGTGCAAA |
| | | AAAGCGGTTAGCTCCTTCGGTCCTCCGATC |
| | | GTTGTCAGAAGTAAGTTGGCCGCAGTGTTA |
| | | TCACTCATGGTTATGGCAGCACTGCATAAT |
| | | TCTCTTACTGTCATGCCATCCGTAAGATGC |
| | | TTTTCTGTGACTGGTGAGTACTCAACCAAG |
| | | TCATTCTGAGAATAGTGTATGCGGCGACCG |
| | | AGTTGCTCTTGCCCGGCGTCAATACGGGAT |
| | | AATACCGCGCCACATAGCAGAACTTTAAAA |
| | | GTGCTCATCATTGGAAAACGTTCTTCGGGG |
| | | CGAAAACTCTCAAGGATCTTACCGCTGTTG |
| | | AGATCCAGTTCGATGTAACCCACTCGTGCA |
| | | CCCAACTGATCTTCAGCATCTTTTACTTTC |
| | | ACCAGCGTTTCTGGGTGAGCAAAAACAGGA |
| | | AGGCAAAATGCCGCAAAAAAGGGAATAAGG |
| | | GCGACACGGAAATGTTGAATACTCATACTC |
| | | TTCCTTTTTCAATATTATTGAAGCATTTAT |
| | | CAGGGTTATTGTCTCATGAGCGGATACATA |
| | | TTTGAATGTATTTAGAAAAATAAACAAATA |
| | | GGGGTTCCGCGCACATTTCCCCGAAAAGTG |
| | | CCACCTGAAATTGTAAACGTTAATATTTTG |
| | | TTAAAATTCGCGTTAAATTTTTGTTAAATC |
| | | AGCTCATTTTTTAACCAATAGGCCGAAATC |
| | | GGCAAAATCCCTTATAAATCAAAAGAATAG |
| | | ACCGAGATAGGGTTGAGTGTTGTTCCAGTT |
| | | TGGAACAAGAGTCCACTATTAAAGAACGTG |
| | | GACTCCAACGTCAAAGGGCGAAAAACCGTC |
| | | TATCAGGGCGATGGCCCACTACGTGAACCA |
| | | TCACCCTAATCAAGTTTTTTGGGGTCGAGG |
| | | TGCCGTAAAGCACTAAATCGGAACCCTAAA |
| | | GGGAGCCCCCGATTTAGAGCTTGACGGGGA |
| | | AAGCCGGCGAACGTGGCGAGAAAGGAAGGG |
| | | AAGAAAGCGAAAGGAGCGGGCGCTAGGGCG |
| | | CTGGCAAGTGTAGCGGTCACGCTGCGCGTA |
| | | ACCACCACACCCGCCGCGCTTAATGCGCCG |
| | | CTACAGGGCGCGTC |
| 17 | AMI119 | CATTCGCCATTCAGGCTGCAAATAAGCGTT |
| | plasmid | GATATTCAGTCAATTACAAACATTAATAAC |
| | | GAAGAGATGACAGAAAAATTTTCATTCTGT |
| | | GACAGAGAAAAAGTAGCCGAAGATGACGGT |
| | | TTGTCACATGGAGTTGGCAGGATGTTTGAT |
| | | TAAAAACATAACAGGAAGAAAAATGCCCCG |
| | | CTGTGGGCGGACAAAATAGTTGGGAACTGG |
| | | GAGGGGTGGAAATGGAGTTTTTAAGGATTA |
| | | TTTAGGGAAGAGTGACAAAATAGATGGGAA |
| | | CTGGGTGTAGCGTCGTAAGCTAATACGAAA |
| | | ATTAAAAATGACAAAATAGTTTGGAACTAG |
| | | ATTTCACTTATCTGGTTCGGATCTCCTAGG |
| | | CTCAAGCAGTGATCAGATCCAGACATGATA |
| | | AGATACATTGATGAGTTTGGACAAACCACA |
| | | ACTAGAATGCAGTGAAAAAAATGCTTTATT |
| | | TGTGAAATTTGTGATGCTATTGCTTTATTT |
| | | GTAACCATTATAAGCTGCAATAAACAAGTT |
| | | AACAACAACAATTGCATTCATTTTATGTTT |
| | | CAGGTTCAGGGGGAGGTGTGGGAGGTTTTT |
| | | TAAAGCAAGTAAAACCTCTACAAATGTGGT |
| | | ATGGCTGATTATGATCCTCTAGTACTTCTC |
| | | GACAAGCTCGGATCCTGGCGCGCTCGCTCG |
| | | CTCACTGAGGCCGCCCGGGCAAAGCCCGGG |
| | | CGTCGGGCGACCTTTGGTCGCCCGGCCTCA |
| | | GTGAGCGAGCGAGCGCGCAGAGAGGGAGTG |
| | | GCCAACTCCATCACTAGGGGTTCCTAGGAA |
| | | GCTGATCTGAATTCGGTACCCGTTACATAA |
| | | CTTACGGTAAATGGCCCGCCTGGCTGACCG |
| | | CCCAACGACCCCCGCCCATTGACGTCAATA |
| | | ATGACGTATGTTCCCATAGTAACGCCAATA |
| | | GGGACTTTCCATTGACGTCAATGGGTGGAG |
| | | TATTTACGGTAAACTGCCCACTTGGCAGTA |
| | | CATCAAGTGTATCATATGCCAAGTACGCCC |
| | | CCTATTGACGTCAATGACGGTAAATGGCCC |
| | | GCCTGGCATTATGCCCAGTACATGACCTTA |
| | | TGGGACTTTCCTACTTGGCAGTACATCTAC |
| | | GTATTAGTCATCGCTATTACCATGGTGATG |
| | | CGGTTTTGGCAGTACATCAATGGGCGTGGA |
| | | TAGCGGTTTGACTCACGGGGATTTCCAAGT |
| | | CTCCACCCCATTGACGTCAATGGGAGTTTG |
| | | TTTTGGCACCAAAATCAACGGGACTTTCCA |
| | | AAATGTCGTAACAACTCCGCCCCATTGACG |
| | | CAAATGGGCGGTAGGCGTGTACGGTGGGAG |
| | | GTCTATATAAGCAGAGCTCGTTTAGTGAAC |
| | | CGTCAGATCGCCTGGAGACGCCATCCACGC |
| | | TGTTTTGACCTCCATAGAAGACACCGGGAC |
| | | CGATCCAGCCTCCGGACTCTAGAGTTAACT |
| | | GGTAAGTTTAGTCTTTTTGTCTTTTATTTC |
| | | AGGTCCCGGATCCGGTGGTGGTGCAAATCA |
| | | AAGAACTGCTCCTCAGTGGATGTTGCCTTT |
| | | ACTTCTAGGCCTGCCGCCACCATGGTGAGC |
| | | TACTGGGACACCGGCGTGCTGCTGTGCGCC |
| | | CTGCTGAGCTGCCTGCTGCTGACCGGCAGC |
| | | AGCAGCGGCAGCGACACCGGCAGGCCTTTC |
| | | GTGGAGATGTACAGCGAGATCCCTGAGATC |
| | | ATCCACATGACCGAGGGCAGGGAGCTGGTG |
| | | ATCCCTTGCAGGGTGACCAGCCCTAACATC |
| | | ACCGTGACCCTGAAGAAGTTCCCTCTGGAC |
| | | ACCCTGATCCCTGACGGCAAGAGGATCATC |
| | | TGGGACAGCAGGAAGGGCTTCATCATCAGC |
| | | AACGCCACCTACAAGGAGATCGGCCTGCTG |
| | | ACCTGCGAGGCCACCGTGAACGGCCACCTG |
| | | TACAAGACCAACTACCTGACCCACAGGCAG |
| | | ACCAACACCATCATCGACGTGGTGCTGAGC |
| | | CCTAGCCACGGCATCGAGCTGAGCGTGGGC |
| | | GAGAAGCTGGTGCTGAACTGCACCGCCAGG |
| | | ACCGAGCTGAACGTGGGCATCGACTTCAAC |
| | | TGGGAGTACCCTAGCAGCAAGCACCAGCAC |
| | | AAGAAGCTGGTGAACAGGGACCTGAAGACC |
| | | CAGAGCGGCAGCGAGATGAAGAAGTTCCTG |
| | | AGCACCCTGACCATCGACGGCGTGACCAGG |
| | | AGCGACCAGGGCCTGTACACCTGCGCCGCC |
| | | AGCAGCGGCCTGATGACCAAGAAGAACAGC |
| | | ACCTTCGTGAGGGTGCACGAGAAGGACAAG |
| | | ACCCACACCTGCCCTCCTTGCCCTGCCCCT |
| | | GAGCTGCTGGGCGGCCCTAGCGTGTTCCTG |
| | | TTCCCTCCTAAGCCTAAGGACACCCTGATG |
| | | ATCAGCAGGACCCCTGAGGTGACCTGCGTG |
| | | GTGGTGGACGTGAGCCACGAGGACCCTGAG |
| | | GTGAAGTTCAACTGGTACGTGGACGGCGTG |
| | | GAGGTGCACAACGCCAAGACCAAGCCTAGG |
| | | GAGGAGCAGTACAACAGCACCTACAGGGTG |
| | | GTGAGCGTGCTGACCGTGCTGCACCAGGAC |
| | | TGGCTGAACGGCAAGGAGTACAAGTGCAAG |
| | | GTGAGCAACAAGGCCCTGCCTGCCCCTATC |
| | | GAGAAGACCATCAGCAAGGCCAAGGGCCAG |
| | | CCTAGGGAGCCTCAGGTGTACACCCTGCCT |
| | | CCTAGCAGGGACGAGCTGACCAAGAACCAG |
| | | GTGAGCCTGACCTGCCTGGTGAAGGGCTTC |
| | | TACCCTAGCGACATCGCCGTGGAGTGGGAG |
| | | AGCAACGGCCAGCCTGAGAACAACTACAAG |
| | | ACCACCCCTCCTGTGCTGGACAGCGACGGC |
| | | AGCTTCTTCCTGTACAGCAAGCTGACCGTG |
| | | GACAAGAGCAGGTGGCAGCAGGGCAACGTG |
| | | TTCAGCTGCAGCGTGATGCACGAGGCCCTG |
| | | CACAACCACTACACCCAGAAGAGCCTGAGC |
| | | CTGAGCCCTGGCAAGTGATTCGAAAATAAA |
| | | ATATCTTTATTTTCATTACATCTGTGTGTT |
| | | GGTTTTTTGTGTGGCATGCTGGGGAGAGAT |
| | | CAACCCCACTCCCTCTCTGCGCGCTCGCTC |
| | | GCTCACTGAGGCCGGGCGACCAAAGGTCGC |
| | | CCGACGCCCGGGCTTTGCCCGGGCGGCCTC |
| | | AGTGAGCGAGCGAGCGCGCAGCAAGCTGTA |
| | | GCCAACCACTAGAACTATAGCTAGAGTCCT |
| | | GGGCGAACAAACGATGCTCGCCTTCCAGAA |
| | | AACCGAGGATGCGAACCACTTCATCCGGGG |
| | | TCAGCACCACCGGCAAGCGCCGCGACGGCC |
| | | GAGGTCTTCCGATCTCCTGAAGCCAGGGCA |
| | | GATCCGTGCACAGCACCTTGCCGTAGAAGA |
| | | ACAGCAAGGCCGCCAATGCCTGACGATGCG |
| | | TGGAGACCGAAACCTTGCGCTCGTTCGCCA |
| | | GCCAGGACAGAAATGCCTCGACTTCGCTGC |
| | | TGCCCAAGGTTGCCGGGTGACGCACACCGT |
| | | GGAAACGGATGAAGGCACGAACCCAGTTGA |
| | | CATAAGCCTGTTCGGTTCGTAAACTGTAAT |
| | | GCAAGTAGCGTATGCGCTCACGCAACTGGT |
| | | CCAGAACCTTGACCGAACGCAGCGGTGGTA |
| | | ACGGCGCAGTGGCGGTTTTCATGGCTTGTT |
| | | ATGACTGTTTTTTTGTACAGTCTATGCCTC |
| | | GGGCATCCAAGCAGCAAGCGCGTTACGCCG |
| | | TGGGTCGATGTTTGATGTTATGGAGCAGCA |
| | | ACGATGTTACGCAGCAGCAACGATGTTACG |
| | | CAGCAGGGCAGTCGCCCTAAAACAAAGTTA |
| | | GGTGGCTCAAGTATGGGCATCATTCGCACA |
| | | TGTAGGCTCGGCCCTGACCAAGTCAAATCC |
| | | ATGCGGGCTGCTCTTGATCTTTTCGGTCGT |
| | | GAGTTCGGAGACGTAGCCACCTACTCCCAA |
| | | CATCAGCCGGACTCCGATTACCTCGGGAAC |
| | | TTGCTCCGTAGTAAGACATTCATCGCGCTT |
| | | GCTGCCTTCGACCAAGAAGCGGTTGTTGGC |
| | | GCTCTCGCGGCTTACGTTCTGCCCAGGTTT |
| | | GAGCAGCCGCGTAGTGAGATCTATATCTAT |
| | | GATCTCGCAGTCTCCGGCGAGCACCGGAGG |
| | | CAGGGCATTGCCACCGCGCTCATCAATCTC |
| | | CTCAAGCATGAGGCCAACGCGCTTGGTGCT |
| | | TATGTGATCTACGTGCAAGCAGATTACGGT |
| | | GACGATCCCGCAGTGGCTCTCTATACAAAG |
| | | TTGGGCATACGGGAAGAAGTGATGCACTTT |
| | | GATATCGACCCAAGTACCGCCACCTAACAA |
| | | TTCGTTCAAGCCGAGATCGGCTTCCCGGCC |
| | | GCGGAGTTGTTCGGTAAATTGTCACAACGC |
| | | CGCGAATATAGTCTTTACCATGCCCTTGGC |
| | | CACGCCCCTCTTTAATACGACGGGCAATTT |
| | | GCACTTCAGAAAATGAAGAGTTTGCTTTAG |
| | | CCATAACAAAAGTCCAGTATGCTTTTTCAC |
| | | AGCATAACTGGACTGATTTCAGTTTACAAC |
| | | TATTCTGTCTAGTTTAAGACTTTATTGTCA |
| | | TAGTTTAGATCTATTTTGTTCAGTTTAAGA |
| | | CTTTATTGTCCGCCCACACCCGCTTACGCA |
| | | GGGCATCCATTTATTACTCAACCGTAACCG |
| | | ATTTTGCCAGGTTACGCGGCTGGTCTGCGG |
| | | TGTGAAATACCGCACAGATGCGTAAGGAGA |
| | | AAATACCGCATCAGGCGCTCTTCCGCTTCC |
| | | TCGCTCACTGACTCGCTGCGCTCGGTCGTT |
| | | CGGCTGCGGCGAGCGGTATCAGCTCACTCA |
| | | AAGGCGGTAATACGGTTATCCACAGAATCA |
| | | GGGGATAACGCAGGAAAGAACATGTGAGCA |
| | | AAAGGCCAGCAAAAGGCCAGGAACCGTAAA |
| | | AAGGCCGCGTTGCTGGCGTTTTTCCATAGG |
| | | CTCCGCCCCCCTGACGAGCATCACAAAAAT |
| | | CGACGCTCAAGTCAGAGGTGGCGAAACCCG |
| | | ACAGGACTATAAAGATACCAGGCGTTTCCC |
| | | CCTGGAAGCTCCCTCGTGCGCTCTCCTGTT |
| | | CCGACCCTGCCGCTTACCGGATACCTGTCC |
| | | GCCTTTCTCCCTTCGGGAAGCGTGGCGCTT |
| | | TCTCAATGCTCACGCTGTAGGTATCTCAGT |
| | | TCGGTGTAGGTCGTTCGCTCCAAGCTGGGC |
| | | TGTGTGCACGAACCCCCCGTTCAGCCCGAC |
| | | CGCTGCGCCTTATCCGGTAACTATCGTCTT |
| | | GAGTCCAACCCGGTAAGACACGACTTATCG |
| | | CCACTGGCAGCAGCCACTGGTAACAGGATT |
| | | AGCAGAGCGAGGTATGTAGGCGGTGCTACA |
| | | GAGTTCTTGAAGTGGTGGCCTAACTACGGC |
| | | TACACTAGAAGGACAGTATTTGGTATCTGC |
| | | GCTCTGCTGAAGCCAGTTACCTTCGGAAAA |
| | | AGAGTTGGTAGCTCTTGATCCGGCAAACAA |
| | | ACCACCGCTGGTAGCGGTGGTTTTTTTGTT |
| | | TGCAAGCAGCAGATTACGCGCAGAAAAAAA |
| | | GGATCTCAAGAAGATCCTTTGATCTTTTCT |
| | | ACGGGGTCTGACGCTCAGTGGAACGAAAAC |
| | | TCACGTTAAGGGATTTTGGTCATGAGATTA |
| | | TCAAAAAGGATCTTCACCTAGATCCTTTTA |
| | | AATTAAAAATGAAGTTTTAAATCAATCTAA |
| | | AGTATATATGAGTAAACTTGGTCTGACAGT |
| | | TACCAATGCTTAATCAGTGAGGCACCTATC |
| | | TCAGCGATCTGTCTATTTCGTTCATCCATA |
| | | GTTGCCTGACTCCCCGTCGTGTAGATAACT |
| | | ACGATACGGGAGGGCTTACCATCTGGCCCC |
| | | AGTGCTGCAATGATACCGCGAGACCCACGC |
| | | TCACCGGCTCCAGATTTATCAGCAATAAAC |
| | | CAGCCAGCCGGAAGGGCCGAGCGCAGAAGT |
| | | GGTCCTGCAACTTTATCCGCCTCCATCCAG |
| | | TCTATTAATTGTTGCCGGGAAGCTAGAGTA |
| | | AGTAGTTCGCCAGTTAATAGTTTGCGCAAC |
| | | GTTGTTGCCATTGCTACAGGCATCGTGGTG |
| | | TCACGCTCGTCGTTTGGTATGGCTTCATTC |
| | | AGCTCCGGTTCCCAACGATCAAGGCGAGTT |
| | | ACATGATCCCCCATGTTGTGCAAAAAAGCG |
| | | GTTAGCTCCTTCGGTCCTCCGATCGTTGTC |
| | | AGAAGTAAGTTGGCCGCAGTGTTATCACTC |
| | | ATGGTTATGGCAGCACTGCATAATTCTCTT |
| | | ACTGTCATGCCATCCGTAAGATGCTTTTCT |
| | | GTGACTGGTGAGTACTCAACCAAGTCATTC |
| | | TGAGAATAGTGTATGCGGCGACCGAGTTGC |
| | | TCTTGCCCGGCGTCAATACGGGATAATACC |
| | | GCGCCACATAGCAGAACTTTAAAAGTGCTC |
| | | ATCATTGGAAAACGTTCTTCGGGGCGAAAA |
| | | CTCTCAAGGATCTTACCGCTGTTGAGATCC |
| | | AGTTCGATGTAACCCACTCGTGCACCCAAC |
| | | TGATCTTCAGCATCTTTTACTTTCACCAGC |
| | | GTTTCTGGGTGAGCAAAAACAGGAAGGCAA |
| | | AATGCCGCAAAAAAGGGAATAAGGGCGACA |
| | | CGGAAATGTTGAATACTCATACTCTTCCTT |
| | | TTTCAATATTATTGAAGCATTTATCAGGGT |
| | | TATTGTCTCATGAGCGGATACATATTTGAA |
| | | TGTATTTAGAAAAATAAACAAATAGGGGTT |
| | | CCGCGCACATTTCCCCGAAAAGTGCCACCT |
| | | GAAATTGTAAACGTTAATATTTTGTTAAAA |
| | | TTCGCGTTAAATTTTTGTTAAATCAGCTCA |
| | | TTTTTTAACCAATAGGCCGAAATCGGCAAA |
| | | ATCCCTTATAAATCAAAAGAATAGACCGAG |
| | | ATAGGGTTGAGTGTTGTTCCAGTTTGGAAC |
| | | AAGAGTCCACTATTAAAGAACGTGGACTCC |
| | | AACGTCAAAGGGCGAAAAACCGTCTATCAG |
| | | GGCGATGGCCCACTACGTGAACCATCACCC |
| | | TAATCAAGTTTTTTGGGGTCGAGGTGCCGT |
| | | AAAGCACTAAATCGGAACCCTAAAGGGAGC |
| | | CCCCGATTTAGAGCTTGACGGGGAAAGCCG |
| | | GCGAACGTGGCGAGAAAGGAAGGGAAGAAA |
| | | GCGAAAGGAGCGGGCGCTAGGGCGCTGGCA |
| | | AGTGTAGCGGTCACGCTGCGCGTAACCACC |
| | | ACACCCGCCGCGCTTAATGCGCCGCTACAG |
| | | GGCGCGTC |
| 18 | AMI120 | CATTCGCCATTCAGGCTGCAAATAAGCGTT |
| | plasmid | GATATTCAGTCAATTACAAACATTAATAAC |
| | | GAAGAGATGACAGAAAAATTTTCATTCTGT |
| | | GACAGAGAAAAAGTAGCCGAAGATGACGGT |
| | | TTGTCACATGGAGTTGGCAGGATGTTTGAT |
| | | TAAAAACATAACAGGAAGAAAAATGCCCCG |
| | | CTGTGGGCGGACAAAATAGTTGGGAACTGG |
| | | GAGGGGTGGAAATGGAGTTTTTAAGGATTA |
| | | TTTAGGGAAGAGTGACAAAATAGATGGGAA |
| | | CTGGGTGTAGCGTCGTAAGCTAATACGAAA |
| | | ATTAAAAATGACAAAATAGTTTGGAACTAG |
| | | ATTTCACTTATCTGGTTCGGATCTCCTAGG |
| | | CTCAAGCAGTGATCAGATCCAGACATGATA |
| | | AGATACATTGATGAGTTTGGACAAACCACA |
| | | ACTAGAATGCAGTGAAAAAAATGCTTTATT |
| | | TGTGAAATTTGTGATGCTATTGCTTTATTT |
| | | GTAACCATTATAAGCTGCAATAAACAAGTT |
| | | AACAACAACAATTGCATTCATTTTATGTTT |
| | | CAGGTTCAGGGGGAGGTGTGGGAGGTTTTT |
| | | TAAAGCAAGTAAAACCTCTACAAATGTGGT |
| | | ATGGCTGATTATGATCCTCTAGTACTTCTC |
| | | GACAAGCTCGGATCCTGGCGCGCTCGCTCG |
| | | CTCACTGAGGCCGCCCGGGCAAAGCCCGGG |
| | | CGTCGGGCGACCTTTGGTCGCCCGGCCTCA |
| | | GTGAGCGAGCGAGCGCGCAGAGAGGGAGTG |
| | | GCCAACTCCATCACTAGGGGTTCCTAGGAA |
| | | GCTGATCTGAATTCGGTACCCGTTACATAA |
| | | CTTACGGTAAATGGCCCGCCTGGCTGACCG |
| | | CCCAACGACCCCCGCCCATTGACGTCAATA |
| | | ATGACGTATGTTCCCATAGTAACGCCAATA |
| | | GGGACTTTCCATTGACGTCAATGGGTGGAG |
| | | TATTTACGGTAAACTGCCCACTTGGCAGTA |
| | | CATCAAGTGTATCATATGCCAAGTACGCCC |
| | | CCTATTGACGTCAATGACGGTAAATGGCCC |
| | | GCCTGGCATTATGCCCAGTACATGACCTTA |
| | | TGGGACTTTCCTACTTGGCAGTACATCTAC |
| | | GTATTAGTCATCGCTATTACCATGGTGATG |
| | | CGGTTTTGGCAGTACATCAATGGGCGTGGA |
| | | TAGCGGTTTGACTCACGGGGATTTCCAAGT |
| | | CTCCACCCCATTGACGTCAATGGGAGTTTG |
| | | TTTTGGCACCAAAATCAACGGGACTTTCCA |
| | | AAATGTCGTAACAACTCCGCCCCATTGACG |
| | | CAAATGGGCGGTAGGCGTGTACGGTGGGAG |
| | | GTCTATATAAGCAGAGCTCGTTTAGTGAAC |
| | | CGTCAGATCGCCTGGAGACGCCATCCACGC |
| | | TGTTTTGACCTCCATAGAAGACACCGGGAC |
| | | CGATCCAGCCTCCGGACTCTAGAGTTAACT |
| | | GGTAAGTTTAGTCTTTTTGTCTTTTATTTC |
| | | AGGTCCCGGATCCGGTGGTGGTGCAAATCA |
| | | AAGAACTGCTCCTCAGTGGATGTTGCCTTT |
| | | ACTTCTAGGCCTGCCGCCACCATGGTGAGC |
| | | TACTGGGACACCGGCGTGCTGCTGTGCGCC |
| | | CTGCTGAGCTGCCTGCTGCTGACCGGCAGC |
| | | AGCAGCGGCAGCGACACCGGCAGGCCCTTC |
| | | GTGGAGATGTACTCCGAGATCCCCGAGATC |
| | | ATCCACATGACCGAGGGCAGGGAGCTGGTG |
| | | ATCCCCTGCAGGGTGACCTCCCCCAACATC |
| | | ACCGTGACCCTGAAGAAGTTCCCCCTGGAC |
| | | ACCCTGATCCCCGACGGCAAGAGGATCATC |
| | | TGGGACTCCAGGAAGGGCTTCATCATCTCC |
| | | AACGCCACCTACAAGGAGATCGGCCTGCTG |
| | | ACCTGCGAGGCCACCGTGAACGGCCACCTG |
| | | TACAAGACCAACTACCTGACCCACAGGCAG |
| | | ACCAACACCATCATCGACGTGGTGCTGTCC |
| | | CCCTCCCACGGCATCGAGCTGTCCGTGGGC |
| | | GAGAAGCTGGTGCTGAACTGCACCGCCAGG |
| | | ACCGAGCTGAACGTGGGCATCGACTTCAAC |
| | | TGGGAGTACCCCTCCTCCAAGCACCAGCAC |
| | | AAGAAGCTGGTGAACAGGGACCTGAAGACC |
| | | CAGTCCGGCTCCGAGATGAAGAAGTTCCTG |
| | | TCCACCCTGACCATCGACGGCGTGACCAGG |
| | | TCCGACCAGGGCCTGTACACCTGCGCCGCC |
| | | TCCTCCGGCCTGATGACCAAGAAGAACTCC |
| | | ACCTTCGTGAGGGTGCACGAGAAGGACAAG |
| | | ACCCACACCTGCCCCCCCTGCCCCGCCCCC |
| | | GAGCTGCTGGGCGGCCCCTCCGTGTTCCTG |
| | | TTCCCCCCCAAGCCCAAGGACACCCTGATG |
| | | ATCTCCAGGACCCCCGAGGTGACCTGCGTG |
| | | GTGGTGGACGTGTCCCACGAGGACCCCGAG |
| | | GTGAAGTTCAACTGGTACGTGGACGGCGTG |
| | | GAGGTGCACAACGCCAAGACCAAGCCCAGG |
| | | GAGGAGCAGTACAACTCCACCTACAGGGTG |
| | | GTGTCCGTGCTGACCGTGCTGCACCAGGAC |
| | | TGGCTGAACGGCAAGGAGTACAAGTGCAAG |
| | | GTGTCCAACAAGGCCCTGCCCGCCCCCATC |
| | | GAGAAGACCATCTCCAAGGCCAAGGGCCAG |
| | | CCCAGGGAGCCCCAGGTGTACACCCTGCCC |
| | | CCCTCCAGGGACGAGCTGACCAAGAACCAG |
| | | GTGTCCCTGACCTGCCTGGTGAAGGGCTTC |
| | | TACCCCTCCGACATCGCCGTGGAGTGGGAG |
| | | TCCAACGGCCAGCCCGAGAACAACTACAAG |
| | | ACCACCCCCCCCGTGCTGGACTCCGACGGC |
| | | TCCTTCTTCCTGTACTCCAAGCTGACCGTG |
| | | GACAAGTCCAGGTGGCAGCAGGGCAACGTG |
| | | TTCTCCTGCTCCGTGATGCACGAGGCCCTG |
| | | CACAACCACTACACCCAGAAGTCCCTGTCC |
| | | CTGTCCCCCGGCAAGTGATTCGAAAATAAA |
| | | ATATCTTTATTTTCATTACATCTGTGTGTT |
| | | GGTTTTTTGTGTGGCATGCTGGGGAGAGAT |
| | | CAACCCCACTCCCTCTCTGCGCGCTCGCTC |
| | | GCTCACTGAGGCCGGGCGACCAAAGGTCGC |
| | | CCGACGCCCGGGCTTTGCCCGGGCGGCCTC |
| | | AGTGAGCGAGCGAGCGCGCAGCAAGCTGTA |
| | | GCCAACCACTAGAACTATAGCTAGAGTCCT |
| | | GGGCGAACAAACGATGCTCGCCTTCCAGAA |
| | | AACCGAGGATGCGAACCACTTCATCCGGGG |
| | | TCAGCACCACCGGCAAGCGCCGCGACGGCC |
| | | GAGGTCTTCCGATCTCCTGAAGCCAGGGCA |
| | | GATCCGTGCACAGCACCTTGCCGTAGAAGA |
| | | ACAGCAAGGCCGCCAATGCCTGACGATGCG |
| | | TGGAGACCGAAACCTTGCGCTCGTTCGCCA |
| | | GCCAGGACAGAAATGCCTCGACTTCGCTGC |
| | | TGCCCAAGGTTGCCGGGTGACGCACACCGT |
| | | GGAAACGGATGAAGGCACGAACCCAGTTGA |
| | | CATAAGCCTGTTCGGTTCGTAAACTGTAAT |
| | | GCAAGTAGCGTATGCGCTCACGCAACTGGT |
| | | CCAGAACCTTGACCGAACGCAGCGGTGGTA |
| | | ACGGCGCAGTGGCGGTTTTCATGGCTTGTT |
| | | ATGACTGTTTTTTTGTACAGTCTATGCCTC |
| | | GGGCATCCAAGCAGCAAGCGCGTTACGCCG |
| | | TGGGTCGATGTTTGATGTTATGGAGCAGCA |
| | | ACGATGTTACGCAGCAGCAACGATGTTACG |
| | | CAGCAGGGCAGTCGCCCTAAAACAAAGTTA |
| | | GGTGGCTCAAGTATGGGCATCATTCGCACA |
| | | TGTAGGCTCGGCCCTGACCAAGTCAAATCC |
| | | ATGCGGGCTGCTCTTGATCTTTTCGGTCGT |
| | | GAGTTCGGAGACGTAGCCACCTACTCCCAA |
| | | CATCAGCCGGACTCCGATTACCTCGGGAAC |
| | | TTGCTCCGTAGTAAGACATTCATCGCGCTT |
| | | GCTGCCTTCGACCAAGAAGCGGTTGTTGGC |
| | | GCTCTCGCGGCTTACGTTCTGCCCAGGTTT |
| | | GAGCAGCCGCGTAGTGAGATCTATATCTAT |
| | | GATCTCGCAGTCTCCGGCGAGCACCGGAGG |
| | | CAGGGCATTGCCACCGCGCTCATCAATCTC |
| | | CTCAAGCATGAGGCCAACGCGCTTGGTGCT |
| | | TATGTGATCTACGTGCAAGCAGATTACGGT |
| | | GACGATCCCGCAGTGGCTCTCTATACAAAG |
| | | TTGGGCATACGGGAAGAAGTGATGCACTTT |
| | | GATATCGACCCAAGTACCGCCACCTAACAA |
| | | TTCGTTCAAGCCGAGATCGGCTTCCCGGCC |
| | | GCGGAGTTGTTCGGTAAATTGTCACAACGC |
| | | CGCGAATATAGTCTTTACCATGCCCTTGGC |
| | | CACGCCCCTCTTTAATACGACGGGCAATTT |
| | | GCACTTCAGAAAATGAAGAGTTTGCTTTAG |
| | | CCATAACAAAAGTCCAGTATGCTTTTTCAC |
| | | AGCATAACTGGACTGATTTCAGTTTACAAC |
| | | TATTCTGTCTAGTTTAAGACTTTATTGTCA |
| | | TAGTTTAGATCTATTTTGTTCAGTTTAAGA |
| | | CTTTATTGTCCGCCCACACCCGCTTACGCA |
| | | GGGCATCCATTTATTACTCAACCGTAACCG |
| | | ATTTTGCCAGGTTACGCGGCTGGTCTGCGG |
| | | TGTGAAATACCGCACAGATGCGTAAGGAGA |
| | | AAATACCGCATCAGGCGCTCTTCCGCTTCC |
| | | TCGCTCACTGACTCGCTGCGCTCGGTCGTT |
| | | CGGCTGCGGCGAGCGGTATCAGCTCACTCA |
| | | AAGGCGGTAATACGGTTATCCACAGAATCA |
| | | GGGGATAACGCAGGAAAGAACATGTGAGCA |
| | | AAAGGCCAGCAAAAGGCCAGGAACCGTAAA |
| | | AAGGCCGCGTTGCTGGCGTTTTTCCATAGG |
| | | CTCCGCCCCCCTGACGAGCATCACAAAAAT |
| | | CGACGCTCAAGTCAGAGGTGGCGAAACCCG |
| | | ACAGGACTATAAAGATACCAGGCGTTTCCC |
| | | CCTGGAAGCTCCCTCGTGCGCTCTCCTGTT |
| | | CCGACCCTGCCGCTTACCGGATACCTGTCC |
| | | GCCTTTCTCCCTTCGGGAAGCGTGGCGCTT |
| | | TCTCAATGCTCACGCTGTAGGTATCTCAGT |
| | | TCGGTGTAGGTCGTTCGCTCCAAGCTGGGC |
| | | TGTGTGCACGAACCCCCCGTTCAGCCCGAC |
| | | CGCTGCGCCTTATCCGGTAACTATCGTCTT |
| | | GAGTCCAACCCGGTAAGACACGACTTATCG |
| | | CCACTGGCAGCAGCCACTGGTAACAGGATT |
| | | AGCAGAGCGAGGTATGTAGGCGGTGCTACA |
| | | GAGTTCTTGAAGTGGTGGCCTAACTACGGC |
| | | TACACTAGAAGGACAGTATTTGGTATCTGC |
| | | GCTCTGCTGAAGCCAGTTACCTTCGGAAAA |
| | | AGAGTTGGTAGCTCTTGATCCGGCAAACAA |
| | | ACCACCGCTGGTAGCGGTGGTTTTTTTGTT |
| | | TGCAAGCAGCAGATTACGCGCAGAAAAAAA |
| | | GGATCTCAAGAAGATCCTTTGATCTTTTCT |
| | | ACGGGGTCTGACGCTCAGTGGAACGAAAAC |
| | | TCACGTTAAGGGATTTTGGTCATGAGATTA |
| | | TCAAAAAGGATCTTCACCTAGATCCTTTTA |
| | | AATTAAAAATGAAGTTTTAAATCAATCTAA |
| | | AGTATATATGAGTAAACTTGGTCTGACAGT |
| | | TACCAATGCTTAATCAGTGAGGCACCTATC |
| | | TCAGCGATCTGTCTATTTCGTTCATCCATA |
| | | GTTGCCTGACTCCCCGTCGTGTAGATAACT |
| | | ACGATACGGGAGGGCTTACCATCTGGCCCC |
| | | AGTGCTGCAATGATACCGCGAGACCCACGC |
| | | TCACCGGCTCCAGATTTATCAGCAATAAAC |
| | | CAGCCAGCCGGAAGGGCCGAGCGCAGAAGT |
| | | GGTCCTGCAACTTTATCCGCCTCCATCCAG |
| | | TCTATTAATTGTTGCCGGGAAGCTAGAGTA |
| | | AGTAGTTCGCCAGTTAATAGTTTGCGCAAC |
| | | GTTGTTGCCATTGCTACAGGCATCGTGGTG |
| | | TCACGCTCGTCGTTTGGTATGGCTTCATTC |
| | | AGCTCCGGTTCCCAACGATCAAGGCGAGTT |
| | | ACATGATCCCCCATGTTGTGCAAAAAAGCG |
| | | GTTAGCTCCTTCGGTCCTCCGATCGTTGTC |
| | | AGAAGTAAGTTGGCCGCAGTGTTATCACTC |
| | | ATGGTTATGGCAGCACTGCATAATTCTCTT |
| | | ACTGTCATGCCATCCGTAAGATGCTTTTCT |
| | | GTGACTGGTGAGTACTCAACCAAGTCATTC |
| | | TGAGAATAGTGTATGCGGCGACCGAGTTGC |
| | | TCTTGCCCGGCGTCAATACGGGATAATACC |
| | | GCGCCACATAGCAGAACTTTAAAAGTGCTC |
| | | ATCATTGGAAAACGTTCTTCGGGGCGAAAA |
| | | CTCTCAAGGATCTTACCGCTGTTGAGATCC |
| | | AGTTCGATGTAACCCACTCGTGCACCCAAC |
| | | TGATCTTCAGCATCTTTTACTTTCACCAGC |
| | | GTTTCTGGGTGAGCAAAAACAGGAAGGCAA |
| | | AATGCCGCAAAAAAGGGAATAAGGGCGACA |
| | | CGGAAATGTTGAATACTCATACTCTTCCTT |
| | | TTTCAATATTATTGAAGCATTTATCAGGGT |
| | | TATTGTCTCATGAGCGGATACATATTTGAA |
| | | TGTATTTAGAAAAATAAACAAATAGGGGTT |
| | | CCGCGCACATTTCCCCGAAAAGTGCCACCT |
| | | GAAATTGTAAACGTTAATATTTTGTTAAAA |
| | | TTCGCGTTAAATTTTTGTTAAATCAGCTCA |
| | | TTTTTTAACCAATAGGCCGAAATCGGCAAA |
| | | ATCCCTTATAAATCAAAAGAATAGACCGAG |
| | | ATAGGGTTGAGTGTTGTTCCAGTTTGGAAC |
| | | AAGAGTCCACTATTAAAGAACGTGGACTCC |
| | | AACGTCAAAGGGCGAAAAACCGTCTATCAG |
| | | GGCGATGGCCCACTACGTGAACCATCACCC |
| | | TAATCAAGTTTTTTGGGGTCGAGGTGCCGT |
| | | AAAGCACTAAATCGGAACCCTAAAGGGAGC |
| | | CCCCGATTTAGAGCTTGACGGGGAAAGCCG |
| | | GCGAACGTGGCGAGAAAGGAAGGGAAGAAA |
| | | GCGAAAGGAGCGGGCGCTAGGGCGCTGGCA |
| | | AGTGTAGCGGTCACGCTGCGCGTAACCACC |
| | | ACACCCGCCGCGCTTAATGCGCCGCTACAG |
| | | GGCGCGTC |
| 19 | AMI130 | CATTCGCCATTCAGGCTGCAAATAAGCGTT |
| | plasmid | GATATTCAGTCAATTACAAACATTAATAAC |
| | | GAAGAGATGACAGAAAAATTTTCATTCTGT |
| | | GACAGAGAAAAAGTAGCCGAAGATGACGGT |
| | | TTGTCACATGGAGTTGGCAGGATGTTTGAT |
| | | TAAAAACATAACAGGAAGAAAAATGCCCCG |
| | | CTGTGGGCGGACAAAATAGTTGGGAACTGG |
| | | GAGGGGTGGAAATGGAGTTTTTAAGGATTA |
| | | TTTAGGGAAGAGTGACAAAATAGATGGGAA |
| | | CTGGGTGTAGCGTCGTAAGCTAATACGAAA |
| | | ATTAAAAATGACAAAATAGTTTGGAACTAG |
| | | ATTTCACTTATCTGGTTCGGATCTCCTAGG |
| | | CTCAAGCAGTGATCAGATCCAGACATGATA |
| | | AGATACATTGATGAGTTTGGACAAACCACA |
| | | ACTAGAATGCAGTGAAAAAAATGCTTTATT |
| | | TGTGAAATTTGTGATGCTATTGCTTTATTT |
| | | GTAACCATTATAAGCTGCAATAAACAAGTT |
| | | AACAACAACAATTGCATTCATTTTATGTTT |
| | | CAGGTTCAGGGGGAGGTGTGGGAGGTTTTT |
| | | TAAAGCAAGTAAAACCTCTACAAATGTGGT |
| | | ATGGCTGATTATGATCCTCTAGTACTTCTC |
| | | GACAAGCTCGGATCCTGGCGCGCTCGCTCG |
| | | CTCACTGAGGCCGCCCGGGCAAAGCCCGGG |
| | | CGTCGGGCGACCTTTGGTCGCCCGGCCTCA |
| | | GTGAGCGAGCGAGCGCGCAGAGAGGGAGTG |
| | | GCCAACTCCATCACTAGGGGTTCCTAGGAA |
| | | GCTGATCTGAATTCGGTACCCGTTACATAA |
| | | CTTACGGTAAATGGCCCGCCTGGCTGACCG |
| | | CCCAACGACCCCCGCCCATTGACGTCAATA |
| | | ATGACGTATGTTCCCATAGTAACGCCAATA |
| | | GGGACTTTCCATTGACGTCAATGGGTGGAG |
| | | TATTTACGGTAAACTGCCCACTTGGCAGTA |
| | | CATCAAGTGTATCATATGCCAAGTACGCCC |
| | | CCTATTGACGTCAATGACGGTAAATGGCCC |
| | | GCCTGGCATTATGCCCAGTACATGACCTTA |
| | | TGGGACTTTCCTACTTGGCAGTACATCTAC |
| | | GTATTAGTCATCGCTATTACCATGGTGATG |
| | | CGGTTTTGGCAGTACATCAATGGGCGTGGA |
| | | TAGCGGTTTGACTCACGGGGATTTCCAAGT |
| | | CTCCACCCCATTGACGTCAATGGGAGTTTG |
| | | TTTTGGCACCAAAATCAACGGGACTTTCCA |
| | | AAATGTCGTAACAACTCCGCCCCATTGACG |
| | | CAAATGGGCGGTAGGCGTGTACGGTGGGAG |
| | | GTCTATATAAGCAGAGCTCGTTTAGTGAAC |
| | | CGTCAGATCGCCTGGAGACGCCATCCACGC |
| | | TGTTTTGACCTCCATAGAAGACACCGGGAC |
| | | CGATCCAGCCTCCGGACTCTAGAGTTAACT |
| | | GGTAAGTTTAGTCTTTTTGTCTTTTATTTC |
| | | AGGTCCCGGATCCGGTGGTGGTGCAAATCA |
| | | AAGAACTGCTCCTCAGTGGATGTTGCCTTT |
| | | ACTTCTAGGCCTGCCGCCACCATGGTGAGC |
| | | TACTGGGACACCGGCGTGCTGCTGTGCGCC |
| | | CTGCTGAGCTGCCTGCTGCTGACCGGCAGC |
| | | AGCAGCGGCAGCGACACCGGCAGGCCGTTC |
| | | GTGGAGATGTACAGCGAGATCCCGGAGATC |
| | | ATCCACATGACCGAGGGCAGGGAGCTGGTG |
| | | ATCCCGTGCAGGGTGACCAGCCCGAACATC |
| | | ACCGTGACCCTGAAGAAGTTCCCGCTGGAC |
| | | ACCCTGATCCCGGACGGCAAGAGGATCATC |
| | | TGGGACAGCAGGAAGGGCTTCATCATCAGC |
| | | AACGCCACCTACAAGGAGATCGGCCTGCTG |
| | | ACCTGCGAGGCCACCGTGAACGGCCACCTG |
| | | TACAAGACCAACTACCTGACCCACAGGCAG |
| | | ACCAACACCATCATCGACGTGGTGCTGAGC |
| | | CCGAGCCACGGCATCGAGCTGAGCGTGGGC |
| | | GAGAAGCTGGTGCTGAACTGCACCGCCAGG |
| | | ACCGAGCTGAACGTGGGCATCGACTTCAAC |
| | | TGGGAGTACCCGAGCAGCAAGCACCAGCAC |
| | | AAGAAGCTGGTGAACAGGGACCTGAAGACC |
| | | CAGAGCGGCAGCGAGATGAAGAAGTTCCTG |
| | | AGCACCCTGACCATCGACGGCGTGACCAGG |
| | | AGCGACCAGGGCCTGTACACCTGCGCCGCC |
| | | AGCAGCGGCCTGATGACCAAGAAGAACAGC |
| | | ACCTTCGTGAGGGTGCACGAGAAGGACAAG |
| | | ACCCACACCTGCCCGCCGTGCCCGGCCCCG |
| | | GAGCTGCTGGGCGGCCCGAGCGTGTTCCTG |
| | | TTCCCGCCGAAGCCGAAGGACACCCTGATG |
| | | ATCAGCAGGACCCCGGAGGTGACCTGCGTG |
| | | GTGGTGGACGTGAGCCACGAGGACCCGGAG |
| | | GTGAAGTTCAACTGGTACGTGGACGGCGTG |
| | | GAGGTGCACAACGCCAAGACCAAGCCGAGG |
| | | GAGGAGCAGTACAACAGCACCTACAGGGTG |
| | | GTGAGCGTGCTGACCGTGCTGCACCAGGAC |
| | | TGGCTGAACGGCAAGGAGTACAAGTGCAAG |
| | | GTGAGCAACAAGGCCCTGCCGGCCCCGATC |
| | | GAGAAGACCATCAGCAAGGCCAAGGGCCAG |
| | | CCGAGGGAGCCGCAGGTGTACACCCTGCCG |
| | | CCGAGCAGGGACGAGCTGACCAAGAACCAG |
| | | GTGAGCCTGACCTGCCTGGTGAAGGGCTTC |
| | | TACCCGAGCGACATCGCCGTGGAGTGGGAG |
| | | AGCAACGGCCAGCCGGAGAACAACTACAAG |
| | | ACCACCCCGCCGGTGCTGGACAGCGACGGC |
| | | AGCTTCTTCCTGTACAGCAAGCTGACCGTG |
| | | GACAAGAGCAGGTGGCAGCAGGGCAACGTG |
| | | TTCAGCTGCAGCGTGATGCACGAGGCCCTG |
| | | CACAACCACTACACCCAGAAGAGCCTGAGC |
| | | CTGAGCCCCGGCAAGTGATTCGAAAATAAA |
| | | ATATCTTTATTTTCATTACATCTGTGTGTT |
| | | GGTTTTTTGTGTGGCATGCTGGGGAGAGAT |
| | | CAACCCCACTCCCTCTCTGCGCGCTCGCTC |
| | | GCTCACTGAGGCCGGGCGACCAAAGGTCGC |
| | | CCGACGCCCGGGCTTTGCCCGGGCGGCCTC |
| | | AGTGAGCGAGCGAGCGCGCAGCAAGCTGTA |
| | | GCCAACCACTAGAACTATAGCTAGAGTCCT |
| | | GGGCGAACAAACGATGCTCGCCTTCCAGAA |
| | | AACCGAGGATGCGAACCACTTCATCCGGGG |
| | | TCAGCACCACCGGCAAGCGCCGCGACGGCC |
| | | GAGGTCTTCCGATCTCCTGAAGCCAGGGCA |
| | | GATCCGTGCACAGCACCTTGCCGTAGAAGA |
| | | ACAGCAAGGCCGCCAATGCCTGACGATGCG |
| | | TGGAGACCGAAACCTTGCGCTCGTTCGCCA |
| | | GCCAGGACAGAAATGCCTCGACTTCGCTGC |
| | | TGCCCAAGGTTGCCGGGTGACGCACACCGT |
| | | GGAAACGGATGAAGGCACGAACCCAGTTGA |
| | | CATAAGCCTGTTCGGTTCGTAAACTGTAAT |
| | | GCAAGTAGCGTATGCGCTCACGCAACTGGT |
| | | CCAGAACCTTGACCGAACGCAGCGGTGGTA |
| | | ACGGCGCAGTGGCGGTTTTCATGGCTTGTT |
| | | ATGACTGTTTTTTTGTACAGTCTATGCCTC |
| | | GGGCATCCAAGCAGCAAGCGCGTTACGCCG |
| | | TGGGTCGATGTTTGATGTTATGGAGCAGCA |
| | | ACGATGTTACGCAGCAGCAACGATGTTACG |
| | | CAGCAGGGCAGTCGCCCTAAAACAAAGTTA |
| | | GGTGGCTCAAGTATGGGCATCATTCGCACA |
| | | TGTAGGCTCGGCCCTGACCAAGTCAAATCC |
| | | ATGCGGGCTGCTCTTGATCTTTTCGGTCGT |
| | | GAGTTCGGAGACGTAGCCACCTACTCCCAA |
| | | CATCAGCCGGACTCCGATTACCTCGGGAAC |
| | | TTGCTCCGTAGTAAGACATTCATCGCGCTT |
| | | GCTGCCTTCGACCAAGAAGCGGTTGTTGGC |
| | | GCTCTCGCGGCTTACGTTCTGCCCAGGTTT |
| | | GAGCAGCCGCGTAGTGAGATCTATATCTAT |
| | | GATCTCGCAGTCTCCGGCGAGCACCGGAGG |
| | | CAGGGCATTGCCACCGCGCTCATCAATCTC |
| | | CTCAAGCATGAGGCCAACGCGCTTGGTGCT |
| | | TATGTGATCTACGTGCAAGCAGATTACGGT |
| | | GACGATCCCGCAGTGGCTCTCTATACAAAG |
| | | TTGGGCATACGGGAAGAAGTGATGCACTTT |
| | | GATATCGACCCAAGTACCGCCACCTAACAA |
| | | TTCGTTCAAGCCGAGATCGGCTTCCCGGCC |
| | | GCGGAGTTGTTCGGTAAATTGTCACAACGC |
| | | CGCGAATATAGTCTTTACCATGCCCTTGGC |
| | | CACGCCCCTCTTTAATACGACGGGCAATTT |
| | | GCACTTCAGAAAATGAAGAGTTTGCTTTAG |
| | | CCATAACAAAAGTCCAGTATGCTTTTTCAC |
| | | AGCATAACTGGACTGATTTCAGTTTACAAC |
| | | TATTCTGTCTAGTTTAAGACTTTATTGTCA |
| | | TAGTTTAGATCTATTTTGTTCAGTTTAAGA |
| | | CTTTATTGTCCGCCCACACCCGCTTACGCA |
| | | GGGCATCCATTTATTACTCAACCGTAACCG |
| | | ATTTTGCCAGGTTACGCGGCTGGTCTGCGG |
| | | TGTGAAATACCGCACAGATGCGTAAGGAGA |
| | | AAATACCGCATCAGGCGCTCTTCCGCTTCC |
| | | TCGCTCACTGACTCGCTGCGCTCGGTCGTT |
| | | CGGCTGCGGCGAGCGGTATCAGCTCACTCA |
| | | AAGGCGGTAATACGGTTATCCACAGAATCA |
| | | GGGGATAACGCAGGAAAGAACATGTGAGCA |
| | | AAAGGCCAGCAAAAGGCCAGGAACCGTAAA |
| | | AAGGCCGCGTTGCTGGCGTTTTTCCATAGG |
| | | CTCCGCCCCCCTGACGAGCATCACAAAAAT |
| | | CGACGCTCAAGTCAGAGGTGGCGAAACCCG |
| | | ACAGGACTATAAAGATACCAGGCGTTTCCC |
| | | CCTGGAAGCTCCCTCGTGCGCTCTCCTGTT |
| | | CCGACCCTGCCGCTTACCGGATACCTGTCC |
| | | GCCTTTCTCCCTTCGGGAAGCGTGGCGCTT |
| | | TCTCAATGCTCACGCTGTAGGTATCTCAGT |
| | | TCGGTGTAGGTCGTTCGCTCCAAGCTGGGC |
| | | TGTGTGCACGAACCCCCCGTTCAGCCCGAC |
| | | CGCTGCGCCTTATCCGGTAACTATCGTCTT |
| | | GAGTCCAACCCGGTAAGACACGACTTATCG |
| | | CCACTGGCAGCAGCCACTGGTAACAGGATT |
| | | AGCAGAGCGAGGTATGTAGGCGGTGCTACA |
| | | GAGTTCTTGAAGTGGTGGCCTAACTACGGC |
| | | TACACTAGAAGGACAGTATTTGGTATCTGC |
| | | GCTCTGCTGAAGCCAGTTACCTTCGGAAAA |
| | | AGAGTTGGTAGCTCTTGATCCGGCAAACAA |
| | | ACCACCGCTGGTAGCGGTGGTTTTTTTGTT |
| | | TGCAAGCAGCAGATTACGCGCAGAAAAAAA |
| | | GGATCTCAAGAAGATCCTTTGATCTTTTCT |
| | | ACGGGGTCTGACGCTCAGTGGAACGAAAAC |
| | | TCACGTTAAGGGATTTTGGTCATGAGATTA |
| | | TCAAAAAGGATCTTCACCTAGATCCTTTTA |
| | | AATTAAAAATGAAGTTTTAAATCAATCTAA |
| | | AGTATATATGAGTAAACTTGGTCTGACAGT |
| | | TACCAATGCTTAATCAGTGAGGCACCTATC |
| | | TCAGCGATCTGTCTATTTCGTTCATCCATA |
| | | GTTGCCTGACTCCCCGTCGTGTAGATAACT |
| | | ACGATACGGGAGGGCTTACCATCTGGCCCC |
| | | AGTGCTGCAATGATACCGCGAGACCCACGC |
| | | TCACCGGCTCCAGATTTATCAGCAATAAAC |
| | | CAGCCAGCCGGAAGGGCCGAGCGCAGAAGT |
| | | GGTCCTGCAACTTTATCCGCCTCCATCCAG |
| | | TCTATTAATTGTTGCCGGGAAGCTAGAGTA |
| | | AGTAGTTCGCCAGTTAATAGTTTGCGCAAC |
| | | GTTGTTGCCATTGCTACAGGCATCGTGGTG |
| | | TCACGCTCGTCGTTTGGTATGGCTTCATTC |
| | | AGCTCCGGTTCCCAACGATCAAGGCGAGTT |
| | | ACATGATCCCCCATGTTGTGCAAAAAAGCG |
| | | GTTAGCTCCTTCGGTCCTCCGATCGTTGTC |
| | | AGAAGTAAGTTGGCCGCAGTGTTATCACTC |
| | | ATGGTTATGGCAGCACTGCATAATTCTCTT |
| | | ACTGTCATGCCATCCGTAAGATGCTTTTCT |
| | | GTGACTGGTGAGTACTCAACCAAGTCATTC |
| | | TGAGAATAGTGTATGCGGCGACCGAGTTGC |
| | | TCTTGCCCGGCGTCAATACGGGATAATACC |
| | | GCGCCACATAGCAGAACTTTAAAAGTGCTC |
| | | ATCATTGGAAAACGTTCTTCGGGGCGAAAA |
| | | CTCTCAAGGATCTTACCGCTGTTGAGATCC |
| | | AGTTCGATGTAACCCACTCGTGCACCCAAC |
| | | TGATCTTCAGCATCTTTTACTTTCACCAGC |
| | | GTTTCTGGGTGAGCAAAAACAGGAAGGCAA |
| | | AATGCCGCAAAAAAGGGAATAAGGGCGACA |
| | | CGGAAATGTTGAATACTCATACTCTTCCTT |
| | | TTTCAATATTATTGAAGCATTTATCAGGGT |
| | | TATTGTCTCATGAGCGGATACATATTTGAA |
| | | TGTATTTAGAAAAATAAACAAATAGGGGTT |
| | | CCGCGCACATTTCCCCGAAAAGTGCCACCT |
| | | GAAATTGTAAACGTTAATATTTTGTTAAAA |
| | | TTCGCGTTAAATTTTTGTTAAATCAGCTCA |
| | | TTTTTTAACCAATAGGCCGAAATCGGCAAA |
| | | ATCCCTTATAAATCAAAAGAATAGACCGAG |
| | | ATAGGGTTGAGTGTTGTTCCAGTTTGGAAC |
| | | AAGAGTCCACTATTAAAGAACGTGGACTCC |
| | | AACGTCAAAGGGCGAAAAACCGTCTATCAG |
| | | GGCGATGGCCCACTACGTGAACCATCACCC |
| | | TAATCAAGTTTTTTGGGGTCGAGGTGCCGT |
| | | AAAGCACTAAATCGGAACCCTAAAGGGAGC |
| | | CCCCGATTTAGAGCTTGACGGGGAAAGCCG |
| | | GCGAACGTGGCGAGAAAGGAAGGGAAGAAA |
| | | GCGAAAGGAGCGGGCGCTAGGGCGCTGGCA |
| | | AGTGTAGCGGTCACGCTGCGCGTAACCACC |
| | | ACACCCGCCGCGCTTAATGCGCCGCTACAG |
| | | GGCGCGTC |
| 20 | Control | CATTCGCCATTCAGGCTGCAAATAAGCGTT |
| | Plasmid | GATATTCAGTCAATTACAAACATTAATAAC |
| | | GAAGAGATGACAGAAAAATTTTCATTCTGT |
| | | GACAGAGAAAAAGTAGCCGAAGATGACGGT |
| | | TTGTCACATGGAGTTGGCAGGATGTTTGAT |
| | | TAAAAACATAACAGGAAGAAAAATGCCCCG |
| | | CTGTGGGCGGACAAAATAGTTGGGAACTGG |
| | | GAGGGGTGGAAATGGAGTTTTTAAGGATTA |
| | | TTTAGGGAAGAGTGACAAAATAGATGGGAA |
| | | CTGGGTGTAGCGTCGTAAGCTAATACGAAA |
| | | ATTAAAAATGACAAAATAGTTTGGAACTAG |
| | | ATTTCACTTATCTGGTTCGGATCTCCTAGG |
| | | CTCAAGCAGTGATCAGATCCAGACATGATA |
| | | AGATACATTGATGAGTTTGGACAAACCACA |
| | | ACTAGAATGCAGTGAAAAAAATGCTTTATT |
| | | TGTGAAATTTGTGATGCTATTGCTTTATTT |
| | | GTAACCATTATAAGCTGCAATAAACAAGTT |
| | | AACAACAACAATTGCATTCATTTTATGTTT |
| | | CAGGTTCAGGGGGAGGTGTGGGAGGTTTTT |
| | | TAAAGCAAGTAAAACCTCTACAAATGTGGT |
| | | ATGGCTGATTATGATCCTCTAGTACTTCTC |
| | | GACAAGCTCGGATCCTGGCGCGCTCGCTCG |
| | | CTCACTGAGGCCGCCCGGGCAAAGCCCGGG |
| | | CGTCGGGCGACCTTTGGTCGCCCGGCCTCA |
| | | GTGAGCGAGCGAGCGCGCAGAGAGGGAGTG |
| | | GCCAACTCCATCACTAGGGGTTCCTAGGAA |
| | | GCTGATCTGAATTCGGTACCCGTTACATAA |
| | | CTTACGGTAAATGGCCCGCCTGGCTGACCG |
| | | CCCAACGACCCCCGCCCATTGACGTCAATA |
| | | ATGACGTATGTTCCCATAGTAACGCCAATA |
| | | GGGACTTTCCATTGACGTCAATGGGTGGAG |
| | | TATTTACGGTAAACTGCCCACTTGGCAGTA |
| | | CATCAAGTGTATCATATGCCAAGTACGCCC |
| | | CCTATTGACGTCAATGACGGTAAATGGCCC |
| | | GCCTGGCATTATGCCCAGTACATGACCTTA |
| | | TGGGACTTTCCTACTTGGCAGTACATCTAC |
| | | GTATTAGTCATCGCTATTACCATGGTGATG |
| | | CGGTTTTGGCAGTACATCAATGGGCGTGGA |
| | | TAGCGGTTTGACTCACGGGGATTTCCAAGT |
| | | CTCCACCCCATTGACGTCAATGGGAGTTTG |
| | | TTTTGGCACCAAAATCAACGGGACTTTCCA |
| | | AAATGTCGTAACAACTCCGCCCCATTGACG |
| | | CAAATGGGCGGTAGGCGTGTACGGTGGGAG |
| | | GTCTATATAAGCAGAGCTCGTTTAGTGAAC |
| | | CGTCAGATCGCCTGGAGACGCCATCCACGC |
| | | TGTTTTGACCTCCATAGAAGACACCGGGAC |
| | | CGATCCAGCCTCCGGACTCTAGAGTTAACT |
| | | GGTAAGTTTAGTCTTTTTGTCTTTTATTTC |
| | | AGGTCCCGGATCCGGTGGTGGTGCAAATCA |
| | | AAGAACTGCTCCTCAGTGGATGTTGCCTTT |
| | | ACTTCTAGGCCTGCGCTCACCATGGTCAGC |
| | | TACTGGGACACCGGGGTCCTGCTGTGCGCG |
| | | CTGCTCAGCTGTCTGCTTCTCACAGGATCT |
| | | AGTTCAGGTTCAGATACAGGTAGACCTTTC |
| | | GTAGAGATGTACAGTGAAATCCCCGAAATT |
| | | ATACACATGACTGAAGGAAGGGAGCTCGTC |
| | | ATTCCCTGCCGGGTTACGTCACCTAACATC |
| | | ACTGTTACTTTAAAAAAGTTTCCACTTGAC |
| | | ACTTTGATCCCTGATGGAAAACGCATAATC |
| | | TGGGACAGTAGAAAGGGCTTCATCATATCA |
| | | AATGCAACGTACAAAGAAATAGGGCTTCTG |
| | | ACCTGTGAAGCAACAGTCAATGGGCATTTG |
| | | TATAAGACAAACTATCTCACACATCGACAA |
| | | ACCAATACAATCATAGATGTCGTTCTGAGT |
| | | CCGTCTCATGGAATTGAACTATCTGTTGGA |
| | | GAAAAGCTTGTCTTAAATTGTACAGCAAGA |
| | | ACTGAACTAAATGTGGGGATTGACTTCAAC |
| | | TGGGAATACCCTTCTTCGAAGCATCAGCAT |
| | | AAGAAACTTGTAAACCGAGACCTAAAAACC |
| | | CAGTCTGGGAGTGAGATGAAGAAATTTTTG |
| | | AGCACCTTAACTATAGATGGTGTAACCCGG |
| | | AGTGACCAAGGATTGTACACCTGTGCAGCA |
| | | TCCAGTGGGCTGATGACCAAGAAGAACAGC |
| | | ACATTTGTCAGGGTCCATGAAAAAGACAAA |
| | | ACTCACACATGCCCACCGTGCCCAGCACCT |
| | | GAACTCCTGGGGGGACCGTCAGTCTTCCTC |
| | | TTCCCCCCAAAACCCAAGGACACCCTCATG |
| | | ATCTCCCGGACCCCTGAGGTCACATGCGTG |
| | | GTGGTGGACGTGAGCCACGAAGACCCTGAG |
| | | GTCAAGTTCAACTGGTACGTGGACGGCGTG |
| | | GAGGTGCATAATGCCAAGACAAAGCCGCGG |
| | | GAGGAGCAGTACAACAGCACGTACCGTGTG |
| | | GTCAGCGTCCTCACCGTCCTGCACCAGGAC |
| | | TGGCTGAATGGCAAGGAGTACAAGTGCAAG |
| | | GTCTCCAACAAAGCCCTCCCAGCCCCCATC |
| | | GAGAAAACCATCTCCAAAGCCAAAGGGCAG |
| | | CCCCGAGAACCACAGGTGTACACCCTGCCC |
| | | CCATCCCGGGATGAGCTGACCAAGAACCAG |
| | | GTCAGCCTGACCTGCCTGGTCAAAGGCTTC |
| | | TATCCCAGCGACATCGCCGTGGAGTGGGAG |
| | | AGCAATGGGCAGCCGGAGAACAACTACAAG |
| | | ACCACGCCTCCCGTGCTGGACTCCGACGGC |
| | | TCCTTCTTCCTCTACAGCAAGCTCACCGTG |
| | | GACAAGAGCAGGTGGCAGCAGGGGAACGTC |
| | | TTCTCATGCTCCGTGATGCATGAGGCTCTG |
| | | CACAACCACTACACGCAGAAGAGCCTCTCC |
| | | CTGTCTCCGGGTAAATGATTCGAAAATAAA |
| | | ATATCTTTATTTTCATTACATCTGTGTGTT |
| | | GGTTTTTTGTGTGGCATGCTGGGGAGAGAT |
| | | CAACCCCACTCCCTCTCTGCGCGCTCGCTC |
| | | GCTCACTGAGGCCGGGCGACCAAAGGTCGC |
| | | CCGACGCCCGGGCTTTGCCCGGGCGGCCTC |
| | | AGTGAGCGAGCGAGCGCGCAGCAAGCTGTA |
| | | GCCAACCACTAGAACTATAGCTAGAGTCCT |
| | | GGGCGAACAAACGATGCTCGCCTTCCAGAA |
| | | AACCGAGGATGCGAACCACTTCATCCGGGG |
| | | TCAGCACCACCGGCAAGCGCCGCGACGGCC |
| | | GAGGTCTTCCGATCTCCTGAAGCCAGGGCA |
| | | GATCCGTGCACAGCACCTTGCCGTAGAAGA |
| | | ACAGCAAGGCCGCCAATGCCTGACGATGCG |
| | | TGGAGACCGAAACCTTGCGCTCGTTCGCCA |
| | | GCCAGGACAGAAATGCCTCGACTTCGCTGC |
| | | TGCCCAAGGTTGCCGGGTGACGCACACCGT |
| | | GGAAACGGATGAAGGCACGAACCCAGTTGA |
| | | CATAAGCCTGTTCGGTTCGTAAACTGTAAT |
| | | GCAAGTAGCGTATGCGCTCACGCAACTGGT |
| | | CCAGAACCTTGACCGAACGCAGCGGTGGTA |
| | | ACGGCGCAGTGGCGGTTTTCATGGCTTGTT |
| | | ATGACTGTTTTTTTGTACAGTCTATGCCTC |
| | | GGGCATCCAAGCAGCAAGCGCGTTACGCCG |
| | | TGGGTCGATGTTTGATGTTATGGAGCAGCA |
| | | ACGATGTTACGCAGCAGCAACGATGTTACG |
| | | CAGCAGGGCAGTCGCCCTAAAACAAAGTTA |
| | | GGTGGCTCAAGTATGGGCATCATTCGCACA |
| | | TGTAGGCTCGGCCCTGACCAAGTCAAATCC |
| | | ATGCGGGCTGCTCTTGATCTTTTCGGTCGT |
| | | GAGTTCGGAGACGTAGCCACCTACTCCCAA |
| | | CATCAGCCGGACTCCGATTACCTCGGGAAC |
| | | TTGCTCCGTAGTAAGACATTCATCGCGCTT |
| | | GCTGCCTTCGACCAAGAAGCGGTTGTTGGC |
| | | GCTCTCGCGGCTTACGTTCTGCCCAGGTTT |
| | | GAGCAGCCGCGTAGTGAGATCTATATCTAT |
| | | GATCTCGCAGTCTCCGGCGAGCACCGGAGG |
| | | CAGGGCATTGCCACCGCGCTCATCAATCTC |
| | | CTCAAGCATGAGGCCAACGCGCTTGGTGCT |
| | | TATGTGATCTACGTGCAAGCAGATTACGGT |
| | | GACGATCCCGCAGTGGCTCTCTATACAAAG |
| | | TTGGGCATACGGGAAGAAGTGATGCACTTT |
| | | GATATCGACCCAAGTACCGCCACCTAACAA |
| | | TTCGTTCAAGCCGAGATCGGCTTCCCGGCC |
| | | GCGGAGTTGTTCGGTAAATTGTCACAACGC |
| | | CGCGAATATAGTCTTTACCATGCCCTTGGC |
| | | CACGCCCCTCTTTAATACGACGGGCAATTT |
| | | GCACTTCAGAAAATGAAGAGTTTGCTTTAG |
| | | CCATAACAAAAGTCCAGTATGCTTTTTCAC |
| | | AGCATAACTGGACTGATTTCAGTTTACAAC |
| | | TATTCTGTCTAGTTTAAGACTTTATTGTCA |
| | | TAGTTTAGATCTATTTTGTTCAGTTTAAGA |
| | | CTTTATTGTCCGCCCACACCCGCTTACGCA |
| | | GGGCATCCATTTATTACTCAACCGTAACCG |
| | | ATTTTGCCAGGTTACGCGGCTGGTCTGCGG |
| | | TGTGAAATACCGCACAGATGCGTAAGGAGA |
| | | AAATACCGCATCAGGCGCTCTTCCGCTTCC |
| | | TCGCTCACTGACTCGCTGCGCTCGGTCGTT |
| | | CGGCTGCGGCGAGCGGTATCAGCTCACTCA |
| | | AAGGCGGTAATACGGTTATCCACAGAATCA |
| | | GGGGATAACGCAGGAAAGAACATGTGAGCA |
| | | AAAGGCCAGCAAAAGGCCAGGAACCGTAAA |
| | | AAGGCCGCGTTGCTGGCGTTTTTCCATAGG |
| | | CTCCGCCCCCCTGACGAGCATCACAAAAAT |
| | | CGACGCTCAAGTCAGAGGTGGCGAAACCCG |
| | | ACAGGACTATAAAGATACCAGGCGTTTCCC |
| | | CCTGGAAGCTCCCTCGTGCGCTCTCCTGTT |
| | | CCGACCCTGCCGCTTACCGGATACCTGTCC |
| | | GCCTTTCTCCCTTCGGGAAGCGTGGCGCTT |
| | | TCTCAATGCTCACGCTGTAGGTATCTCAGT |
| | | TCGGTGTAGGTCGTTCGCTCCAAGCTGGGC |
| | | TGTGTGCACGAACCCCCCGTTCAGCCCGAC |
| | | CGCTGCGCCTTATCCGGTAACTATCGTCTT |
| | | GAGTCCAACCCGGTAAGACACGACTTATCG |
| | | CCACTGGCAGCAGCCACTGGTAACAGGATT |
| | | AGCAGAGCGAGGTATGTAGGCGGTGCTACA |
| | | GAGTTCTTGAAGTGGTGGCCTAACTACGGC |
| | | TACACTAGAAGGACAGTATTTGGTATCTGC |
| | | GCTCTGCTGAAGCCAGTTACCTTCGGAAAA |
| | | AGAGTTGGTAGCTCTTGATCCGGCAAACAA |
| | | ACCACCGCTGGTAGCGGTGGTTTTTTTGTT |
| | | TGCAAGCAGCAGATTACGCGCAGAAAAAAA |
| | | GGATCTCAAGAAGATCCTTTGATCTTTTCT |
| | | ACGGGGTCTGACGCTCAGTGGAACGAAAAC |
| | | TCACGTTAAGGGATTTTGGTCATGAGATTA |
| | | TCAAAAAGGATCTTCACCTAGATCCTTTTA |
| | | AATTAAAAATGAAGTTTTAAATCAATCTAA |
| | | AGTATATATGAGTAAACTTGGTCTGACAGT |
| | | TACCAATGCTTAATCAGTGAGGCACCTATC |
| | | TCAGCGATCTGTCTATTTCGTTCATCCATA |
| | | GTTGCCTGACTCCCCGTCGTGTAGATAACT |
| | | ACGATACGGGAGGGCTTACCATCTGGCCCC |
| | | AGTGCTGCAATGATACCGCGAGACCCACGC |
| | | TCACCGGCTCCAGATTTATCAGCAATAAAC |
| | | CAGCCAGCCGGAAGGGCCGAGCGCAGAAGT |
| | | GGTCCTGCAACTTTATCCGCCTCCATCCAG |
| | | TCTATTAATTGTTGCCGGGAAGCTAGAGTA |
| | | AGTAGTTCGCCAGTTAATAGTTTGCGCAAC |
| | | GTTGTTGCCATTGCTACAGGCATCGTGGTG |
| | | TCACGCTCGTCGTTTGGTATGGCTTCATTC |
| | | AGCTCCGGTTCCCAACGATCAAGGCGAGTT |
| | | ACATGATCCCCCATGTTGTGCAAAAAAGCG |
| | | GTTAGCTCCTTCGGTCCTCCGATCGTTGTC |
| | | AGAAGTAAGTTGGCCGCAGTGTTATCACTC |
| | | ATGGTTATGGCAGCACTGCATAATTCTCTT |
| | | ACTGTCATGCCATCCGTAAGATGCTTTTCT |
| | | GTGACTGGTGAGTACTCAACCAAGTCATTC |
| | | TGAGAATAGTGTATGCGGCGACCGAGTTGC |
| | | TCTTGCCCGGCGTCAATACGGGATAATACC |
| | | GCGCCACATAGCAGAACTTTAAAAGTGCTC |
| | | ATCATTGGAAAACGTTCTTCGGGGCGAAAA |
| | | CTCTCAAGGATCTTACCGCTGTTGAGATCC |
| | | AGTTCGATGTAACCCACTCGTGCACCCAAC |
| | | TGATCTTCAGCATCTTTTACTTTCACCAGC |
| | | GTTTCTGGGTGAGCAAAAACAGGAAGGCAA |
| | | AATGCCGCAAAAAAGGGAATAAGGGCGACA |
| | | CGGAAATGTTGAATACTCATACTCTTCCTT |
| | | TTTCAATATTATTGAAGCATTTATCAGGGT |
| | | TATTGTCTCATGAGCGGATACATATTTGAA |
| | | TGTATTTAGAAAAATAAACAAATAGGGGTT |
| | | CCGCGCACATTTCCCCGAAAAGTGCCACCT |
| | | GAAATTGTAAACGTTAATATTTTGTTAAAA |
| | | TTCGCGTTAAATTTTTGTTAAATCAGCTCA |
| | | TTTTTTAACCAATAGGCCGAAATCGGCAAA |
| | | ATCCCTTATAAATCAAAAGAATAGACCGAG |
| | | ATAGGGTTGAGTGTTGTTCCAGTTTGGAAC |
| | | AAGAGTCCACTATTAAAGAACGTGGACTCC |
| | | AACGTCAAAGGGCGAAAAACCGTCTATCAG |
| | | GGCGATGGCCCACTACGTGAACCATCACCC |
| | | TAATCAAGTTTTTTGGGGTCGAGGTGCCGT |
| | | AAAGCACTAAATCGGAACCCTAAAGGGAGC |
| | | CCCCGATTTAGAGCTTGACGGGGAAAGCCG |
| | | GCGAACGTGGCGAGAAAGGAAGGGAAGAAA |
| | | GCGAAAGGAGCGGGCGCTAGGGCGCTGGCA |
| | | AGTGTAGCGGTCACGCTGCGCGTAACCACC |
| | | ACACCCGCCGCGCTTAATGCGCCGCTACAG |
| | | GGCGCGTC |
| 21 | AMI059 | ATGGTATCCTATTGGGATACGGGTGTTCTC |
| | coding | TTGTGTGCACTCCTTTCCTGTCTCCTGCTC |
| | sequence | ACTGGATCTTCTTCTGGGTCTGATACTGGT |
| | | AGACCTTTTGTAGAAATGTATTCAGAAATT |
| | | CCGGAAATAATTCATATGACAGAAGGACGA |
| | | GAACTCGTTATACCATGTCGCGTCACGTCC |
| | | CCTAATATTACTGTTACGCTCAAGAAGTTT |
| | | CCTCTCGATACACTTATTCCAGATGGGAAA |
| | | CGCATAATTTGGGATTCACGCAAAGGGTTT |
| | | ATTATTAGTAACGCAACGTATAAAGAAATT |
| | | GGGCTGCTCACATGTGAAGCTACGGTAAAT |
| | | GGGCATCTTTATAAAACAAATTATTTGACT |
| | | CATCGGCAAACTAATACTATTATCGATGTA |
| | | GTACTCTCCCCATCCCATGGTATTGAATTG |
| | | TCAGTTGGGGAGAAGTTGGTATTGAATTGT |
| | | ACTGCACGGACAGAACTCAACGTTGGTATT |
| | | GATTTTAATTGGGAATATCCATCATCTAAA |
| | | CATCAGCATAAGAAGTTGGTAAATCGTGAT |
| | | CTCAAAACTCAAAGTGGGTCCGAAATGAAG |
| | | AAGTTTCTGTCCACACTTACGATTGATGGG |
| | | GTCACTAGAAGTGATCAAGGGCTCTATACG |
| | | TGTGCAGCATCTAGTGGGTTGATGACAAAG |
| | | AAGAATTCAACTTTTGTTCGTGTCCATGAA |
| | | AAGGATAAAACACATACTTGTCCACCGTGT |
| | | CCTGCGCCAGAACTTCTCGGTGGTCCATCC |
| | | GTCTTTCTCTTTCCACCTAAACCAAAAGAT |
| | | ACTTTGATGATTTCACGGACTCCAGAAGTA |
| | | ACATGTGTTGTCGTTGATGTATCACACGAA |
| | | GATCCAGAAGTCAAATTTAATTGGTATGTT |
| | | GATGGTGTAGAAGTTCATAATGCGAAGACA |
| | | AAACCACGAGAAGAACAATACAATAGTACA |
| | | TATCGGGTAGTATCCGTCTTGACTGTACTT |
| | | CACCAAGATTGGCTTAATGGGAAAGAATAC |
| | | AAATGTAAAGTTTCTAATAAAGCTCTTCCT |
| | | GCGCCGATCGAAAAGACAATTTCCAAAGCA |
| | | AAAGGTCAACCTCGGGAACCTCAAGTTTAT |
| | | ACGCTCCCACCATCACGGGATGAACTCACT |
| | | AAGAATCAAGTATCCTTGACTTGTCTCGTA |
| | | AAAGGGTTTTATCCTTCAGATATTGCTGTA |
| | | GAATGGGAATCCAATGGGCAACCAGAAAAT |
| | | AATTATAAAACAACACCACCTGTTCTTGAT |
| | | TCAGATGGTTCATTCTTTCTCTATTCCAAA |
| | | CTTACTGTCGATAAATCACGCTGGCAACAA |
| | | GGTAATGTTTTCTCTTGTTCCGTCATGCAT |
| | | GAAGCACTCCATAATCACTATACGCAAAAG |
| | | TCTCTCTCTCTCTCACCAGGTAAATAA |
| 22 | AMI066 | ATGGAGTTCGGCCTGAGCTGGCTGTTCCTG |
| | coding | GTGGCCATCCTTAAGGGCGTGCAGTGCGAT |
| | sequence | ACTGGTAGACCTTTTGTAGAAATGTATTCA |
| | | GAAATTCCGGAAATAATTCATATGACAGAA |
| | | GGACGAGAACTCGTTATACCATGTCGCGTC |
| | | ACGTCCCCTAATATTACTGTTACGCTCAAG |
| | | AAGTTTCCTCTCGATACACTTATTCCAGAT |
| | | GGGAAACGCATAATTTGGGATTCACGCAAA |
| | | GGGTTTATTATTAGTAACGCAACGTATAAA |
| | | GAAATTGGGCTGCTCACATGTGAAGCTACG |
| | | GTAAATGGGCATCTTTATAAAACAAATTAT |
| | | TTGACTCATCGGCAAACTAATACTATTATC |
| | | GATGTAGTACTCTCCCCATCCCATGGTATT |
| | | GAATTGTCAGTTGGGGAGAAGTTGGTATTG |
| | | AATTGTACTGCACGGACAGAACTCAACGTT |
| | | GGTATTGATTTTAATTGGGAATATCCATCA |
| | | TCTAAACATCAGCATAAGAAGTTGGTAAAT |
| | | CGTGATCTCAAAACTCAAAGTGGGTCCGAA |
| | | ATGAAGAAGTTTCTGTCCACACTTACGATT |
| | | GATGGGGTCACTAGAAGTGATCAAGGGCTC |
| | | TATACGTGTGCAGCATCTAGTGGGTTGATG |
| | | ACAAAGAAGAATTCAACTTTTGTTCGTGTC |
| | | CATGAAAAGGATAAAACACATACTTGTCCA |
| | | CCGTGTCCTGCGCCAGAACTTCTCGGTGGT |
| | | CCATCCGTCTTTCTCTTTCCACCTAAACCA |
| | | AAAGATACTTTGATGATTTCACGGACTCCA |
| | | GAAGTAACATGTGTTGTCGTTGATGTATCA |
| | | CACGAAGATCCAGAAGTCAAATTTAATTGG |
| | | TATGTTGATGGTGTAGAAGTTCATAATGCG |
| | | AAGACAAAACCACGAGAAGAACAATACAAT |
| | | AGTACATATCGGGTAGTATCCGTCTTGACT |
| | | GTACTTCACCAAGATTGGCTTAATGGGAAA |
| | | GAATACAAATGTAAAGTTTCTAATAAAGCT |
| | | CTTCCTGCGCCGATCGAAAAGACAATTTCC |
| | | AAAGCAAAAGGTCAACCTCGGGAACCTCAA |
| | | GTTTATACGCTCCCACCATCACGGGATGAA |
| | | CTCACTAAGAATCAAGTATCCTTGACTTGT |
| | | CTCGTAAAAGGGTTTTATCCTTCAGATATT |
| | | GCTGTAGAATGGGAATCCAATGGGCAACCA |
| | | GAAAATAATTATAAAACAACACCACCTGTT |
| | | CTTGATTCAGATGGTTCATTCTTTCTCTAT |
| | | TCCAAACTTACTGTCGATAAATCACGCTGG |
| | | CAACAAGGTAATGTTTTCTCTTGTTCCGTC |
| | | ATGCATGAAGCACTCCATAATCACTATACG |
| | | CAAAAGTCTCTCTCTCTCTCACCAGGTAAA |
| | | TAA |
| 23 | AMI067 | ATGGTGAGCTACTGGGACACCGGCGTGCTG |
| | coding | CTGTGCGCCCTGCTGAGCTGCCTGCTGCTG |
| | sequence | ACCGGCAGCAGCAGCGGCAGCGACACCGGC |
| | | AGACCCTTCGTGGAGATGTACAGCGAGATC |
| | | CCCGAGATCATCCACATGACCGAGGGCAGA |
| | | GAGCTGGTGATCCCCTGCAGAGTGACCAGC |
| | | CCCAACATCACCGTGACCCTGAAGAAGTTC |
| | | CCCCTGGACACCCTGATCCCCGACGGCAAG |
| | | AGAATCATCTGGGACAGCAGAAAGGGCTTC |
| | | ATCATCAGCAACGCCACCTACAAGGAGATC |
| | | GGCCTGCTGACCTGCGAGGCCACCGTGAAC |
| | | GGCCACCTGTACAAGACCAACTACCTGACC |
| | | CACAGACAGACCAACACCATCATCGACGTG |
| | | GTGCTGAGCCCCAGCCACGGCATCGAGCTG |
| | | AGCGTGGGCGAGAAGCTGGTGCTGAACTGC |
| | | ACCGCCAGAACCGAGCTGAACGTGGGCATC |
| | | GACTTCAACTGGGAGTACCCCAGCAGCAAG |
| | | CACCAGCACAAGAAGCTGGTGAACAGAGAC |
| | | CTGAAGACCCAGAGCGGCAGCGAGATGAAG |
| | | AAGTTCCTGAGCACCCTGACCATCGACGGC |
| | | GTGACCAGAAGCGACCAGGGCCTGTACACC |
| | | TGCGCCGCCAGCAGCGGCCTGATGACCAAG |
| | | AAGAACAGCACCTTCGTGAGAGTGCACGAG |
| | | AAGGACAAGACCCACACCTGCCCCCCCTGC |
| | | CCCGCCCCCGAGCTGCTGGGCGGCCCCAGC |
| | | GTGTTCCTGTTCCCCCCCAAGCCCAAGGAC |
| | | ACCCTGATGATCAGCAGAACCCCCGAGGTG |
| | | ACCTGCGTGGTGGTGGACGTGAGCCACGAG |
| | | GACCCCGAGGTGAAGTTCAACTGGTACGTG |
| | | GACGGCGTGGAGGTGCACAACGCCAAGACC |
| | | AAGCCCAGAGAGGAGCAGTACAACAGCACC |
| | | TACAGAGTGGTGAGCGTGCTGACCGTGCTG |
| | | CACCAGGACTGGCTGAACGGCAAGGAGTAC |
| | | AAGTGCAAGGTGAGCAACAAGGCCCTGCCC |
| | | GCCCCCATCGAGAAGACCATCAGCAAGGCC |
| | | AAGGGCCAGCCCAGAGAGCCCCAGGTGTAC |
| | | ACCCTGCCCCCCAGCAGAGACGAGCTGACC |
| | | AAGAACCAGGTGAGCCTGACCTGCCTGGTG |
| | | AAGGGCTTCTACCCCAGCGACATCGCCGTG |
| | | GAGTGGGAGAGCAACGGCCAGCCCGAGAAC |
| | | AACTACAAGACCACCCCCCCCGTGCTGGAC |
| | | AGCGACGGCAGCTTCTTCCTGTACAGCAAG |
| | | CTGACCGTGGACAAGAGCAGATGGCAGCAG |
| | | GGCAACGTGTTCAGCTGCAGCGTGATGCAC |
| | | GAGGCCCTGCACAACCACTACACCCAGAAG |
| | | AGCCTGAGCCTGAGCCCCGGCAAGTGA |
| 24 | AMI068 | ATGGAGTTCGGCCTGAGCTGGCTGTTCCTG |
| | coding | GTGGCCATCCTTAAGGGCGTGCAGTGCGAC |
| | sequence | ACCGGCAGACCCTTCGTGGAGATGTACAGC |
| | | GAGATCCCCGAGATCATCCACATGACCGAG |
| | | GGCAGAGAGCTGGTGATCCCCTGCAGAGTG |
| | | ACCAGCCCCAACATCACCGTGACCCTGAAG |
| | | AAGTTCCCCCTGGACACCCTGATCCCCGAC |
| | | GGCAAGAGAATCATCTGGGACAGCAGAAAG |
| | | GGCTTCATCATCAGCAACGCCACCTACAAG |
| | | GAGATCGGCCTGCTGACCTGCGAGGCCACC |
| | | GTGAACGGCCACCTGTACAAGACCAACTAC |
| | | CTGACCCACAGACAGACCAACACCATCATC |
| | | GACGTGGTGCTGAGCCCCAGCCACGGCATC |
| | | GAGCTGAGCGTGGGCGAGAAGCTGGTGCTG |
| | | AACTGCACCGCCAGAACCGAGCTGAACGTG |
| | | GGCATCGACTTCAACTGGGAGTACCCCAGC |
| | | AGCAAGCACCAGCACAAGAAGCTGGTGAAC |
| | | AGAGACCTGAAGACCCAGAGCGGCAGCGAG |
| | | ATGAAGAAGTTCCTGAGCACCCTGACCATC |
| | | GACGGCGTGACCAGAAGCGACCAGGGCCTG |
| | | TACACCTGCGCCGCCAGCAGCGGCCTGATG |
| | | ACCAAGAAGAACAGCACCTTCGTGAGAGTG |
| | | CACGAGAAGGACAAGACCCACACCTGCCCC |
| | | CCCTGCCCCGCCCCCGAGCTGCTGGGCGGC |
| | | CCCAGCGTGTTCCTGTTCCCCCCCAAGCCC |
| | | AAGGACACCCTGATGATCAGCAGAACCCCC |
| | | GAGGTGACCTGCGTGGTGGTGGACGTGAGC |
| | | CACGAGGACCCCGAGGTGAAGTTCAACTGG |
| | | TACGTGGACGGCGTGGAGGTGCACAACGCC |
| | | AAGACCAAGCCCAGAGAGGAGCAGTACAAC |
| | | AGCACCTACAGAGTGGTGAGCGTGCTGACC |
| | | GTGCTGCACCAGGACTGGCTGAACGGCAAG |
| | | GAGTACAAGTGCAAGGTGAGCAACAAGGCC |
| | | CTGCCCGCCCCCATCGAGAAGACCATCAGC |
| | | AAGGCCAAGGGCCAGCCCAGAGAGCCCCAG |
| | | GTGTACACCCTGCCCCCCAGCAGAGACGAG |
| | | CTGACCAAGAACCAGGTGAGCCTGACCTGC |
| | | CTGGTGAAGGGCTTCTACCCCAGCGACATC |
| | | GCCGTGGAGTGGGAGAGCAACGGCCAGCCC |
| | | GAGAACAACTACAAGACCACCCCCCCCGTG |
| | | CTGGACAGCGACGGCAGCTTCTTCCTGTAC |
| | | AGCAAGCTGACCGTGGACAAGAGCAGATGG |
| | | CAGCAGGGCAACGTGTTCAGCTGCAGCGTG |
| | | ATGCACGAGGCCCTGCACAACCACTACACC |
| | | CAGAAGAGCCTGAGCCTGAGCCCCGGCAAG |
| | | TGA |
| 25 | AMI119 | ATGGTGAGCTACTGGGACACCGGCGTGCTG |
| | coding | CTGTGCGCCCTGCTGAGCTGCCTGCTGCTG |
| | sequence | ACCGGCAGCAGCAGCGGCAGCGACACCGGC |
| | | AGGCCTTTCGTGGAGATGTACAGCGAGATC |
| | | CCTGAGATCATCCACATGACCGAGGGCAGG |
| | | GAGCTGGTGATCCCTTGCAGGGTGACCAGC |
| | | CCTAACATCACCGTGACCCTGAAGAAGTTC |
| | | CCTCTGGACACCCTGATCCCTGACGGCAAG |
| | | AGGATCATCTGGGACAGCAGGAAGGGCTTC |
| | | ATCATCAGCAACGCCACCTACAAGGAGATC |
| | | GGCCTGCTGACCTGCGAGGCCACCGTGAAC |
| | | GGCCACCTGTACAAGACCAACTACCTGACC |
| | | CACAGGCAGACCAACACCATCATCGACGTG |
| | | GTGCTGAGCCCTAGCCACGGCATCGAGCTG |
| | | AGCGTGGGCGAGAAGCTGGTGCTGAACTGC |
| | | ACCGCCAGGACCGAGCTGAACGTGGGCATC |
| | | GACTTCAACTGGGAGTACCCTAGCAGCAAG |
| | | CACCAGCACAAGAAGCTGGTGAACAGGGAC |
| | | CTGAAGACCCAGAGCGGCAGCGAGATGAAG |
| | | AAGTTCCTGAGCACCCTGACCATCGACGGC |
| | | GTGACCAGGAGCGACCAGGGCCTGTACACC |
| | | TGCGCCGCCAGCAGCGGCCTGATGACCAAG |
| | | AAGAACAGCACCTTCGTGAGGGTGCACGAG |
| | | AAGGACAAGACCCACACCTGCCCTCCTTGC |
| | | CCTGCCCCTGAGCTGCTGGGCGGCCCTAGC |
| | | GTGTTCCTGTTCCCTCCTAAGCCTAAGGAC |
| | | ACCCTGATGATCAGCAGGACCCCTGAGGTG |
| | | ACCTGCGTGGTGGTGGACGTGAGCCACGAG |
| | | GACCCTGAGGTGAAGTTCAACTGGTACGTG |
| | | GACGGCGTGGAGGTGCACAACGCCAAGACC |
| | | AAGCCTAGGGAGGAGCAGTACAACAGCACC |
| | | TACAGGGTGGTGAGCGTGCTGACCGTGCTG |
| | | CACCAGGACTGGCTGAACGGCAAGGAGTAC |
| | | AAGTGCAAGGTGAGCAACAAGGCCCTGCCT |
| | | GCCCCTATCGAGAAGACCATCAGCAAGGCC |
| | | AAGGGCCAGCCTAGGGAGCCTCAGGTGTAC |
| | | ACCCTGCCTCCTAGCAGGGACGAGCTGACC |
| | | AAGAACCAGGTGAGCCTGACCTGCCTGGTG |
| | | AAGGGCTTCTACCCTAGCGACATCGCCGTG |
| | | GAGTGGGAGAGCAACGGCCAGCCTGAGAAC |
| | | AACTACAAGACCACCCCTCCTGTGCTGGAC |
| | | AGCGACGGCAGCTTCTTCCTGTACAGCAAG |
| | | CTGACCGTGGACAAGAGCAGGTGGCAGCAG |
| | | GGCAACGTGTTCAGCTGCAGCGTGATGCAC |
| | | GAGGCCCTGCACAACCACTACACCCAGAAG |
| | | AGCCTGAGCCTGAGCCCTGGCAAGTGA |
| 26 | AMI120 | ATGGTGAGCTACTGGGACACCGGCGTGCTG |
| | coding | CTGTGCGCCCTGCTGAGCTGCCTGCTGCTG |
| | sequence | ACCGGCAGCAGCAGCGGCAGCGACACCGGC |
| | | AGGCCCTTCGTGGAGATGTACTCCGAGATC |
| | | CCCGAGATCATCCACATGACCGAGGGCAGG |
| | | GAGCTGGTGATCCCCTGCAGGGTGACCTCC |
| | | CCCAACATCACCGTGACCCTGAAGAAGTTC |
| | | CCCCTGGACACCCTGATCCCCGACGGCAAG |
| | | AGGATCATCTGGGACTCCAGGAAGGGCTTC |
| | | ATCATCTCCAACGCCACCTACAAGGAGATC |
| | | GGCCTGCTGACCTGCGAGGCCACCGTGAAC |
| | | GGCCACCTGTACAAGACCAACTACCTGACC |
| | | CACAGGCAGACCAACACCATCATCGACGTG |
| | | GTGCTGTCCCCCTCCCACGGCATCGAGCTG |
| | | TCCGTGGGCGAGAAGCTGGTGCTGAACTGC |
| | | ACCGCCAGGACCGAGCTGAACGTGGGCATC |
| | | GACTTCAACTGGGAGTACCCCTCCTCCAAG |
| | | CACCAGCACAAGAAGCTGGTGAACAGGGAC |
| | | CTGAAGACCCAGTCCGGCTCCGAGATGAAG |
| | | AAGTTCCTGTCCACCCTGACCATCGACGGC |
| | | GTGACCAGGTCCGACCAGGGCCTGTACACC |
| | | TGCGCCGCCTCCTCCGGCCTGATGACCAAG |
| | | AAGAACTCCACCTTCGTGAGGGTGCACGAG |
| | | AAGGACAAGACCCACACCTGCCCCCCCTGC |
| | | CCCGCCCCCGAGCTGCTGGGCGGCCCCTCC |
| | | GTGTTCCTGTTCCCCCCCAAGCCCAAGGAC |
| | | ACCCTGATGATCTCCAGGACCCCCGAGGTG |
| | | ACCTGCGTGGTGGTGGACGTGTCCCACGAG |
| | | GACCCCGAGGTGAAGTTCAACTGGTACGTG |
| | | GACGGCGTGGAGGTGCACAACGCCAAGACC |
| | | AAGCCCAGGGAGGAGCAGTACAACTCCACC |
| | | TACAGGGTGGTGTCCGTGCTGACCGTGCTG |
| | | CACCAGGACTGGCTGAACGGCAAGGAGTAC |
| | | AAGTGCAAGGTGTCCAACAAGGCCCTGCCC |
| | | GCCCCCATCGAGAAGACCATCTCCAAGGCC |
| | | AAGGGCCAGCCCAGGGAGCCCCAGGTGTAC |
| | | ACCCTGCCCCCCTCCAGGGACGAGCTGACC |
| | | AAGAACCAGGTGTCCCTGACCTGCCTGGTG |
| | | AAGGGCTTCTACCCCTCCGACATCGCCGTG |
| | | GAGTGGGAGTCCAACGGCCAGCCCGAGAAC |
| | | AACTACAAGACCACCCCCCCCGTGCTGGAC |
| | | TCCGACGGCTCCTTCTTCCTGTACTCCAAG |
| | | CTGACCGTGGACAAGTCCAGGTGGCAGCAG |
| | | GGCAACGTGTTCTCCTGCTCCGTGATGCAC |
| | | GAGGCCCTGCACAACCACTACACCCAGAAG |
| | | TCCCTGTCCCTGTCCCCCGGCAAGTGA |
| 27 | AMI130 | ATGGTGAGCTACTGGGACACCGGCGTGCTG |
| | coding | CTGTGCGCCCTGCTGAGCTGCCTGCTGCTG |
| | sequence | ACCGGCAGCAGCAGCGGCAGCGACACCGGC |
| | | AGGCCGTTCGTGGAGATGTACAGCGAGATC |
| | | CCGGAGATCATCCACATGACCGAGGGCAGG |
| | | GAGCTGGTGATCCCGTGCAGGGTGACCAGC |
| | | CCGAACATCACCGTGACCCTGAAGAAGTTC |
| | | CCGCTGGACACCCTGATCCCGGACGGCAAG |
| | | AGGATCATCTGGGACAGCAGGAAGGGCTTC |
| | | ATCATCAGCAACGCCACCTACAAGGAGATC |
| | | GGCCTGCTGACCTGCGAGGCCACCGTGAAC |
| | | GGCCACCTGTACAAGACCAACTACCTGACC |
| | | CACAGGCAGACCAACACCATCATCGACGTG |
| | | GTGCTGAGCCCGAGCCACGGCATCGAGCTG |
| | | AGCGTGGGCGAGAAGCTGGTGCTGAACTGC |
| | | ACCGCCAGGACCGAGCTGAACGTGGGCATC |
| | | GACTTCAACTGGGAGTACCCGAGCAGCAAG |
| | | CACCAGCACAAGAAGCTGGTGAACAGGGAC |
| | | CTGAAGACCCAGAGCGGCAGCGAGATGAAG |
| | | AAGTTCCTGAGCACCCTGACCATCGACGGC |
| | | GTGACCAGGAGCGACCAGGGCCTGTACACC |
| | | TGCGCCGCCAGCAGCGGCCTGATGACCAAG |
| | | AAGAACAGCACCTTCGTGAGGGTGCACGAG |
| | | AAGGACAAGACCCACACCTGCCCGCCGTGC |
| | | CCGGCCCCGGAGCTGCTGGGCGGCCCGAGC |
| | | GTGTTCCTGTTCCCGCCGAAGCCGAAGGAC |
| | | ACCCTGATGATCAGCAGGACCCCGGAGGTG |
| | | ACCTGCGTGGTGGTGGACGTGAGCCACGAG |
| | | GACCCGGAGGTGAAGTTCAACTGGTACGTG |
| | | GACGGCGTGGAGGTGCACAACGCCAAGACC |
| | | AAGCCGAGGGAGGAGCAGTACAACAGCACC |
| | | TACAGGGTGGTGAGCGTGCTGACCGTGCTG |
| | | CACCAGGACTGGCTGAACGGCAAGGAGTAC |
| | | AAGTGCAAGGTGAGCAACAAGGCCCTGCCG |
| | | GCCCCGATCGAGAAGACCATCAGCAAGGCC |
| | | AAGGGCCAGCCGAGGGAGCCGCAGGTGTAC |
| | | ACCCTGCCGCCGAGCAGGGACGAGCTGACC |
| | | AAGAACCAGGTGAGCCTGACCTGCCTGGTG |
| | | AAGGGCTTCTACCCGAGCGACATCGCCGTG |
| | | GAGTGGGAGAGCAACGGCCAGCCGGAGAAC |
| | | AACTACAAGACCACCCCGCCGGTGCTGGAC |
| | | AGCGACGGCAGCTTCTTCCTGTACAGCAAG |
| | | CTGACCGTGGACAAGAGCAGGTGGCAGCAG |
| | | GGCAACGTGTTCAGCTGCAGCGTGATGCAC |
| | | GAGGCCCTGCACAACCACTACACCCAGAAG |
| | | AGCCTGAGCCTGAGCCCCGGCAAGTGA |
| 28 | Control | ATGGTCAGCTACTGGGACACCGGGGTCCTG |
| | coding | CTGTGCGCGCTGCTCAGCTGTCTGCTTCTC |
| | sequence | ACAGGATCTAGTTCAGGTTCAGATACAGGT |
| | (reference | AGACCTTTCGTAGAGATGTACAGTGAAATC |
| | VEGF-Trap) | CCCGAAATTATACACATGACTGAAGGAAGG |
| | | GAGCTCGTCATTCCCTGCCGGGTTACGTCA |
| | | CCTAACATCACTGTTACTTTAAAAAAGTTT |
| | | CCACTTGACACTTTGATCCCTGATGGAAAA |
| | | CGCATAATCTGGGACAGTAGAAAGGGCTTC |
| | | ATCATATCAAATGCAACGTACAAAGAAATA |
| | | GGGCTTCTGACCTGTGAAGCAACAGTCAAT |
| | | GGGCATTTGTATAAGACAAACTATCTCACA |
| | | CATCGACAAACCAATACAATCATAGATGTC |
| | | GTTCTGAGTCCGTCTCATGGAATTGAACTA |
| | | TCTGTTGGAGAAAAGCTTGTCTTAAATTGT |
| | | ACAGCAAGAACTGAACTAAATGTGGGGATT |
| | | GACTTCAACTGGGAATACCCTTCTTCGAAG |
| | | CATCAGCATAAGAAACTTGTAAACCGAGAC |
| | | CTAAAAACCCAGTCTGGGAGTGAGATGAAG |
| | | AAATTTTTGAGCACCTTAACTATAGATGGT |
| | | GTAACCCGGAGTGACCAAGGATTGTACACC |
| | | TGTGCAGCATCCAGTGGGCTGATGACCAAG |
| | | AAGAACAGCACATTTGTCAGGGTCCATGAA |
| | | AAAGACAAAACTCACACATGCCCACCGTGC |
| | | CCAGCACCTGAACTCCTGGGGGGACCGTCA |
| | | GTCTTCCTCTTCCCCCCAAAACCCAAGGAC |
| | | ACCCTCATGATCTCCCGGACCCCTGAGGTC |
| | | ACATGCGTGGTGGTGGACGTGAGCCACGAA |
| | | GACCCTGAGGTCAAGTTCAACTGGTACGTG |
| | | GACGGCGTGGAGGTGCATAATGCCAAGACA |
| | | AAGCCGCGGGAGGAGCAGTACAACAGCACG |
| | | TACCGTGTGGTCAGCGTCCTCACCGTCCTG |
| | | CACCAGGACTGGCTGAATGGCAAGGAGTAC |
| | | AAGTGCAAGGTCTCCAACAAAGCCCTCCCA |
| | | GCCCCCATCGAGAAAACCATCTCCAAAGCC |
| | | AAAGGGCAGCCCCGAGAACCACAGGTGTAC |
| | | ACCCTGCCCCCATCCCGGGATGAGCTGACC |
| | | AAGAACCAGGTCAGCCTGACCTGCCTGGTC |
| | | AAAGGCTTCTATCCCAGCGACATCGCCGTG |
| | | GAGTGGGAGAGCAATGGGCAGCCGGAGAAC |
| | | AACTACAAGACCACGCCTCCCGTGCTGGAC |
| | | TCCGACGGCTCCTTCTTCCTCTACAGCAAG |
| | | CTCACCGTGGACAAGAGCAGGTGGCAGCAG |
| | | GGGAACGTCTTCTCATGCTCCGTGATGCAT |
| | | GAGGCTCTGCACAACCACTACACGCAGAAG |
| | | AGCCTCTCCCTGTCTCCGGGTAAATGA |
| 30 | Amino acid | DTGRPFVEMYSEIPEIIHMTEGRELVIPCR |
| | sequence of | VTSPNITVTLKKFPLDTLIPDGKRIIWDSR |
| | aflibercept | KGFIISNATYKEIGLLTCEATVNGHLYKTN |
| | (encoded by | YLTHRQTNTIIDVVLSPSHGIELSVGEKLV |
| | AMI120) | LNCTARTELNVGIDENWEYPSSKHQHKKLV |
| | | NRDLKTQSGSEMKKFLSTLTIDGVTRSDQG |
| | | LYTCAASSGLMTKKNSTFVRVHEKDKTHTC |
| | | PPCPAPELLGGPSVFLFPPKPKDTLMISRT |
| | | PEVTCVVVDVSHEDPEVKFNWYVDGVEVHN |
| | | AKTKPREEQYNSTYRVVSVLTVLHQDWLNG |
| | | KEYKCKVSNKALPAPIEKTISKAKGQPREP |
| | | QVYTLPPSRDELTKNQVSLTCLVKGFYPSD |
| | | IAVEWESNGQPENNYKTTPPVLDSDGSFFL |
| | | YSKLTVDKSRWQQGNVFSCSVMHEALHNHY |
| | | TQKSLSLSPGK* |
| 31 | Nucleic acid | GACACCGGCAGGCCCTTCGTGGAGATGTAC |
| | sequence | TCCGAGATCCCCGAGATCATCCACATGACC |
| | encoding | GAGGGCAGGGAGCTGGTGATCCCCTGCAGG |
| | aflibercept | GTGACCTCCCCCAACATCACCGTGACCCTG |
| | (AMI120) | AAGAAGTTCCCCCTGGACACCCTGATCCCC |
| | | GACGGCAAGAGGATCATCTGGGACTCCAGG |
| | | AAGGGCTTCATCATCTCCAACGCCACCTAC |
| | | AAGGAGATCGGCCTGCTGACCTGCGAGGCC |
| | | ACCGTGAACGGCCACCTGTACAAGACCAAC |
| | | TACCTGACCCACAGGCAGACCAACACCATC |
| | | ATCGACGTGGTGCTGTCCCCCTCCCACGGC |
| | | ATCGAGCTGTCCGTGGGCGAGAAGCTGGTG |
| | | CTGAACTGCACCGCCAGGACCGAGCTGAAC |
| | | GTGGGCATCGACTTCAACTGGGAGTACCCC |
| | | TCCTCCAAGCACCAGCACAAGAAGCTGGTG |
| | | AACAGGGACCTGAAGACCCAGTCCGGCTCC |
| | | GAGATGAAGAAGTTCCTGTCCACCCTGACC |
| | | ATCGACGGCGTGACCAGGTCCGACCAGGGC |
| | | CTGTACACCTGCGCCGCCTCCTCCGGCCTG |
| | | ATGACCAAGAAGAACTCCACCTTCGTGAGG |
| | | GTGCACGAGAAGGACAAGACCCACACCTGC |
| | | CCCCCCTGCCCCGCCCCCGAGCTGCTGGGC |
| | | GGCCCCTCCGTGTTCCTGTTCCCCCCCAAG |
| | | CCCAAGGACACCCTGATGATCTCCAGGACC |
| | | CCCGAGGTGACCTGCGTGGTGGTGGACGTG |
| | | TCCCACGAGGACCCCGAGGTGAAGTTCAAC |
| | | TGGTACGTGGACGGCGTGGAGGTGCACAAC |
| | | GCCAAGACCAAGCCCAGGGAGGAGCAGTAC |
| | | AACTCCACCTACAGGGTGGTGTCCGTGCTG |
| | | ACCGTGCTGCACCAGGACTGGCTGAACGGC |
| | | AAGGAGTACAAGTGCAAGGTGTCCAACAAG |
| | | GCCCTGCCCGCCCCCATCGAGAAGACCATC |
| | | TCCAAGGCCAAGGGCCAGCCCAGGGAGCCC |
| | | CAGGTGTACACCCTGCCCCCCTCCAGGGAC |
| | | GAGCTGACCAAGAACCAGGTGTCCCTGACC |
| | | TGCCTGGTGAAGGGCTTCTACCCCTCCGAC |
| | | ATCGCCGTGGAGTGGGAGTCCAACGGCCAG |
| | | CCCGAGAACAACTACAAGACCACCCCCCCC |
| | | GTGCTGGACTCCGACGGCTCCTTCTTCCTG |
| | | TACTCCAAGCTGACCGTGGACAAGTCCAGG |
| | | TGGCAGCAGGGCAACGTGTTCTCCTGCTCC |
| | | GTGATGCACGAGGCCCTGCACAACCACTAC |
| | | ACCCAGAAGTCCCTGTCCCTGTCCCCCGGC |
| | | AAGTGA |
| 70 | Reference | atgtacagaa tgcagctgct |
| | nucleic acid | gctgctgatc gccctgagcc |
| | sequence | tggccctggt gaccaacagc |
| | encoding | agcgacaccg gcagaccctt |
| | VEGF-Trap | cgtggagatg tacagcgaga |
| | | tccccgagat catccacatg |
| | | accgagggca gagagctggt |
| | | gatcccctgc agagtgacca |
| | | gccccaacat caccgtgacc |
| | | ctgaagaagt tccccctgga |
| | | caccctgate cccgacggca |
| | | agagaatcat ctgggacagc |
| | | agaaagggct tcatcatcag |
| | | caacgccacc tacaaggaga |
| | | toggcctgct gacctgcgag |
| | | gccaccgtga acggccacct |
| | | gtacaagacc aactacctga |
| | | cccacagaca gaccaacacc |
| | | atcatcgacg tggtgctgag |
| | | ccccagccac ggcatcgagc |
| | | tgagcgtggg cgagaagctg |
| | | gtgctgaact gcaccgccag |
| | | aaccgagctg aacgtgggca |
| | | tegacttcaa ctgggagtac |
| | | cccagcagca agcaccagca |
| | | caagaagctg gtgaacagag |
| | | acctgaagac ccagagcggc |
| | | agcgagatga agaagttcct |
| | | gagcaccctg accatcgacg |
| | | gcgtgaccag aagcgaccag |
| | | ggcctgtaca cctgcgccgc |
| | | cagcageggc ctgatgacca |
| | | agaagaacag caccttcgtg |
| | | agagtgcacg agaaggacaa |
| | | gacccacacc tgccccccct |
| | | goccegcccc cgagctgctg |
| | | ggcggcccca gegtgttect |
| | | gttccccccc aagcccaagg |
| | | acaccctgat gatcagcaga |
| | | acccccgagg tgacctgcgt |
| | | ggtggtggac gtgagccacg |
| | | aggaccccga ggtgaagttc |
| | | aactggtacg tggacggcgt |
| | | ggaggtgcac aacgccaaga |
| | | ccaagcccag agaggagcag |
| | | tacaacagca cctacagagt |
| | | ggtgagegtg ctgaccgtgc |
| | | tgcaccagga ctggctgaac |
| | | ggcaaggagt acaagtgcaa |
| | | ggtgagcaac aaggccctgc |
| | | cegcccccat cgagaagacc |
| | | atcagcaagg ccaagggcca |
| | | gcccagagag ccccaggtgt |
| | | acaccctgcc ccccagcaga |
| | | gacgagctga ccaagaacca |
| | | ggtgagectg acctgcctgg |
| | | tgaagggctt ctaccccagc |
| | | gacatcgccg tggagtggga |
| | | gagcaacgge cagcccgaga |
| | | acaactacaa gaccaccccc |
| | | cccgtgctgg acagcgacgg |
| | | cagcttcttc ctgtacagca |
| | | agctgaccgt ggacaagagc |
| | | agatggcagc agggcaacgt |
| | | gttcagctgc agcgtgatgc |
| | | acgaggccct gcacaaccac |
| | | tacacccaga agagcctgag |
| | | cctgagcccc ggc |

1. A non-naturally occurring nucleic acid comprising a sequence encoding a biologic comprising an anti-angiogenic agent, said sequence comprising a modification in a coding region of the sequence as compared to an otherwise comparable sequence lacking the modification in the coding region, said modification comprising a replacement of at least four non-AGG arginine codons with AGG.

2. The non-naturally occurring nucleic acid of claim 1, wherein the sequence that encodes the anti-angiogenic agent further comprises a second modification.

3. The non-naturally occurring nucleic acid of claim 2, wherein the second modification is in at least one codon of the coding region of the sequence, and wherein the second modification is selected from the group consisting of:
(a) replacement of at least one non-CCC proline codon with CCC;
(b) replacement of at least one non-TCC serine codon with TCC;
(c) replacement of at least one non-CCG proline codon with CCG; and
(d) any combination of (a)-(c).

4.-10. (canceled)

11. The non-naturally occurring nucleic acid of claim 3, wherein the at least one non-CCC proline codon of (a) is CCT; the at least one non-TCC serine codon of (b) is AGC; the at least one non-CCG proline codon of (c) is CCC, or any combination thereof.

12. The non-naturally occurring nucleic acid of claim 1, wherein the anti-angiogenic agent is selected from the group consisting of: a VEGF inhibitor, a multi-tyrosine kinase inhibitor, a receptor tyrosine kinase inhibitor, an inhibitor of Akt phosphorylation, a PDGF-1 inhibitor, a PDGF-2 inhibitor, a NP-1 inhibitor, a NP-2 inhibitor, a Del 1 inhibitor, and an integrin inhibitor.

13. The non-naturally occurring nucleic acid of claim 12, wherein the anti-angiogenic agent comprises the VEGF inhibitor, and wherein the VEGF inhibitor is a non-antibody inhibitor.

14. The non-naturally occurring nucleic acid of claim 13, wherein the non-antibody inhibitor is a fusion protein that comprises human VEGF receptors 1 and 2.

15. The non-naturally occurring nucleic acid of claim 14, wherein the fusion protein comprises VEGF-Trap or a modified version thereof.

16. The non-naturally occurring nucleic acid of claim 1, wherein the non-naturally occurring nucleic acid further comprises a signal peptide.

17. The non-naturally occurring nucleic acid of claim 16, wherein the signal peptide is selected from the group consisting of: human antibody heavy chain (Vh), human antibody light chain (Vl), and VEGF-Trap.

18.-19. (canceled)

20. The non-naturally occurring nucleic acid of claim 1, wherein the non-naturally occurring nucleic acid further comprises an intronic sequence.

21. The non-naturally occurring nucleic acid of claim 20, wherein the intronic sequence is selected from the group consisting of: hCMV intron A, adenovirus tripartite leader sequence intron, SV40 intron, hamster EF-1 alpha gene intron 1, intervening sequence intron, human growth hormone intron, and human beta globin intron.

22. (canceled)

23. The non-naturally occurring nucleic acid of claim 1, wherein the non-naturally occurring nucleic acid further comprises a promoter.

24. The non-naturally occurring nucleic acid of claim 23, wherein the promoter is selected from the group consisting of: a cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, a Ca2+/calmodulin-dependent protein kinase II (CaMKIIa) promoter, a GAL1 promoter, a GAL 10 promoter, a TEF1 promoter, a glyceraldehyde 3-phosphage dehydrogenase (GDS) promoter, an ADH1 promoter, a CaMV35S promoter, a Ubi promoter, a human polymerase III RNA (H1) promoter, a U6 promoter, a polyadenylated construct thereof, and any combination thereof.

25.-30. (canceled)

31. The non-naturally occurring nucleic acid of claim 1, wherein the non-AGG arginine codon is AGA.

32.-65. (canceled)

66. The non-naturally occurring nucleic acid of claim 1, wherein the nucleic acid comprises a viral vector sequence.

67. (canceled)

68. The non-naturally occurring nucleic acid of claim 66, wherein the viral vector sequence comprises an AAV vector sequence, and wherein the AAV vector sequence is of serotype AAV1, AAV2, AAV3, AAV4, AAV5, AAV6, AAV7, AAV8, AAV9, AAV10, AAV11, AAV12, or any combination thereof.

69.-71. (canceled)

72. The non-naturally occurring nucleic acid of claim 1, wherein the non-naturally occurring nucleic acid comprises at least about 60% sequence identity or similarity with any one of SEQ ID NOS: 13-19, 21-27, 31, 62, 64, 66, or 68.

73.-92. (canceled)

93. An adeno-associated viral (AAV) particle comprising the non-naturally occurring nucleic acid of claim 1.

94.-101. (canceled)

102. A method of treating a disease or condition in a subject in need thereof, the method comprising administering an effective amount of the non-naturally occurring nucleic acid of claim 1 to the subject in need thereof, thereby treating the disease or condition.

103.-150. (canceled)


SEQUENCE LISTING (70 sequences)

SEQ ID NO: 1 - DNA, 40 bp, synthetic construct
  misc_feature 1..40: note = Description of Artificial Sequence: Synthetic primer
  source 1..40: mol_type = other DNA
atccagcctccggactctagagttaactggtaagtttagt

SEQ ID NO: 2 - DNA, 40 bp, synthetic construct
  misc_feature 1..40: note = Description of Artificial Sequence: Synthetic primer
  source 1..40: mol_type = other DNA
tggggttgatctctccccagcatgccacacaaaaaaccaa

SEQ ID NO: 3 - DNA, 49 bp, synthetic construct
  misc_feature 1..49: note = Description of Artificial Sequence: Synthetic primer
  source 1..49: mol_type = other DNA
tacatttctacaaaaggtctaccagtatcgcactgcacgcccttaagga

SEQ ID NO: 4 - DNA, 22 bp, synthetic construct
  misc_feature 1..22: note = Description of Artificial Sequence: Synthetic primer
  source 1..22: mol_type = other DNA
gatactggtagaccttttgtag

SEQ ID NO: 5 - DNA, 48 bp, synthetic construct
  misc_feature 1..48: note = Description of Artificial Sequence: Synthetic primer
  source 1..48: mol_type = other DNA
gttgcctttacttctaggcctgccgccaccatggtgagctactgggac

SEQ ID NO: 6 - DNA, 49 bp, synthetic construct
  misc_feature 1..49: note = Description of Artificial Sequence: Synthetic primer
  source 1..49: mol_type = other DNA
taatgaaaataaagatattttattttcgaatcacttgccggggctcagg

SEQ ID NO: 7 - DNA, 22 bp, synthetic construct
  misc_feature 1..22: note = Description of Artificial Sequence: Synthetic primer
  source 1..22: mol_type = other DNA
gacaccggcagacccttcgtgg

SEQ ID NO: 8 - DNA, 40 bp, synthetic construct
  misc_feature 1..40: note = Description of Artificial Sequence: Synthetic primer
  source 1..40: mol_type = other DNA
acgaagggtctgccggtgtcgcactgcacgcccttaagga

SEQ ID NO: 9 - DNA, 49 bp, synthetic construct; identical to SEQ ID NO: 6
  misc_feature 1..49: note = Description of Artificial Sequence: Synthetic primer
  source 1..49: mol_type = other DNA

SEQ ID NO: 10 - DNA, 49 bp, synthetic construct
  misc_feature 1..49: note = Description of Artificial Sequence: Synthetic primer
  source 1..49: mol_type = other DNA
taatgaaaataaagatattttattttcgaatcacttgccagggctcagg

SEQ ID NO: 11 - DNA, 49 bp, synthetic construct
  misc_feature 1..49: note = Description of Artificial Sequence: Synthetic primer
  source 1..49: mol_type = other DNA
taatgaaaataaagatattttattttcgaatcacttgccgggggacagg

SEQ ID NO: 12 - protein, 458 aa, synthetic construct
  REGION 1..458: note = Description of Artificial Sequence: Synthetic polypeptide
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 6971 bp, synthetic construct
  misc_feature 1..6971: note = Description of Artificial Sequence: Synthetic polynucleotide
  source 1..6971: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtatcctattgggatacgggtgttctcttgtgtgcactcctttcctgtctcctgctcactggatcttcttctgggtctgatactggtagaccttttgtagaaatgtattcagaaattccggaaataattcatatgacagaaggacgagaactcgttataccatgtcgcgtcacgtcccctaatattactgttacgctcaagaagtttcctctcgatacacttattccagatgggaaacgcataatttgggattcacgcaaagggtttattattagtaacgcaacgtataaagaaattgggctgctcacatgtgaagctacggtaaatgggcatctttataaaacaaattatttgactcatcggcaaactaatactattatcgatgtagtactctccccatcccatggtattgaattgtcagttggggagaagttggtattgaattgtactgcacggacagaactcaacgttggtattgattttaattgggaatatccatcatctaaacatcagcataagaagttggtaaatcgtgatctcaaaactcaaagtgggtccgaaatgaagaagtttctgtccacacttacgattgatggggtcactagaagtgatcaagggctctatacgtgtgcagcatctagtgggttgatgacaaagaagaattcaacttttgttcgtgtccatgaaaaggataaaacacatacttgtccaccgtgtcctgcgccagaacttctcggtggtccatccgtctttctctttccacctaaaccaaaagatactttgatgatttcacggactccagaagtaacatgtgttgtcgttgatgtatcacacgaagatccagaagtcaaatttaattggtatgttgatggtgtagaagttcataatgcgaagacaaaaccacgagaagaacaatacaatagtacatatcgggtagtatccgtcttgactgtacttcaccaagattggcttaatgggaaagaatacaaatgtaaagtttctaataaagctcttcctgcgccgatcgaaaagacaatttccaaagcaaaaggtcaacctcgggaacctcaagtttatacgctcccaccatcacgggatgaactcactaagaatcaagtatccttgacttgtctcgtaaaagggttttatccttcagatattgctgtagaatgggaatccaatgggcaaccagaaaataattataaaacaacaccacctgttcttgattcagatggttcattctttctctattccaaacttactgtcgataaatcacgctggcaacaaggtaatgttttctcttgttccgtcatgcatgaagcactccataatcactatacgcaaaagtctctctctctctcaccaggtaaataataattcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 14 - DNA, 6947 bp, synthetic construct
  misc_feature 1..6947: note = Description of Artificial Sequence: Synthetic polynucleotide
  source 1..6947: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgcgatactggtagaccttttgtagaaatgtattcagaaattccggaaataattcatatgacagaaggacgagaactcgttataccatgtcgcgtcacgtcccctaatattactgttacgctcaagaagtttcctctcgatacacttattccagatgggaaacgcataatttgggattcacgcaaagggtttattattagtaacgcaacgtataaagaaattgggctgctcacatgtgaagctacggtaaatgggcatctttataaaacaaattatttgactcatcggcaaactaatactattatcgatgtagtactctccccatcccatggtattgaattgtcagttggggagaagttggtattgaattgtactgcacggacagaactcaacgttggtattgattttaattgggaatatccatcatctaaacatcagcataagaagttggtaaatcgtgatctcaaaactcaaagtgggtccgaaatgaagaagtttctgtccacacttacgattgatggggtcactagaagtgatcaagggctctatacgtgtgcagcatctagtgggttgatgacaaagaagaattcaacttttgttcgtgtccatgaaaaggataaaacacatacttgtccaccgtgtcctgcgccagaacttctcggtggtccatccgtctttctctttccacctaaaccaaaagatactttgatgatttcacggactccagaagtaacatgtgttgtcgttgatgtatcacacgaagatccagaagtcaaatttaattggtatgttgatggtgtagaagttcataatgcgaagacaaaaccacgagaagaacaatacaatagtacatatcgggtagtatccgtcttgactgtacttcaccaagattggcttaatgggaaagaatacaaatgtaaagtttctaataaagctcttcctgcgccgatcgaaaagacaatttccaaagcaaaaggtcaacctcgggaacctcaagtttatacgctcccaccatcacgggatgaactcactaagaatcaagtatccttgacttgtctcgtaaaagggttttatccttcagatattgctgtagaatgggaatccaatgggcaaccagaaaataattataaaacaacaccacctgttcttgattcagatggttcattctttctctattccaaacttactgtcgataaatcacgctggcaacaaggtaatgttttctcttgttccgtcatgcatgaagcactccataatcactatacgcaaaagtctctctctctctcaccaggtaaataataattcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 15 - DNA, 6968 bp, synthetic construct
  misc_feature 1..6968: note = Description of Artificial Sequence: Synthetic polynucleotide
  source 1..6968: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcagacccttcgtggagatgtacagcgagatccccgagatcatccacatgaccgagggcagagagctggtgatcccctgcagagtgaccagccccaacatcaccgtgaccctgaagaagttccccctggacaccctgatccccgacggcaagagaatcatctgggacagcagaaagggcttcatcatcagcaacgccacctacaaggagatcggcctgctgacctgcgaggccaccgtgaacggccacctgtacaagaccaactacctgacccacagacagaccaacaccatcatcgacgtggtgctgagccccagccacggcatcgagctgagcgtgggcgagaagctggtgctgaactgcaccgccagaaccgagctgaacgtgggcatcgacttcaactgggagtaccccagcagcaagcaccagcacaagaagctggtgaacagagacctgaagacccagagcggcagcgagatgaagaagttcctgagcaccctgaccatcgacggcgtgaccagaagcgaccagggcctgtacacctgcgccgccagcagcggcctgatgaccaagaagaacagcaccttcgtgagagtgcacgagaaggacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaagtgattcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 16 - DNA, 6944 bp, synthetic construct
  misc_feature 1..6944: note = Description of Artificial Sequence: Synthetic polynucleotide
  source 1..6944: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgcgacaccggcagacccttcgtggagatgtacagcgagatccccgagatcatccacatgaccgagggcagagagctggtgatcccctgcagagtgaccagccccaacatcaccgtgaccctgaagaagttccccctggacaccctgatccccgacggcaagagaatcatctgggacagcagaaagggcttcatcatcagcaacgccacctacaaggagatcggcctgctgacctgcgaggccaccgtgaacggccacctgtacaagaccaactacctgacccacagacagaccaacaccatcatcgacgtggtgctgagccccagccacggcatcgagctgagcgtgggcgagaagctggtgctgaactgcaccgccagaaccgagctgaacgtgggcatcgacttcaactgggagtaccccagcagcaagcaccagcacaagaagctggtgaacagagacctgaagacccagagcggcagcgagatgaagaagttcctgagcaccctgaccatcgacggcgtgaccagaagcgaccagggcctgtacacctgcgccgccagcagcggcctgatgaccaagaagaacagcaccttcgtgagagtgcacgagaaggacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaagtgattcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 17 - DNA, 6968 bp, synthetic construct
  misc_feature 1..6968: note = Description of Artificial Sequence: Synthetic polynucleotide
  source 1..6968: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcctttcgtggagatgtacagcgagatccctgagatcatccacatgaccgagggcagggagctggtgatcccttgcagggtgaccagccctaacatcaccgtgaccctgaagaagttccctctggacaccctgatccctgacggcaagaggatcatctgggacagcaggaagggcttcatcatcagcaacgccacctacaaggagatcggcctgctgacctgcgaggccaccgtgaacggccacctgtacaagaccaactacctgacccacaggcagaccaacaccatcatcgacgtggtgctgagccctagccacggcatcgagctgagcgtgggcgagaagctggtgctgaactgcaccgccaggaccgagctgaacgtgggcatcgacttcaactgggagtaccctagcagcaagcaccagcacaagaagctggtgaacagggacctgaagacccagagcggcagcgagatgaagaagttcctgagcaccctgaccatcgacggcgtgaccaggagcgaccagggcctgtacacctgcgccgccagcagcggcctgatgaccaagaagaacagcaccttcgtgagggtgcacgagaaggacaagacccacacctgccctccttgccctgcccctgagctgctgggcggccctagcgtgttcctgttccctcctaagcctaaggacaccctgatgatcagcaggacccctgaggtgacctgcgtggtggtggacgtgagccacgaggaccctgaggtgaagttcaactggtacgtggacggcgtggaggtgcacaacgccaagaccaagcctagggaggagcagtacaacagcacctacagggtggtgagcgtgctgaccgtgctgcaccaggactggctgaacggcaaggagtacaagtgcaaggtgagcaacaaggccctgcctgcccctatcgagaagaccatcagcaaggccaagggccagcctagggagcctcaggtgtacaccctgcctcctagcagggacgagctgaccaagaaccaggtgagcctgacctgcctggtgaagggcttctaccctagcgacatcgccgtggagtgggagagcaacggccagcctgagaacaactacaagaccacccctcctgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccctggcaagtgattcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 18 - DNA, 6968 bp, synthetic construct
  misc_feature 1..6968: note = Description of Artificial Sequence: Synthetic polynucleotide
  source 1..6968: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgattcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 19 - DNA, 6968 bp, synthetic construct
  misc_feature 1..6968: note = Description of Artificial Sequence: Synthetic polynucleotide
  source 1..6968: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggccgttcgtggagatgtacagcgagatcccggagatcatccacatgaccgagggcagggagctggtgatcccgtgcagggtgaccagcccgaacatcaccgtgaccctgaagaagttcccgctggacaccctgatcccggacggcaagaggatcatctgggacagcaggaagggcttcatcatcagcaacgccacctacaaggagatcggcctgctgacctgcgaggccaccgtgaacggccacctgtacaagaccaactacctgacccacaggcagaccaacaccatcatcgacgtggtgctgagcccgagccacggcatcgagctgagcgtgggcgagaagctggtgctgaactgcaccgccaggaccgagctgaacgtgggcatcgacttcaactgggagtacccgagcagcaagcaccagcacaagaagctggtgaacagggacctgaagacccagagcggcagcgagatgaagaagttcctgagcaccctgaccatcgacggcgtgaccaggagcgaccagggcctgtacacctgcgccgccagcagcggcctgatgaccaagaagaacagcaccttcgtgagggtgcacgagaaggacaagacccacacctgcccgccgtgcccggccccggagctgctgggcggcccgagcgtgttcctgttcccgccgaagccgaaggacaccctgatgatcagcaggaccccggaggtgacctgcgtggtggtggacgtgagccacgaggacccggaggtgaagttcaactggtacgtggacggcgtggaggtgcacaacgccaagaccaagccgagggaggagcagtacaacagcacctacagggtggtgagcgtgctgaccgtgctgcaccaggactggctgaacggcaaggagtacaagtgcaaggtgagcaacaaggccctgccggccccgatcgagaagaccatcagcaaggccaagggccagccgagggagccgcaggtgtacaccctgccgccgagcagggacgagctgaccaagaaccaggtgagcctgacctgcctggtgaagggcttctacccgagcgacatcgccgtggagtgggagagcaacggccagccggagaacaactacaagaccaccccgccggtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagtgattcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 20 - DNA, 6968 bp, synthetic construct
  misc_feature 1..6968: note = Description of Artificial Sequence: Synthetic polynucleotide
  source 1..6968: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgcgctcaccatggtcagctactgggacaccggggtcctgctgtgcgcgctgctcagctgtctgcttctcacaggatctagttcaggttcagatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtcgttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgattcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 21 - DNA, 1377 bp, synthetic construct
  misc_feature 1..1377: note = Description of Artificial Sequence: Synthetic polynucleotide
  source 1..1377: mol_type = other DNA
atggtatcctattgggatacgggtgttctcttgtgtgcactcctttcctgtctcctgctcactggatcttcttctgggtctgatactggtagaccttttgtagaaatgtattcagaaattccggaaataattcatatgacagaaggacgagaactcgttataccatgtcgcgtcacgtcccctaatattactgttacgctcaagaagtttcctctcgatacacttattccagatgggaaacgcataatttgggattcacgcaaagggtttattattagtaacgcaacgtataaagaaattgggctgctcacatgtgaagctacggtaaatgggcatctttataaaacaaattatttgactcatcggcaaactaatactattatcgatgtagtactctccccatcccatggtattgaattgtcagttggggagaagttggtattgaattgtactgcacggacagaactcaacgttggtattgattttaattgggaatatccatcatctaaacatcagcataagaagttggtaaatcgtgatctcaaaactcaaagtgggtccgaaatgaagaagtttctgtccacacttacgattgatggggtcactagaagtgatcaagggctctatacgtgtgcagcatctagtgggttgatgacaaagaagaattcaacttttgttcgtgtccatgaaaaggataaaacacatacttgtccaccgtgtcctgcgccagaacttctcggtggtccatccgtctttctctttccacctaaaccaaaagatactttgatgatttcacggactccagaagtaacatgtgttgtcgttgatgtatcacacgaagatccagaagtcaaatttaattggtatgttgatggtgtagaagttcataatgcgaagacaaaaccacgagaagaacaatacaatagtacatatcgggtagtatccgtcttgactgtacttcaccaagattggcttaatgggaaagaatacaaatgtaaagtttctaataaagctcttcctgcgccgatcgaaaagacaatttccaaagcaaaaggtcaacctcgggaacctcaagtttatacgctcccaccatcacgggatgaactcactaagaatcaagtatccttgacttgtctcgtaaaagggttttatccttcagatattgctgtagaatgggaatccaatgggcaaccagaaaataattataaaacaacaccacctgttcttgattcagatggttcattctttctctattccaaacttactgtcgataaatcacgctggcaacaaggtaatgttttctcttgttccgtcatgcatgaagcactccataatcactatacgcaaaagtctctctctctctcaccaggtaaataa

SEQ ID NO: 22 - DNA, 1353 bp, synthetic construct
  misc_feature 1..1353: note = Description of Artificial Sequence: Synthetic polynucleotide
  source 1..1353: mol_type = other DNA
atggagttcggcctgagctggctgttcctggtggccatccttaagggcgtgcagtgcgatactggtagaccttttgtagaaatgtattcagaaattccggaaataattcatatgacagaaggacgagaactcgttataccatgtcgcgtcacgtcccctaatattactgttacgctcaagaagtttcctctcgatacacttattccagatgggaaacgcataatttgggattcacgcaaagggtttattattagtaacgcaacgtataaagaaattgggctgctcacatgtgaagctacggtaaatgggcatctttataaaacaaattatttgactcatcggcaaactaatactattatcgatgtagtactctccccatcccatggtattgaattgtcagttggggagaagttggtattgaattgtactgcacggacagaactcaacgttggtattgattttaattgggaatatccatcatctaaacatcagcataagaagttggtaaatcgtgatctcaaaactcaaagtgggtccgaaatgaagaagtttctgtccacacttacgattgatggggtcactagaagtgatcaagggctctatacgtgtgcagcatctagtgggttgatgacaaagaagaattcaacttttgttcgtgtccatgaaaaggataaaacacatacttgtccaccgtgtcctgcgccagaacttctcggtggtccatccgtctttctctttccacctaaaccaaaagatactttgatgatttcacggactccagaagtaacatgtgttgtcgttgatgtatcacacgaagatccagaagtcaaatttaattggtatgttgatggtgtagaagttcataatgcgaagacaaaaccacgagaagaacaatacaatagtacatatcgggtagtatccgtcttgactgtacttcaccaagattggcttaatgggaaagaatacaaatgtaaagtttctaataaagctcttcctgcgccgatcgaaaagacaatttccaaagcaaaaggtcaacctcgggaacctcaagtttatacgctcccaccatcacgggatgaactcactaagaatcaagtatccttgacttgtctcgtaaaagggttttatccttcagatattgctgtagaatgggaatccaatgggcaaccagaaaataattataaaacaacaccacctgttcttgattcagatggttcattctttctctattccaaacttactgtcgataaatcacgctggcaacaaggtaatgttttctcttgttccgtcatgcatgaagcactccataatcactatacgcaaaagtctctctctctctcaccaggtaaataa

SEQ ID NO: 23 - DNA, 1377 bp, synthetic construct
  misc_feature 1..1377: note = Description of Artificial Sequence: Synthetic polynucleotide
  source 1..1377: mol_type = other DNA
atggtgagctactgggacaccggcgtgctgctgtgcgccctgctgagctgcctgctgctgaccggcagcagcagcggcagcgacaccggcagacccttcgtggagatgtacagcgagatccccgagatcatccacatgaccgagggcagagagctggtgatcccctgcagagtgaccagccccaacatcaccgtgaccctgaagaagttccccctggacaccctgatccccgacggcaagagaatcatctgggacagcagaaagggcttcatcatcagcaacgccacctacaaggagatcggcctgctgacctgcgaggccaccgtgaacggccacctgtacaagaccaactacctgacccacagacagaccaacaccatcatcgacgtggtgctgagccccagccacggcatcgagctgagcgtgggcgagaagctggtgctgaactgcaccgccagaaccgagctgaacgtgggcatcgacttcaactgggagtaccccagcagcaagcaccagcacaagaagctggtgaacagagacctgaagacccagagcggcagcgagatgaagaagttcctgagcaccctgaccatcgacggcgtgaccagaagcgaccagggcctgtacacctgcgccgccagcagcggcctgatgaccaagaagaacagcaccttcgtgagagtgcacgagaaggacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaagtga

SEQ ID NO: 24 - DNA, 1353 bp, synthetic construct
  misc_feature 1..1353: note = Description of Artificial Sequence: Synthetic polynucleotide
  source 1..1353: mol_type = other DNA
atggagttcggcctgagctggctgttcctggtggccatccttaagggcgtgcagtgcgacaccggcagacccttcgtggagatgtacagcgagatccccgagatcatccacatgaccgagggcagagagctggtgatcccctgcagagtgaccagccccaacatcaccgtgaccctgaagaagttccccctggacaccctgatccccgacggcaagagaatcatctgggacagcagaaagggcttcatcatcagcaacgccacctacaaggagatcggcctgctgacctgcgaggccaccgtgaacggccacctgtacaagaccaactacctgacccacagacagaccaacaccatcatcgacgtggtgctgagccccagccacggcatcgagctgagcgtgggcgagaagctggtgctgaactgcaccgccagaaccgagctgaacgtgggcatcgacttcaactgggagtaccccagcagcaagcaccagcacaagaagctggtgaacagagacctgaagacccagagcggcagcgagatgaagaagttcctgagcaccctgaccatcgacggcgtgaccagaagcgaccagggcctgtacacctgcgccgccagcagcggcctgatgaccaagaagaacagcaccttcgtgagagtgcacgagaaggacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaagtga

SEQ ID NO: 25 - DNA, 1377 bp, synthetic construct
  misc_feature 1..1377: note = Description of Artificial Sequence: Synthetic polynucleotide
  source 1..1377: mol_type = other DNA
atggtgagctactgggacaccggcgtgctgctgtgcgccctgctgagctgcctgctgctgaccggcagcagcagcggcagcgacaccggcaggcctttcgtggagatgtacagcgagatccctgagatcatccacatgaccgagggcagggagctggtgatcccttgcagggtgaccagccctaacatcaccgtgaccctgaagaagttccctctggacaccctgatccctgacggcaagaggatcatctgggacagcaggaagggcttcatcatcagcaacgccacctacaaggagatcggcctgctgacctgcgaggccaccgtgaacggccacctgtacaagaccaactacctgacccacaggcagaccaacaccatcatcgacgtggtgctgagccctagccacggcatcgagctgagcgtgggcgagaagctggtgctgaactgcaccgccaggaccgagctgaacgtgggcatcgacttcaactgggagtaccctagcagcaagcaccagcacaagaagctggtgaacagggacctgaagacccagagcggcagcgagatgaagaagttcctgagcaccctgaccatcgacggcgtgaccaggagcgaccagggcctgtacacctgcgccgccagcagcggcctgatgaccaagaagaacagcaccttcgtgagggtgcacgagaaggacaagacccacacctgccctccttgccctgcccctgagctgctgggcggccctagcgtgttcctgttccctcctaagcctaaggacaccctgatgatcagcaggacccctgaggtgacctgcgtggtggtggacgtgagccacgaggaccctgaggtgaagttcaactggtacgtggacggcgtggaggtgcacaacgccaagaccaagcctagggaggagcagtacaacagcacctacagggtggtgagcgtgctgaccgtgctgcaccaggactggctgaacggcaaggagtacaagtgcaaggtgagcaacaaggccctgcctgcccctatcgagaagaccatcagcaaggccaagggccagcctagggagcctcaggtgtacaccctgcctcctagcagggacgagctgaccaagaaccaggtgagcctgacctgcctggtgaagggcttctaccctagcgacatcgccgtggagtgggagagcaacggccagcctgagaacaactacaagaccacccctcctgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccctggcaagtga

SEQ ID NO: 26 - DNA, 1377 bp, synthetic construct
  misc_feature 1..1377: note = Description of Artificial Sequence: Synthetic polynucleotide
  source 1..1377: mol_type = other DNA
atggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtga

SEQ ID NO: 27 - DNA, 1377 bp, synthetic construct
  misc_feature 1..1377: note = Description of Artificial Sequence: Synthetic polynucleotide
  source 1..1377: mol_type = other DNA
atggtgagctactgggacaccggcgtgctgctgtgcgccctgctgagctgcctgctgctgaccggcagcagcagcggcagcgacaccggcaggccgttcgtggagatgtacagcgagatcccggagatcatccacatgaccgagggcagggagctggtgatcccgtgcagggtgaccagcccgaacatcaccgtgaccctgaagaagttcccgctggacaccctgatcccggacggcaagaggatcatctgggacagcaggaagggcttcatcatcagcaacgccacctacaaggagatcggcctgctgacctgcgaggccaccgtgaacggccacctgtacaagaccaactacctgacccacaggcagaccaacaccatcatcgacgtggtgctgagcccgagccacggcatcgagctgagcgtgggcgagaagctggtgctgaactgcaccgccaggaccgagctgaacgtgggcatcgacttcaactgggagtacccgagcagcaagcaccagcacaagaagctggtgaacagggacctgaagacccagagcggcagcgagatgaagaagttcctgagcaccctgaccatcgacggcgtgaccaggagcgaccagggcctgtacacctgcgccgccagcagcggcctgatgaccaagaagaacagcaccttcgtgagggtgcacgagaaggacaagacccacacctgcccgccgtgcccggccccggagctgctgggcggcccgagcgtgttcctgttcccgccgaagccgaaggacaccctgatgatcagcaggaccccggaggtgacctgcgtggtggtggacgtgagccacgaggacccggaggtgaagttcaactggtacgtggacggcgtggaggtgcacaacgccaagaccaagccgagggaggagcagtacaacagcacctacagggtggtgagcgtgctgaccgtgctgcaccaggactggctgaacggcaaggagtacaagtgcaaggtgagcaacaaggccctgccggccccgatcgagaagaccatcagcaaggccaagggccagccgagggagccgcaggtgtacaccctgccgccgagcagggacgagctgaccaagaaccaggtgagcctgacctgcctggtgaagggcttctacccgagcgacatcgccgtggagtgggagagcaacggccagccggagaacaactacaagaccaccccgccggtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagtga

SEQ ID NO: 28 - DNA, 1377 bp, synthetic construct
  misc_feature 1..1377: note = Description of Artificial Sequence: Synthetic polynucleotide
  source 1..1377: mol_type = other DNA
atggtcagctactgggacaccggggtcctgctgtgcgcgctgctcagctgtctgcttctcacaggatctagttcaggttcagatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtcgttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 29 - skipped

SEQ ID NO: 30 - protein, 431 aa, synthetic construct
  REGION 1..431: note = Description of Artificial Sequence: Synthetic polypeptide
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1296 bp, synthetic construct
  misc_feature 1..1296: note = Description of Artificial Sequence: Synthetic polynucleotide
  source 1..1296: mol_type = other DNA
  CDS 1..1293
gacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtga

SEQ ID NO: 32-39 - skipped (8 entries)

SEQ ID NO: 40 - protein, 18 aa, synthetic construct
  REGION 1..18: note = Description of Artificial Sequence: Synthetic peptide
TPSGLALGETTRPATTTQ

SEQ ID NO: 41 - protein, 18 aa, synthetic construct
  REGION 1..18: note = Description of Artificial Sequence: Synthetic peptide
QRGNLALGETTKPARQAA

SEQ ID NO: 42 - protein, 16 aa, synthetic construct
  REGION 1..16: note = Description of Artificial Sequence: Synthetic peptide
NTPSALGETTKPGTTT

SEQ ID NO: 43 - protein, 16 aa, synthetic construct
  REGION 1..16: note = Description of Artificial Sequence: Synthetic peptide
NTPSALGETTKPGTTT

SEQ ID NO: 44 - protein, 18 aa, synthetic construct
  REGION 1..18: note = Description of Artificial Sequence: Synthetic peptide
QRGNLKLGQTTKPKRQAA

SEQ ID NO: 45 - protein, 18 aa, synthetic construct
  REGION 1..18: note = Description of Artificial Sequence: Synthetic peptide
QRGNLALGQTTKPKRQAA

SEQ ID NO: 46 - protein, 18 aa, synthetic construct
  REGION 1..18: note = Description of Artificial Sequence: Synthetic peptide
QRGNLKLGQTTKPARQAA

SEQ ID NO: 47 - protein, 18 aa, synthetic construct
  REGION 1..18: note = Description of Artificial Sequence: Synthetic peptide
QRGNLALGQTTKPARQAA

SEQ ID NO: 48 - protein, 18 aa, synthetic construct
  REGION 1..18: note = Description of Artificial Sequence: Synthetic peptide
TPSGLALGQTTKPATTTQ

SEQ ID NO: 49 - protein, 18 aa, synthetic construct
  REGION 1..18: note = Description of Artificial Sequence: Synthetic peptide
QRGNLALGQTTEPARQAA

SEQ ID NO: 50 - protein, 18 aa, synthetic construct
  REGION 1..18: note = Description of Artificial Sequence: Synthetic peptide
RGNRLALGQTTKPAQAAT

SEQ ID NO: 51 - protein, 18 aa, synthetic construct
  REGION 1..18: note = Description of Artificial Sequence: Synthetic peptide
NLQRLALGQTTKPAGNRQ

SEQ ID NO: 52 - protein, 18 aa, synthetic construct
  REGION 1..18: note = Description of Artificial Sequence: Synthetic peptide
QRGNVALGQTTKPARQAA

SEQ ID NO: 53 - protein, 18 aa, synthetic construct
  REGION 1..18: note = Description of Artificial Sequence: Synthetic peptide
LQRGLALGESTARGNRQA

SEQ ID NO: 54 - protein, 18 aa, synthetic construct
  REGION 1..18: note = Description of Artificial Sequence: Synthetic peptide
LQRGLALGETSKRANRQA

SEQ ID NO: 55 - protein, 18 aa, synthetic construct
  REGION 1..18: note = Description of Artificial Sequence: Synthetic peptide
LQRGLALGQSTKPANRQA

SEQ ID NO: 56 - protein, 18 aa, synthetic construct
  REGION 1..18: note = Description of Artificial Sequence: Synthetic peptide
NTPSLALGQTTKPAGTTT

SEQ ID NO: 57 - protein, 18 aa, synthetic construct
  REGION 1..18: note = Description of Artificial Sequence: Synthetic peptide
TPSGLALGQTTKPATTTQ

SEQ ID NO: 58 - protein, 28 aa, synthetic construct
  REGION 1..28: note = Description of Artificial Sequence: Synthetic peptide
QRGNLALGQTTKPALALGQTTKPARQAA

SEQ ID NO: 59 - protein, 18 aa, synthetic construct
  REGION 1..18: note = Description of Artificial Sequence: Synthetic peptide
QRGNVKLGQTTKPARQAA

SEQ ID NO: 60 - skipped

SEQ ID NO: 61 - protein, 449 aa, synthetic construct
  REGION 1..449: note = Description of Artificial Sequence: Synthetic polypeptide
EFGLSWLFLVAILKGVQCDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - DNA, 1353 bp, synthetic construct; identical to SEQ ID NO: 24
  misc_feature 1..1353: note = Description of Artificial Sequence: Synthetic polynucleotide
  source 1..1353: mol_type = other DNA
  CDS 4..1350

SEQ ID NO: 63 - protein, 458 aa, synthetic construct; identical to SEQ ID NO: 12
  REGION 1..458: note = Description of Artificial Sequence: Synthetic polypeptide

SEQ ID NO: 64 - DNA, 1377 bp, synthetic construct; identical to SEQ ID NO: 25
  misc_feature 1..1377: note = Description of Artificial Sequence: Synthetic polynucleotide
  source 1..1377: mol_type = other DNA
  CDS 1..1374

SEQ ID NO: 65 - protein, 458 aa, synthetic construct; identical to SEQ ID NO: 12
  REGION 1..458: note = Description of Artificial Sequence: Synthetic polypeptide

SEQ ID NO: 66 - DNA, 1377 bp, synthetic construct; identical to SEQ ID NO: 26
  misc_feature 1..1377: note = Description of Artificial Sequence: Synthetic polynucleotide
  source 1..1377: mol_type = other DNA
  CDS 1..1374

SEQ ID NO: 67 - protein, 458 aa, synthetic construct; identical to SEQ ID NO: 12
  REGION 1..458: note = Description of Artificial Sequence: Synthetic polypeptide

SEQ ID NO: 68 - DNA, 1377 bp, synthetic construct; identical to SEQ ID NO: 27
  misc_feature 1..1377: note = Description of Artificial Sequence: Synthetic polynucleotide
  source 1..1377: mol_type = other DNA
  CDS 1..1374

SEQ ID NO: 69 - RNA, 27 nt, synthetic construct
  misc_feature 1..27: note = Description of Artificial Sequence: Synthetic oligonucleotide
  source 1..27: mol_type = other RNA
cgcaatcagtgaatgcttatacatccg

SEQ ID NO: 70 - DNA, 1353 bp, synthetic construct
  misc_feature 1..1353: note = Description of Artificial Sequence: Synthetic polynucleotide
  source 1..1353: mol_type = other DNA
atgtacagaatgcagctgctgctgctgatcgccctgagcctggccctggtgaccaacagcagcgacaccggcagacccttcgtggagatgtacagcgagatccccgagatcatccacatgaccgagggcagagagctggtgatcccctgcagagtgaccagccccaacatcaccgtgaccctgaagaagttccccctggacaccctgatccccgacggcaagagaatcatctgggacagcagaaagggcttcatcatcagcaacgccacctacaaggagatcggcctgctgacctgcgaggccaccgtgaacggccacctgtacaagaccaactacctgacccacagacagaccaacaccatcatcgacgtggtgctgagccccagccacggcatcgagctgagcgtgggcgagaagctggtgctgaactgcaccgccagaaccgagctgaacgtgggcatcgacttcaactgggagtaccccagcagcaagcaccagcacaagaagctggtgaacagagacctgaagacccagagcggcagcgagatgaagaagttcctgagcaccctgaccatcgacggcgtgaccagaagcgaccagggcctgtacacctgcgccgccagcagcggcctgatgaccaagaagaacagcaccttcgtgagagtgcacgagaaggacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggc